(19) United States
(12) Patent Application Publication
(10) Pub. No.: US 2016/0320395 A1
(43) Pub. Date: Nov. 3, 2016

(54) MATERIALS AND METHODS FOR DIAGNOSIS AND PROGNOSIS OF LIVER CANCER

(71) Applicants: ELECTROPHORETICS LIMITED, Surrey (GB); KING'S COLLEGE HOSPITAL NHS FOUNDATION TRUST, London (GB)

(72) Inventors: Malcolm Andrew Ward, Surrey (GB); Ian Hugo Pike, Surrey (GB); David James Britton, Surrey (GB); Vikram Mitra, Surrey (GB); Nigel David Heaton, London (GB); Yoh Zen, London (GB); Alberto Quaglia, London (GB)

(73) Assignees: Electrophoretics Limited, Surrey (GB); King's College Hospital NHS Foundation Trust, London (GB)

(21) Appl. No.: 15/036,773

(22) PCT Filed: Nov. 13, 2014
(86) PCT No.: PCT/GB2014/053368

§ 371 Date: May 13, 2016

(30) Foreign Application Priority Data
Nov. 13, 2013 (GB) 1320061.3

(51) Int. Cl. G01N 33/574; G06F 19/18

(52) U.S. Cl. CPC G01N 33/57438; G06F 19/18; G01N 2800/52; G01N 2800/60

(57) ABSTRACT

The invention relates to materials and methods for diagnosing liver tumor types, and assessing patient prognosis. Specifically, but not exclusively, the invention concerns the determination of marker protein which enable primary liver tumors to be identified and classified according to the latest WHO classification. Particularly, the invention provides potential markers proteins which allow non-neoplastic and neoplastic hepatocytes and biliary epithelial cells to be distinguished. This allows grading of tumor differentiation to be refined and differential diagnosis of primary liver tumors and pathogenesis of sub-types of cholangiocarcinoma.

### FIELD OF THE INVENTION

The invention relates to materials and methods for diagnosing tumor types, and assessing patient prognosis. In particular, the invention concerns the determination of marker proteins which enable primary liver tumors to be identified and classified.

### BACKGROUND OF THE INVENTION

The liver is a complex organ capable of regeneration after damage. It is highly structured with a number of specialised cells required to form amongst other features, the bile ducts and liver parenchyma. The most common cell type is the hepatocyte that forms the bulk of the liver parenchyma. Cholangiocytes are a much less common cell type forming the bile ducts of the intrahepatic biliary tree.

Primary liver tumors are classified into epithelial, mesenchymal, germ cell, lymphoid and of mixed or uncertain origin accordingly to the latest WHO classification [1]. Epithelial tumors are the commonest, and generally divided into hepatocellular and cholangiocellular due to their phenotypic similarity to hepatocytes and biliary epithelium, respectively and presumed derivation. Hepatocellular carcinoma (HCC) and cholangiocarcinoma (CC) are the most common malignant types. HCC is the fifth most common cancer worldwide, and usually develops in the context of chronic liver disease [2]. CC can arise from any portion of the intrahepatic biliary tree, and is classified into peripheral and hilar/perihilar based on the predominant location, probable different biological characteristics, and pathogenesis. This classification is supported by an association with risk factors such as viral hepatitis or alcoholic liver disease in peripheral CC. In contrast, multi-step carcinogenesis through intraepithelial neoplasia often in the context of a chronic cholangiopathy (e.g. primary sclerosing cholangitis (PSC)) appears to be behind the development of hilar/perihilar CC [3].

Some primary carcinomas show a mixed phenotype, with areas of hepatocellular differentiation alternating with areas of cholangiocellular differentiation.

An origin from hepatic progenitor cells has been proposed for these tumors, on the broader basis of the cancer stem cell theory that all primary liver tumors and in particular the epithelial ones may be part of a phenotypic spectrum with “pure” HCC and CC at either end, and mixed cancers somewhere in the middle [4, 5]. In this respect, we reported recently that local ablation therapy with transarterial chemoembolization (TACE) is associated with cholangiocellular differentiation in HCC [6]. A potential explanation for this observation is that TACE provides selection pressure in favor of a minor progenitor cell population that is resistant to TACE and capable of multipotent differentiation including biliary lineage. The hepatocellular and cholangiocellular/progenitor cell components were identified by single or double immunostainings or gene expression analysis (RT-PCR) from microdissected tissue, using a relatively limited number of known conventional markers [6]. More markers are required to help better define the details of the phenotype and pathogenesis of the different HCC/CC components of post-TACE tumors, their similarities to their normal and typical malignant counterparts, and aid in diagnosis, prognosis and potentially identify new selective therapeutic targets and predictive markers.

Liquid chromatography-mass spectrometry (LC-MS/MS) based proteomics has proven to be superior over conventional biochemical methods at identifying and quantifying thousands of marker proteins extracted from complex samples including cultured cells and clinical tissue [7-8]. Recently, the application of mass spectrometry based proteomic analysis on formalin fixed paraffin embedded (FFPE) tissue has gained particular focus because of the enormous collections of highly characterized FFPE tissue derived from both human and model organisms [9-10] and its compatibility with Laser Capture Microdissection to enrich tumor cell populations from heterogenous tissue sections.

Large scale global proteomic analysis of laser microdissected FFPE tissue has been successfully employed to discover differentially expressed marker proteins between histological tissue types that can serve as novel protein biomarkers of disease [10-13]. Many of these studies utilized label free quantitative proteomic strategies, such as spectral counting and signal intensities of peptide precursor ions. Both approaches benefit from reduced spectral complexity, enhanced analytical depth and good linear dynamic range (over two orders of magnitude for spectral counting) and consequently provide a high level of quantitative proteome coverage [11-15].

Standard liver histology and immunohistochemistry for tumor marker proteins have provided some means of differentiating between HCC and CC but are prone to inter-operator variability and lack of sensitivity. There remains therefore, a need for more informative markers for characterising liver tumors in terms of the predominant cellular type—hepatocytes or cholangiocytes—and potentially incorporating molecular markers of drug responsiveness. Such biomarkers of tumor cell lineage can provide an aid to earlier diagnosis, prognostic monitoring of disease, optimised treatment selection and may potentially identify new selective therapeutic targets for future drug development.

### SUMMARY OF THE INVENTION

The present invention, therefore, provides for novel biomarkers for use in the classification of primary liver tumors and particularly distinction between hepatocellular carcinoma and cholangiocellular carcinoma. Such classification allows treatment regimens and prognosis to be specifically tailored to the patient.

In a first aspect, the present invention provides for a method of determining the cellular phenotype of a liver tissue sample said method comprising
(1) extracting marker proteins from said liver tissue sample;
(2) determining expression levels of a plurality of marker proteins in said sample, wherein said plurality of marker proteins are selected from a biomarker panel as represented by any one of Table 1A, Tables 2 to 11; optionally, repeating step (2) with a different plurality of marker proteins selected from a biomarker panel as represented by any one of Table 1A, Tables 2 to 11;
(3) comparing said determined expression levels with reference expression levels for said plurality of marker proteins in known cellular phenotypes, thereby determining the cellular phenotype of the liver tissue sample.

In a second aspect, there is provided a method of identifying the cellular phenotype of a liver cell, said method comprising
(1) determining expression levels of a plurality of marker proteins in said liver cell;
(2) comparing said expression levels with reference set of expression levels for said plurality of marker proteins, said reference levels representing a cellular phenotype;
(3) identifying the cellular phenotype of the liver cell based on the comparison between the expression levels of the marker proteins in the liver cell and the reference expression levels;
wherein the plurality of marker proteins are selected from a biomarker panel as represented by any one of Table 1A, Tables 2 to 10 or Table 11.

In embodiments of these aspects, the cellular phenotype is selected from normal liver epithelium cells (hepatocytes), normal biliary epithelium cells (cholangiocytes), hepatocellular carcinoma cells, peripheral cholangiocellular carcinoma cells or hilar cholangiocellular carcinoma cells.

In some other embodiments the methods further comprise comparing said expression levels with a second reference set of expression levels representing a second cellular phenotype.

In some embodiments, the liver cell is a liver tumor cell.

In some embodiments, the biomarker panel is represented by Table 5 and/or Table 7 and the cellular phenotype is selected from hepatocellular carcinoma cells and cholangiocellular carcinoma cells, preferably the plurality of marker proteins is selected from part A of Table 5.

In some other embodiments, the liver tumor cell is obtained from a liver tumor biopsy sample, preferably obtained from a patient having previously been treated with transarterial chemoembolization.

In yet some other embodiments, the plurality of marker proteins are selected from Table 7, preferably the plurality of marker proteins are selected from Table 7 part A.

In yet some other embodiments of these aspect, the step of determining the expression levels of a plurality of marker proteins comprises
(a) contacting the liver cell or the liver tissue sample with a plurality of binding members, wherein each binding member selectively binds to one of said plurality of marker proteins or nucleic acid sequences encoding said marker proteins; and
(b) detecting and/or quantifying a complex formed by said specific binding members and marker proteins or nucleic acid sequences encoding said marker proteins.

The specific binding member is an antibody or antibody fragment which selectively binds to one of said plurality of marker proteins or a nucleic acid sequence which selectively binds to nucleic acid encoding one of said plurality of marker proteins.

Optionally, the specific binding member is an aptamer or the binding member is immobilised on a solid support.

In some other embodiments of these aspects, the step of determining expression levels of a plurality of marker proteins is performed by mass spectrometry or by Selected Reaction Monitoring using one or more transitions for protein derived peptides; and comparing the peptide levels in the liver cell or the liver tissue sample under test with peptide levels previously determined to represent a cellular phenotype.

Preferably, comparing the peptide levels includes determining the amount of protein derived peptides from the liver cell or the liver tissue sample with known amounts of corresponding synthetic peptides, wherein the synthetic peptides are identical in sequence to the peptides obtained from the liver cell or the liver tissue sample except for a label. More preferably, the label is a tag of a different mass or a heavy isotope.

In a third aspect, the present invention provides for a method for the diagnosis or prognostic monitoring of a liver tumor in an individual, said method comprising
(a) determining the presence or level of expression of a plurality of marker proteins selected from a biomarker panel as represented by any one of Tables 2 to 11, in a liver tumor cell obtained from said individual;
(b) identifying the cellular phenotype of the liver tumor cell; and
(c) selecting a diagnosis or prognosis based on the cellular phenotype of the liver tumor cell.

In a fourth aspect, the present invention provides for a method for determining a treatment regimen for an individual having a liver tumor, said method comprising
(a) determining the presence or level of expression of a plurality of marker proteins selected from a biomarker panel as represented by any one of Tables 2 to 11, in a liver tumor cell obtained from said individual;
(b) identifying the cellular phenotype of the liver tumor cell; and
(c) selecting a treatment regimen based on the cellular phenotype of the liver tumor cell.

In some embodiments of these third and fourth aspects, the liver tumor cell is from a liver tumor biopsy.

In some other embodiments of these aspects, the biomarker panel is represented by Table 5, preferably by Part A of Table 5.

In some further embodiments of these aspects the individual had previously been treated with transarterial chemoembolization. Preferably, the biomarker panel is represented by Table 7, more preferably by Part A of Table 7.

In a fifth aspect, the present invention provides for a method of diagnosing liver cancer in an individual comprising detecting one or more marker proteins or fragments thereof selected from Table 1A, Tables 2 to 11 in a blood, tissue, saliva or urine sample obtained from said individual. Preferably, said one or more protein markers or fragments thereof are detecting using a specific binding member, more preferably said binding member is an antibody specific for said one or marker protein.

In some embodiments of all these aspects, the plurality of marker proteins are selected from any one of Collagen alpha 1 (XVIII) chain, Plastin-3, AKR1B10, Fibronectin, Beta 3 tubulin, Asporin, 14-3-3 protein eta or Dihydropyrimidinase-related protein 3 or combinations thereof, preferably the plurality of marker proteins comprises AKR1B10 and/or Beta 3 tubulin.

In another aspect, the present invention provides for the use of one or more marker proteins selected from Table 1A, Tables 2 to 11 as a diagnostic marker for liver cancer.

In yet another aspect, the present invention provides for a method for diagnosing recurrent or primary liver tumor in a subject, the method comprising determining the presence or absence of one or more marker proteins selected from the group consisting of Collagen alpha 1 (XVIII) chain, Plastin-3, AKR1B10, Fibronectin, Beta 3 tubulin, Asporin, 14-3-3 protein eta, and Dihydropyrimidinase-related protein 3 in a sample. Preferably, the liver tumor is selected from the group consisting of hepatocellular carcinoma, peripheral cholangiocellular carcinoma or hilar cholangiocellular carcinoma cells.

In one embodiment of this aspect the marker protein is Beta 3 tubulin and/or AKR1B10, preferably Beta 3 tubulin.

In another embodiment the sample is selected from any one of blood, plasma, serum, liver tissue, liver cells or combinations thereof, preferably the sample is liver tissue, optionally formalin-fixed paraffin-embedded liver tissue section.

In another embodiment, the determining the presence or absence of one or more marker proteins in the sample is performed by either Immunohistochemistry (IHC) or mass spectrometry.

In another aspect the invention provides for a kit for diagnosing recurrent or primary liver tumor in a subject, the kit comprising reagents for determining the presence or absence of one or more marker proteins selected from the group consisting of Collagen alpha 1 (XVIII) chain, Plastin-3, AKR1B10, Fibronectin, Beta 3 tubulin, Asporin, 14-3-3 protein eta, and Dihydropyrimidinase-related protein 3 in a sample. Preferably, the liver tumor is selected from the group consisting of hepatocellular carcinoma, peripheral cholangiocellular carcinoma or hilar cholangiocellular carcinoma cells.

In one embodiment, the marker protein is Beta 3 tubulin and/or AKR1B10, preferably Beta 3 tubulin.

In another embodiment, the kit comprises reagents suitable for preparing the sample, wherein the sample is selected from any one of blood, plasma, serum, liver tissue, liver cells or combinations thereof.

In yet another embodiment, the sample is liver tissue and the kit comprises reagents suitable for preparing liver tissue, optionally for preparing formalin-fixed paraffin-embedded liver tissue sections.

In another embodiment, the determining the presence or absence of one or more marker proteins in the sample is performed by either Immunohistochemistry.

In yet another aspect, the present invention provides for a kit for use in determining the cellular phenotype of a liver cell, said kit allowing the user to determine the presence or level of expression of a plurality of analytes selected from proteins or fragments thereof provided in biomarker panels as represented by any one of Table 1A, Tables 2 to 11, a plurality of antibodies against said marker proteins and a plurality of nucleic acid molecules encoding said marker proteins or fragments thereof, in a cell under test; the kit comprising
(a) a solid support having a plurality of binding members, each capable of binding to one of the analytes immobilised thereon;
(b) a developing agent comprising a label; and, optionally
(c) one or more components selected from the group consisting of washing solutions, diluents and buffers.

The present invention also provides for a kit for use in determining the cellular phenotype of a liver cell in vitro, said kit allowing the user to determine the presence or level of expression of a plurality of proteins or fragments thereof provided in biomarker panels represented by Table 1A, Tables 2 to 11, in a cell under test; the kit comprising
(a) a set of reference peptides in an assay compatible format wherein each peptide in the set is uniquely representative of one of the plurality of marker proteins provided in any one of Table 1A, Tables 2 to 11; and, optionally
(b) one or more components selected from the group consisting of washing solutions, diluents and buffers.

In yet a further aspect, the present invention provides for a kit for the diagnosis, prognostic monitoring of a liver tumor in an individual or for determining a treatment regimen for an individual having a liver tumor, the kit comprising
(a) a solid support having a plurality of binding members immobilised thereon, wherein each binding member selectively binds to a protein selected from a biomarker panel as represented by any one of Table 1A, Tables 2 to 11; or a nucleic acid encoding the protein or fragment thereof;
(b) a developing agent comprising a label; and
(c) one or more components selected from washing solutions, diluents and buffers.

Preferably, the biomarker panel is represented by Table 5 or by Part A of Table 4 or by Table 7 or Part A of Table 7.

In yet a further aspect, the present invention provides for a plurality of synthetic peptides each having a sequence identical to a fragment of one of a plurality of proteins selected from a biomarker panel selected from any one of Table 1A, Tables 2 to 11, said fragment resulting from digestion of the protein by trypsin, ArgC, AspN or Lys-C digestion, wherein one or more of the plurality of synthetic peptides comprises a label, optionally for the use in Selective Reaction Monitoring. Preferably, the label is a heavy isotope.

The present invention also provides for a liver cellular classification system comprising a liver cellular classification apparatus and an information communication terminal apparatus, said liver cellular classification apparatus including a control component and a memory component, said apparatuses being communicatively connected to each other via a network;
(1) wherein the information communication terminal apparatus includes
(1a) a protein data sending unit that transmits the protein data derived from a liver tissue sample of a subject to the liver cellular classification apparatus;
(1b) a result-receiving unit that receives the result of the liver cellular classification of the subject transmitted from the liver cellular classification apparatus;
(2) wherein the liver cellular classification apparatus includes
(2a) a protein data-receiving unit that receives protein data derived from the liver tissue sample of the subject transmitted from the information communication terminal apparatus;
(2b) a data comparison unit which compares the data from the data-receiving unit with the data stored in the memory unit;
(2c) a classifier unit that determines the class (e.g. cellular phenotype) of the liver tissue of the subject, based on the results of the data comparison unit; and
(2d) a classification result-sending unit that transmits the classification result of the subject obtained by the classifier unit to the information communication terminal apparatus; and
wherein the memory unit contains protein expression level data of at least one protein selected from any one or more of Table 1A, Tables 2 to 10 or Table 11.

Preferably, the memory unit contains data of a plurality of proteins selected from Table 5 or Table 11 and wherein the classification is between Hepatocellular carcinoma and peripheral cholangiocarcinoma; alternatively the memory unit contains data of a plurality of proteins selected from Table 7 or Table 11 and wherein the classification is between Hepatocellular carcinoma and cholangiocarcinoma in post-TACE liver tumors.

In some embodiments, the liver cellular classification system according to the invention is connected to an apparatus for determining the protein expression levels in a liver tissue sample, preferably the apparatus can process multiple samples using liquid chromatography-mass spectrometry (LC-MS/MS).

In yet a further aspect of the present invention, there is provided a liver tissue cellular classification program that makes an information processing apparatus including a control component and a memory component execute a method of determining and/or classifying the liver tissue of a subject, the method comprising:
(i) a comparing step of comparing data based on the protein expression levels of at least one (preferably a plurality) protein selected from any one of Table 1A, Tables 2 to 11 obtained of a subject with the protein expression level data stored in the memory component; and
(ii) a classifying step for classifying the liver tissue cells of said subject, based on the comparison calculated at the comparing step; and wherein said tissue is classified into phenotypes including normal (hepatocytes, cholangiocytes), hepatocellular carcinoma, truly mixed hepatocholangiocellular carcinoma (pre or post TACE therapy), peripheral cholangiocarcinoma, Hilar cholangiocarcinoma (with or without primary sclerosing cholangitis), or metastatic colo-rectal carcinoma. Preferably, the liver tissue cellular classification program of claim 48 recorded thereon.

### BRIEF DESCRIPTION OF THE DRAWINGS

FIG. 1. Overall workflow. Overall data analysis workflow; Spectrum files (0), Spectrum Selector (1), Sequest (2), Percolator (3), Mascot (4), Event Detector (5), Precursors Ions Area Detector (6), Peptide Area Quantified (7), Peptide Identification at 1% FDR+ spectral counting (8), Peptide Matrix with Area under the Curve (AUC)(9), Peptide data matrix with spectral count information (10), Statistical validation (11; 12) and final list (13).

FIG. 2. Venn-Diagram. The diagram shows comparison of the two quantitation methods (left: spectral count; right: Area under the Curve) for marker proteins with unique and shared peptides. The numbers shown the marker proteins found to be significantly modulated in each quantification method and those common to both, across all comparisons made in this study.

FIG. 3. Principal component analysis (PCA) for identifying outliers and groups/clusters nested within the datasets (A) for the Area under the Curve (AUC) and (B) for the spectral counts. Each small triangle represents the PC score along the first two PC components for each of the samples. The naming convention of the samples is batch number tissue type number.

FIG. 4. Validation of protein up-regulation through Volcano plots. (a) Volcano plots for AKR1B10 (upper left panel, normal liver parenchyma (1) vs. HCC (2); upper right panel, normal liver parenchyma (1) vs. normal bile duct (9)) and Tubulin-beta 3 chain (lower left panel, normal liver parenchyma (1) vs. peripheral CC (5); lower right panel, peripheral CC (5) vs. normal bile duct (9).

FIG. 5. Validation of protein up-regulation through Immuno-hystochemical staining (IHC). (1): tissue type 1 (normal liver); (2) tissue type 2 (HCC); (3) tissue type 9 (Normal bile duct) and (4) tissue type 5 (peripheral CC) AKR1B10 is diffusely expressed in HCC, while its expression is only patchy or weak in normal liver parenchyma and peripheral CC. AKR1B10 is also diffusely positive in normal bile duct. Immunostaining for Tubulin-beta 3 chain on normal liver, HCC, normal bile duct, and peripheral CC. The expression of Tubulin-beta 3 chain appears to be specific for peripheral CC.

FIG. 6. Normalised spectral counts for Collagen alpha 1 (XVIII) chain (1), Plastin-3 (2), AKR1B10 (3), Fibronectin (4), Beta 3 tubulin (5), Asporin (6) and Dihydropyrimidinase-related protein 3 (7).

FIG. 7. Table 11 shows a list of marker proteins (467) with both unique and shared peptide sequences.

### DEFINITIONS

The term “plurality of marker proteins” means at least two marker proteins as disclosed herein.

The term “marker protein” includes all biologically relevant forms of the protein identified, including post-translational modifications. For example, the marker protein can be present in a glycosylated, phosphorylated, multimeric, fragmented or precursor form. A marker protein fragment may be naturally occurring or, for example, enzymatically generated and the biologically active function of the full marker protein. Fragments will typically be at least about 10 amino acids, usually at least about 50 amino acids in length, and can be as long as 300 amino acids in length or longer.

The term “cellular phenotype” refers to the characteristics or traits of a cell or group of cells. Cellular phenotype refers to the cells anatomical location, morphology, development, biochemical or physiological properties, behaviour, and products of biochemistry/behaviour. Cellular phenotype results from the expression of cell genes as well as the influence of environmental factors and the interactions between the two.

The term “liver tissue sample” include, but is not limited to, a specimen of liver tissue removed by resection or core needle biopsy.

The term “expression level” refers to the relative amount of protein in a liver tissue sample, for example as determined by LC-MS/MS label free quantification approaches such as area under the curve and spectral counting.

The term “comparing” means measuring the relative amount of a protein or proteins in a sample relative to other samples (for example protein amounts stored in our database).

The term “reference set” refers to the samples (for example in our database) used as classifiers (e.g. classic examples or HCC, or CC). These classifiers can be used to help diagnosis of non-classic specimens from new cases.

The term “reference level”, “reference set of expression level”; “reference expression level” and “reference amount” are used herein as synonyms and refers to a pre-determined level, which may, for example be provided in the form of an accessible data record from a public database.

The term “antibody” includes polyclonal antiserum, monoclonal antibodies, fragments of antibodies such as single chain and Fab fragments, and genetically engineered antibodies. The antibodies may be chimeric or of a single species.

The terms “marker protein” and “biomarker”, which are used interchangeably herein, include all biologically relevant forms of the protein identified, including post-translational modifications. For example, the marker protein can be present in a glycosylated, phosphorylated, multimeric or precursor form.

The term “control” refers to a cultured cell line, primary culture of cells taken from a human or animal subject, or biopsy material taken from a human or animal subject that is free of HCC or CC.

The term “antibody array” or “antibody microarray” means an array of unique addressable elements on a continuous solid surface whereby at each unique addressable element an antibody with defined specificity for an antigen is immobilised in a manner allowing its subsequent capture of the target antigen and subsequent detection of the extent of such binding. Each unique addressable element is spaced from all other unique addressable elements on the solid surface so that the binding and detection of specific antigens does not interfere with any adjacent such unique addressable element.

The term “bead suspension array” means an aqueous suspension of one or more identifiably distinct particles whereby each particle contains coding features relating to its size and colour or fluorescent signature and to which all of the beads of a particular combination of such coding features is coated with an antibody with a defined specificity for an antigen in a manner allowing its subsequent capture of the target antigen and subsequent detection of the extent of such binding. Examples of such arrays can be found at www.uminexcorp.com where application of the xMAP® bead suspension array on the Luminex® 100™ System is described.

The terms “selected reaction monitoring”, “SRM” and “MRM” means a mass spectrometry assay whereby precursor ions of known mass-to-charge ratio representing known biomarkers are preferentially targeted for analysis by tandem mass spectrometry in an ion trap or triple quadrupole mass spectrometer.

During the analysis the parent ion is fragmented and the number of daughter ions of a second predefined mass-to-charge ratio is counted. Typically, an equivalent precursor ion bearing a predefined number of stable isotope substitutions but otherwise chemically identical to the target ion is included in the method to act as a quantitative internal standard.

“Differential expression”, as used herein, refers to at least one recognisable difference in protein expression. It may be a quantitatively measurable, semi-quantitatively estimable or qualitatively detectable difference in tissue protein expression. Thus, a differentially expressed protein may be strongly expressed in tissue in one cellular phenotype (e.g. HCC) and less strongly expressed or not expressed at all in another cellular phenotype (e.g. CC). Further, expression may be regarded as differential if the protein undergoes any recognisable change such as cleavage or post-translational modification between two cellular phenotypes under comparison.

The term “isolated” means throughout this specification, that the marker protein, antibody or polynucleotide, as the case may be, exists in a physical milieu distinct from that in which it may occur in nature.

As used herein, the term “subject” includes any human or non-human animal. The term “non-human animal” includes all vertebrates, e.g., mammals and non-mammals, such as non-human primates, sheep, dogs, cats, horses, cows, chickens, amphibians, reptiles, etc.

The term “treat”, “treating”, “treatment”, “prevent”, “preventing” or “prevention” includes therapeutic treatments, prophylactic treatments and applications in which one reduces the risk that a subject will develop a disorder or other risk factor. Treatment does not require the complete curing of a disorder and encompasses the reduction of the symptoms or underlying risk factors.

Abbreviations

LMD: laser microdissection; TACE: trans-arterial chemo-embolization; HCC: hepatocellular carcinoma; CC: cholangiocellular carcinoma

PSC: primary sclerosing cholangitis; FFPE: Formalin Fixed Paraffin embedded;

AUC: area under the curve; PSM: peptide spectrum match.

### DETAILED DESCRIPTION

The inventors have identified marker proteins that demonstrate statistically significant differences in protein expression levels between different cellular phenotypes of liver cells, including liver tumor cells. In particular, the inventors have determined marker proteins having different expression levels between components (HCC and CC) of post-TACE HCC, Often cases diagnosed with HCC are then treated with transarterial chemoembolization (TACE), however tumors generally come back, but no longer show the classic HCC phenotype, having some regions that look classic HCC, some that look classic CC, and some which are undefinable. The present invention allows for the identification of marker proteins more specific for HCC than CC, or vice versa, in a patient that has already undergone TACE. The inventors have further explored their similarities or dissimilarities compared to their normal and typical malignant counterparts. The inventors also found significant differences in other tissue type comparisons. These differentially expressed marker proteins provide useful biomarkers to help diagnosing tumor types, assessing patient prognosis and determining appropriate treatment regimens.

The identification of marker protein sets (or biomarker panels) specific to the hepatocellular and cholangiocellular phenotype of post-TACE mixed tumors, and their similarity to their normal and typical neoplastic counterparts confirms that the differentiation process is truly divergent, despite a probable origin from a common progenitor. Of equal importance is the identification by the inventors of marker proteins differentially expressed between normal and neoplastic hepatocytes and biliary epithelial cells, as they provide new markers of malignant transformation or tumor differentiation; and between HCC and peripheral CC, which often overlap in both clinical presentation, and appearance on imaging and histology (22, 23).

### 1. MARKER PROTEINS AND METHODS OF USING THEREOF

The present invention provides herein marker proteins which are differentially expressed between two cell types tested and allow a particular cellular phenotype to be determined.

Table 1A shows the preferred marker proteins (including their synonyms) according to the invention, namely Beta 3 tubulin, AKR1B10, Collagen alpha 1 (XVIII) chain, Plastin-3, Fibronectin, Asporin, 14-3-3 protein eta and Dihydropyrimidinase-related protein 3.

TABLE 1A
Preferred marker proteins

| | | Abbreviation(s)/ | Tissue of |
|---|---|---|---|
| Protein | Name | synonyms (Syn) | upregulation |
| Q13509 | Tubulin | TUBB3 | peripheral |
| | beta-3 chain | Syn: Beta-3 | CC |
| | | tubulin; β-3 tubulin; | |
| | | tubulin-β-3; TUBB4 | |
| O60218 | Aldo-keto reductase | AKR1B10 | HCC |
| | family 1 | Syn: AKR1B11 | |
| | member B10 | | |
| P39060 | Collagen alpha- | COL18A1 | HCC |
| | 1(XVIII) chain | | |
| | | Syn: Collagen α-1 | |
| | | (XVIII) | |
| P13797 | Plastin-3 | PLS3 | Hilar CC |
| P02751 | Fibronectin | FN1 | HCC |
| | | Syn: FN | |
| Q9BXN1 | Asporin | Syn: PLAP1; SLRR1C | Hilar CC |
| Q04917 | 14-3-3 protein eta | YWHAH | HCC pre- |
| | | Syn: YWHA1; 14-3-3 | TACE |
| | | protein ε | |
| Q14195 | Dihydropyrimidinase- | DPYSL3 | HCC |
| | related protein 3 | Syn: CRMP4; DRP3; | without psc |
| | | ULIP; ULIP1 | |

Pre-TACE: pre-trans-arterial chemo-embolization;
HCC: hepatocellular carcinoma;
CC: cholangiocellular carcinoma;
PSC: primary sclerosing cholangitis;

Table 1B indicates the numbers of proteins that showed statistically significant differential expression levels between two types of liver tissues (p-value <0.05 and Log 2 [fold change]≧2 or ≦−2) using shared and unique peptides. These numbers illustrate the number of differentially modulated proteins that were common to both area under the curve and spectral counting datasets per tissue type comparison (467 proteins common to both).

TABLE 1B
Number of proteins showing statistically significant differential
expression between types of liver tissue.

| Tissue Type | 2 | 3 | 4 | 5 | 6 | 7 | 8 | 9 |
|---|---|---|---|---|---|---|---|---|
| 1 | 11 | 37 | 203 | 236 | 269 | 248 | 258 | 212 |
| 2 | | 2 | 125 | 165 | 195 | 175 | 188 | 153 |
| 3 | | | 95 | 130 | 151 | 140 | 163 | 125 |
| 4 | | | | 5 | 15 | 17 | 33 | 31 |
| 5 | | | | | 14 | 34 | 29 | 37 |
| 6 | | | | | | 5 | 63 | 32 |
| 7 | | | | | | | 51 | 43 |
| 8 | | | | | | | 0 | 53 |

1: Normal liver;
2: HCC;
3: Post-TACE HCC, hepatocellular area;
4: post-TACE HCC, cholangiocellular area;
5: peripheral CC;
6: hilar CC without PSC;
7: hilar CC with PSC;
8: metastatic colorectal carcinoma.

The marker proteins indicated in the Tables 2 to 10 allow the following cell types to be distinguished:—

Table 2: Normal hepatocytes from HCC.
Table 3: Peripheral cholangiocarcinoma from normal bile duct.
Table 4: Hilar cholangiocarcinoma from normal bile duct.
Table 5: Hepatocellular carcinoma from peripheral cholangiocarcinoma.
Table 6: Hepatocytes from cholangiocytes.
Table 7: Hepatocellular carcinoma and cholangiocarcinoma in post-TACE liver tumors.
Table 8: Peripheral cholangiocarcinoma from metastatic colorectal cancer.
Table 9: Hilar cholangiocarcinoma from hilar cholangiocarcinoma with primary sclerosing cholangitis.
Table 10: Hilar cholangiocarcinoma from metastatic colorectal cancer.

This determination provides clinicians for the first time with knowledge of the cellular phenotype of the liver tumor and as a result, accurate decisions regarding type and assessment of treatment, and prognosis can be provided. For each Table, all negative values for effect size (g), relate to marker proteins that were present at a lower concentration in the first tissue type versus the second tissue type. All positive values for effect size (g), relate to marker proteins that were present in higher concentration in the first tissue type versus the second tissue type.

The statistical significance for each protein regulation is shown as a p-value calculated after performing an unrelated t-test comparing the number of spectral counts for each protein between the two named tissue types. Hedges' g unbiased standardized effect size estimates were calculated, along with 95% confidence intervals for these estimates. Values of g<0.2 are regarded as very small differences, g=0.5 average differences, g>0.8 regarded as large differences. Unstandardized effect size estimates (i.e., differences in the mean spectral counts in two compared tissue types) were also calculated, along with 95% confidence intervals for these estimates. The tables displayed these unbiased standardized effect size estimates and the unstandardized effect size estimates. Q-values (adjusted p values) provide a more stringent measure of statistical significance than p-values and were computed using a direct False Discovery Rate approach. Individual Q-values are not shown here but all marker proteins with q-values 0.05 are listed in section A of each tables 2 to 10, while all marker proteins with p-values 0.05 are displayed in section B of each table 2 to 10.

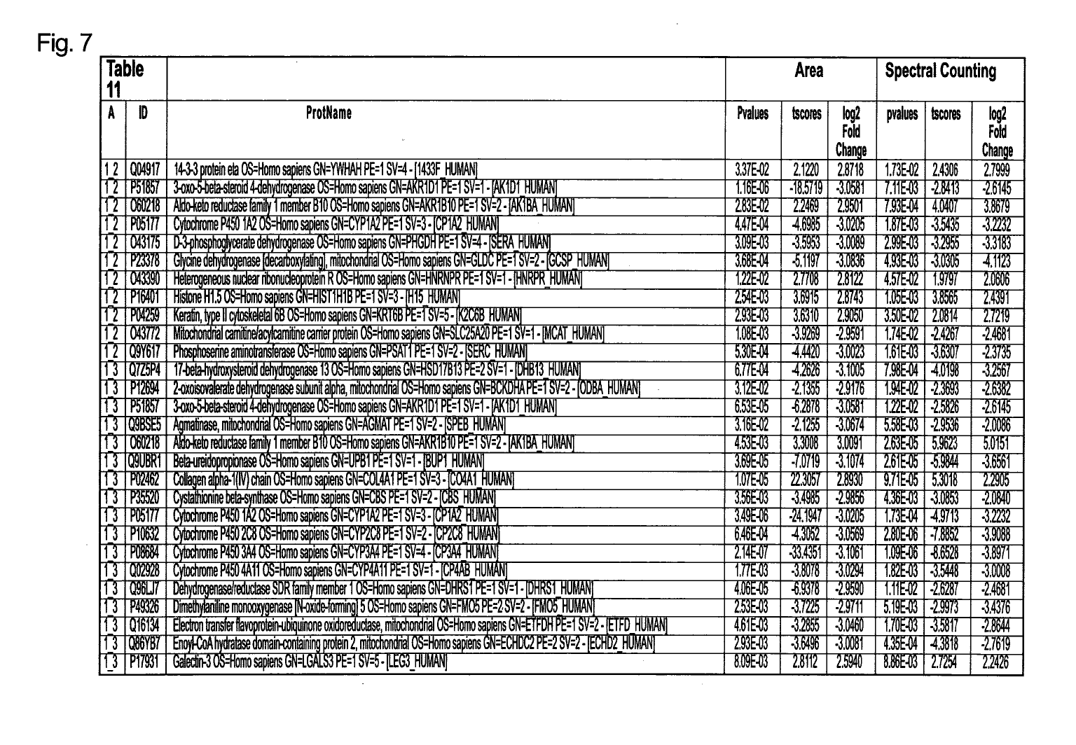

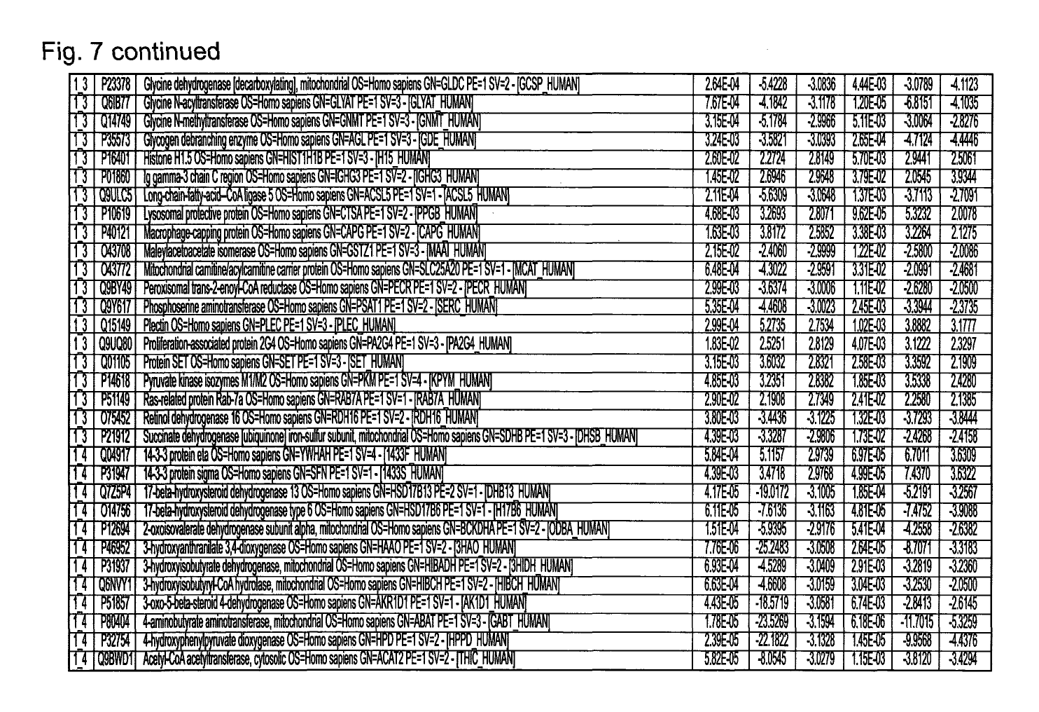

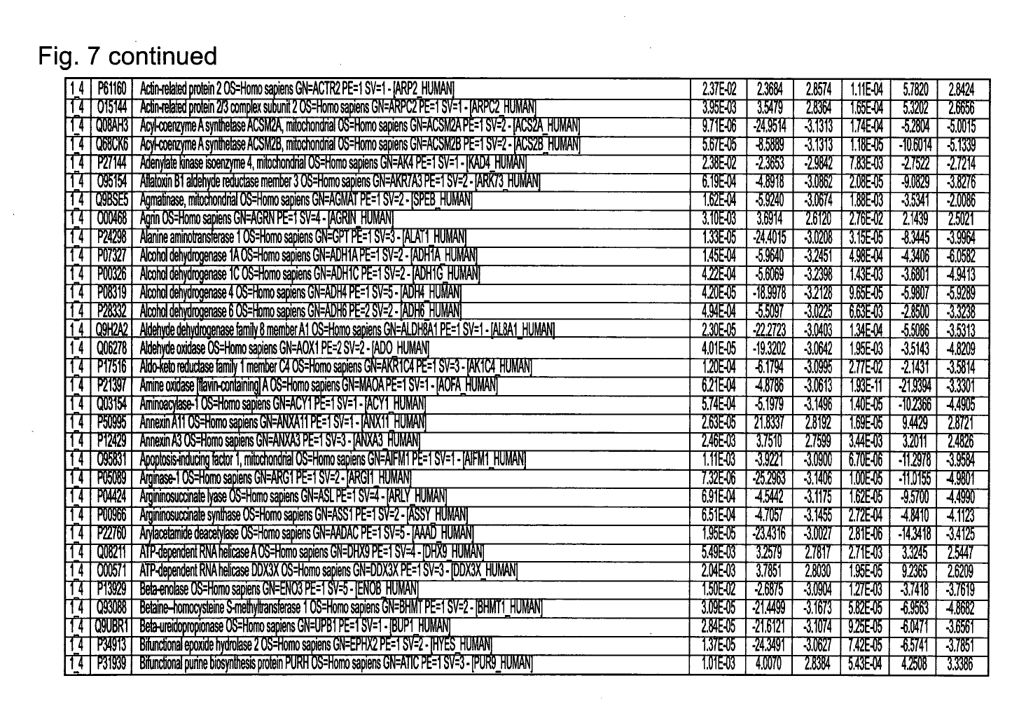

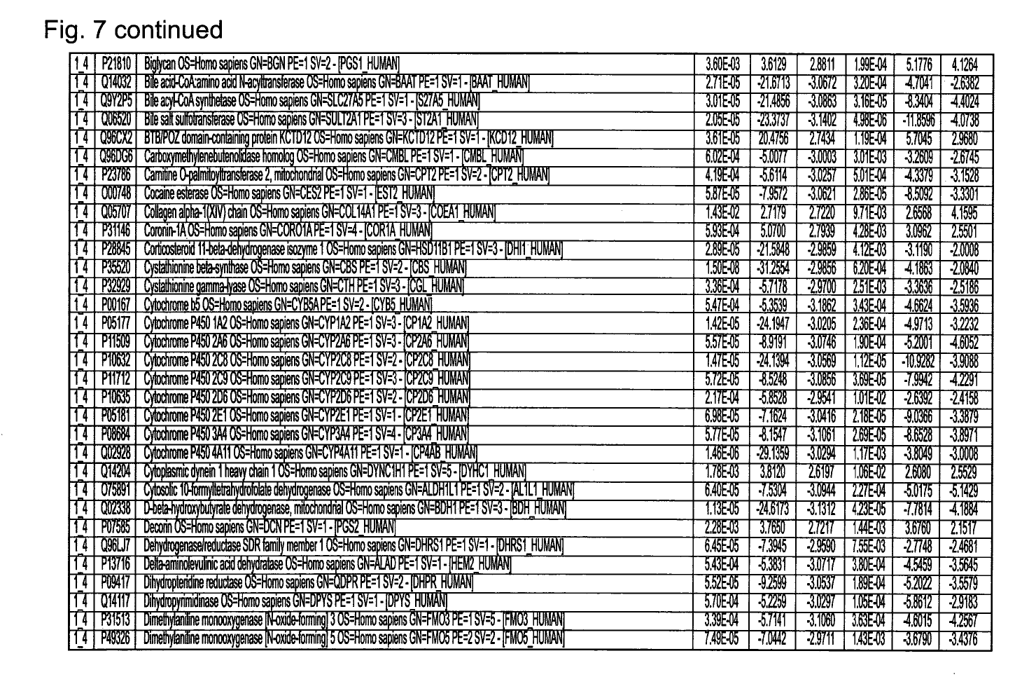

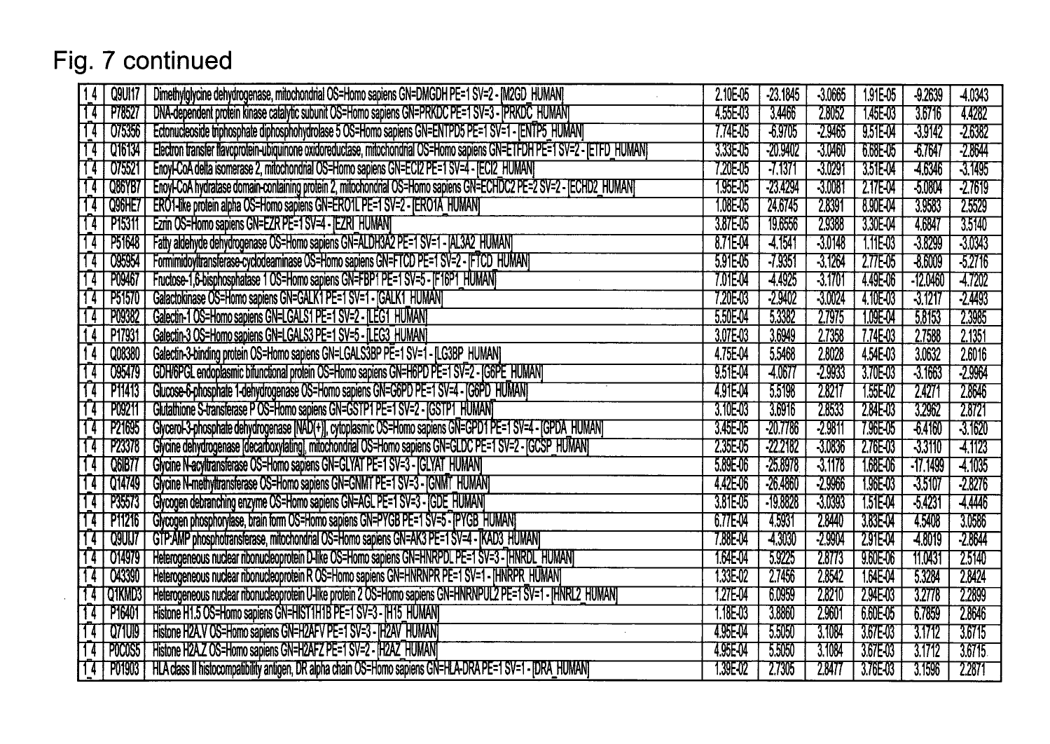

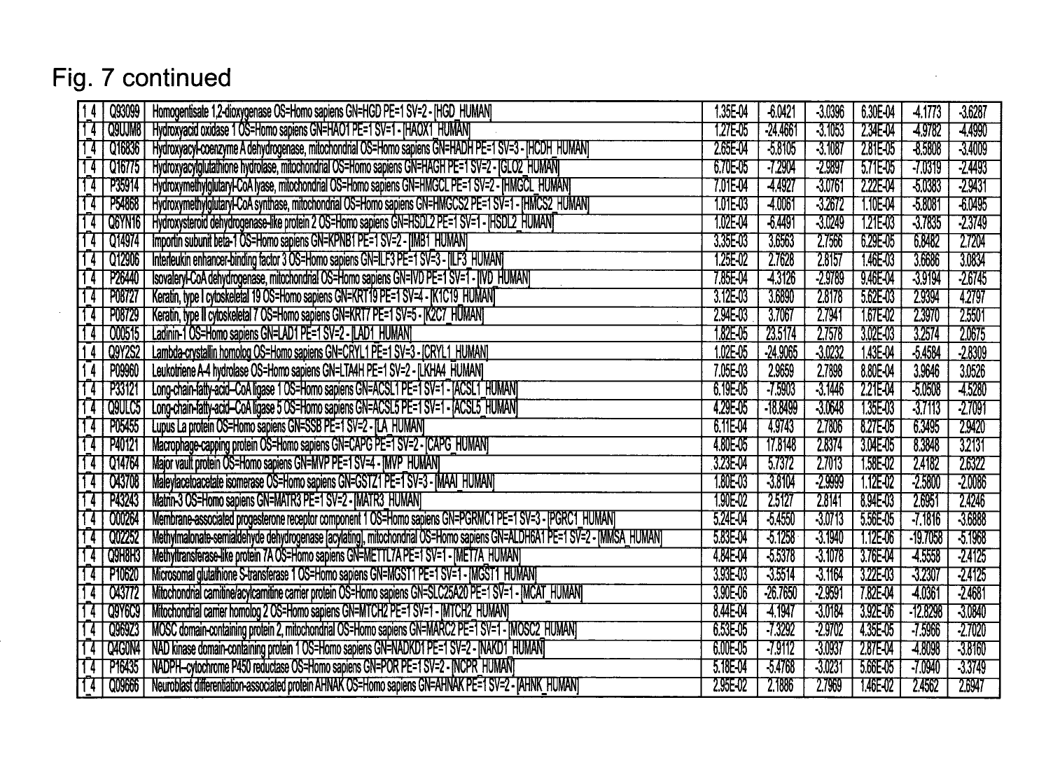

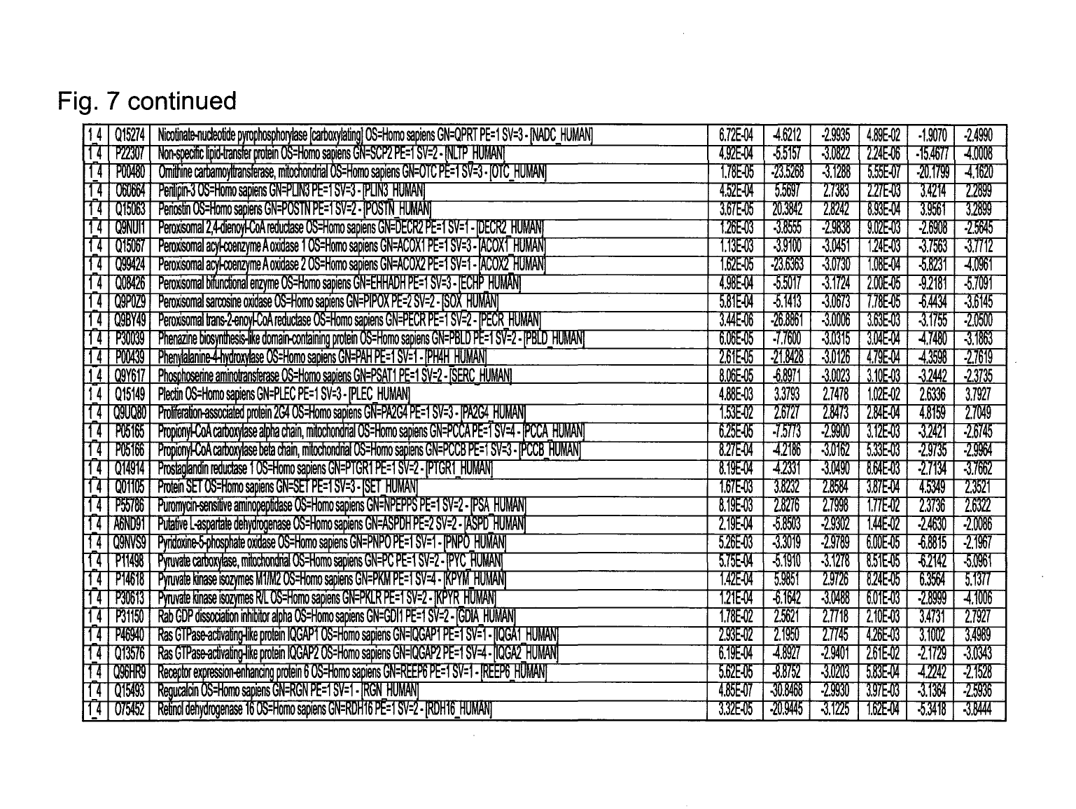

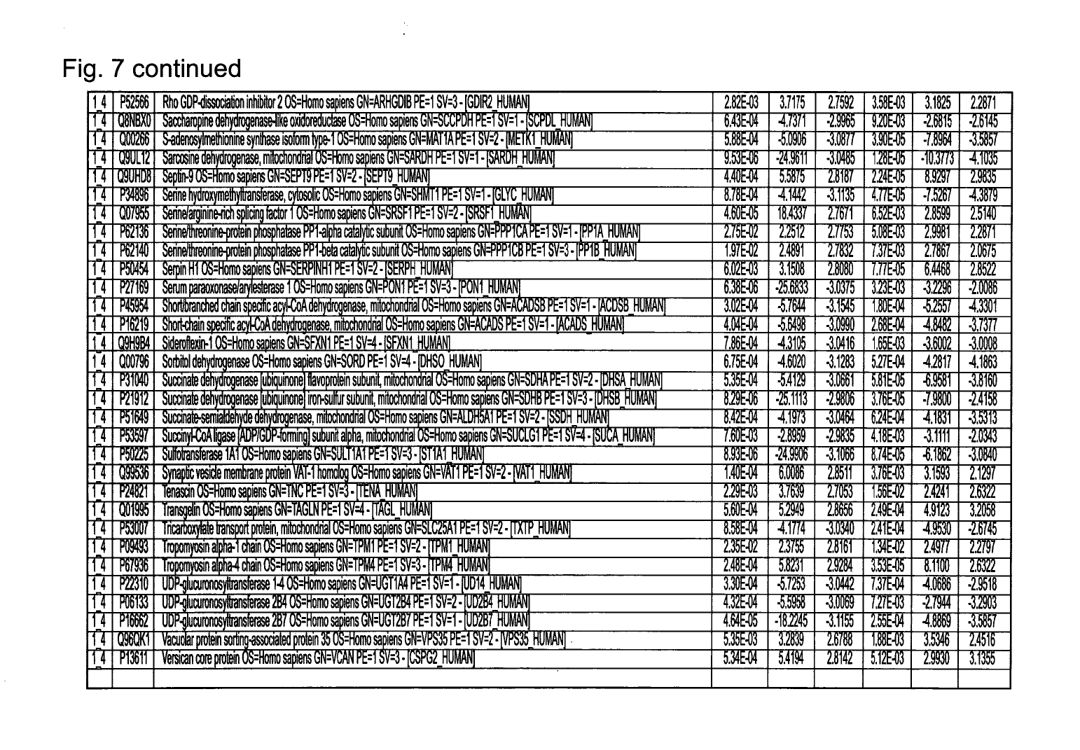

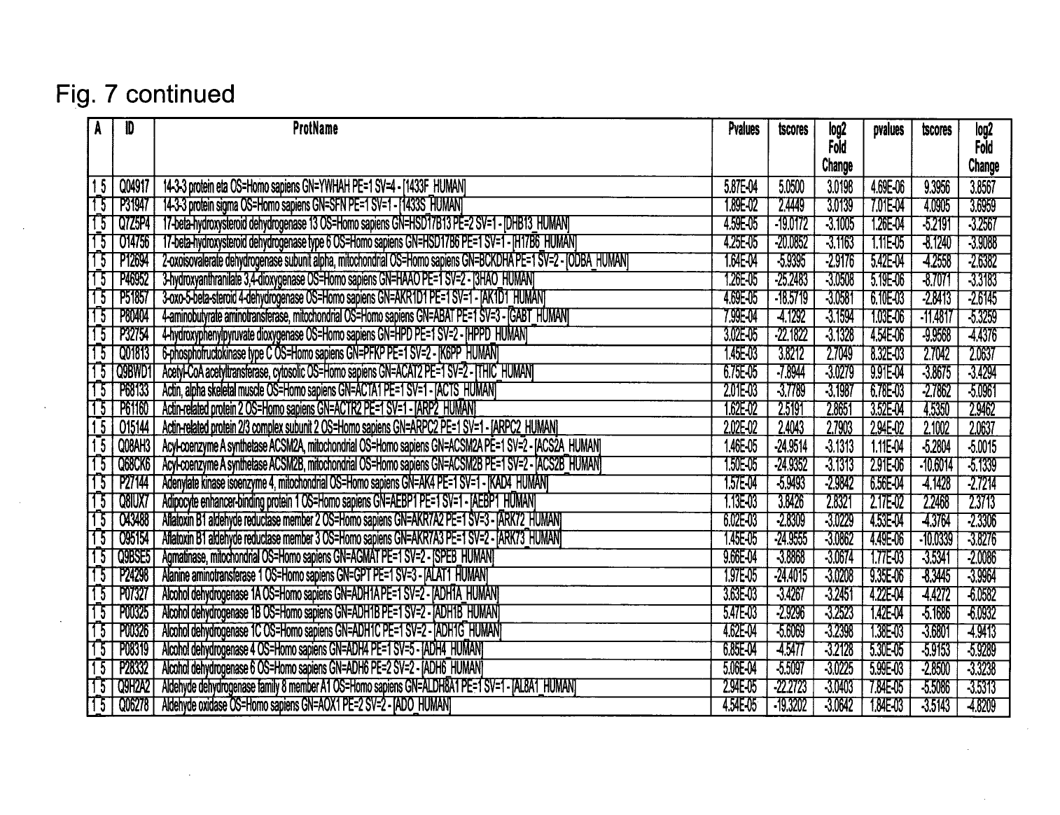

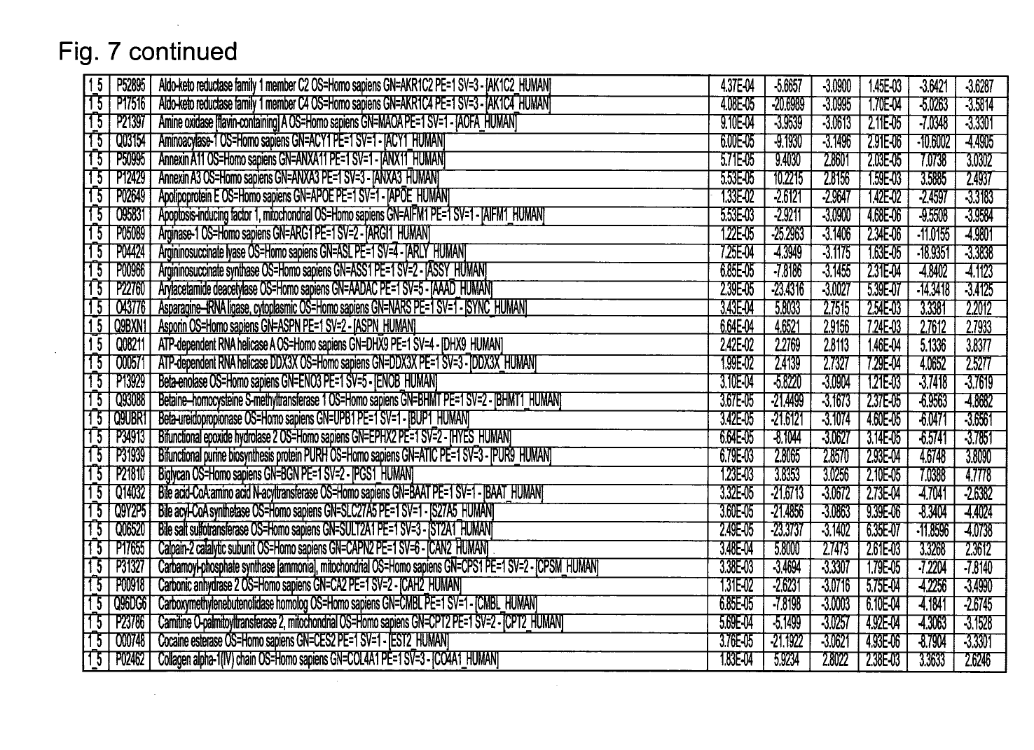

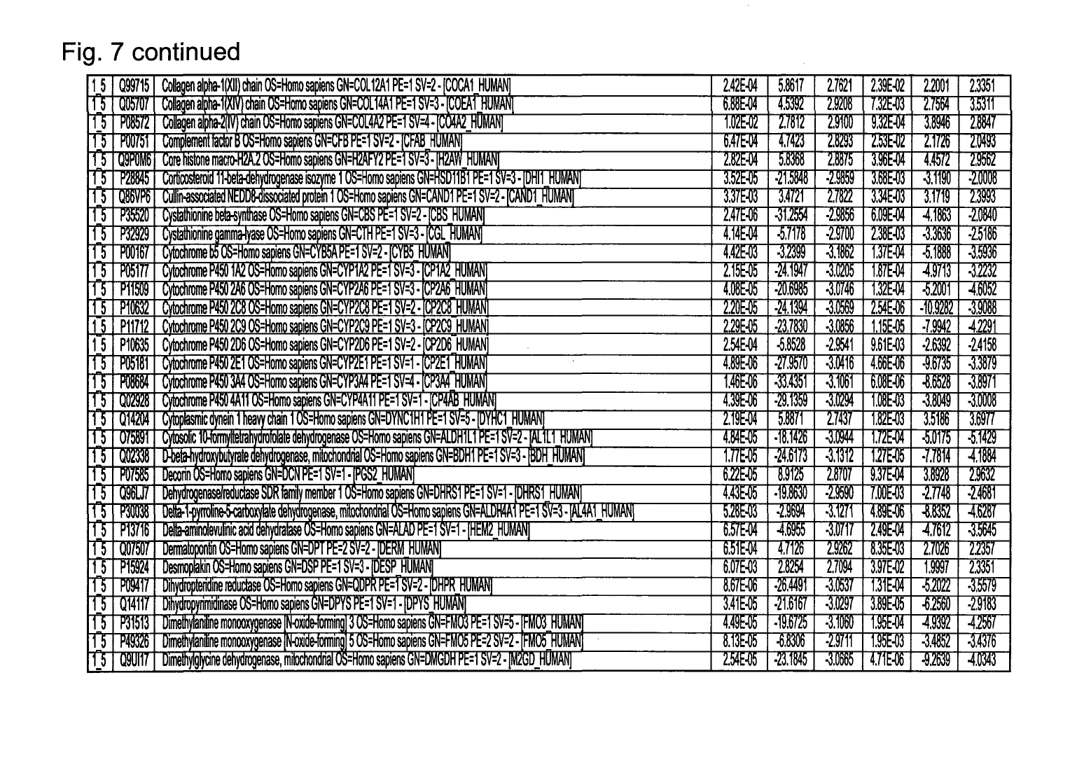

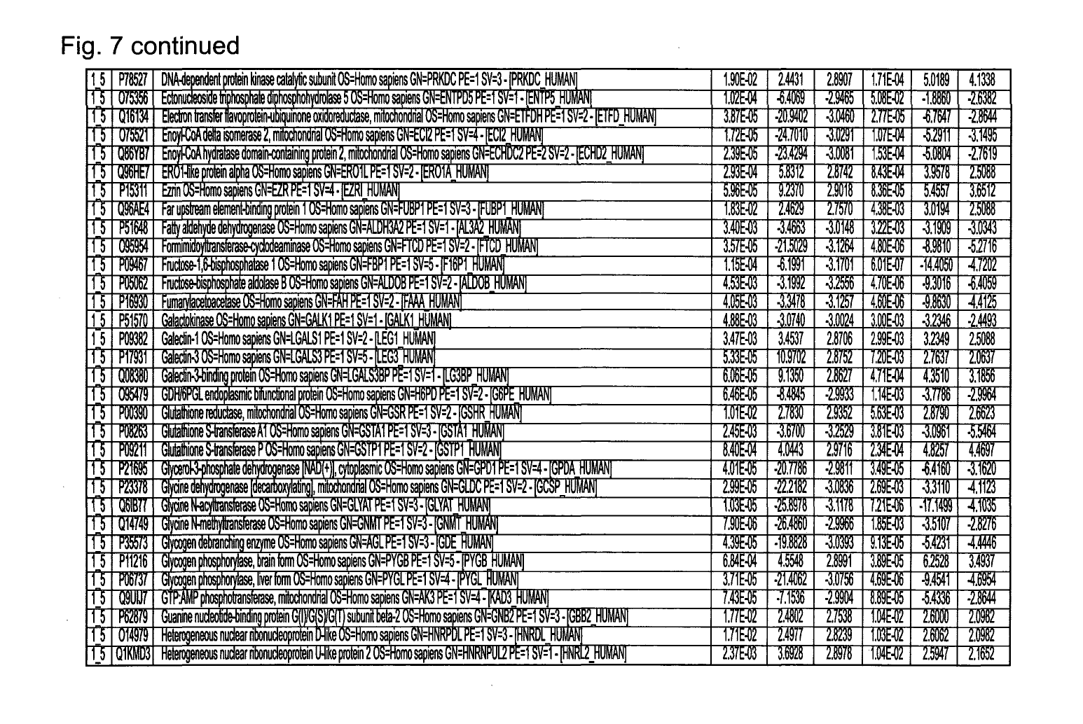

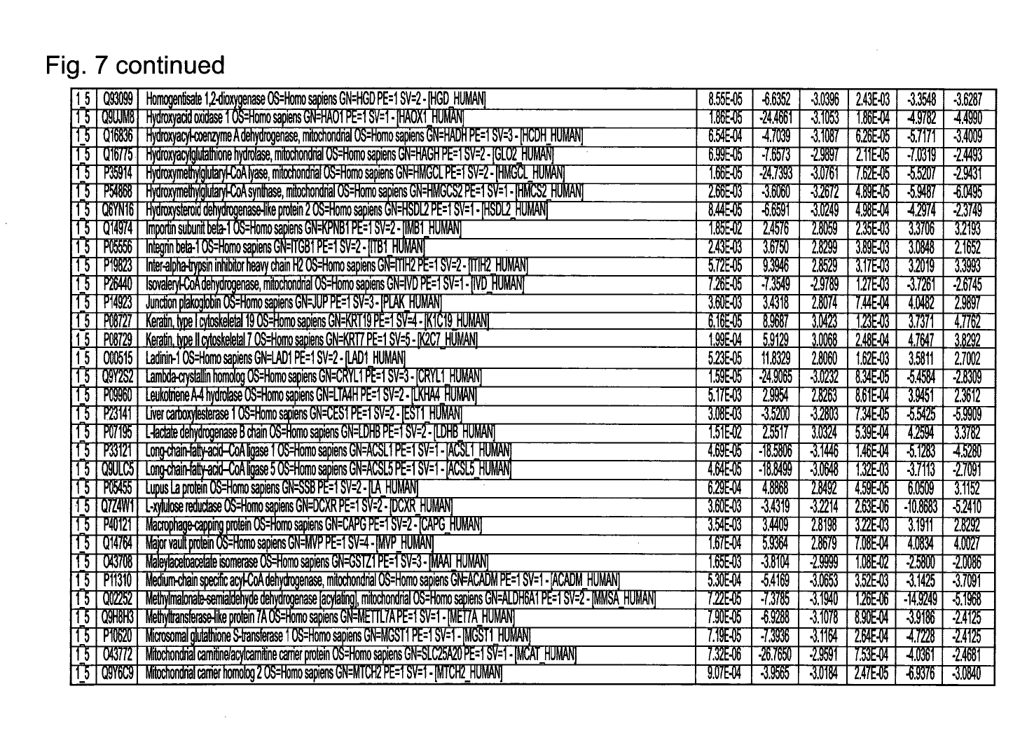

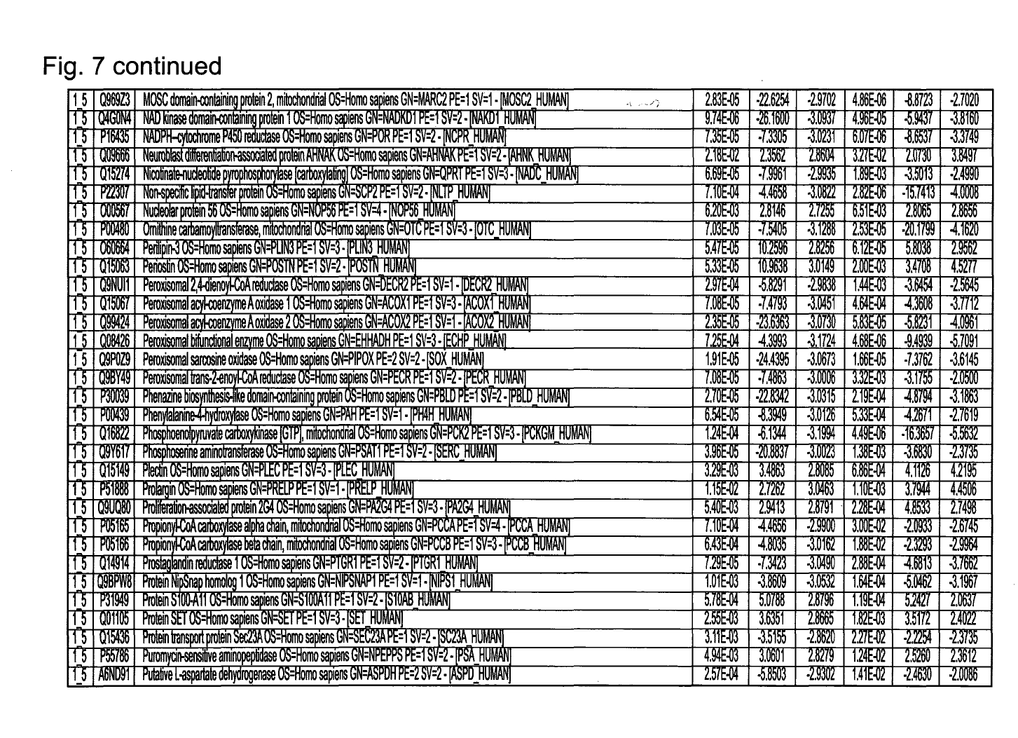

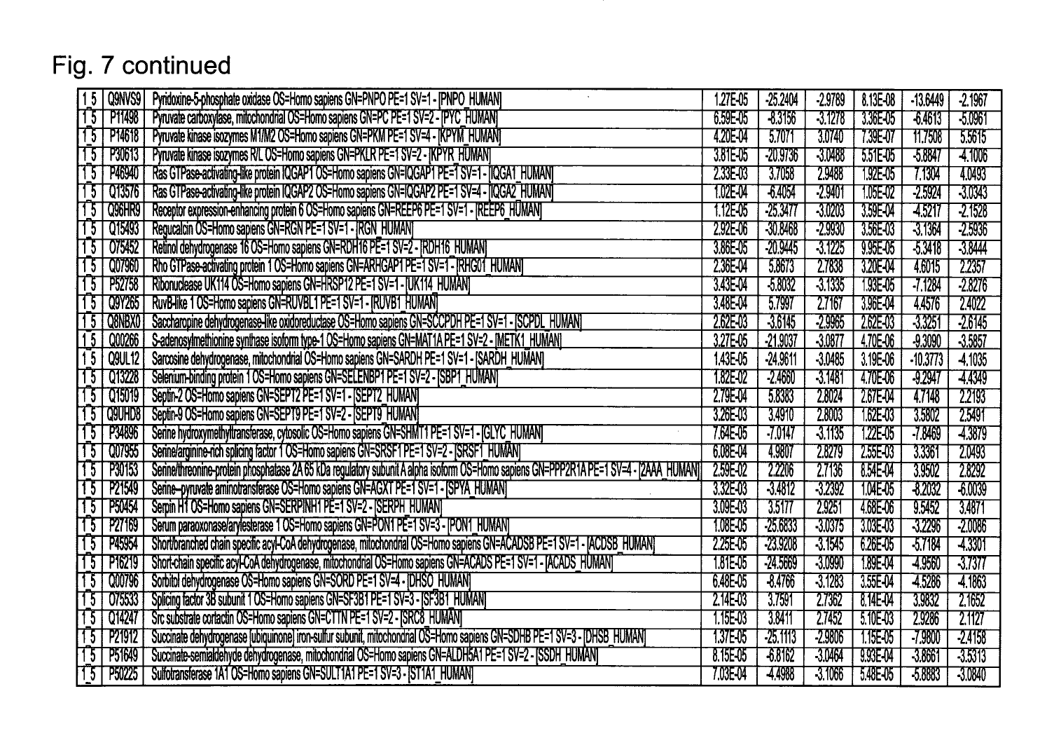

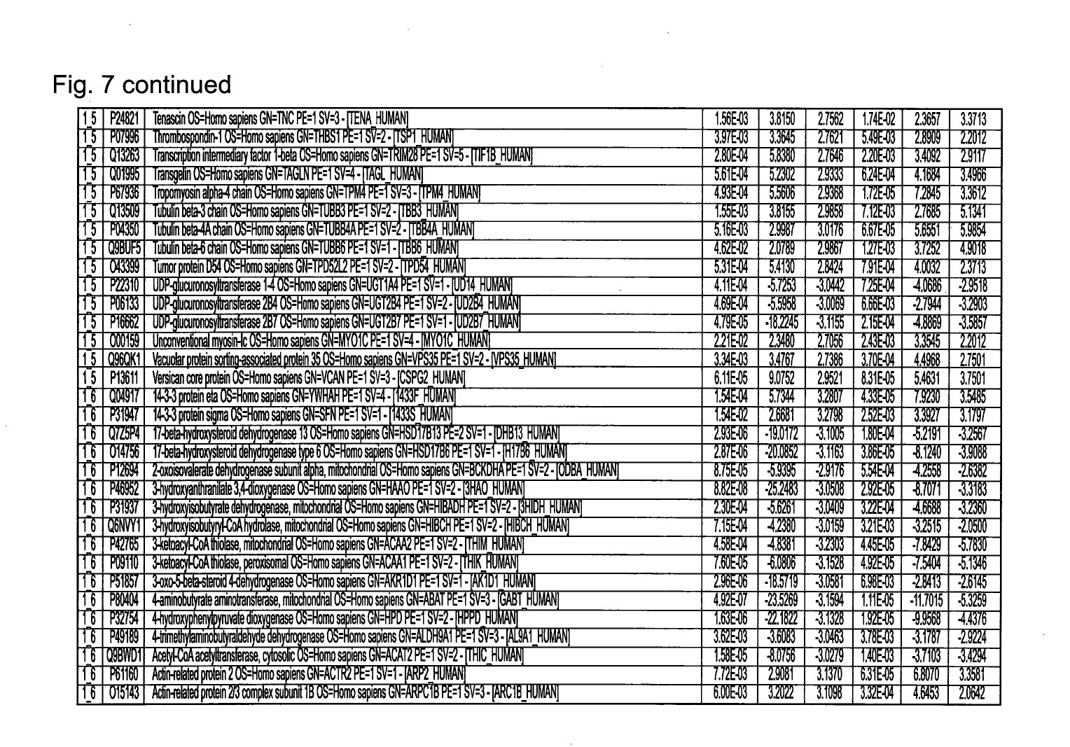

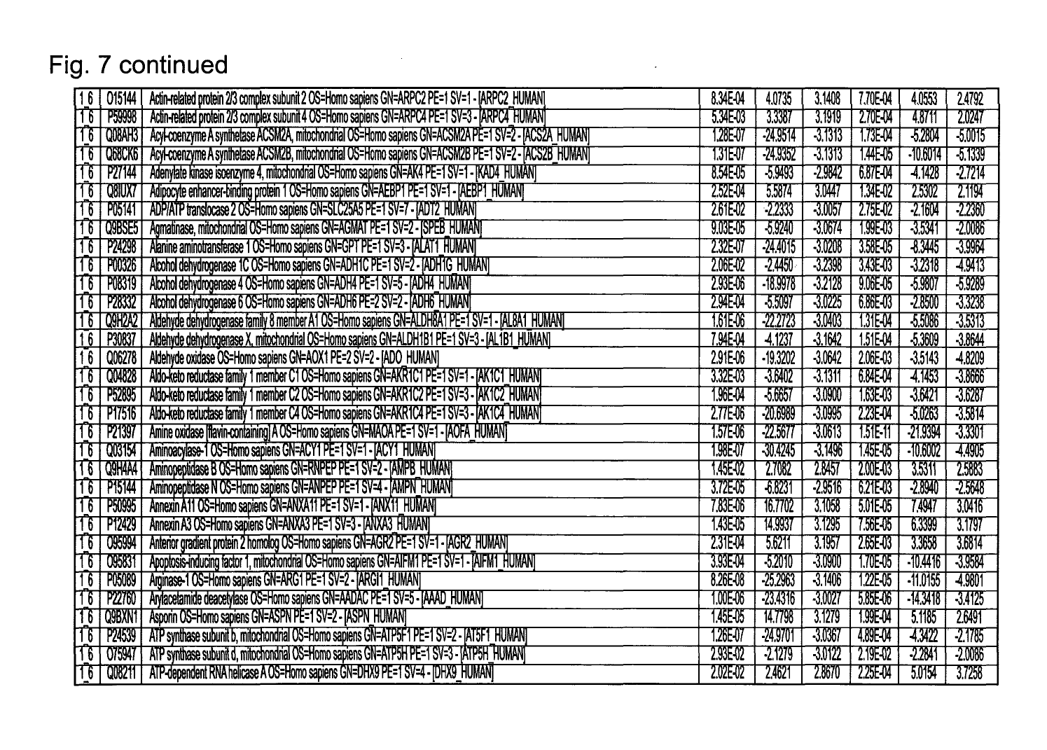

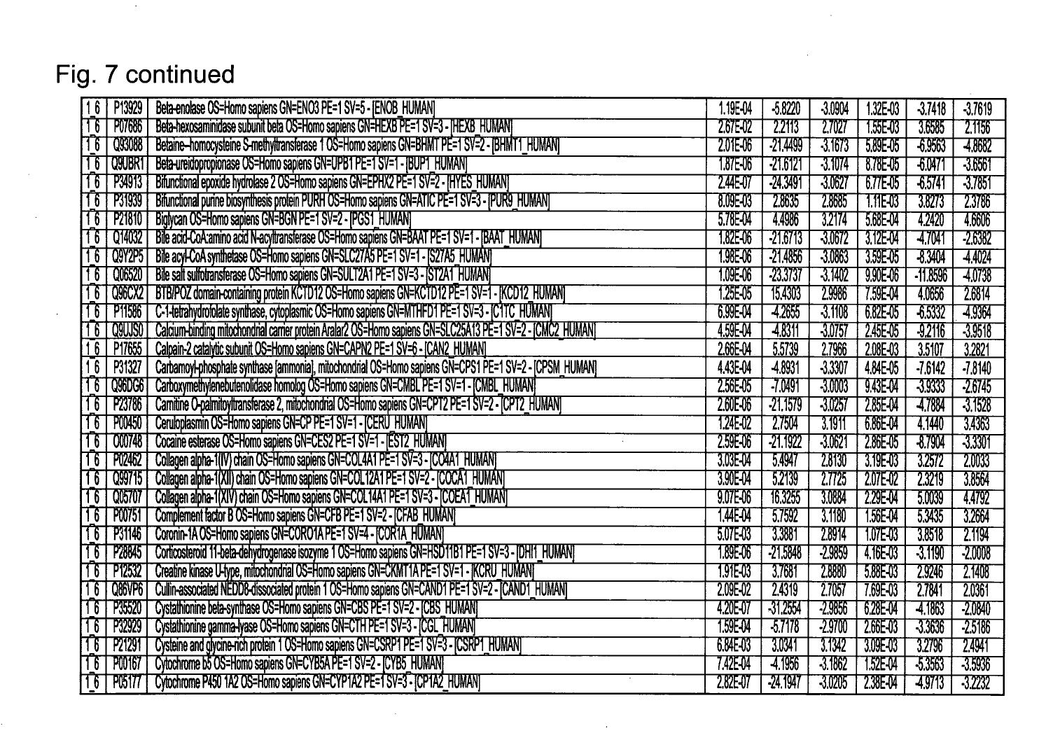

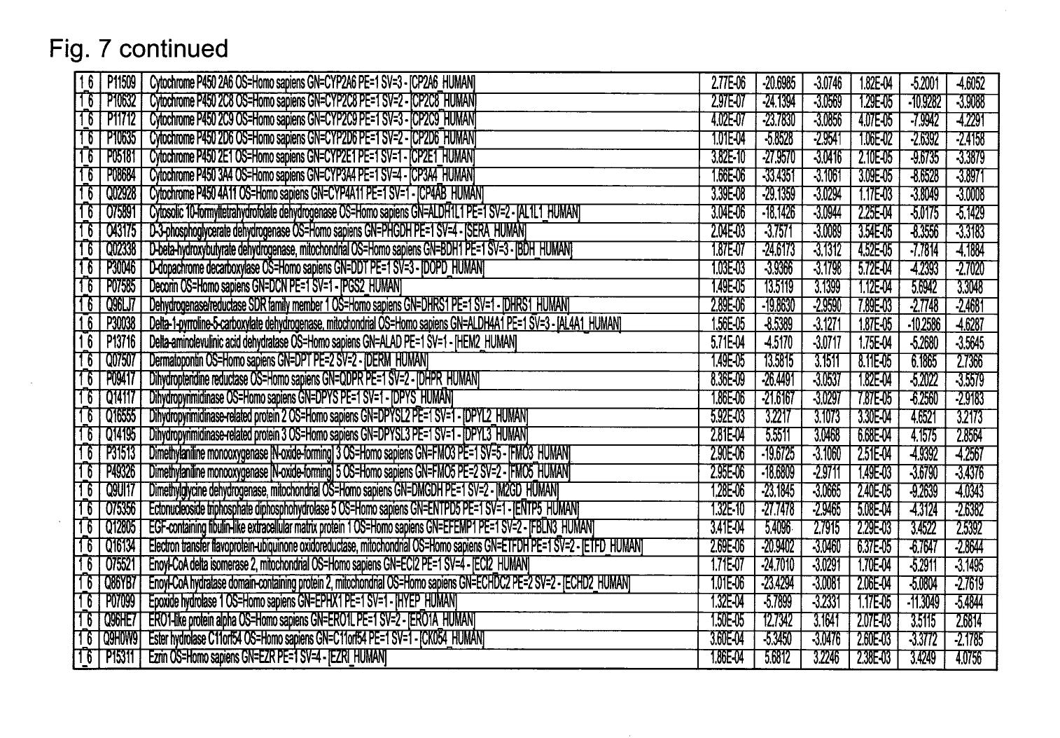

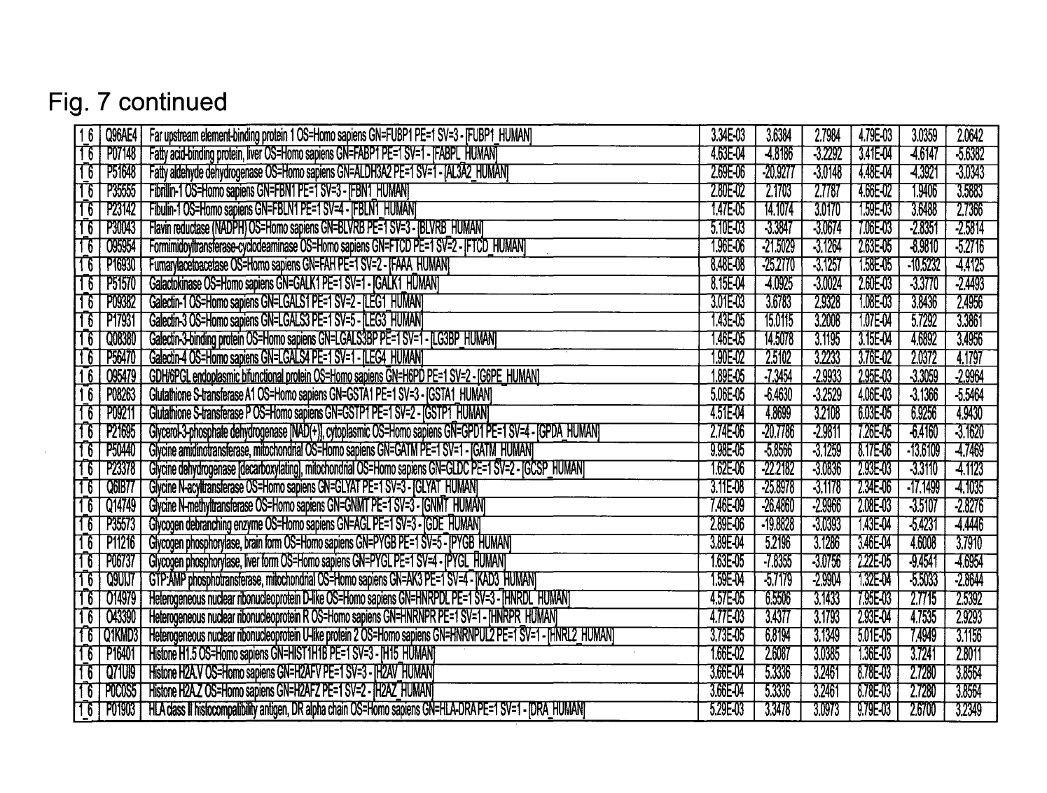

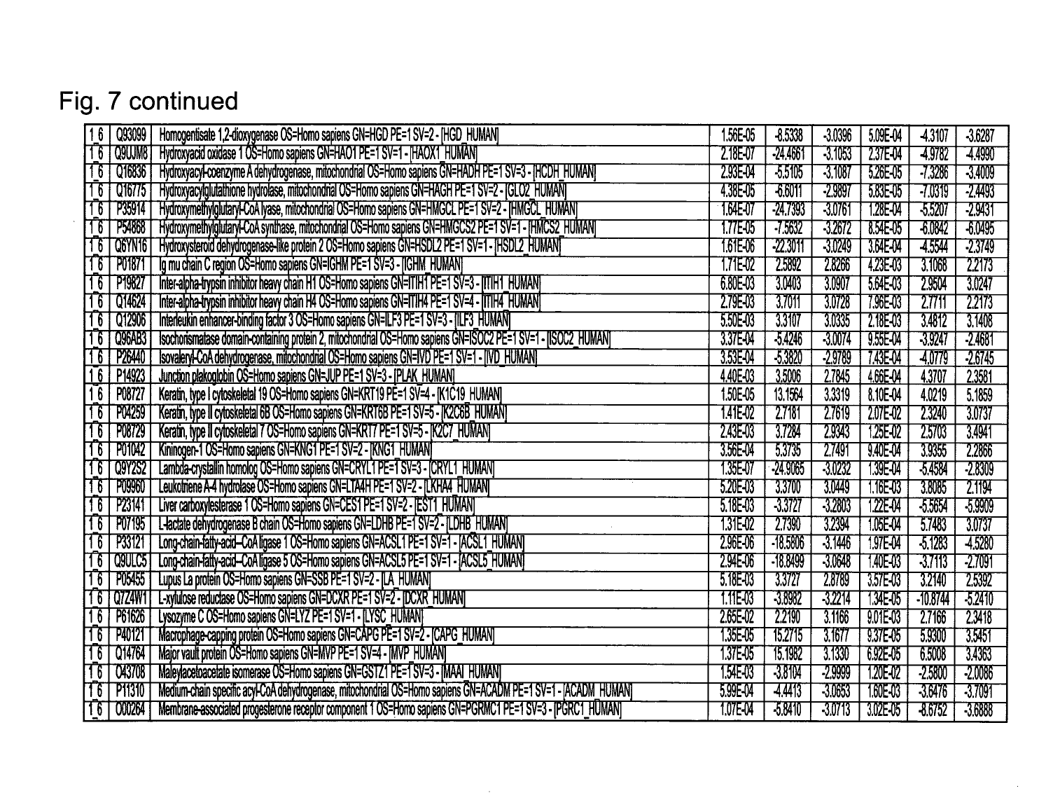

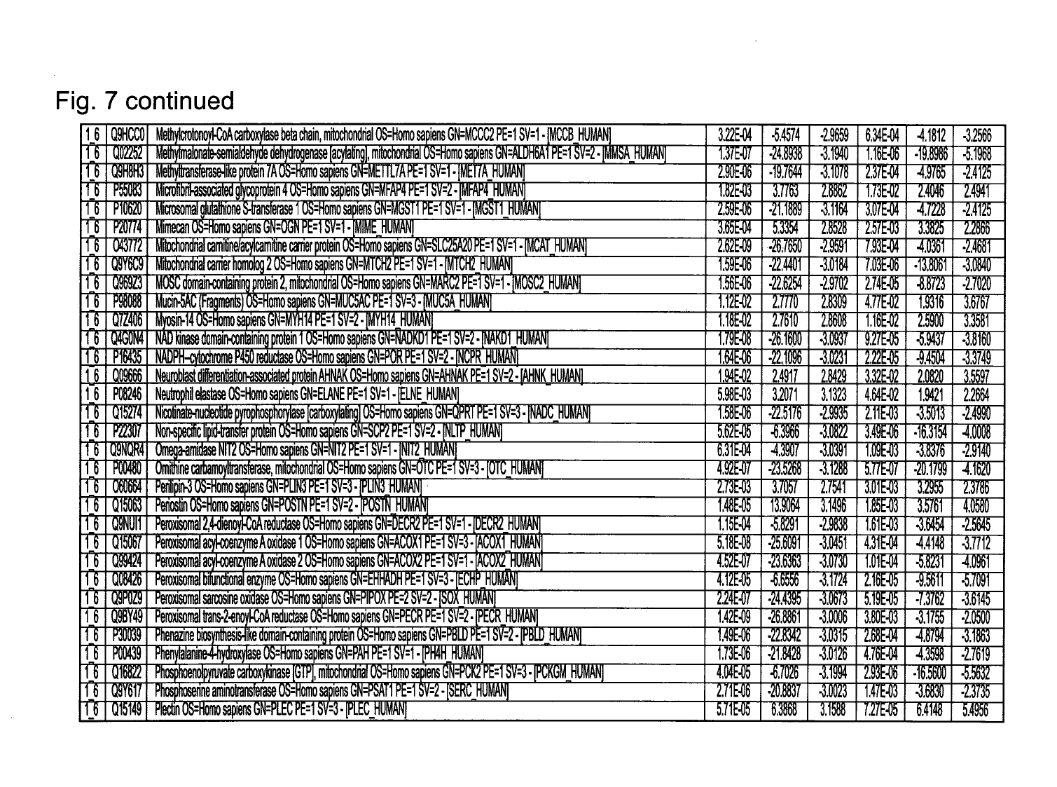

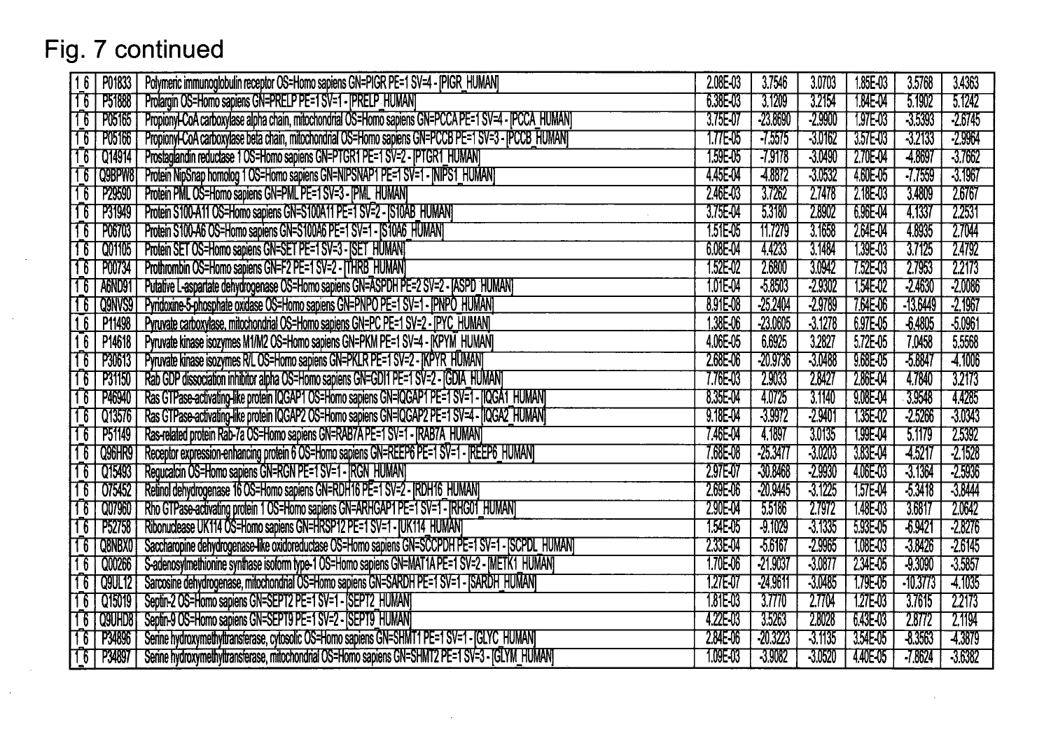

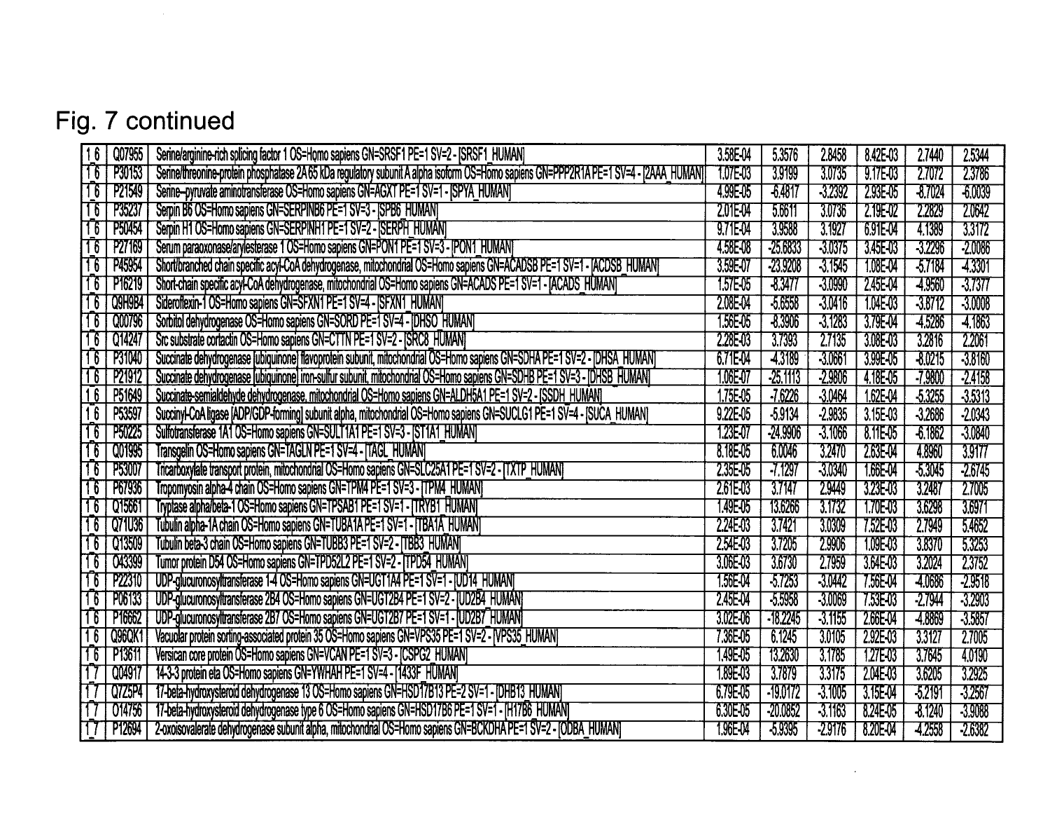

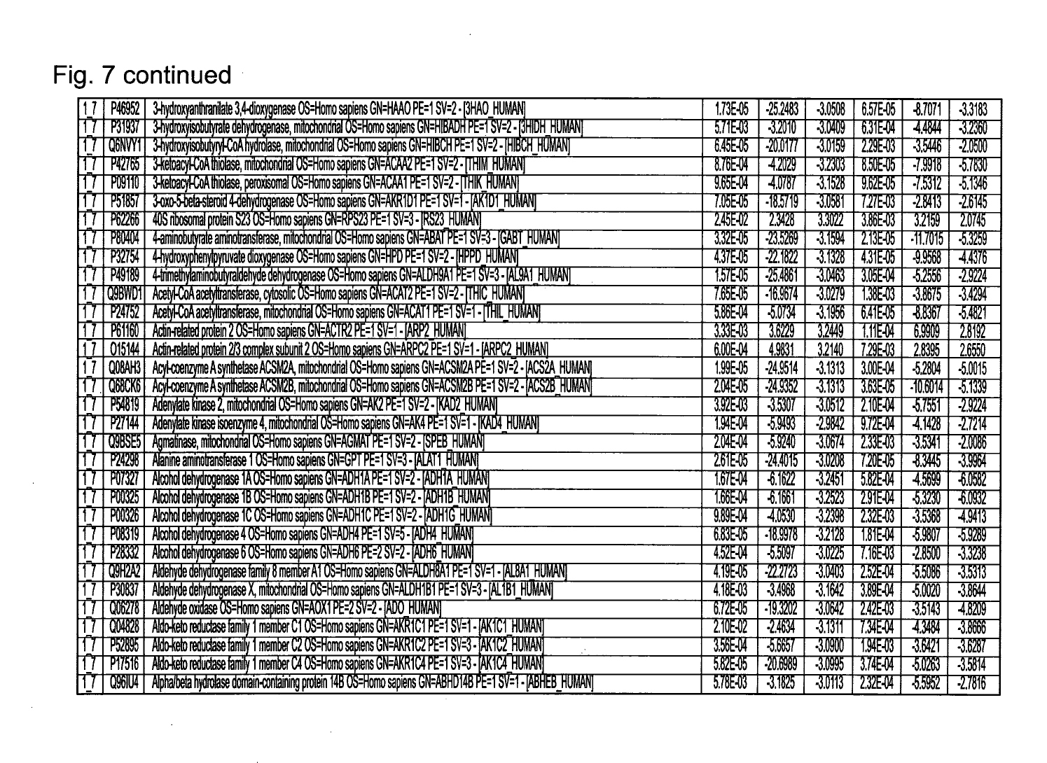

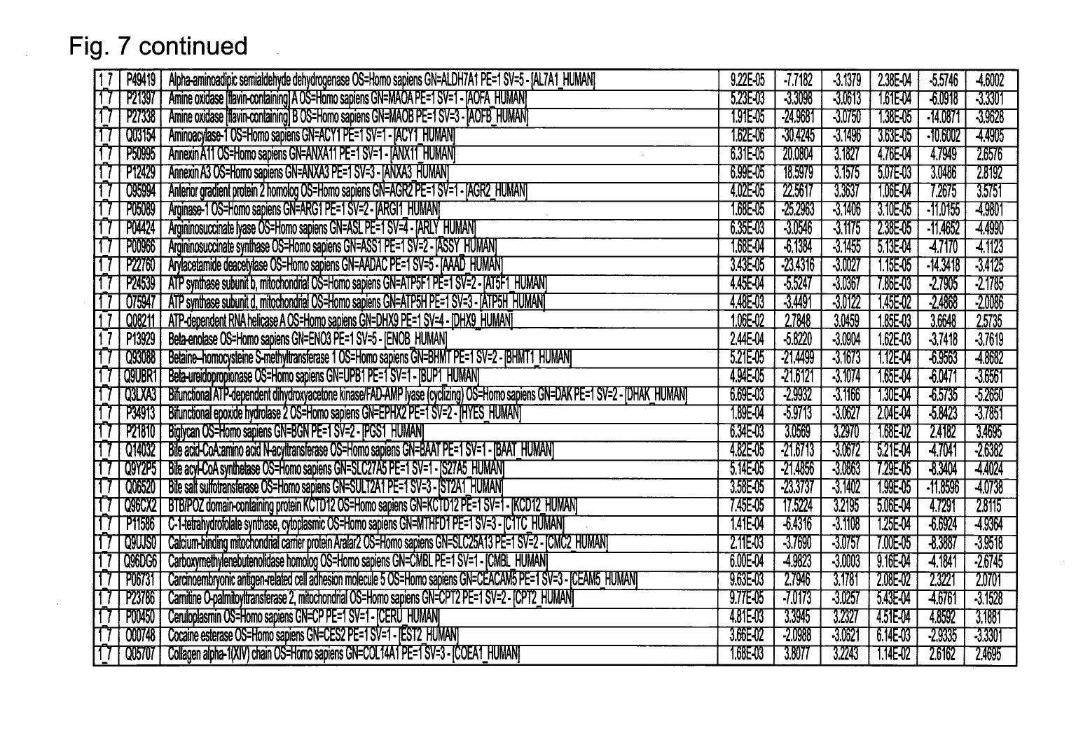

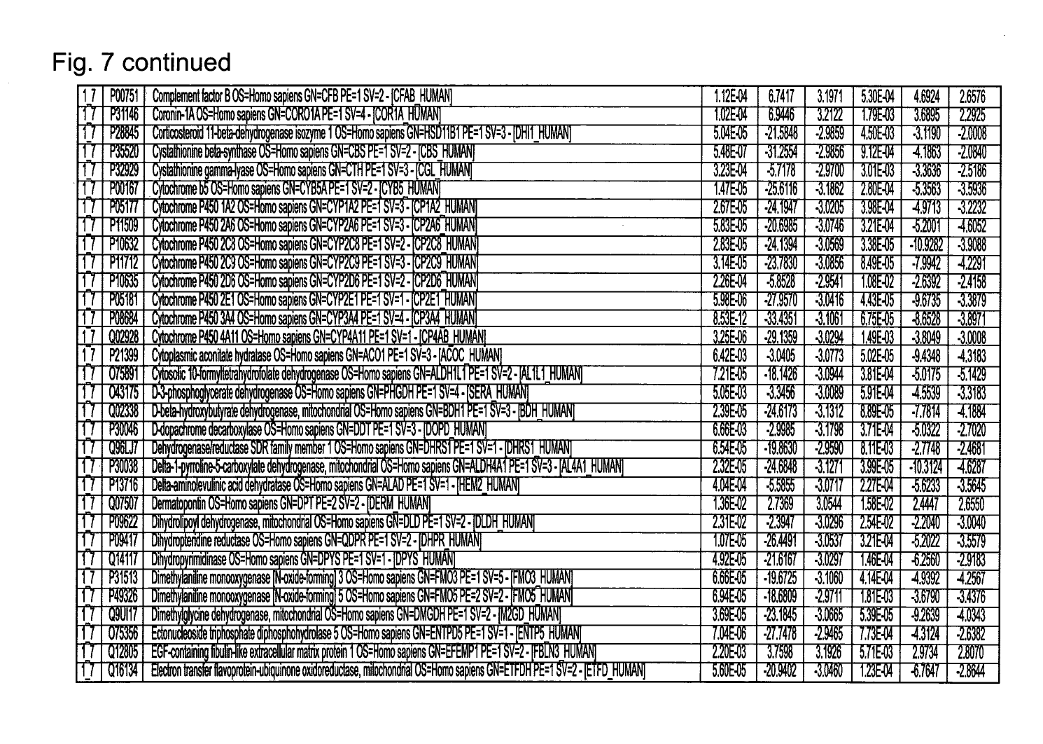

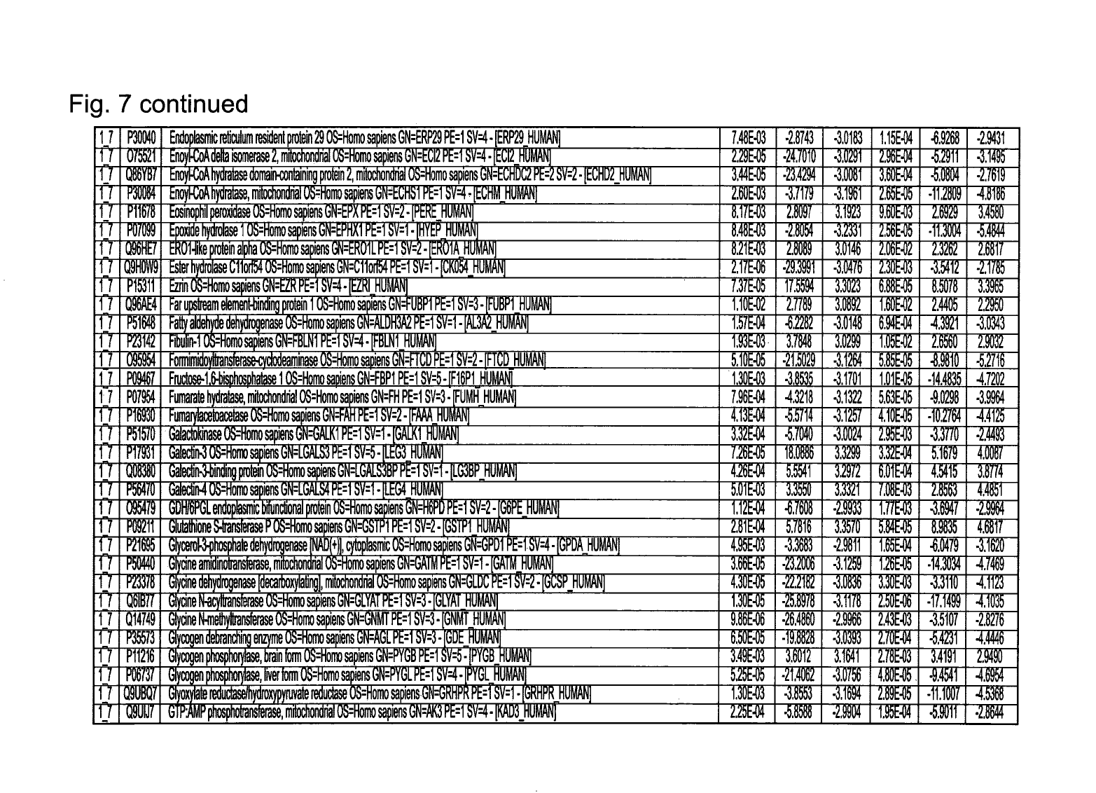

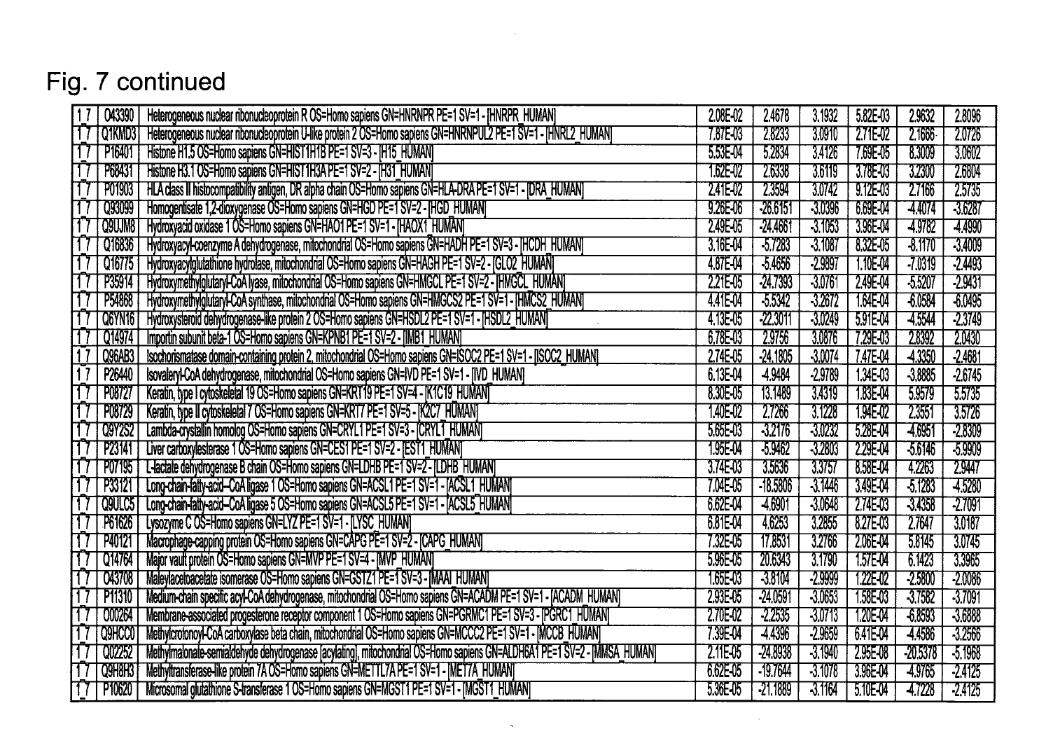

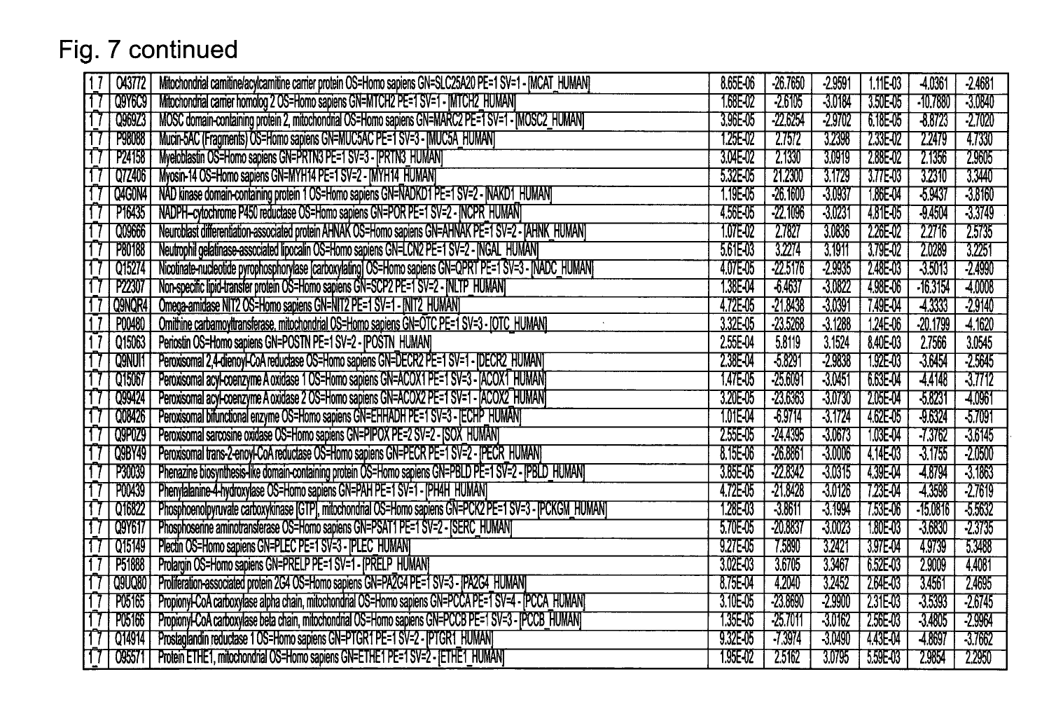

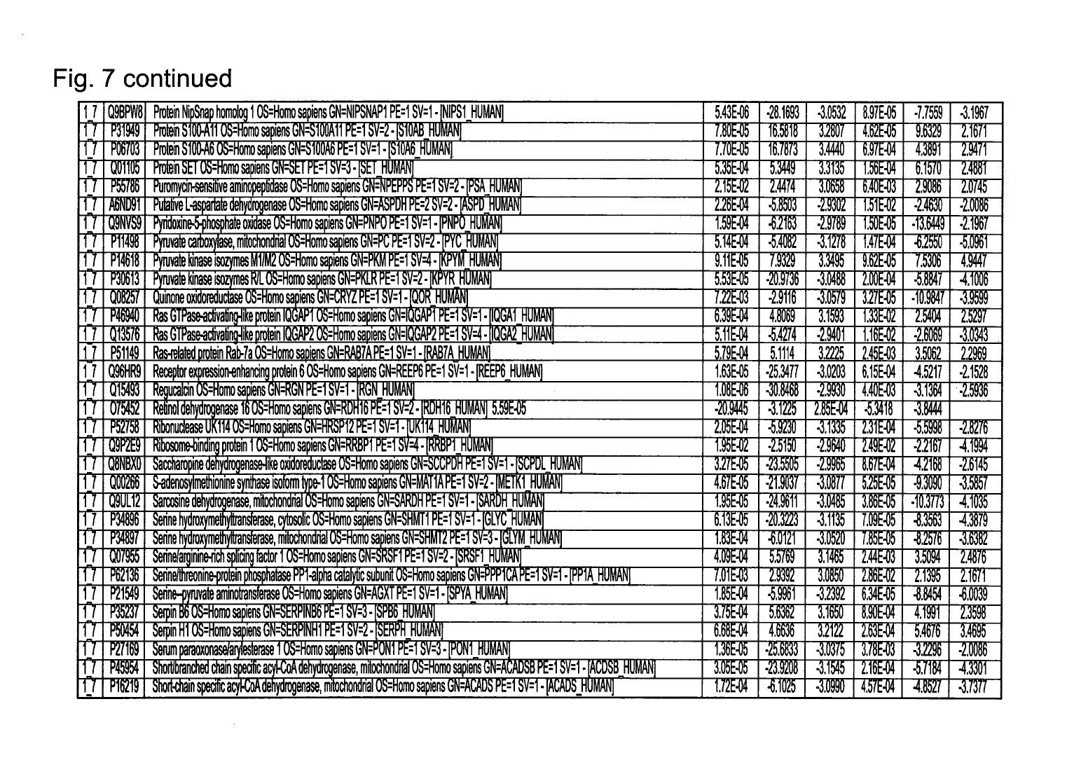

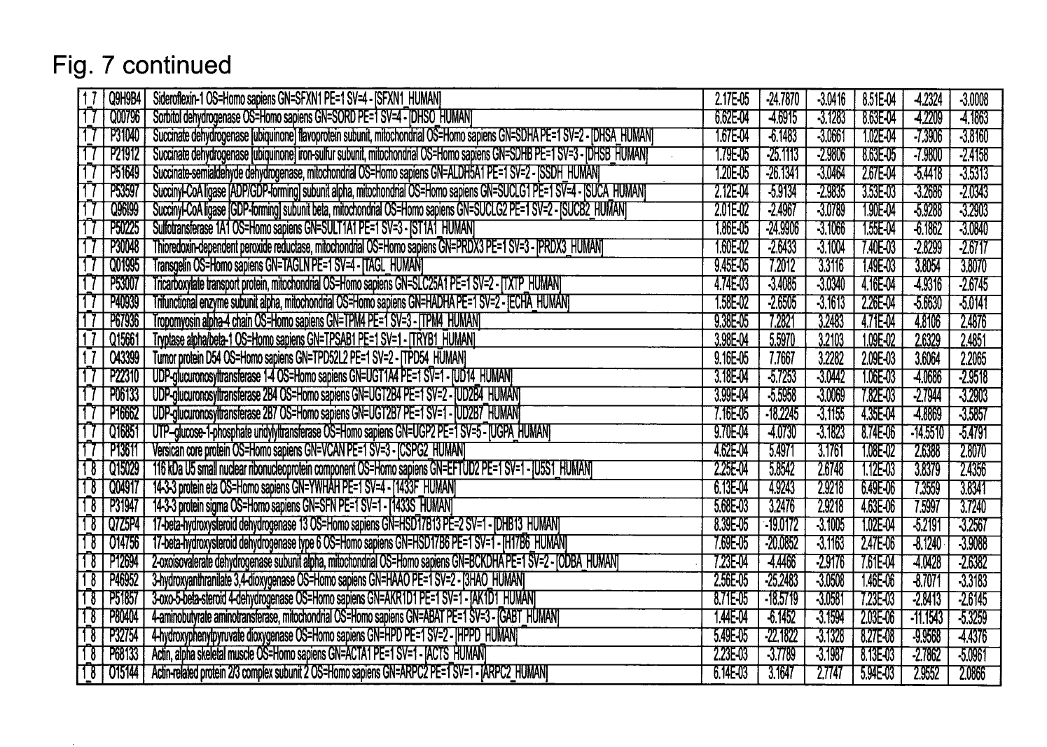

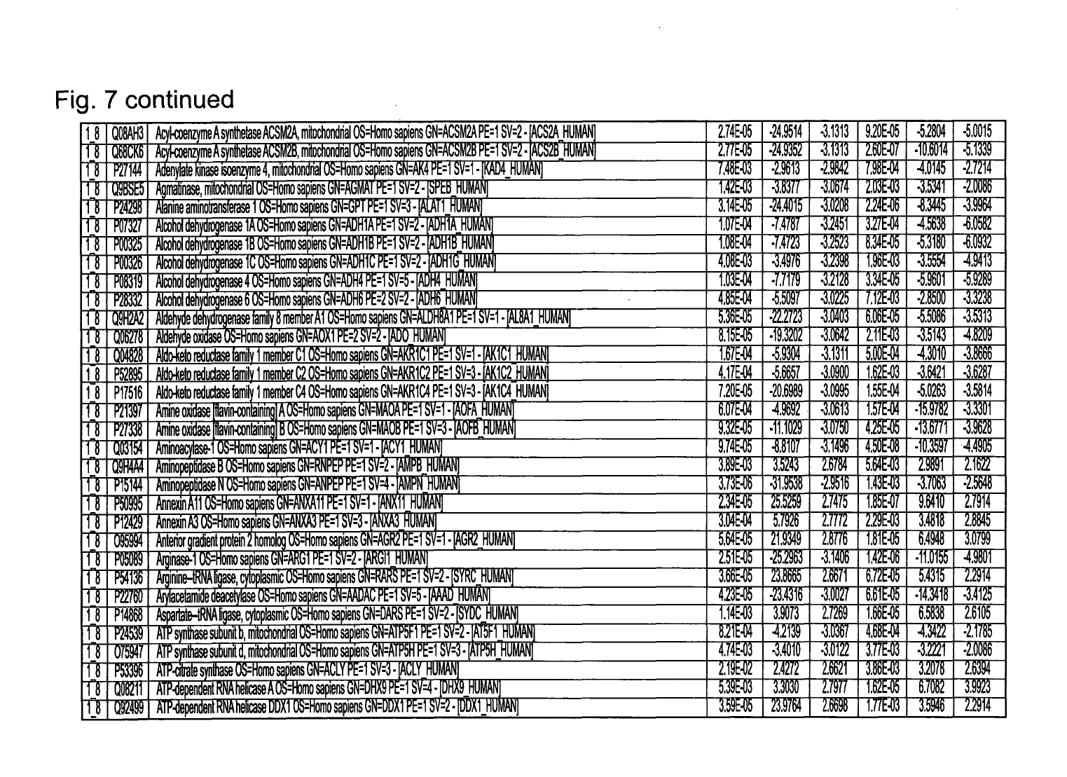

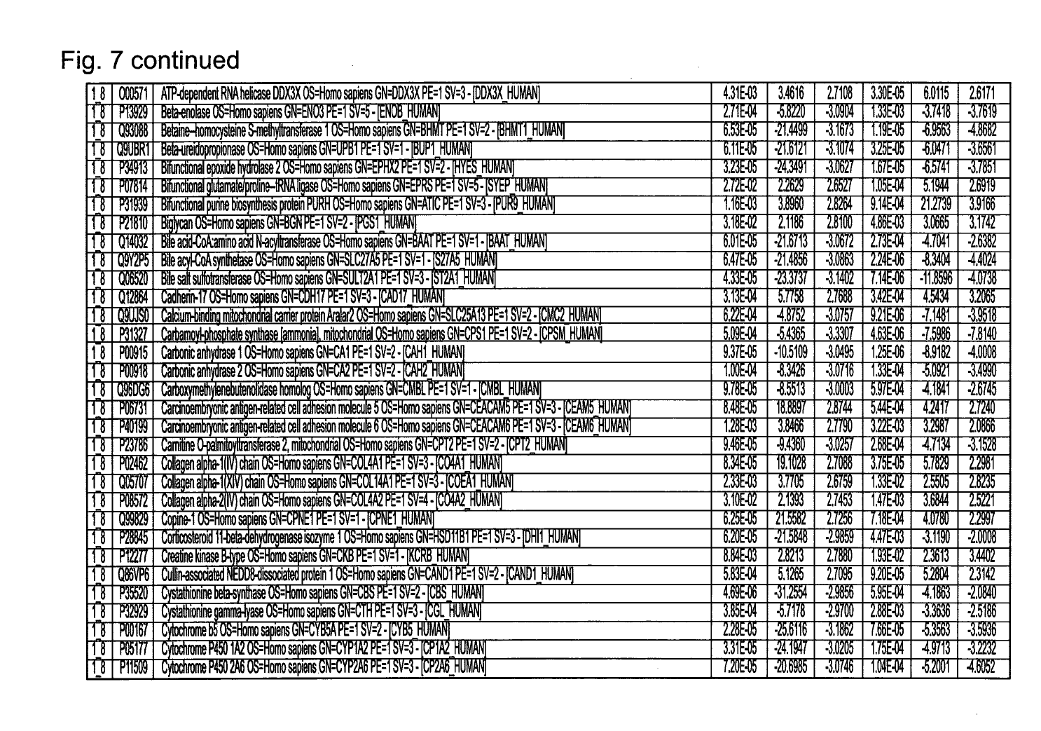

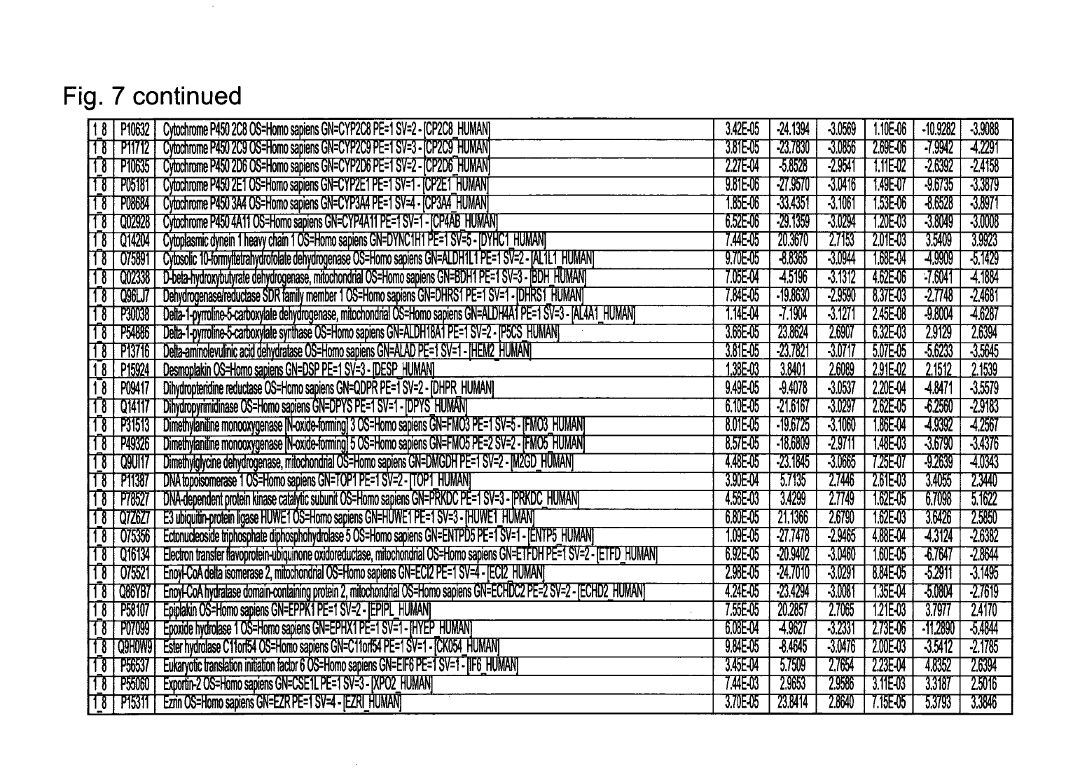

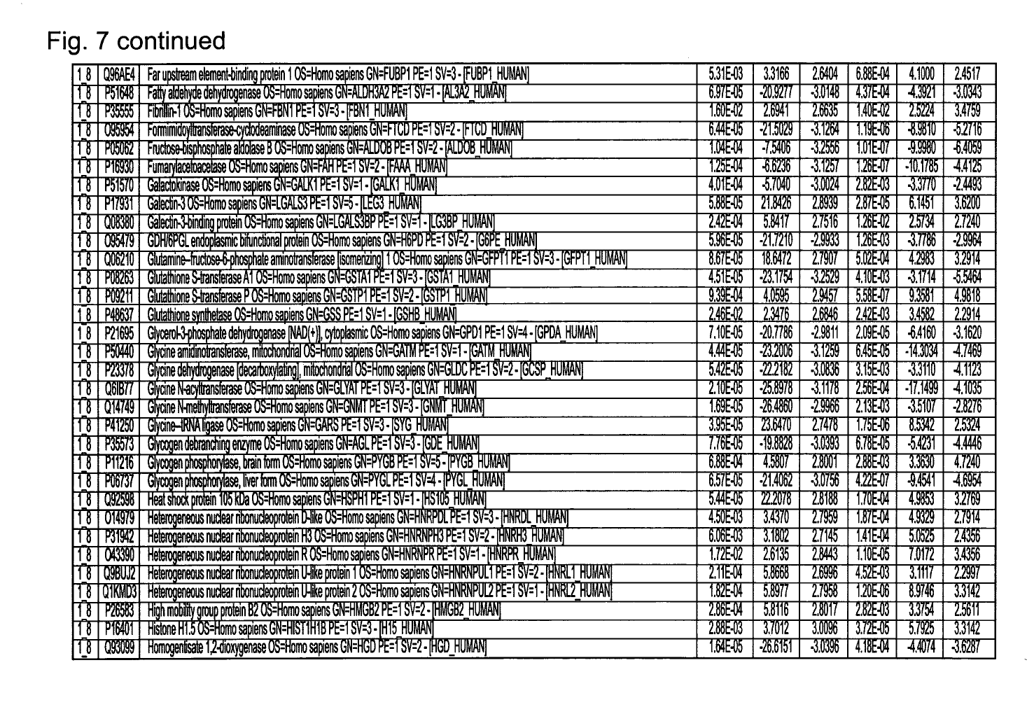

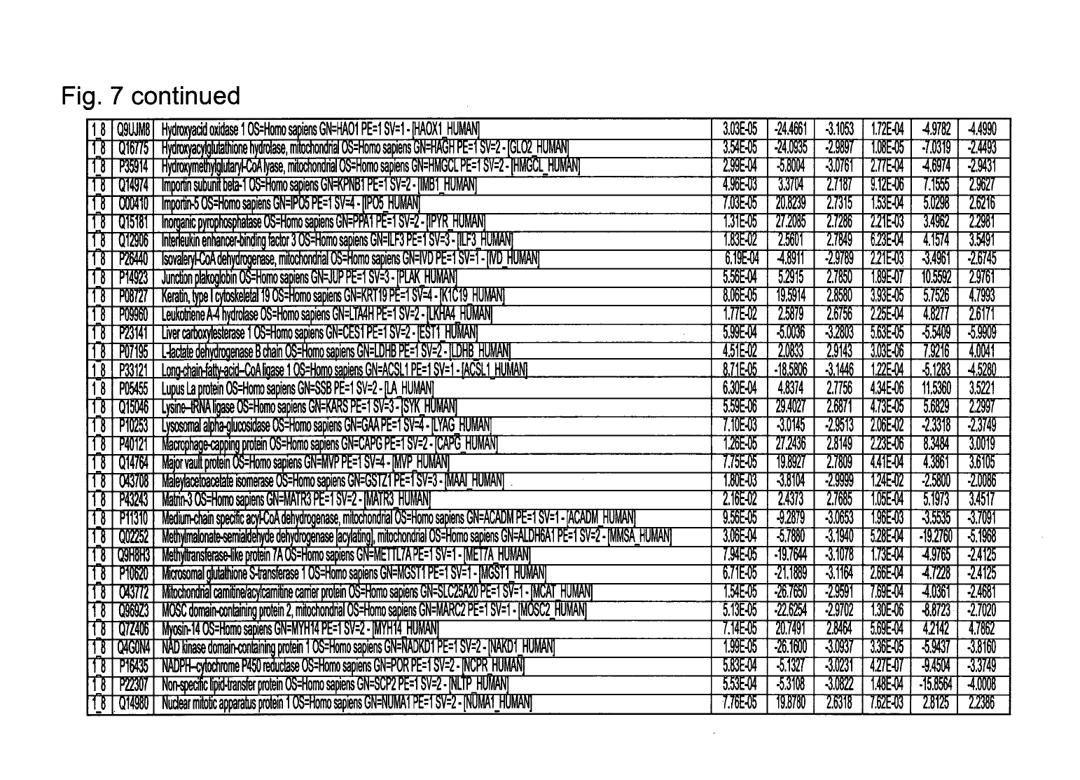

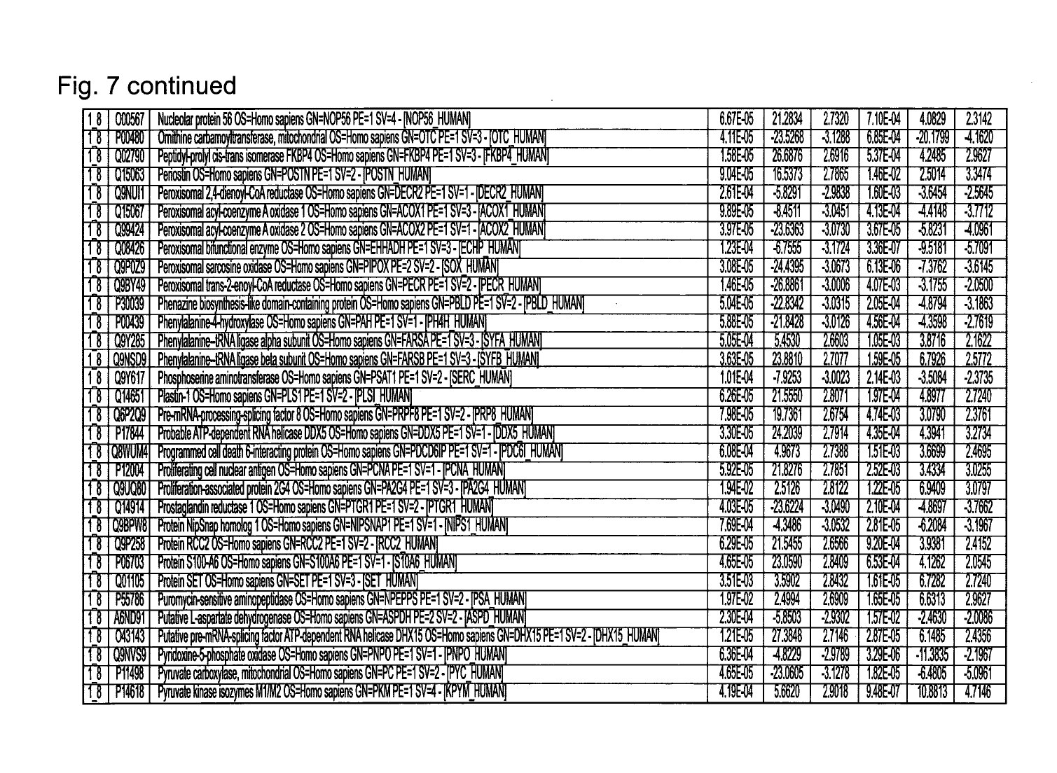

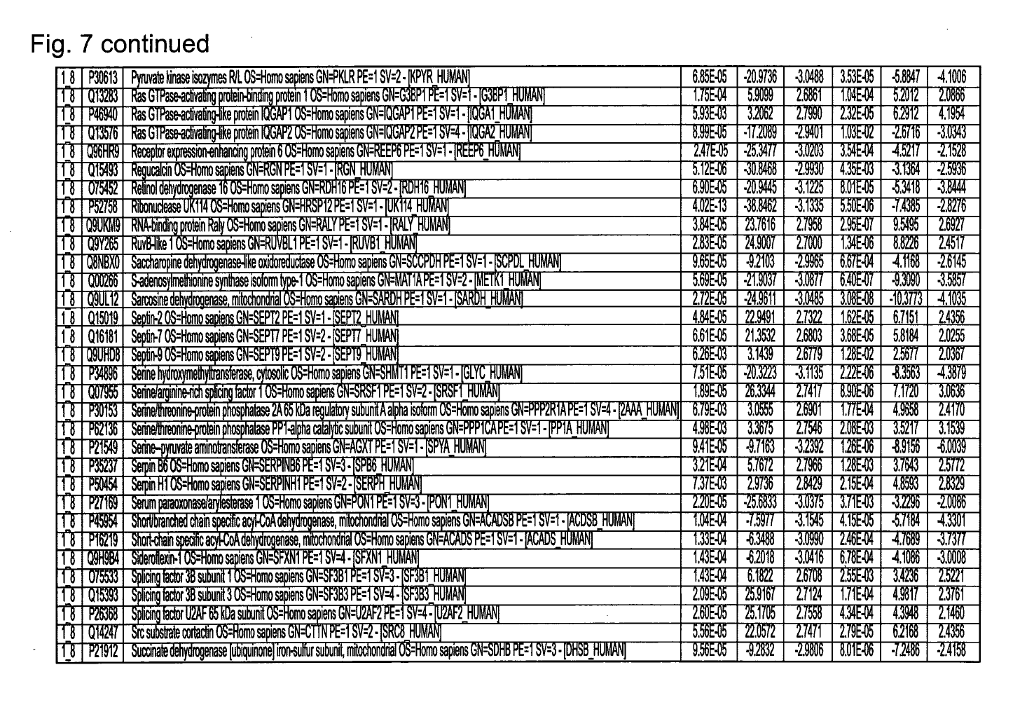

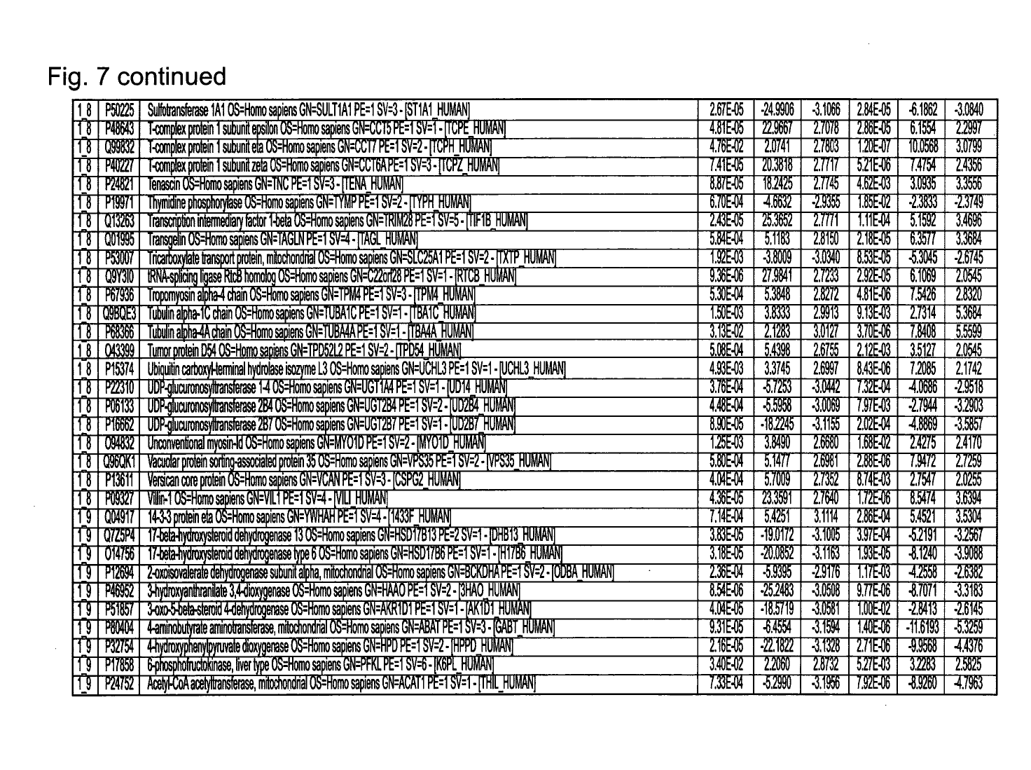

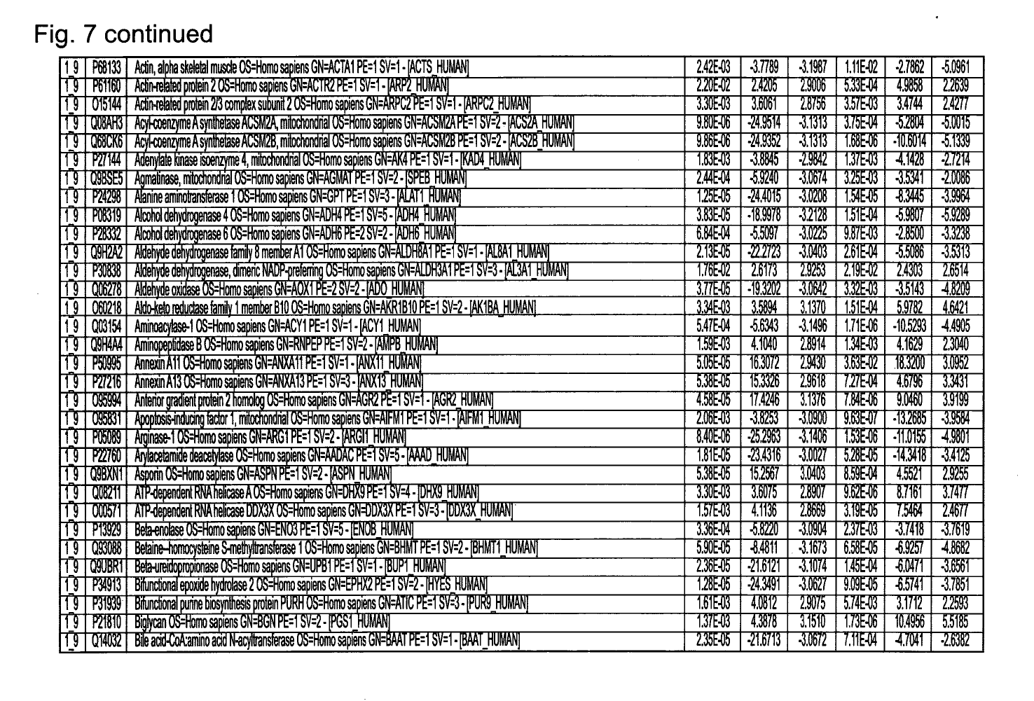

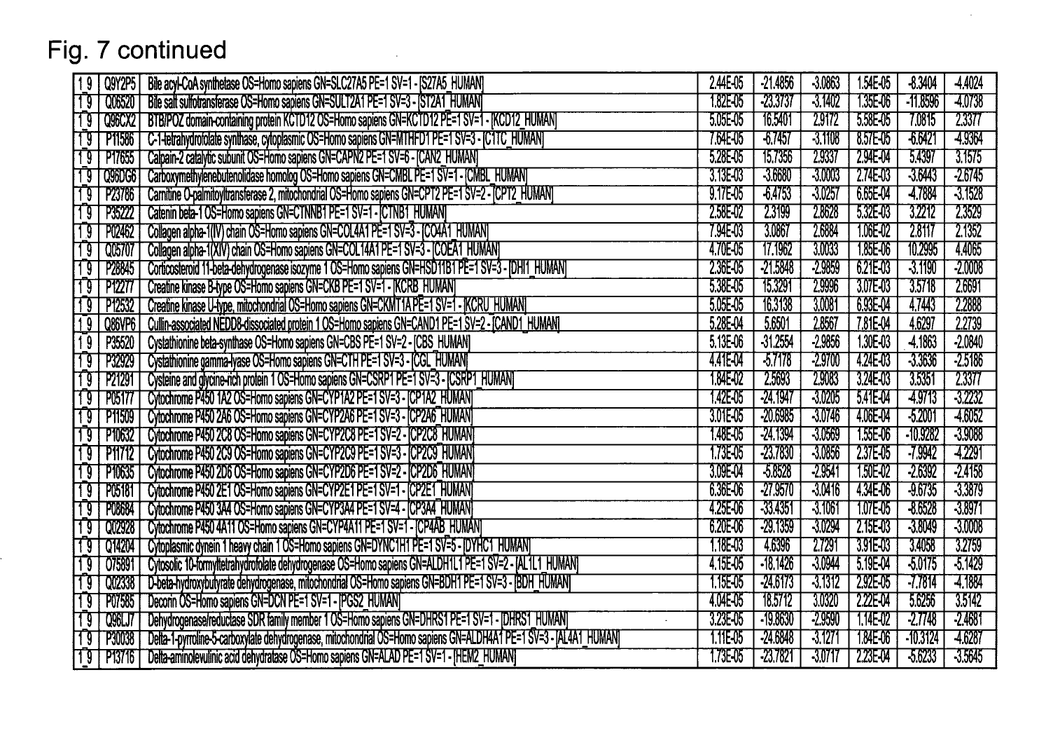

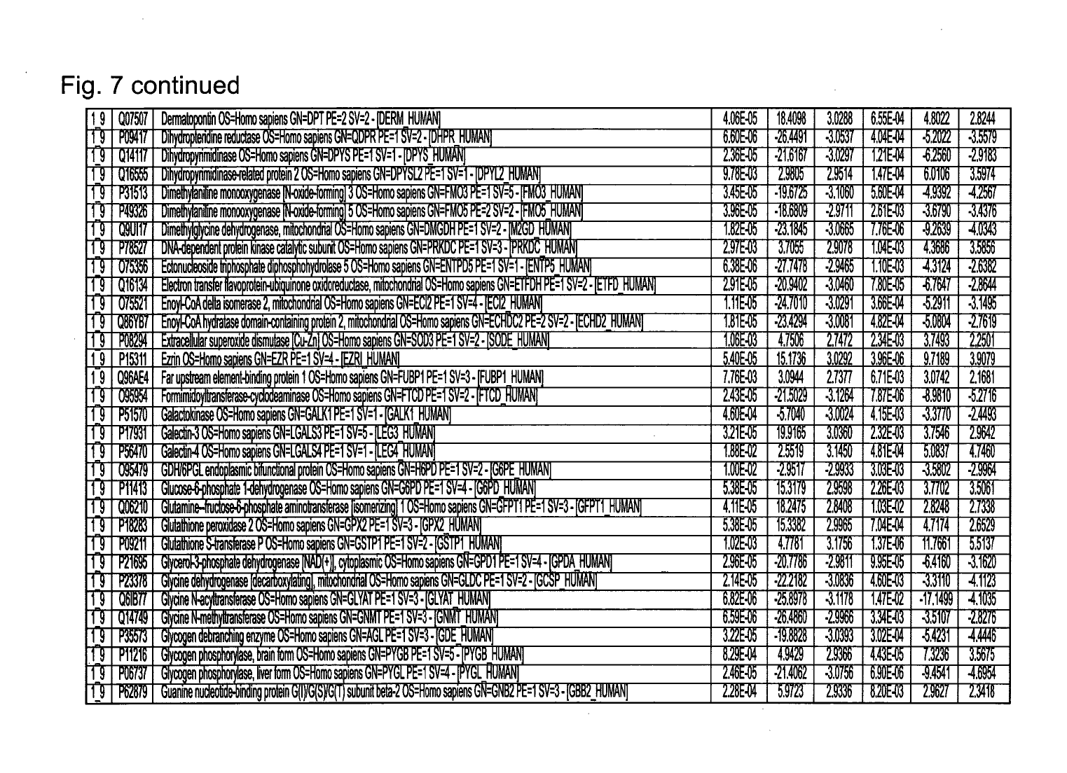

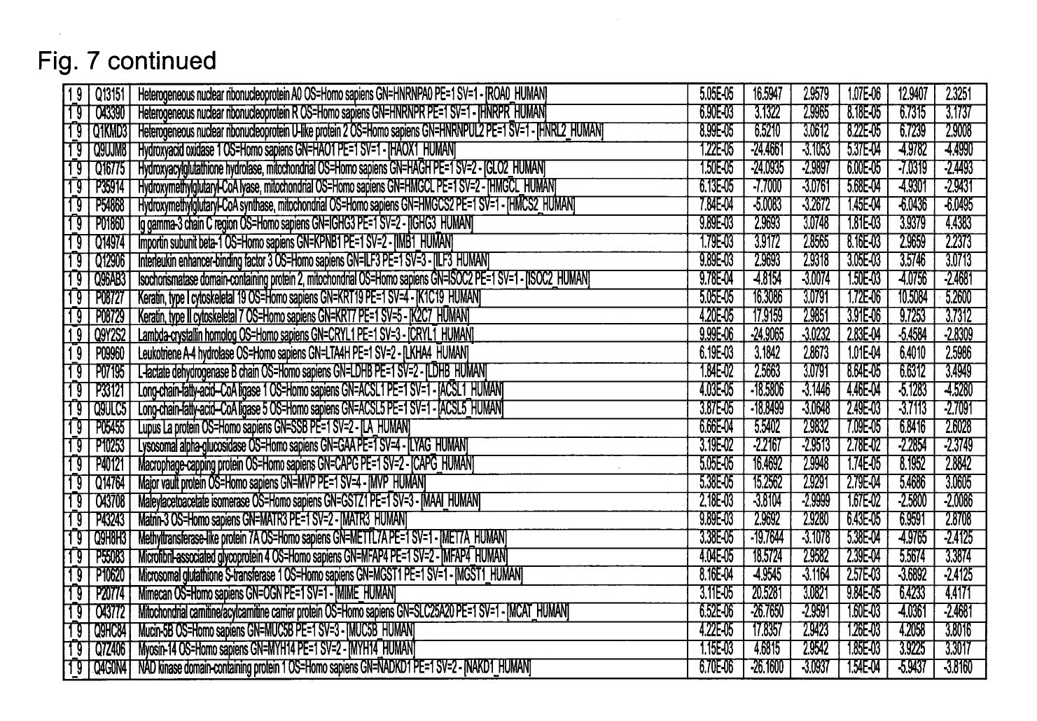

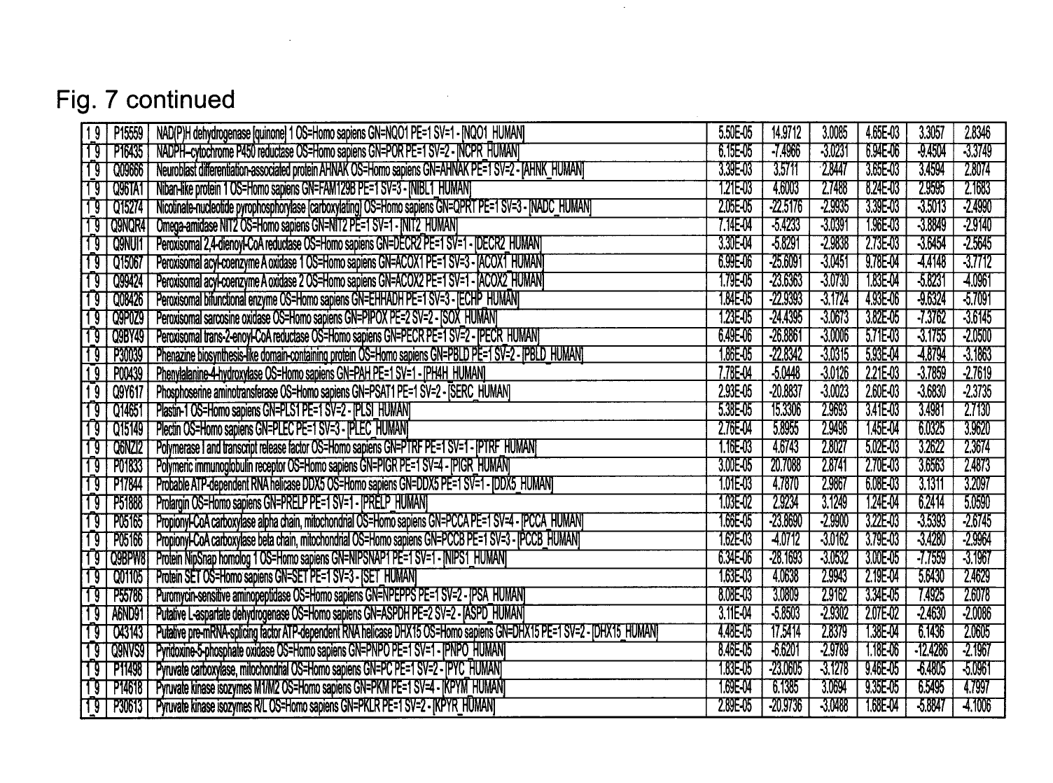

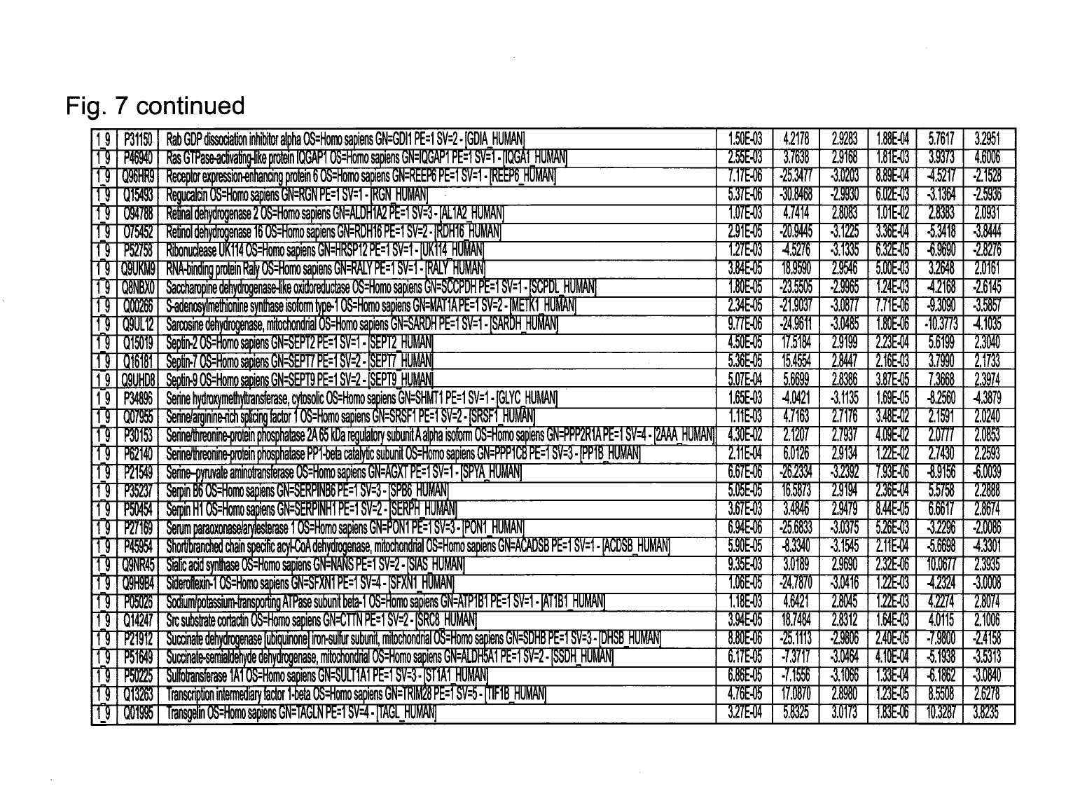

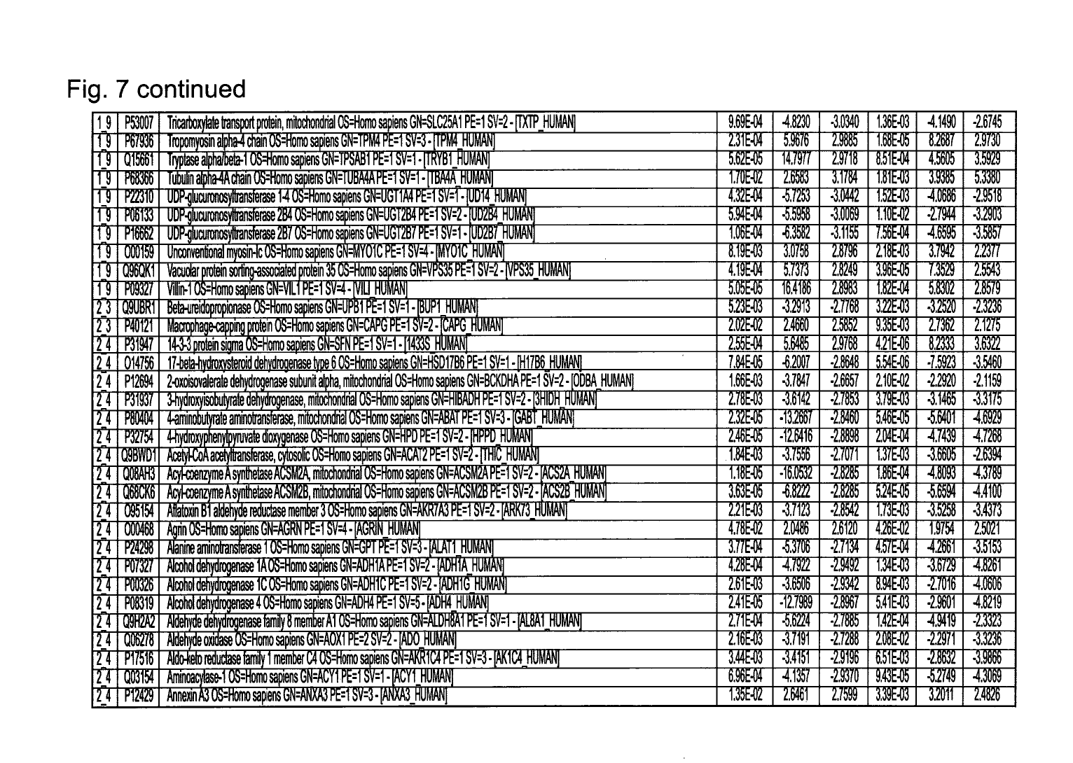

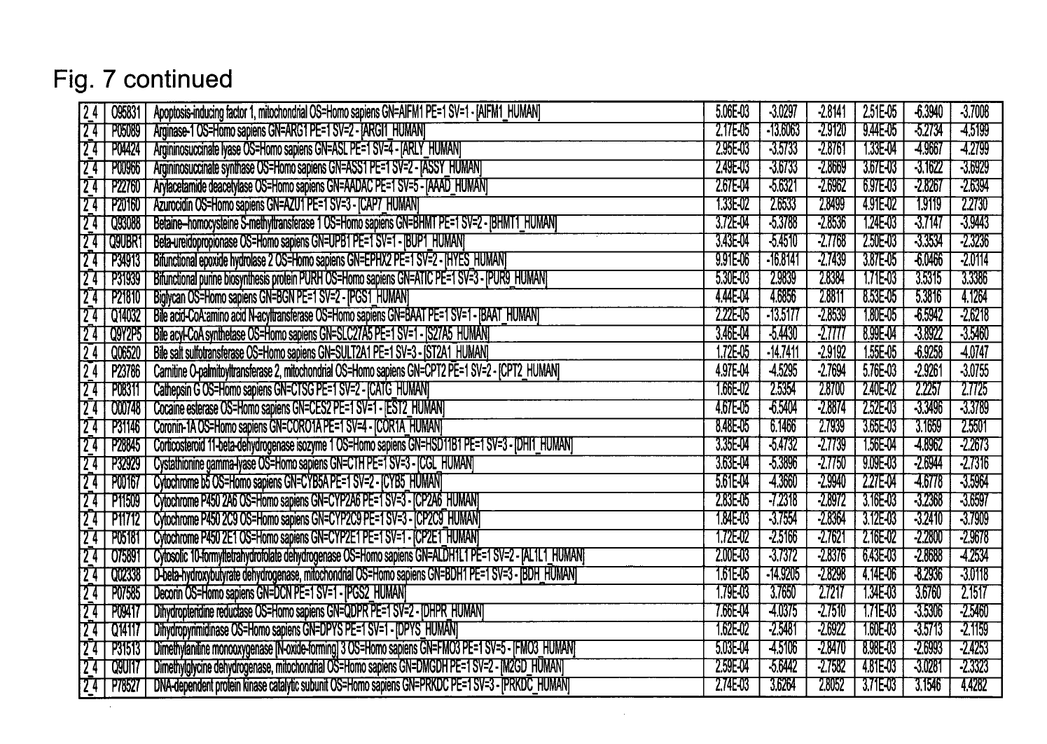

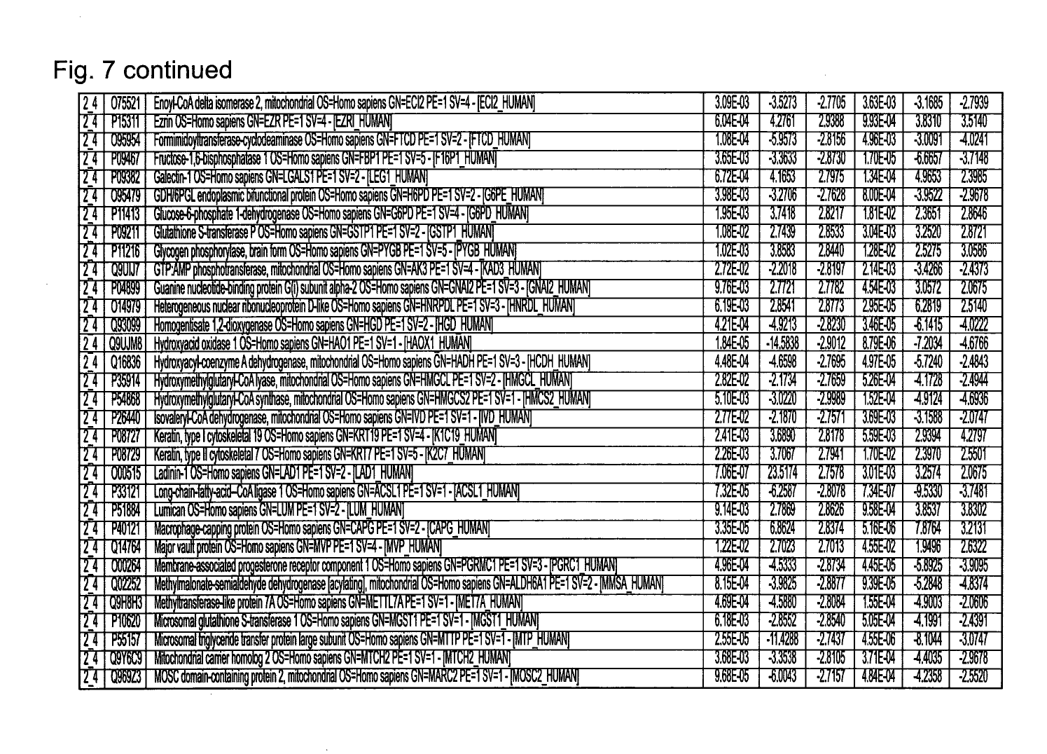

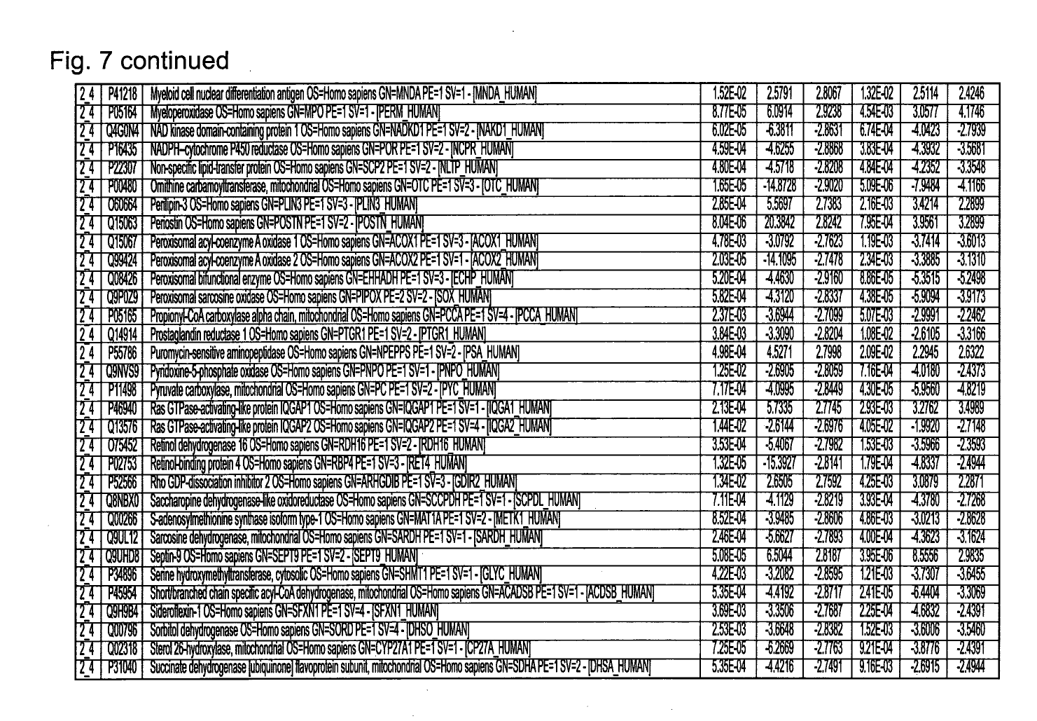

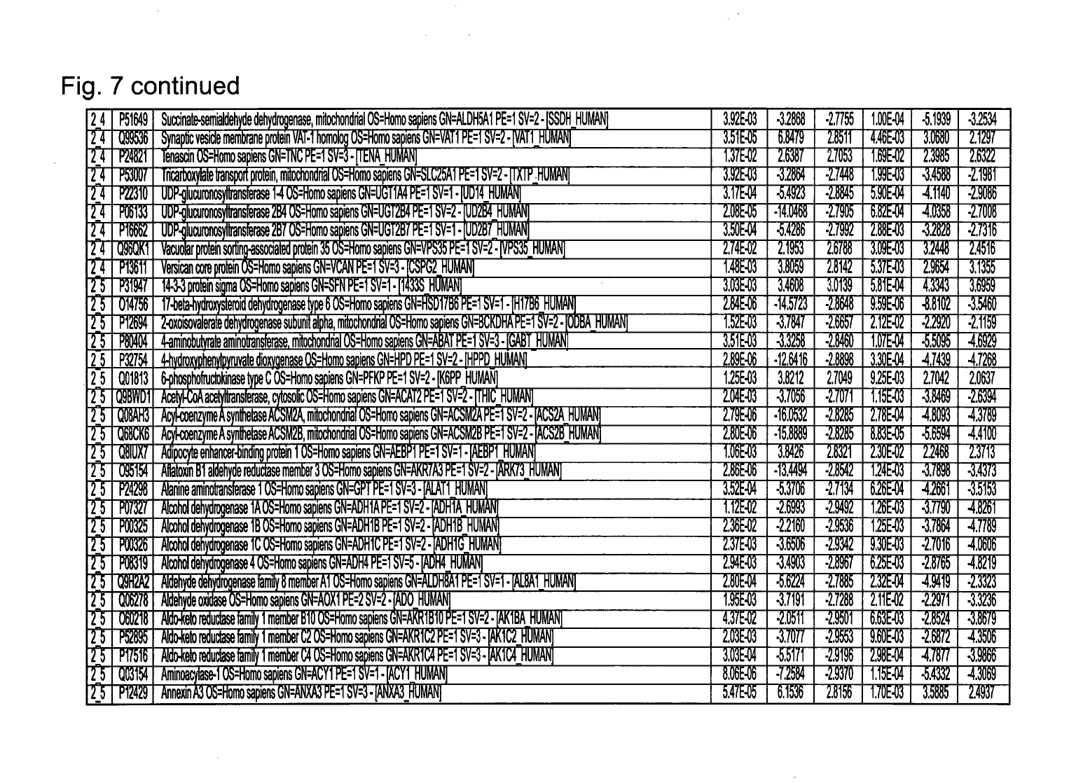

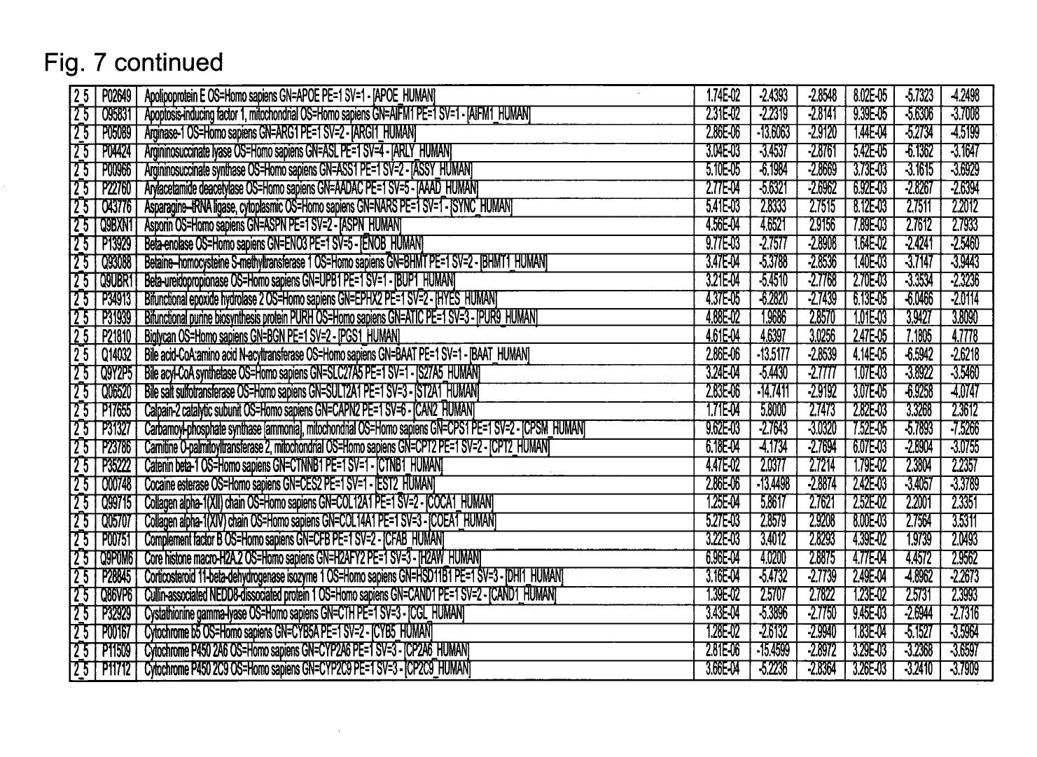

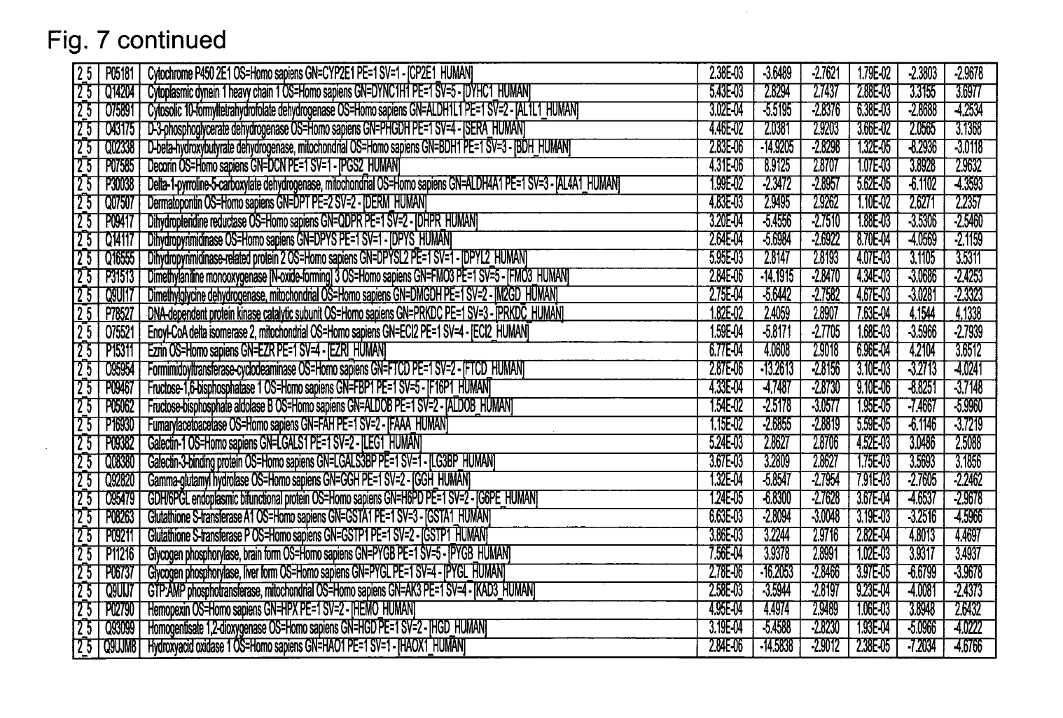

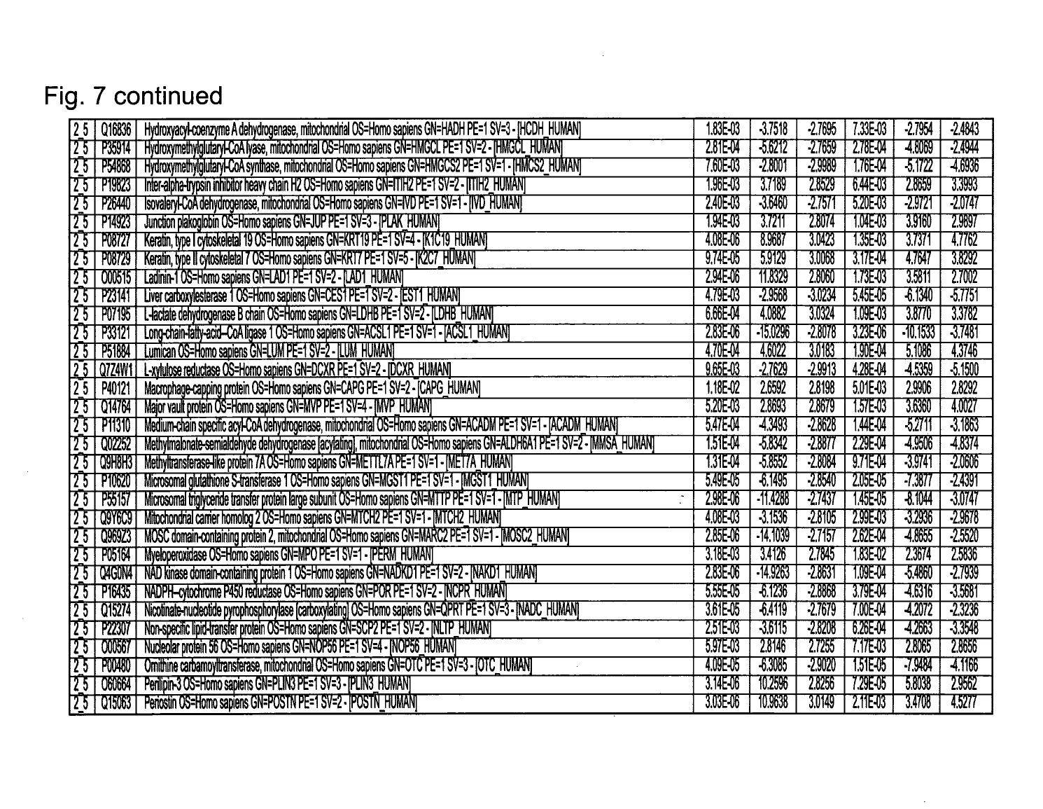

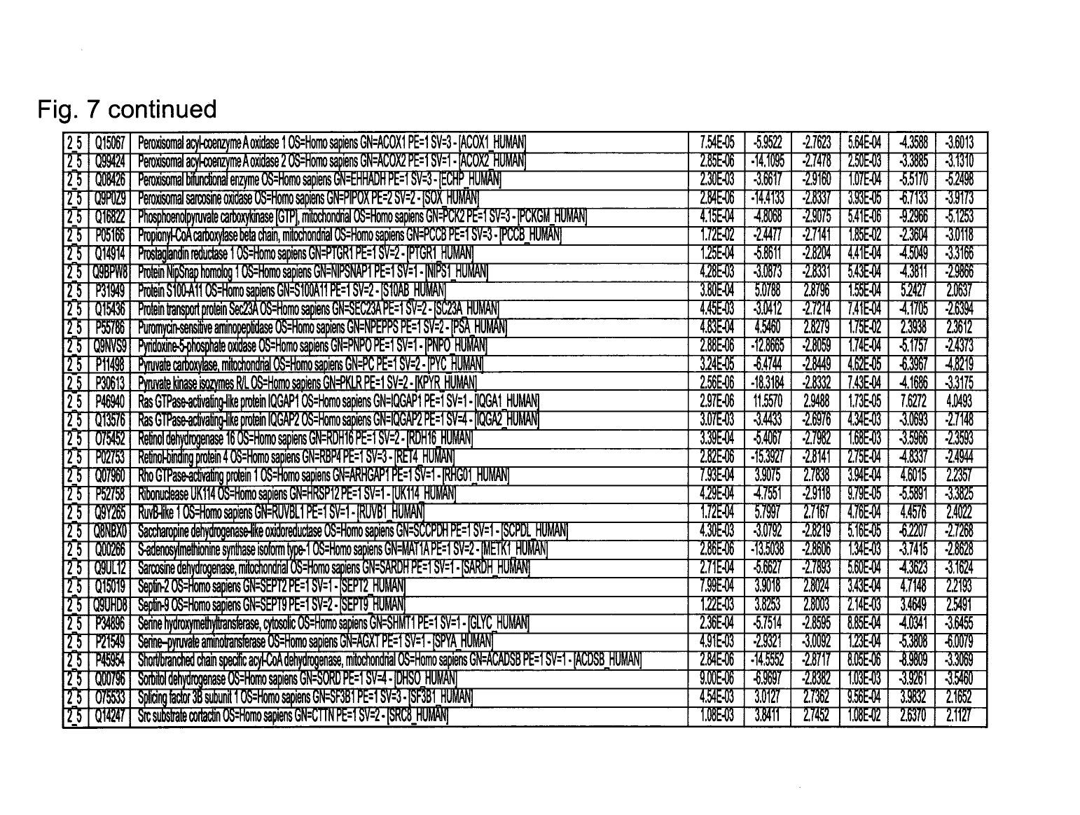

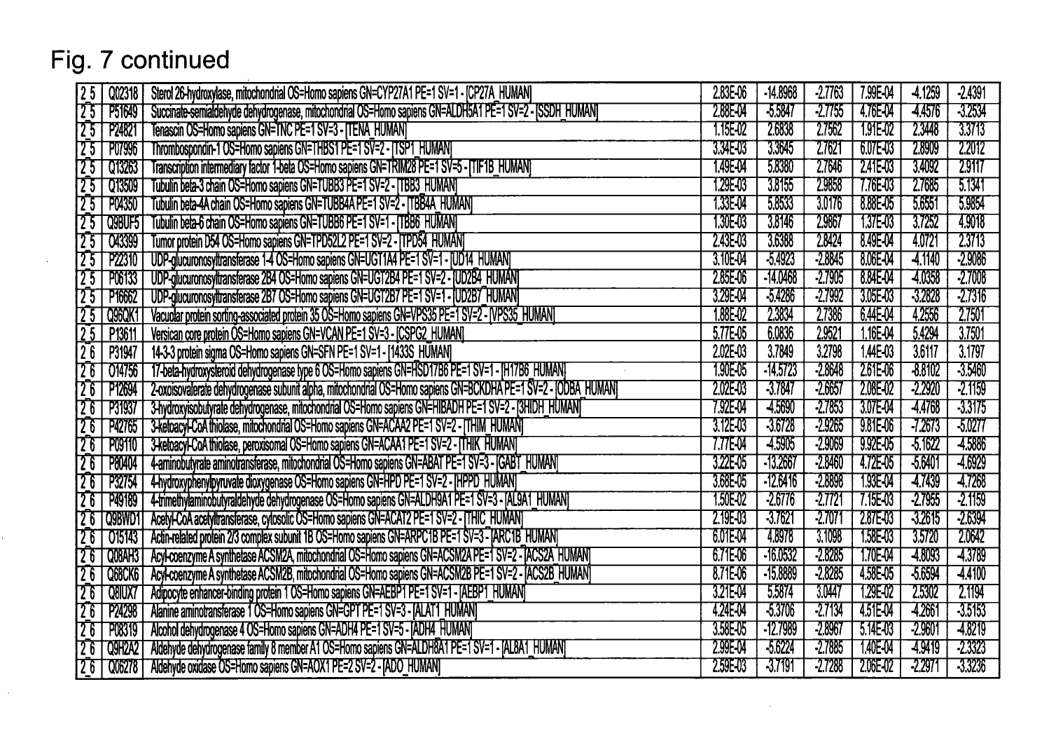

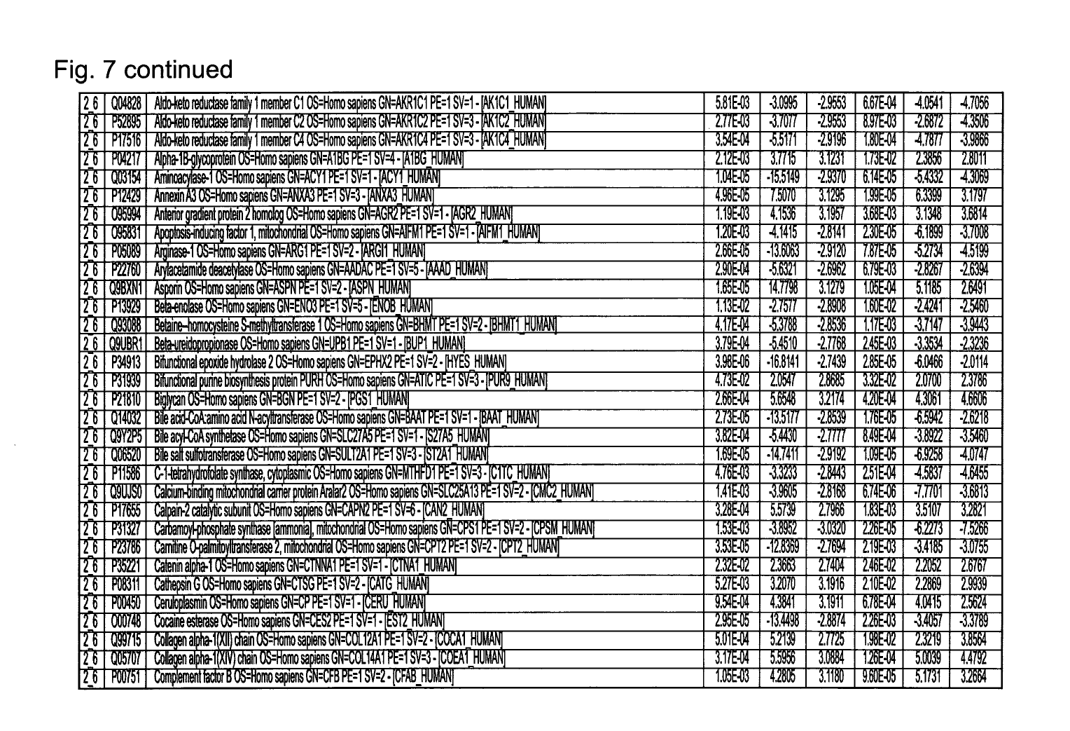

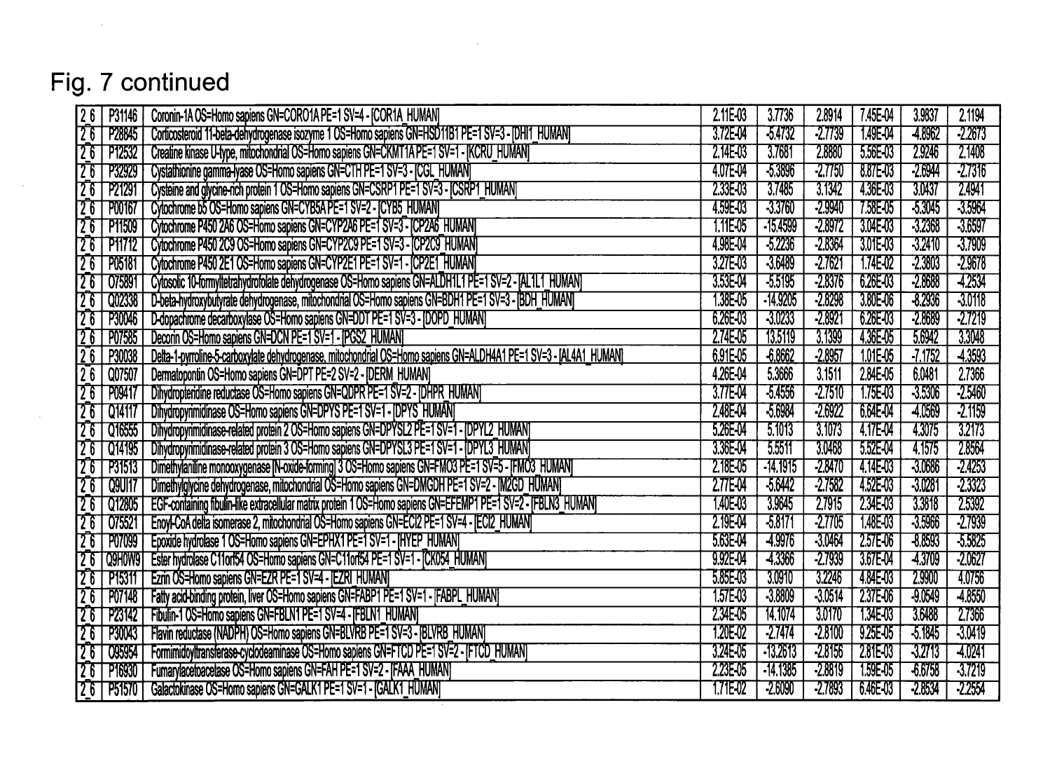

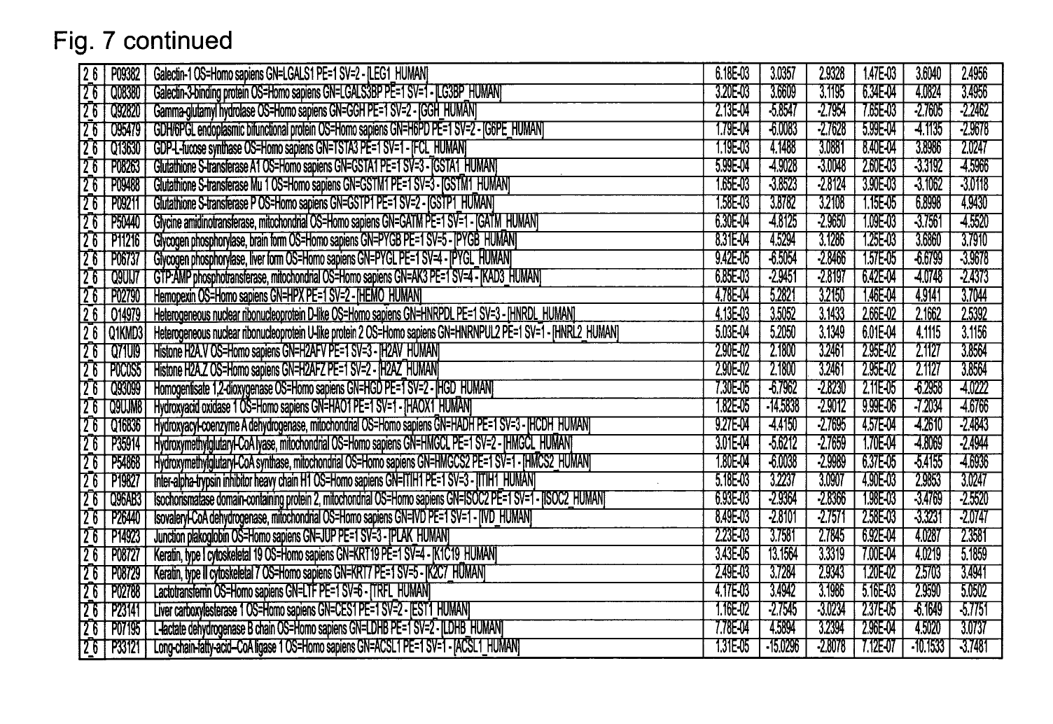

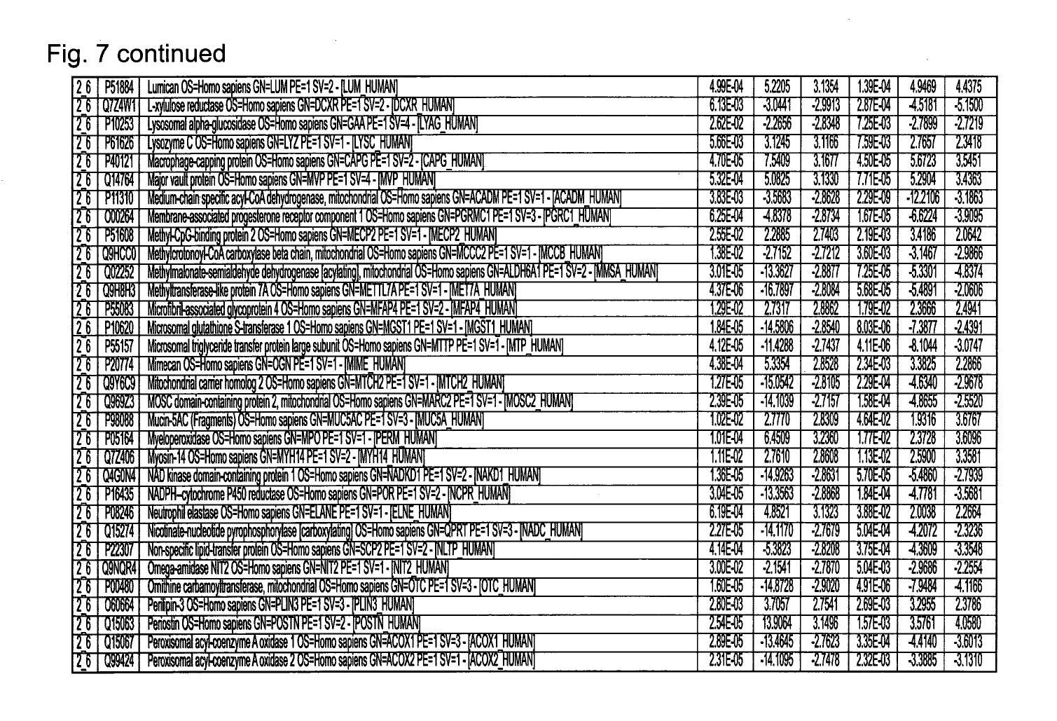

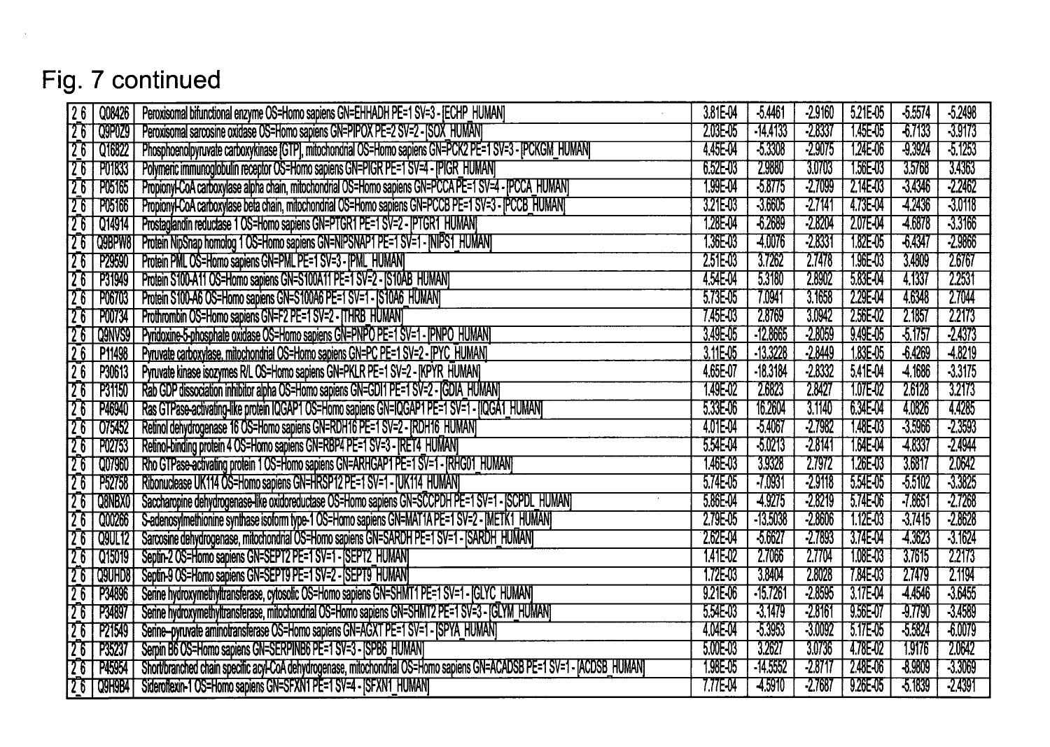

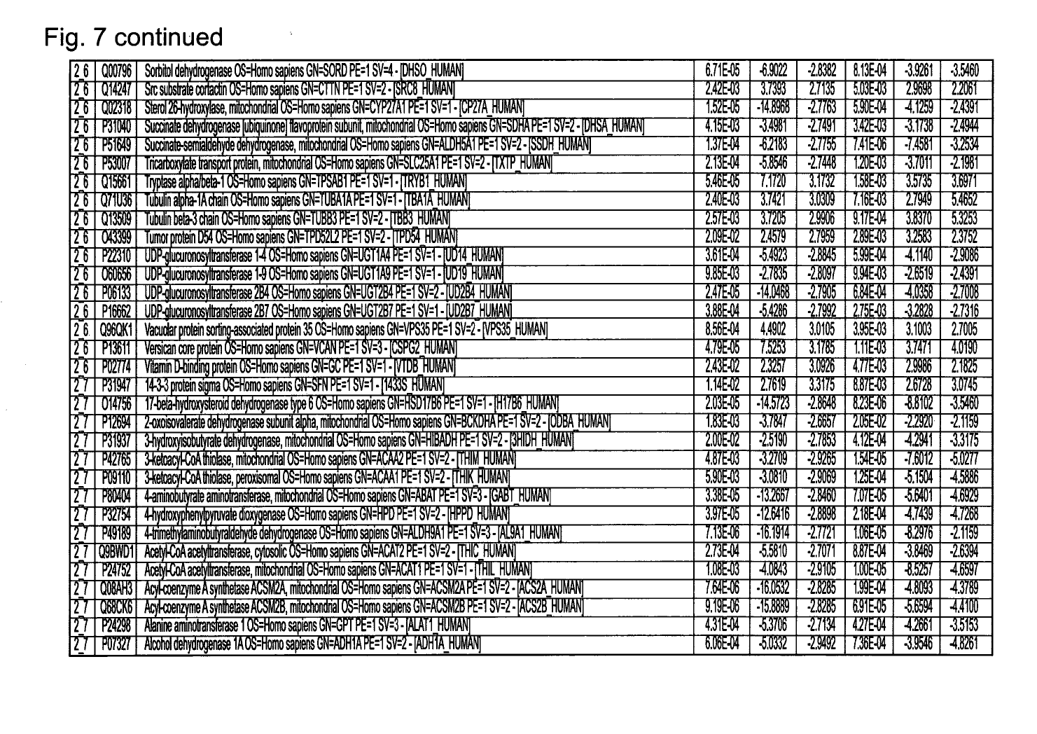

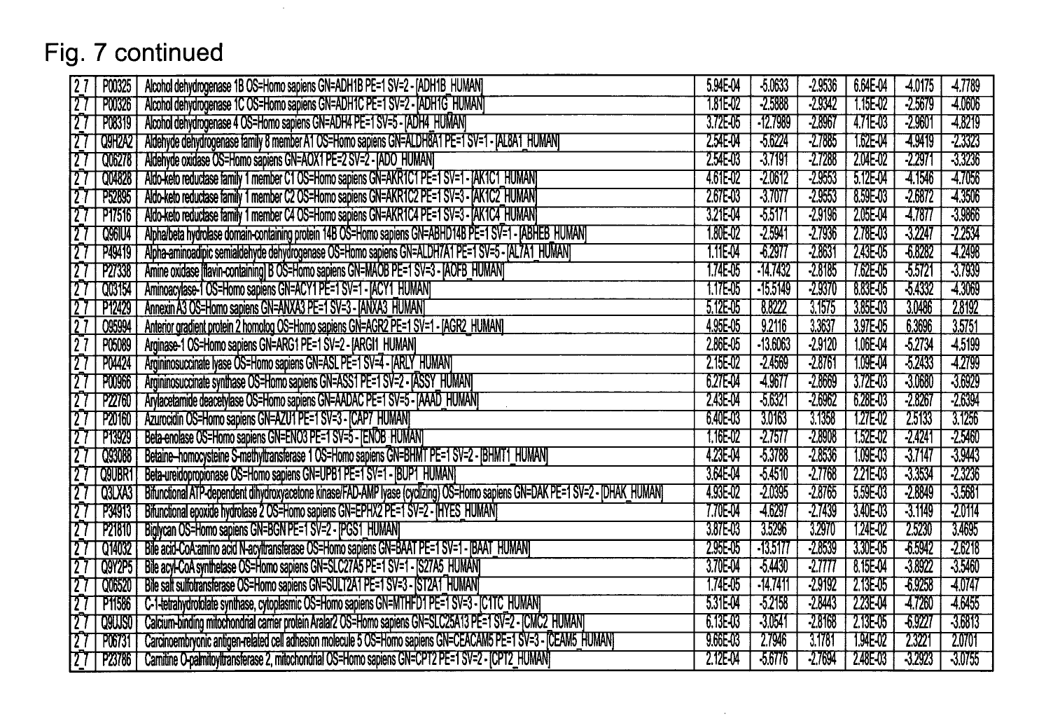

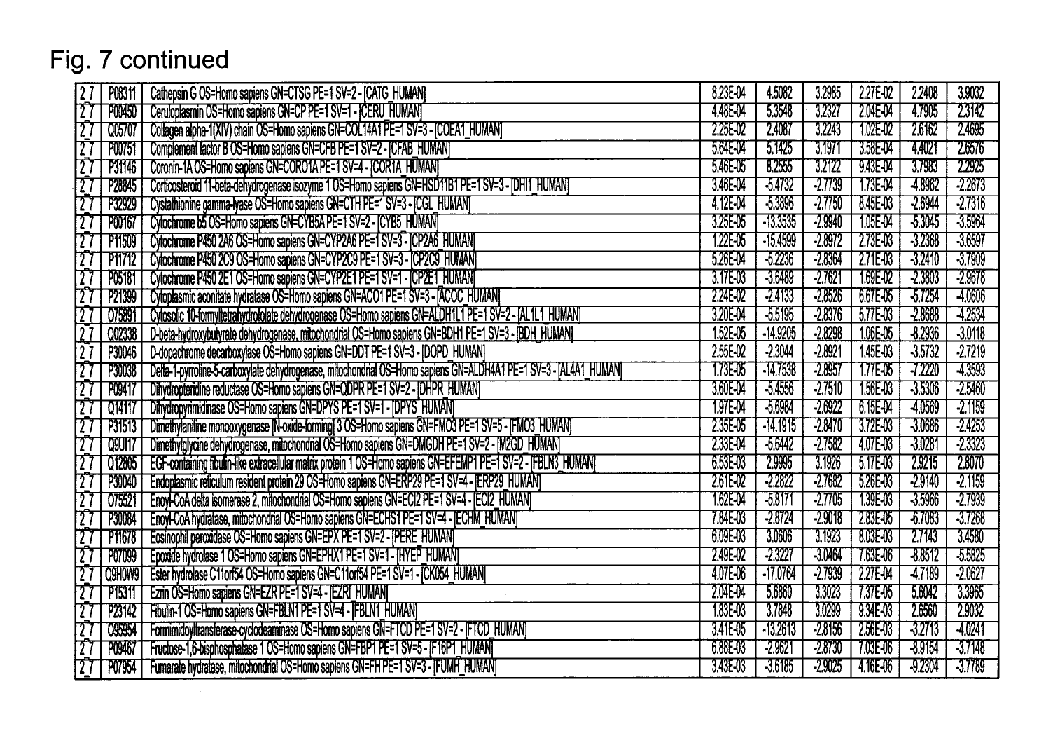

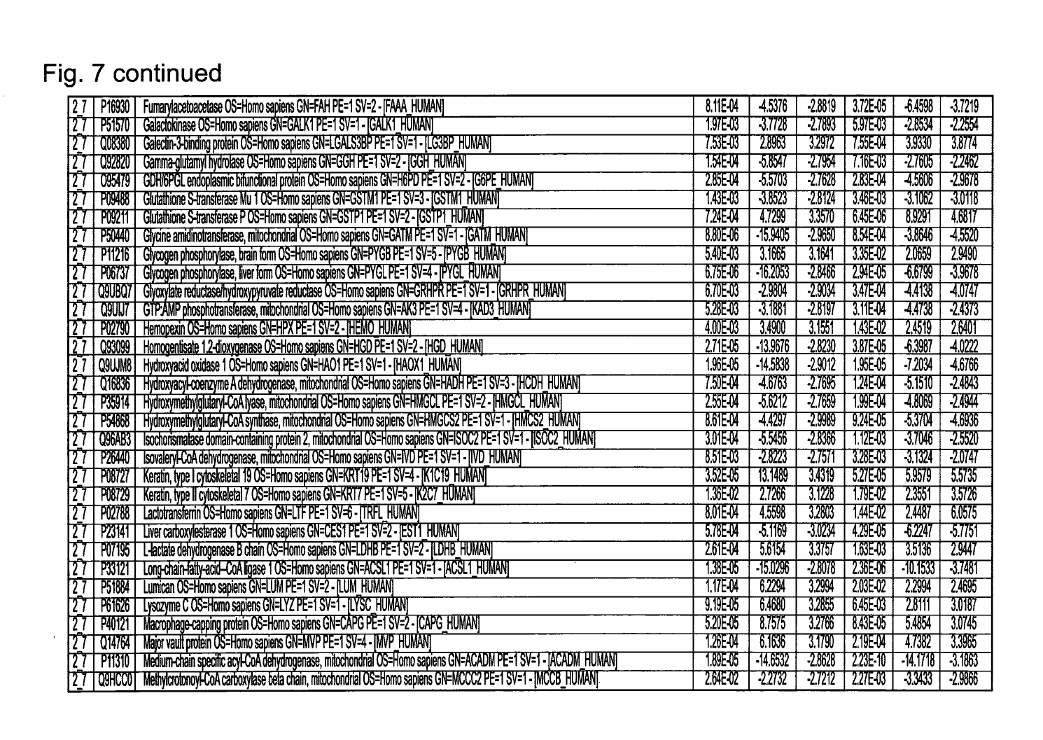

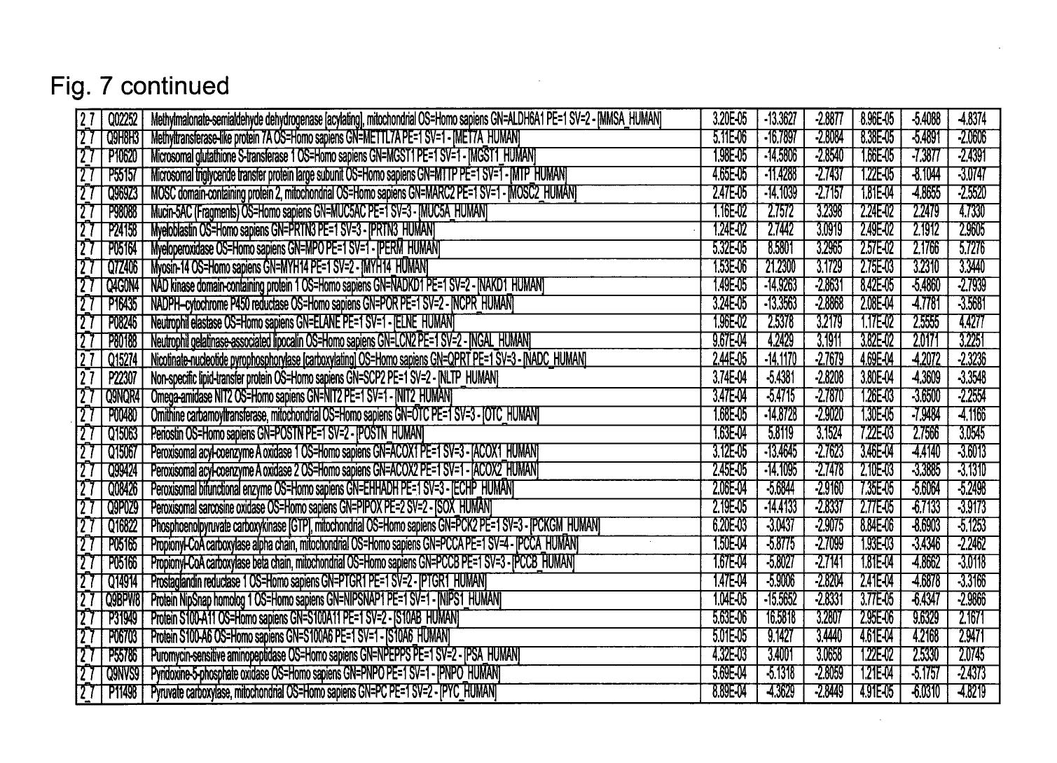

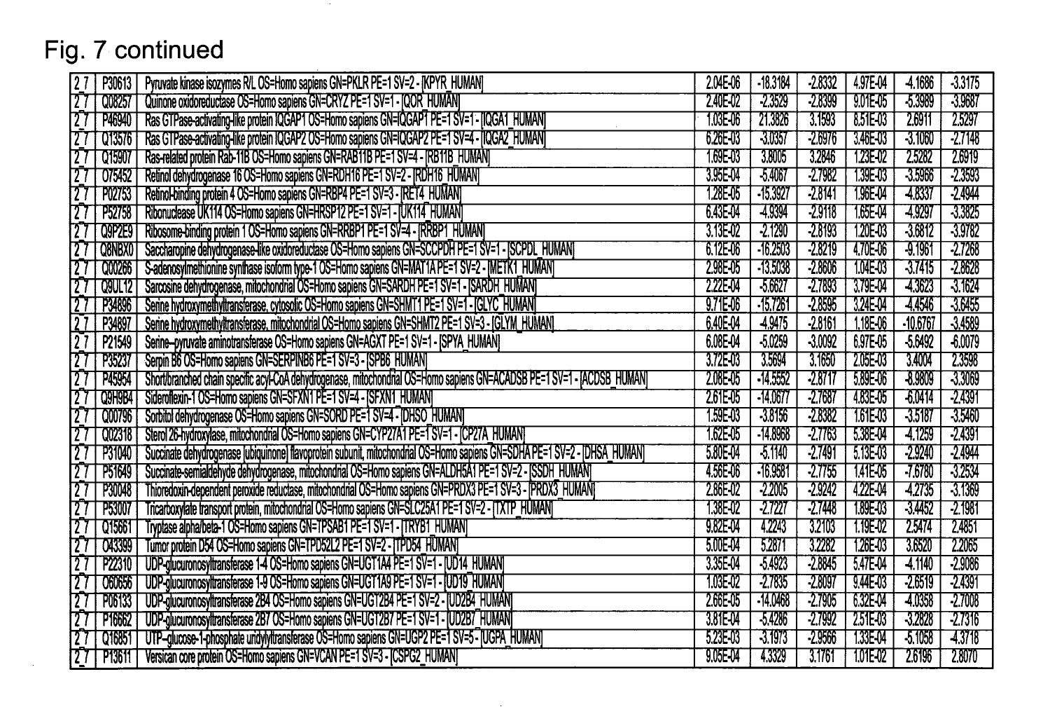

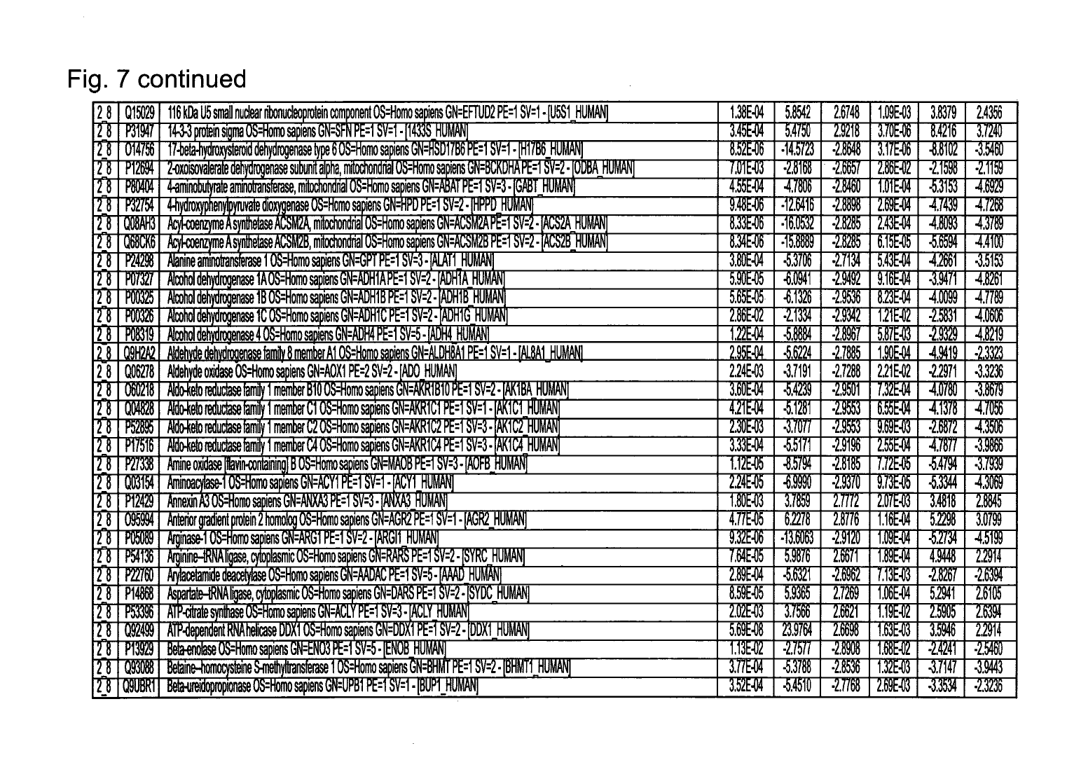

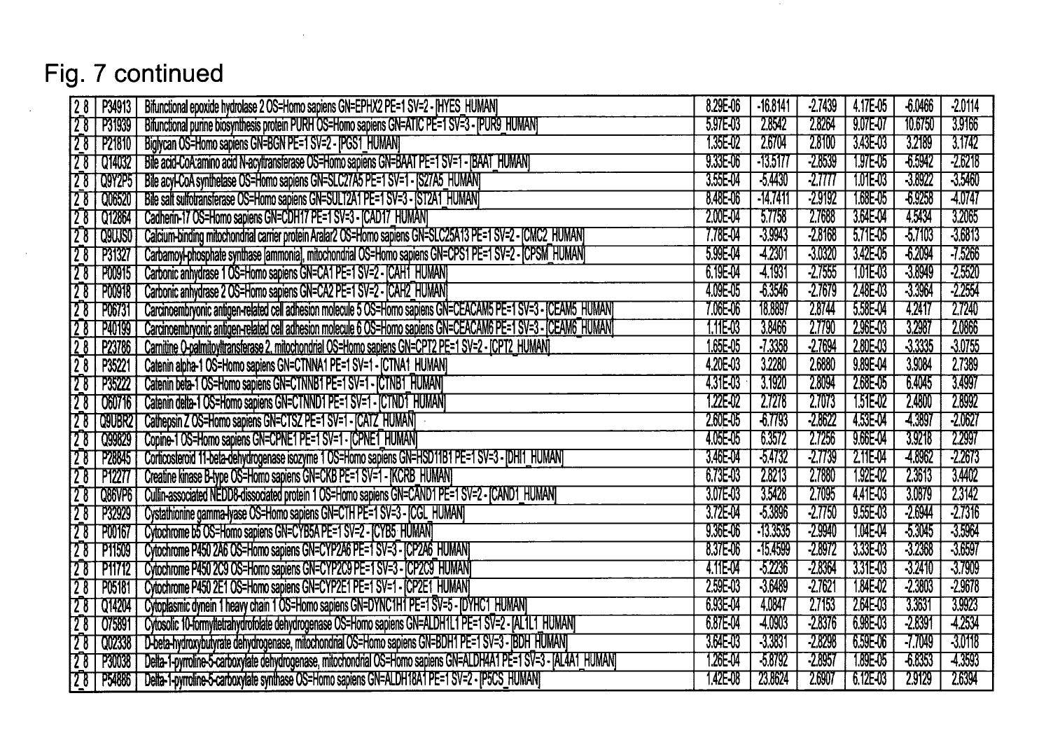

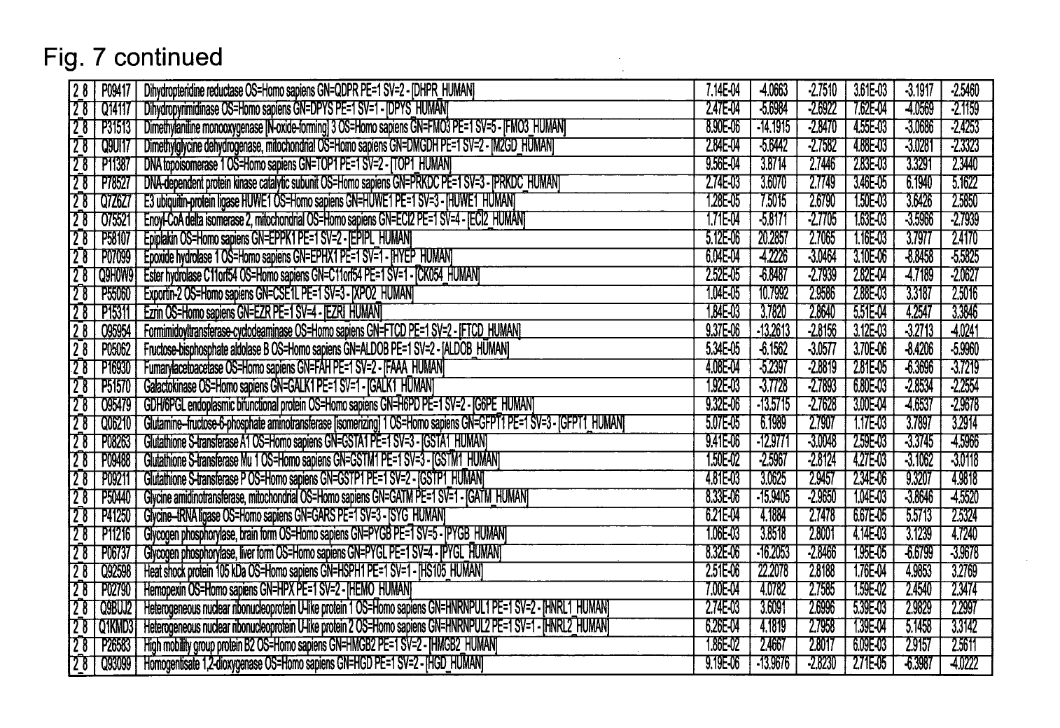

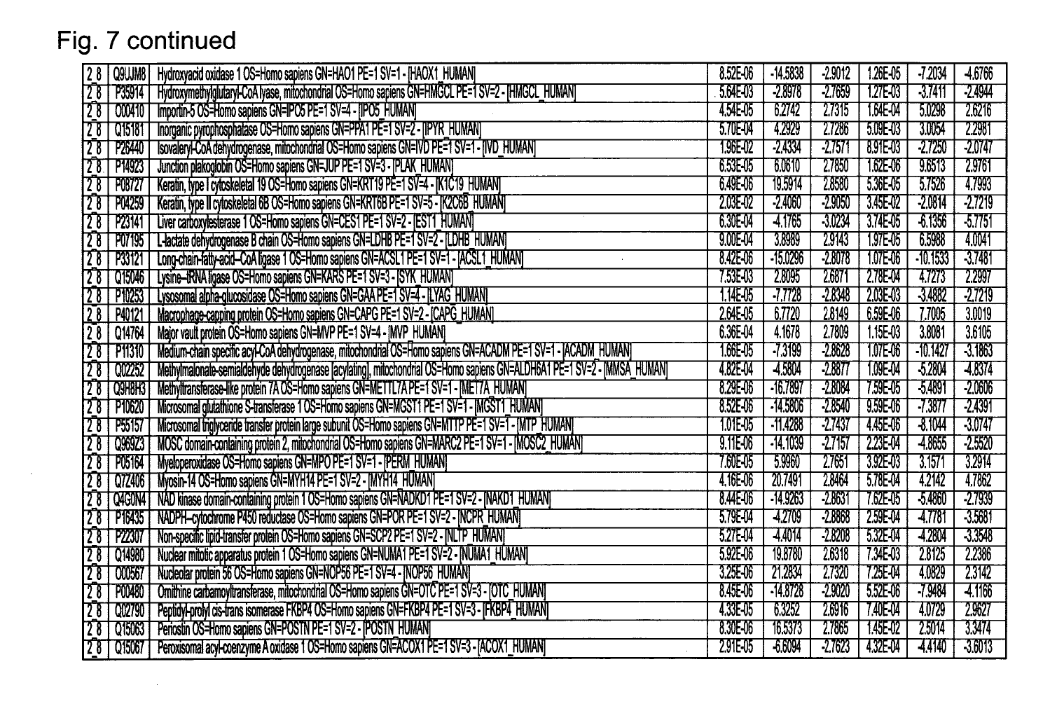

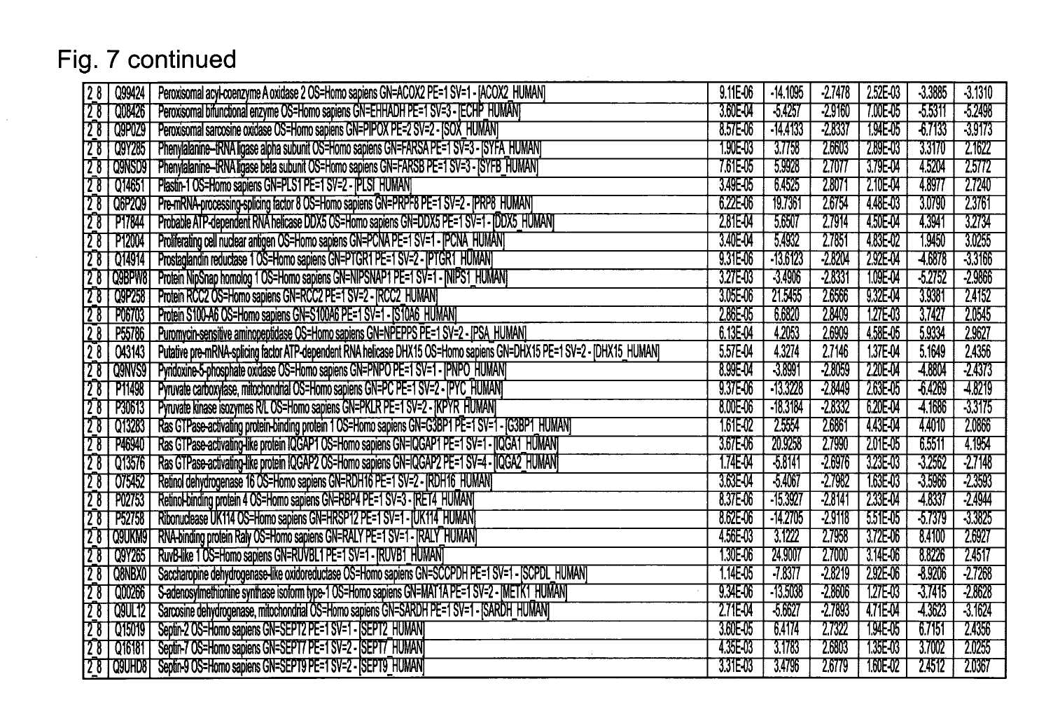

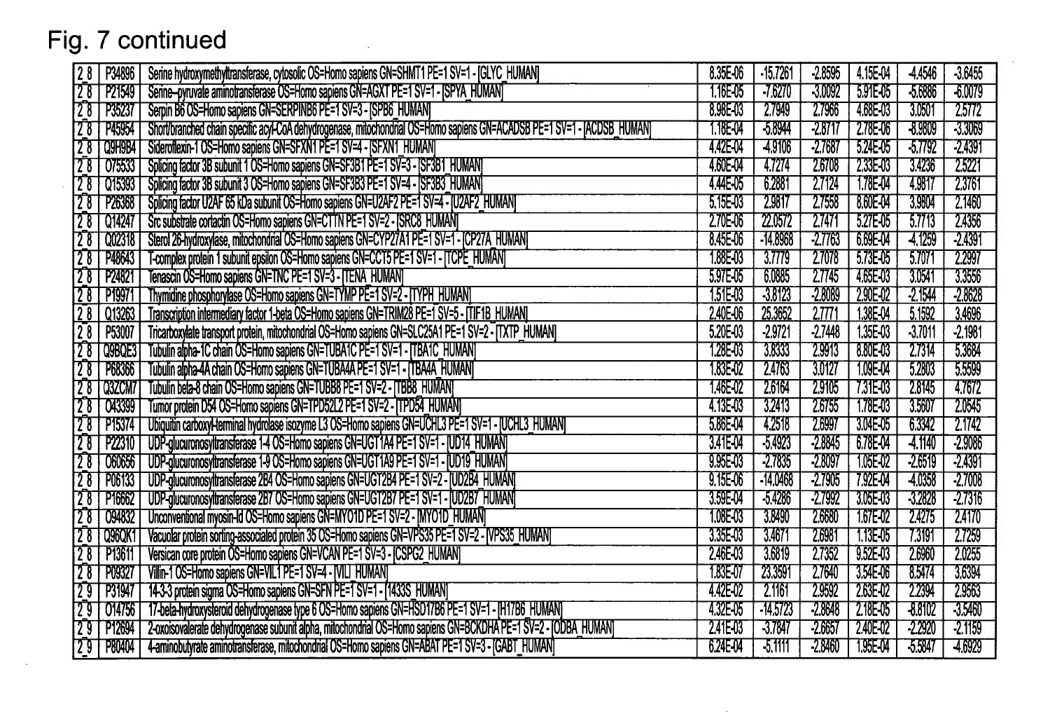

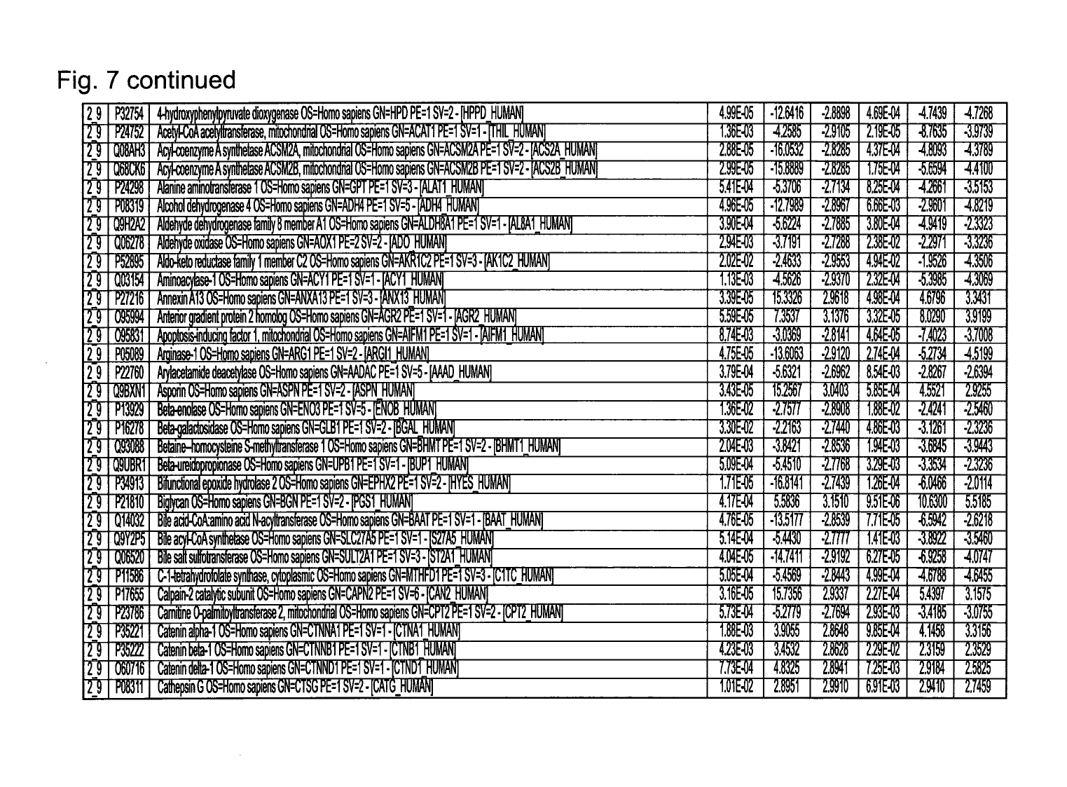

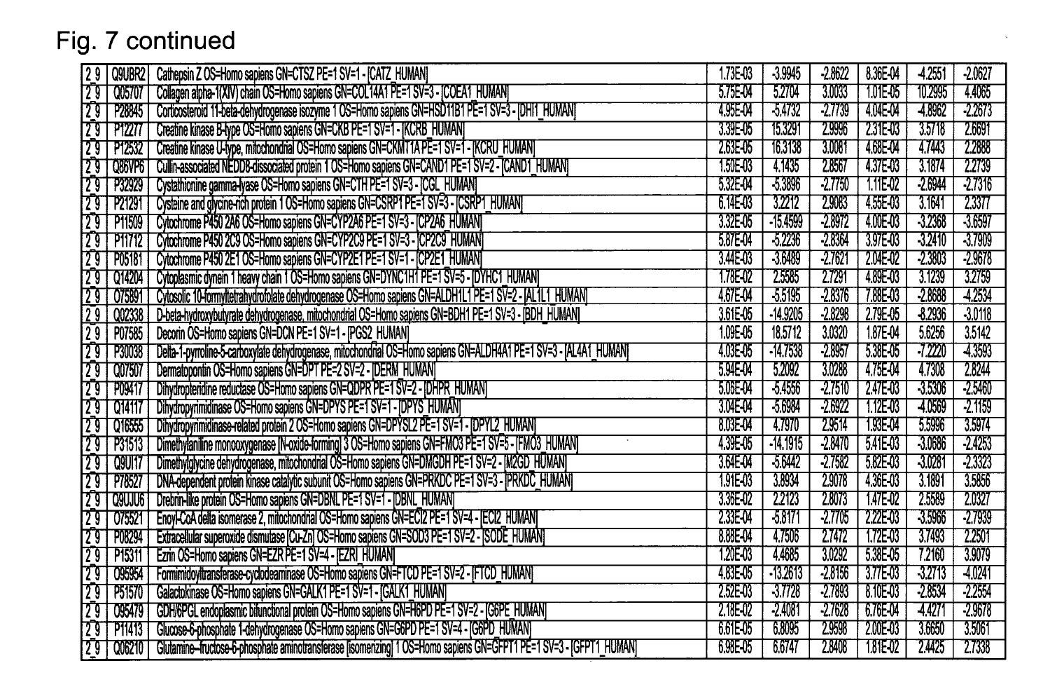

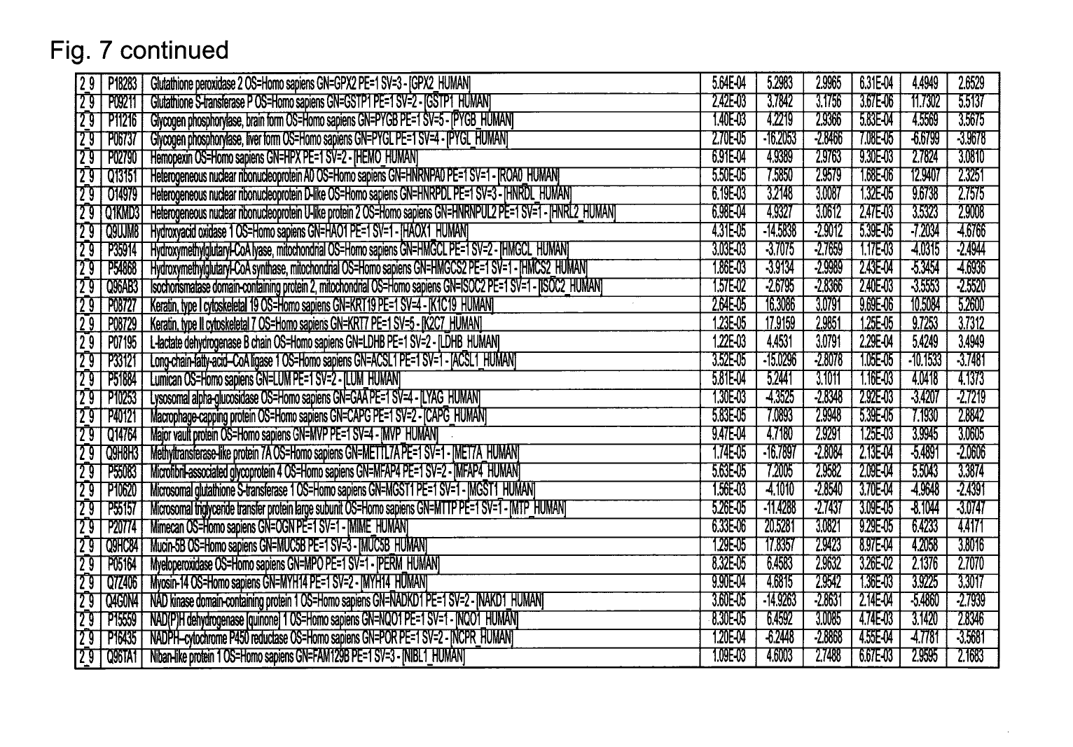

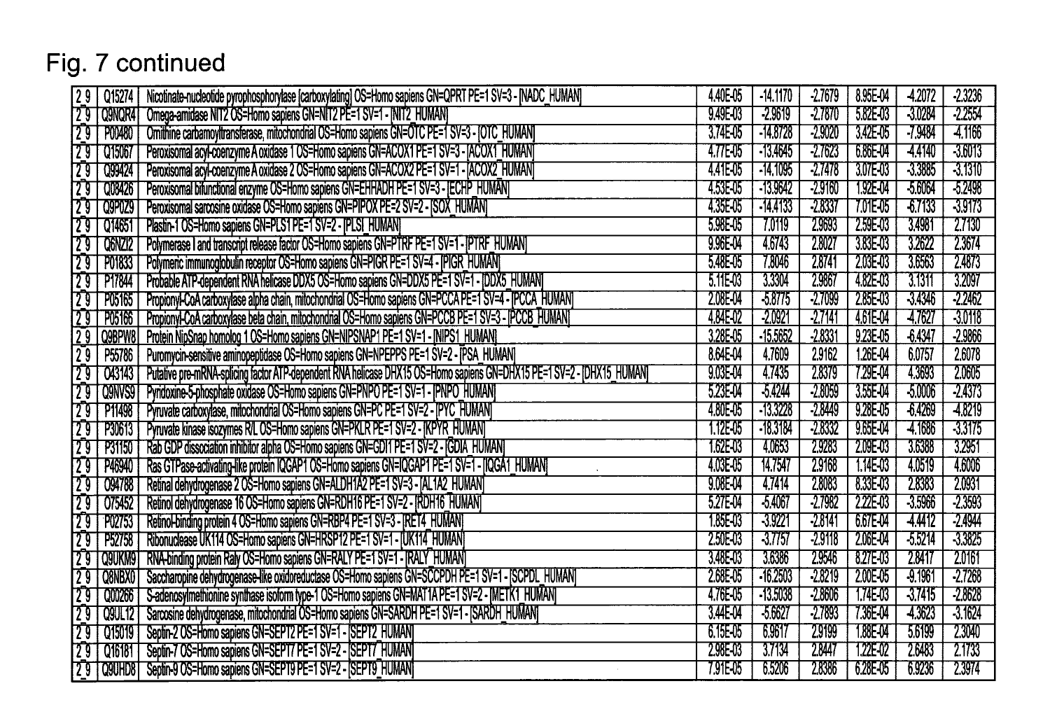

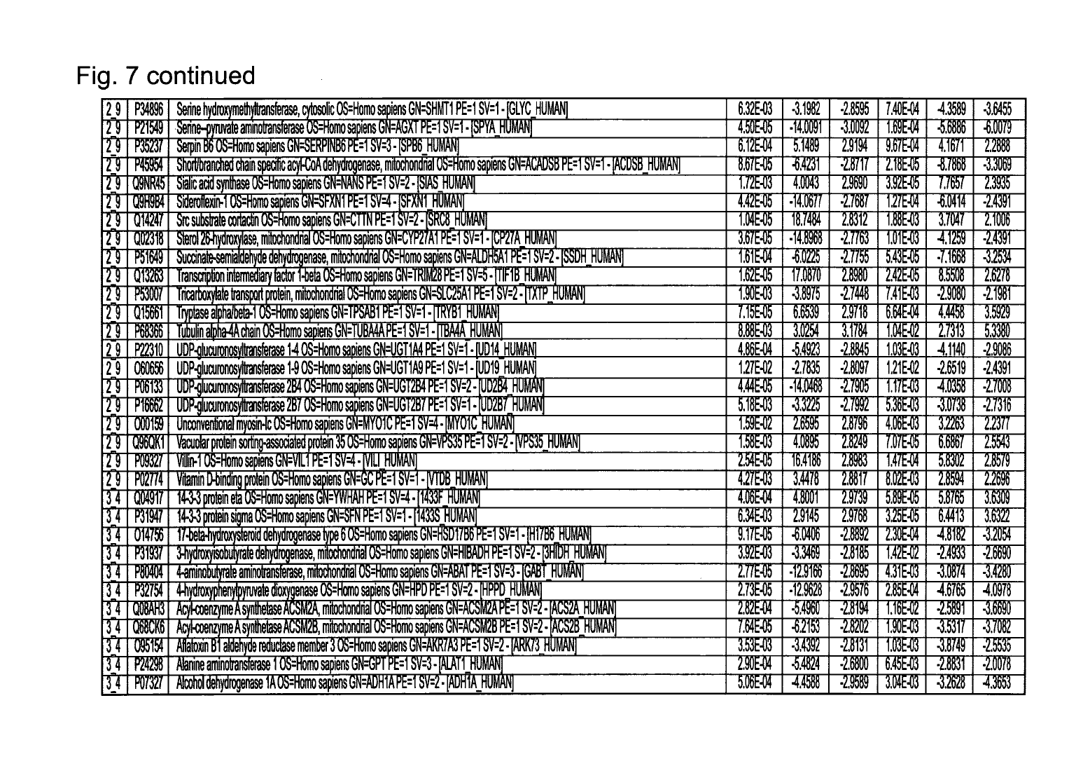

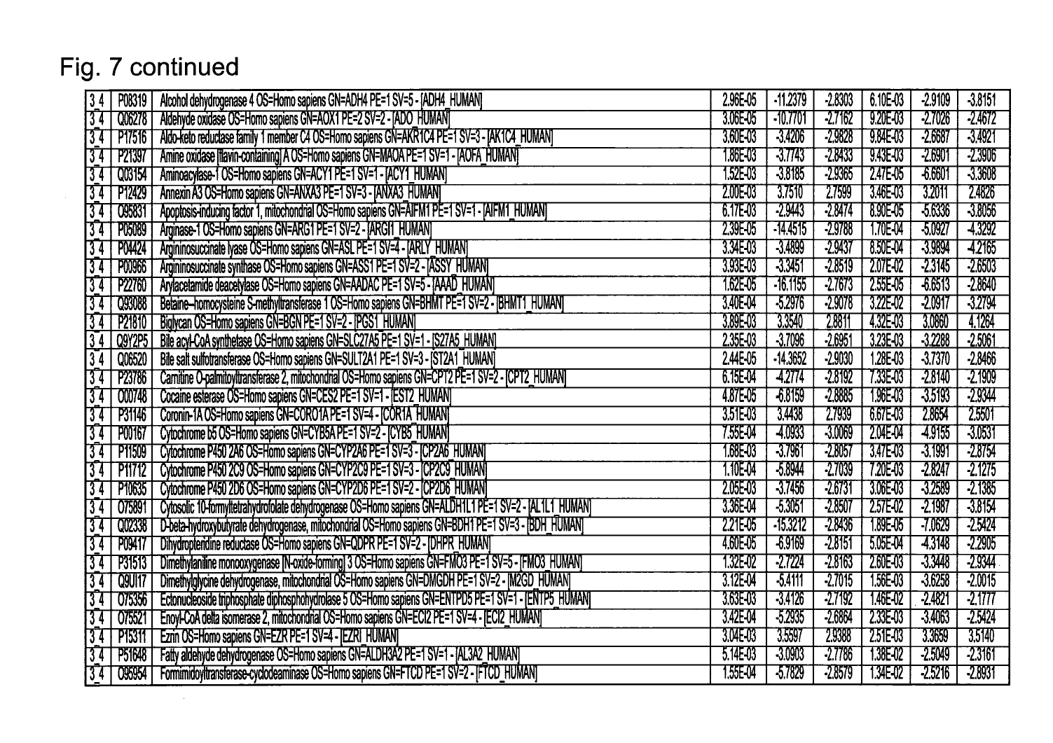

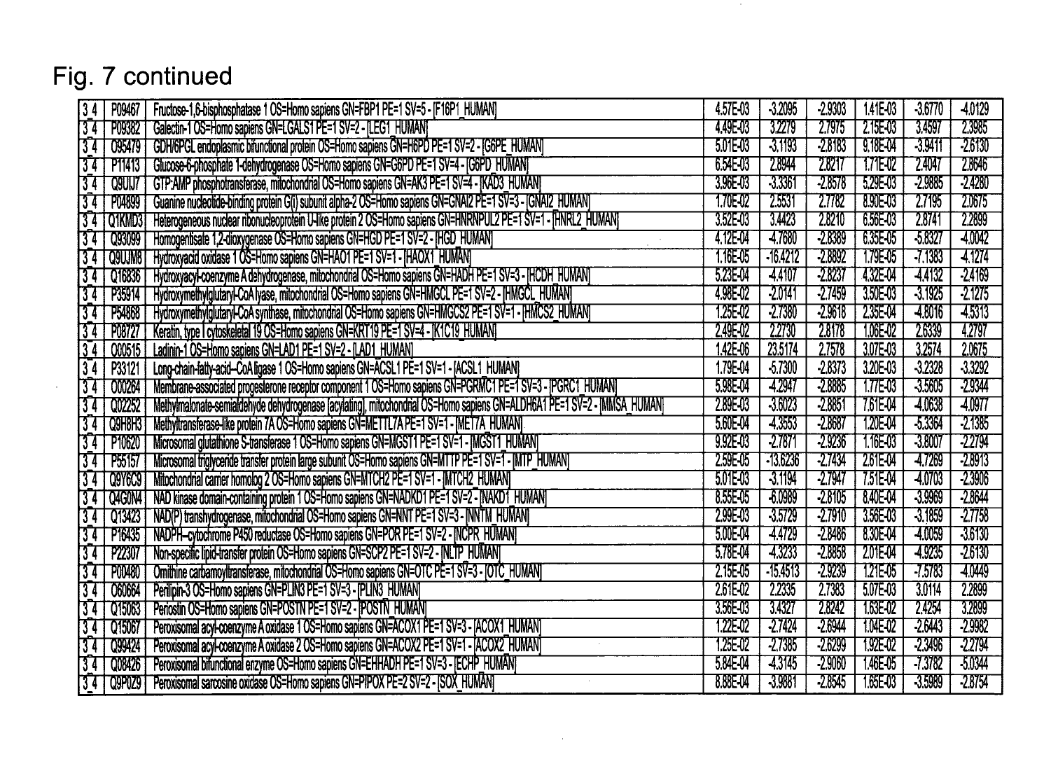

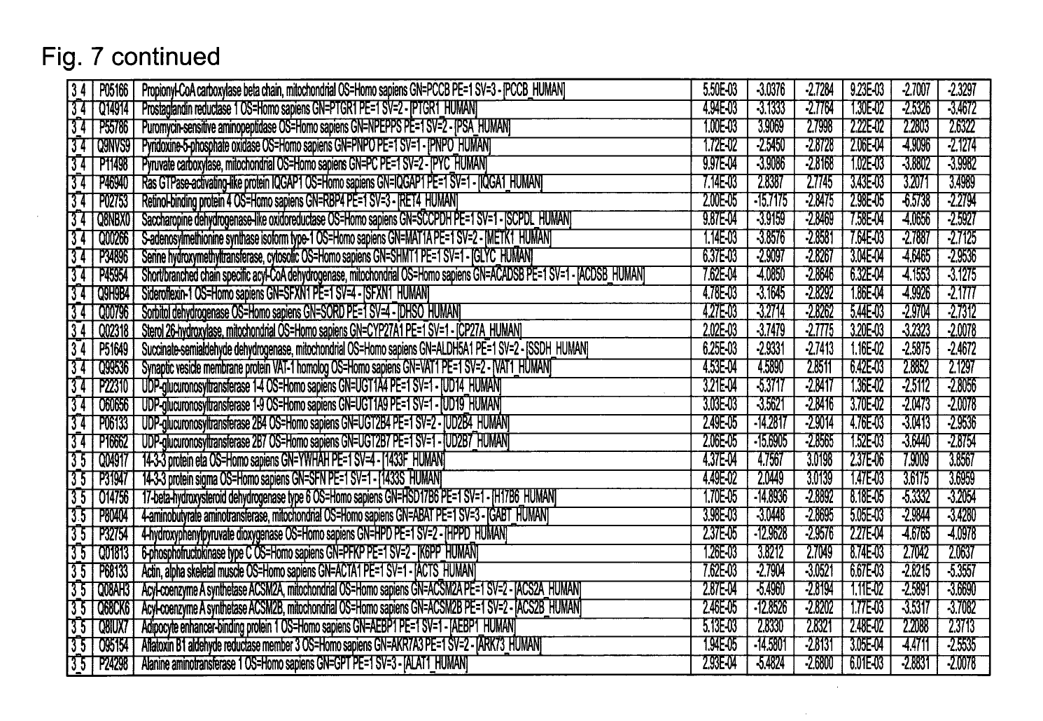

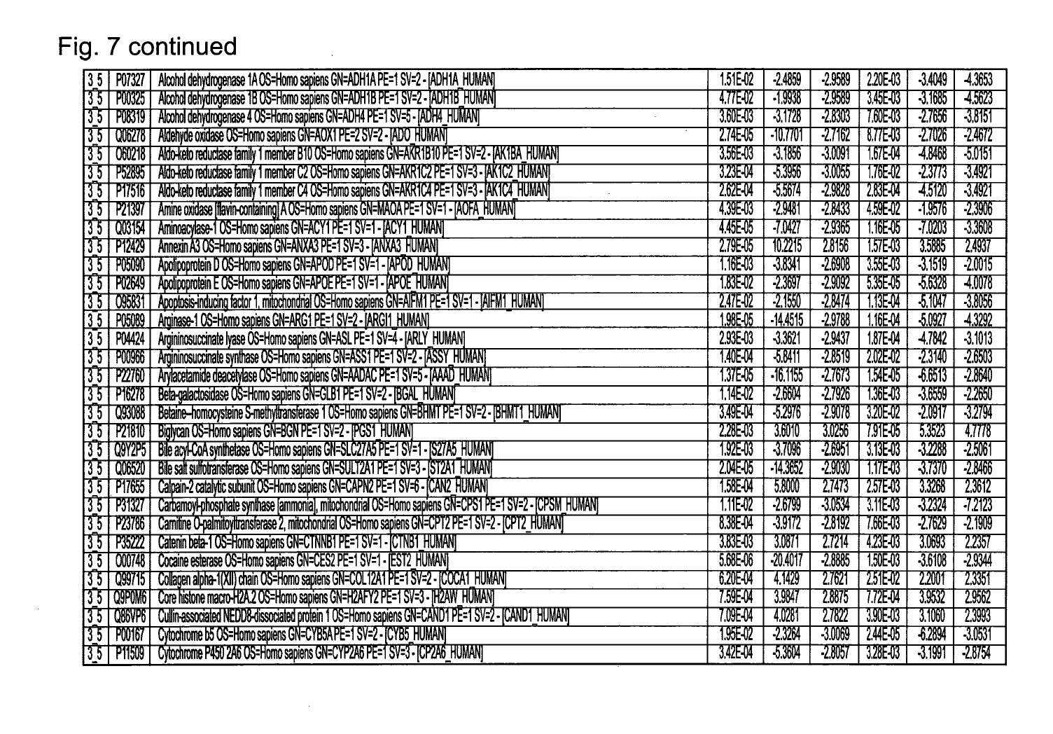

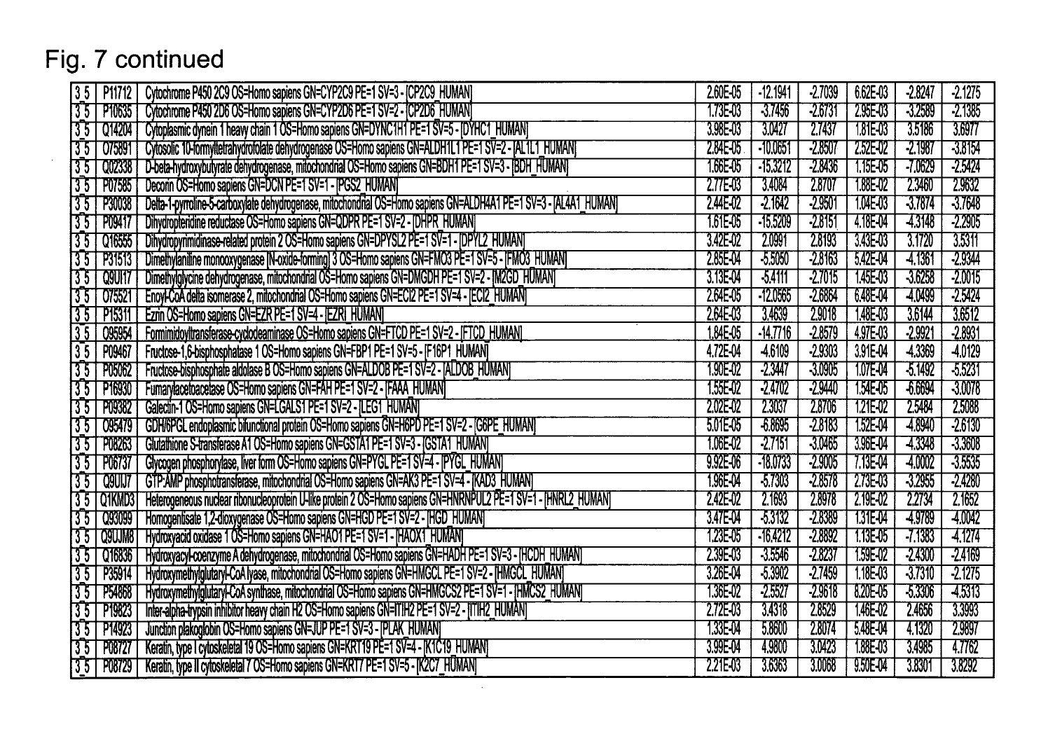

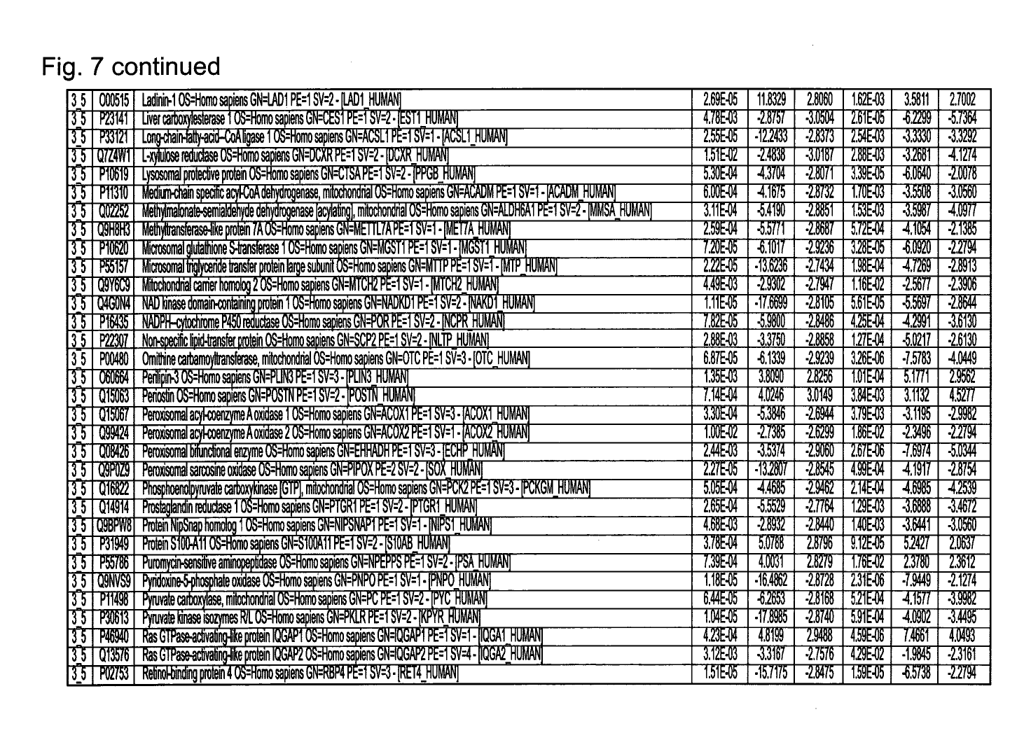

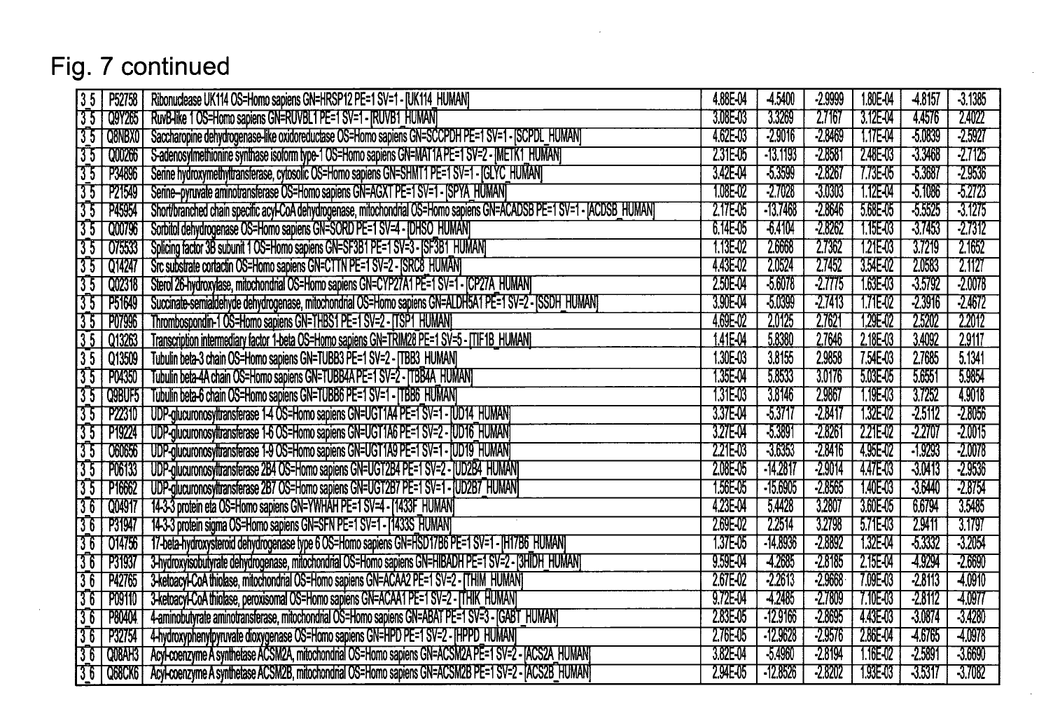

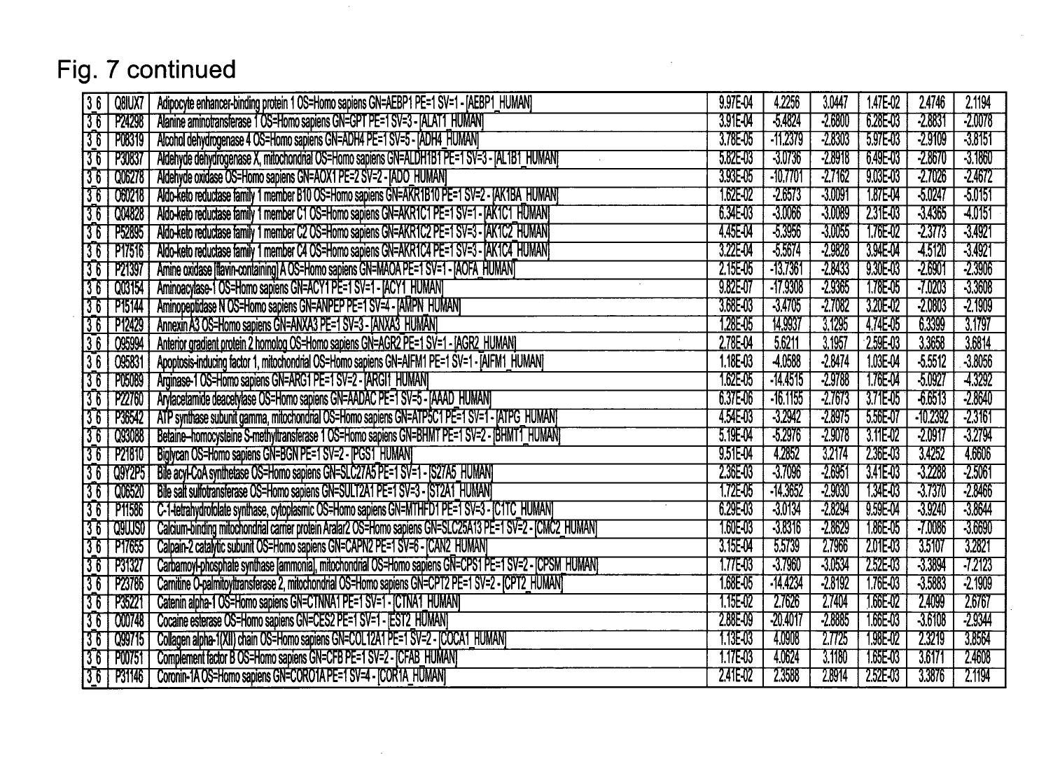

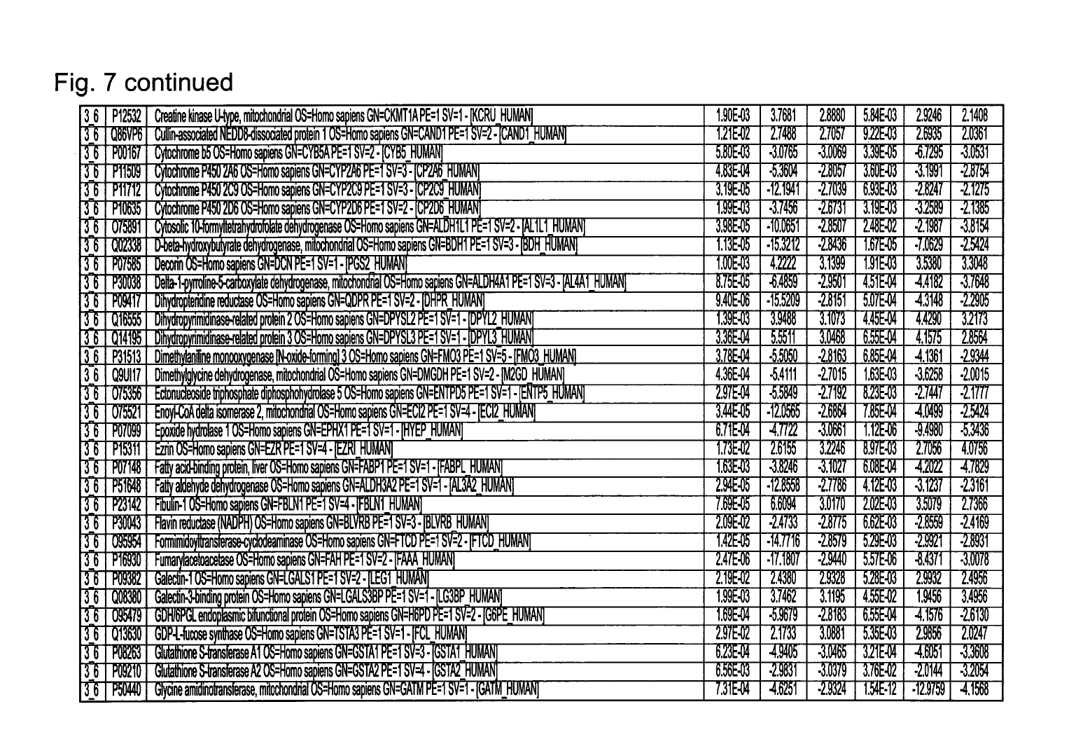

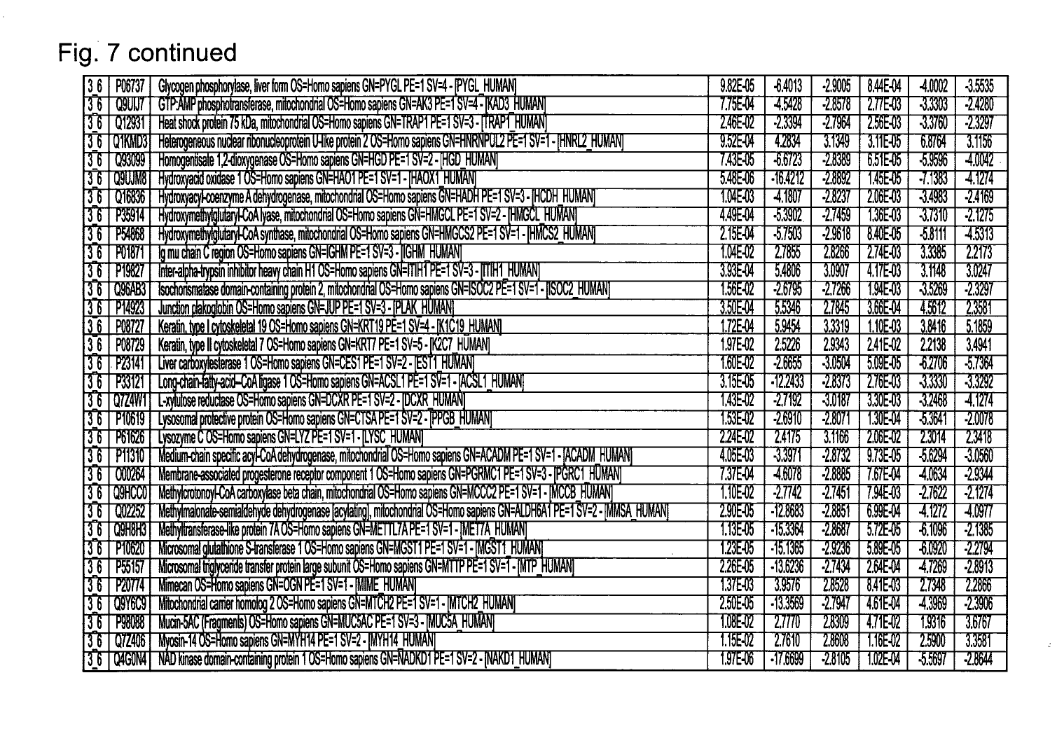

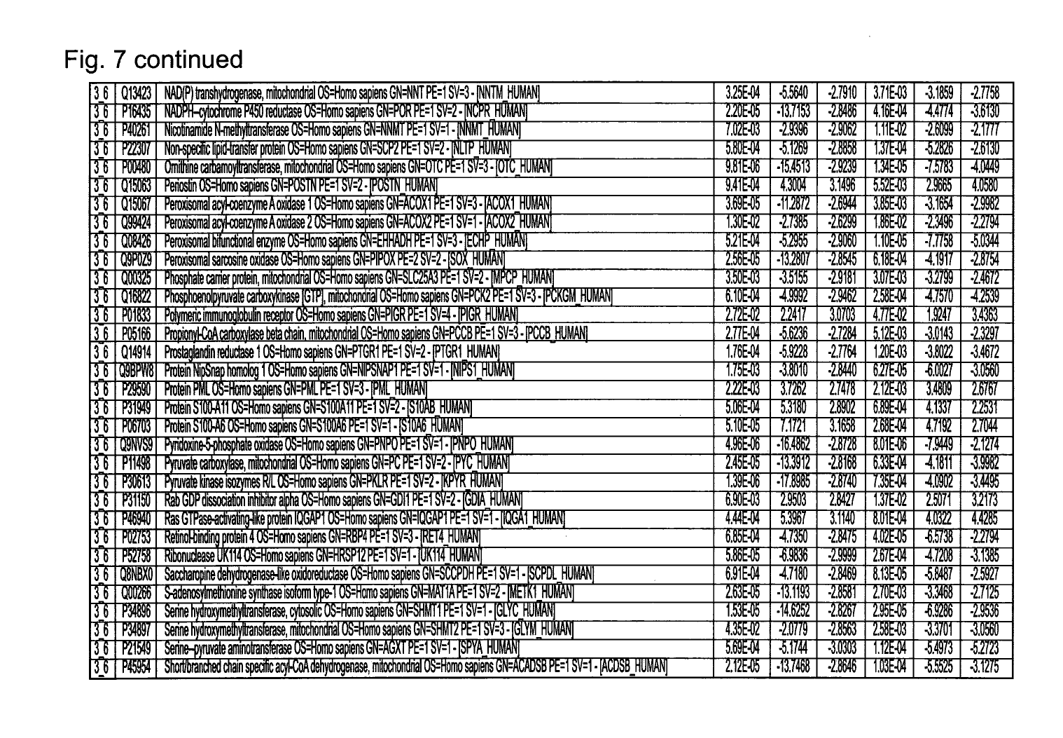

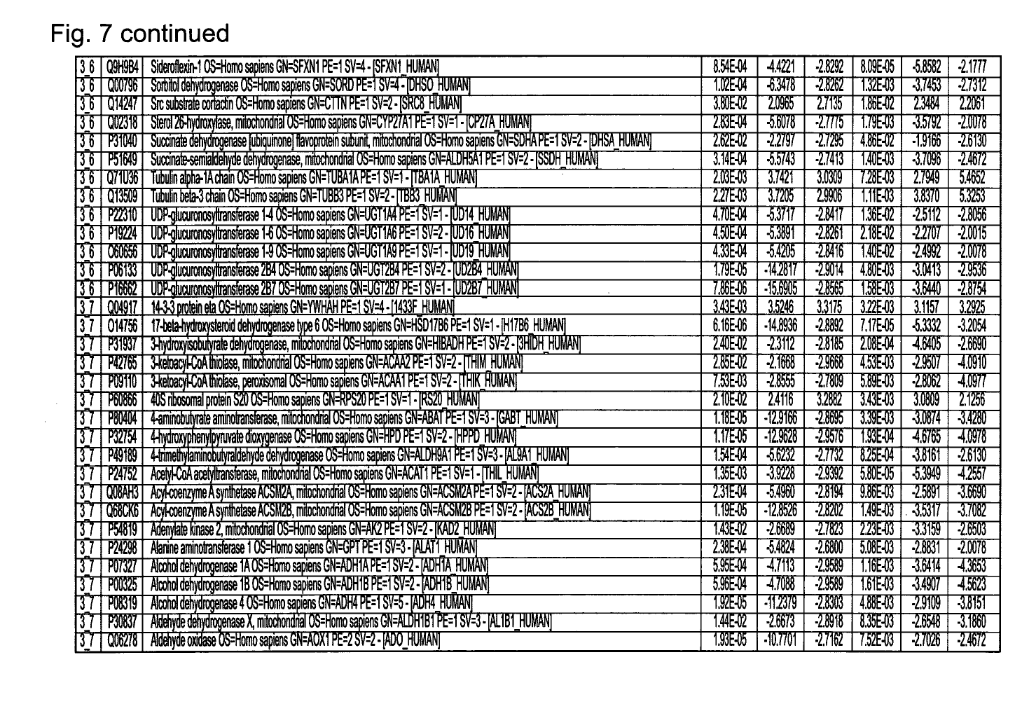

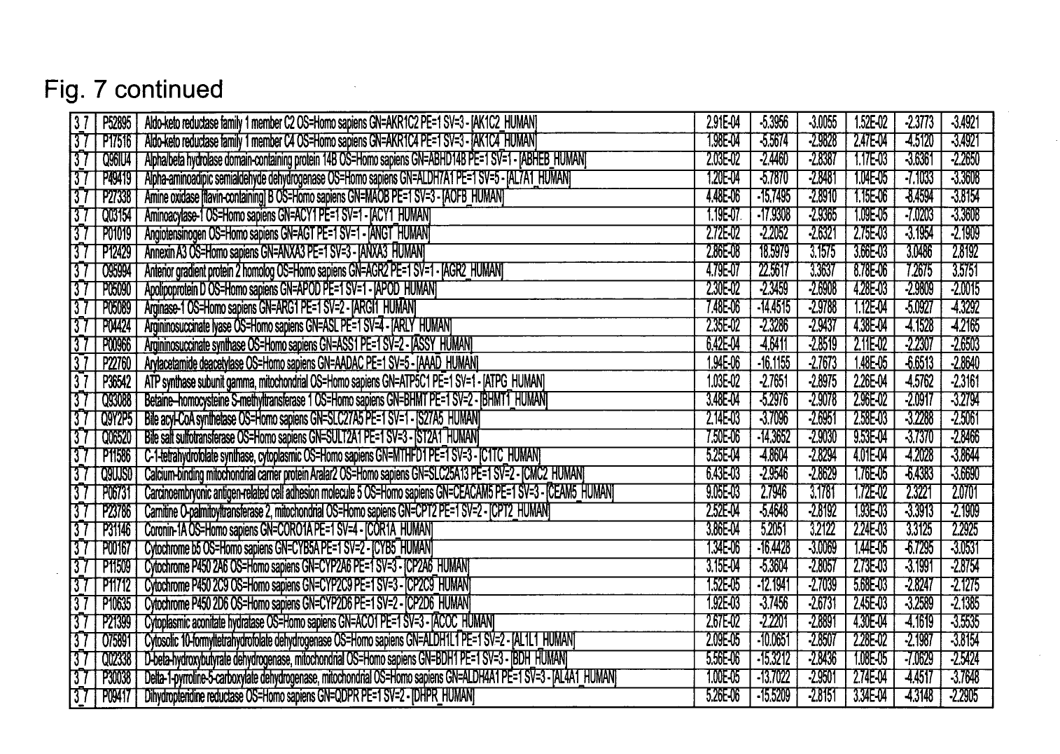

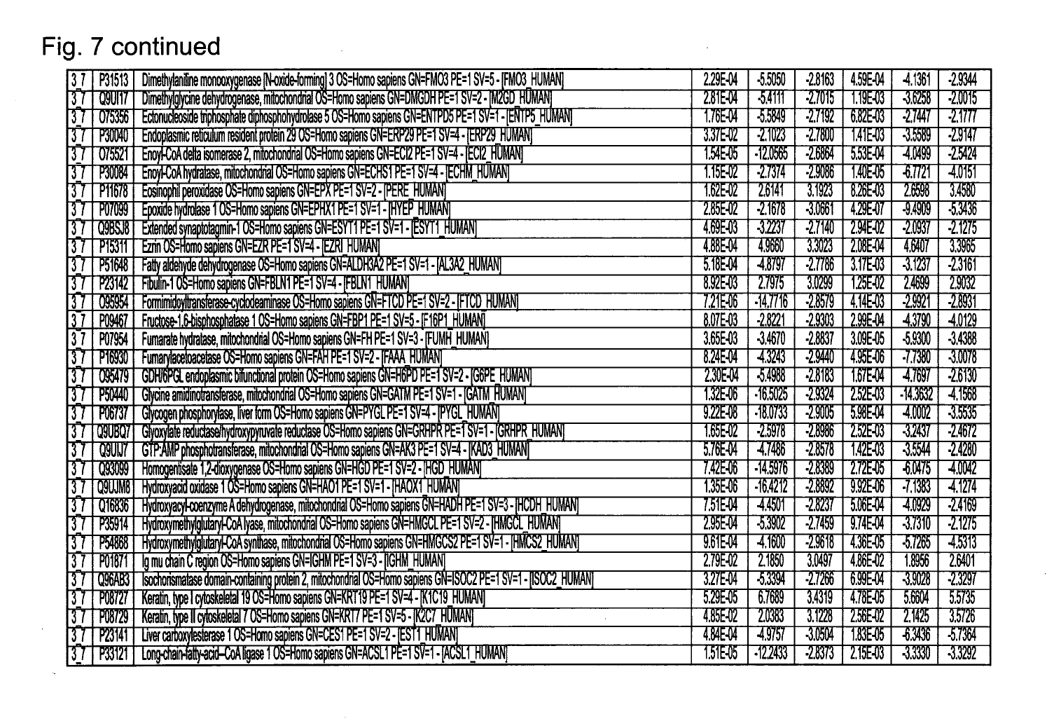

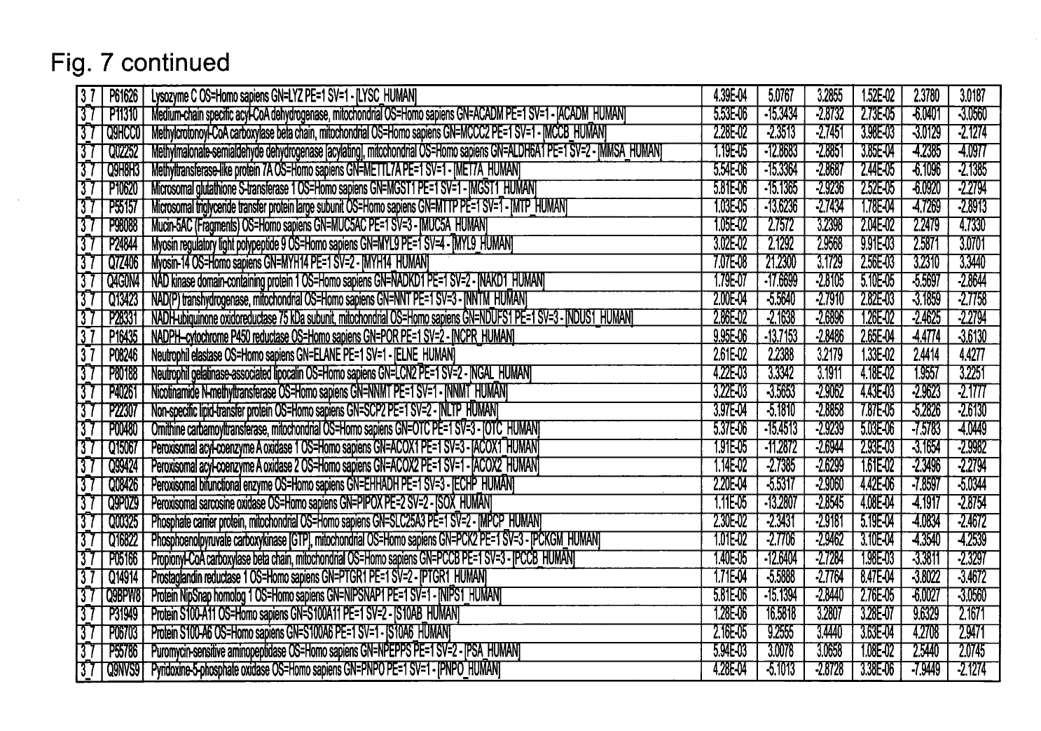

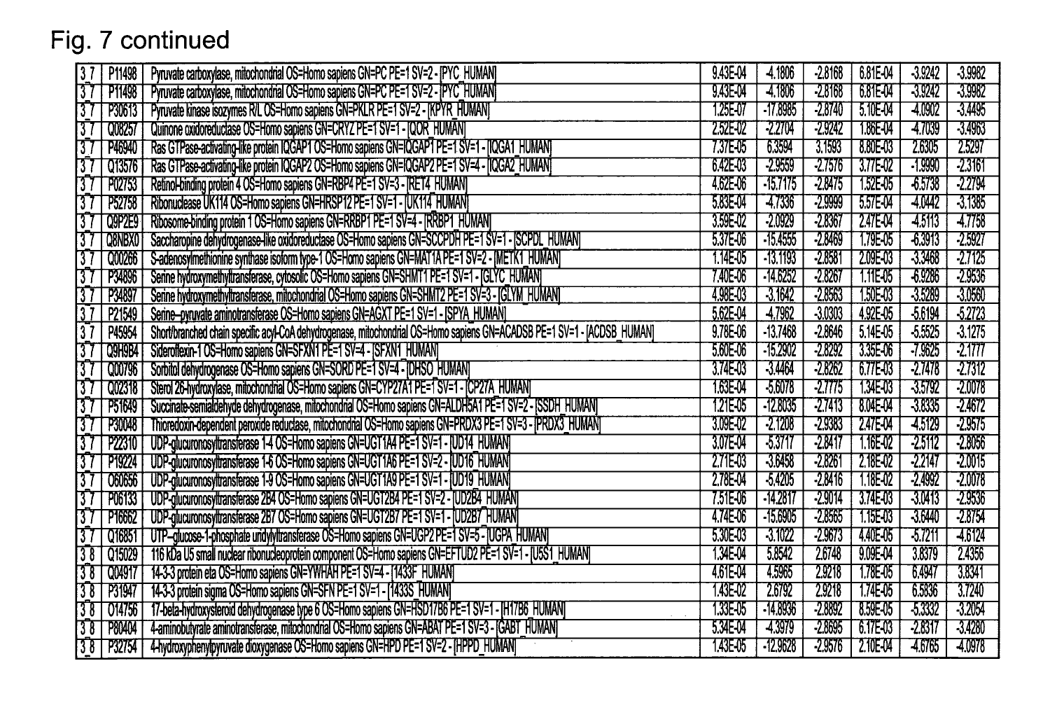

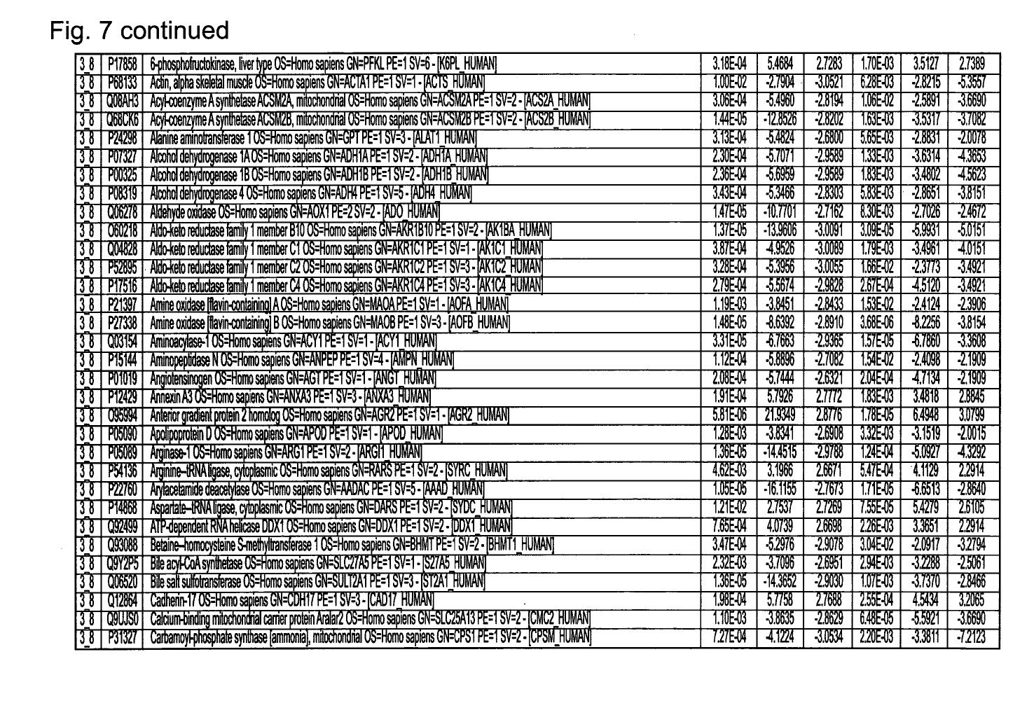

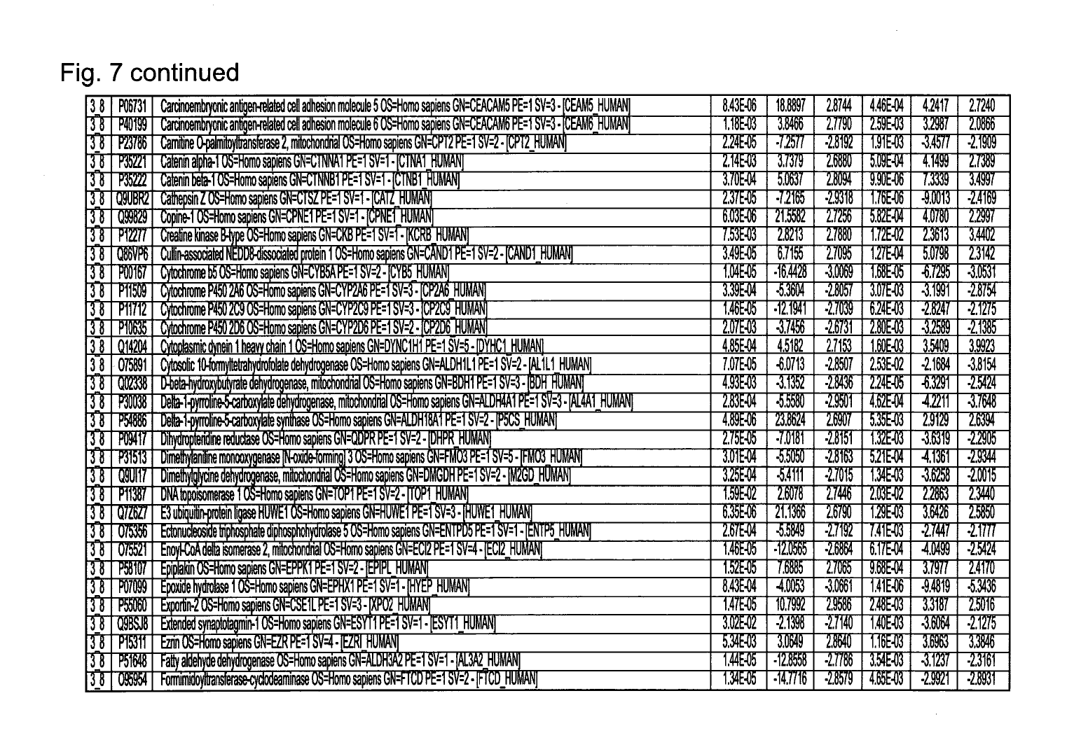

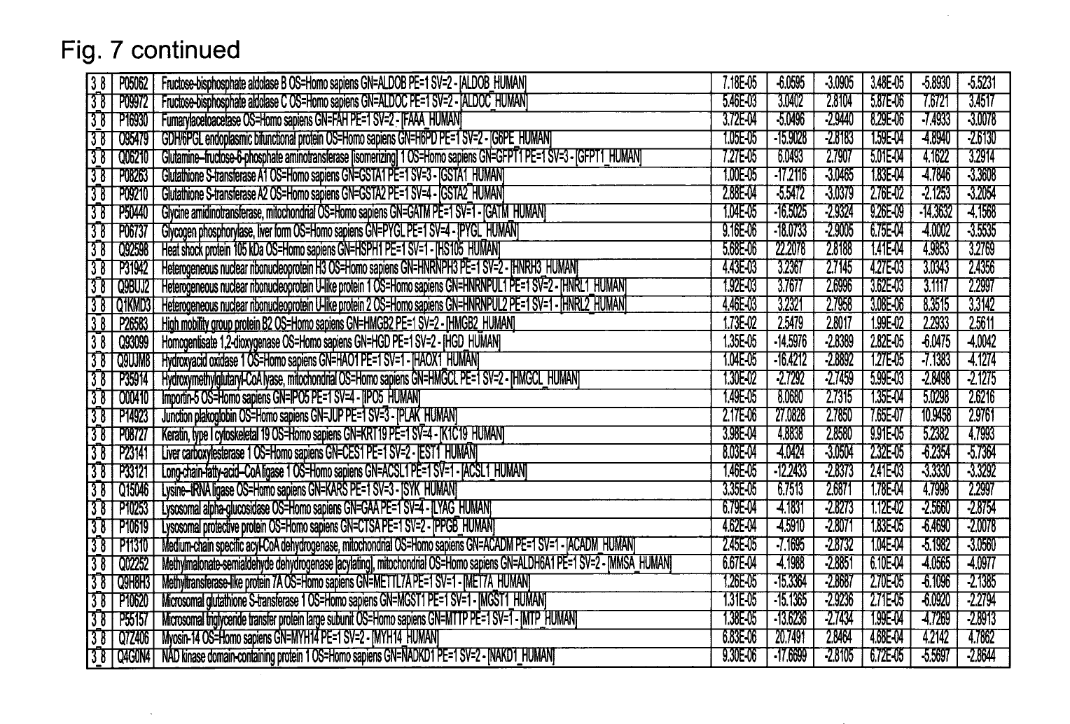

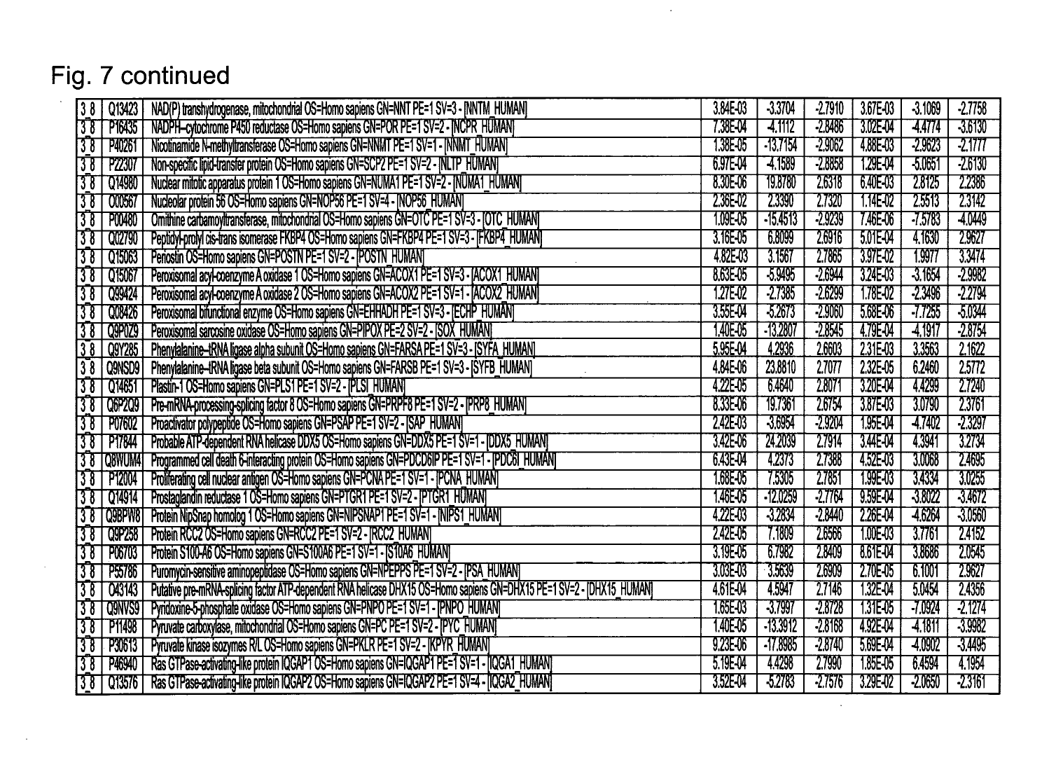

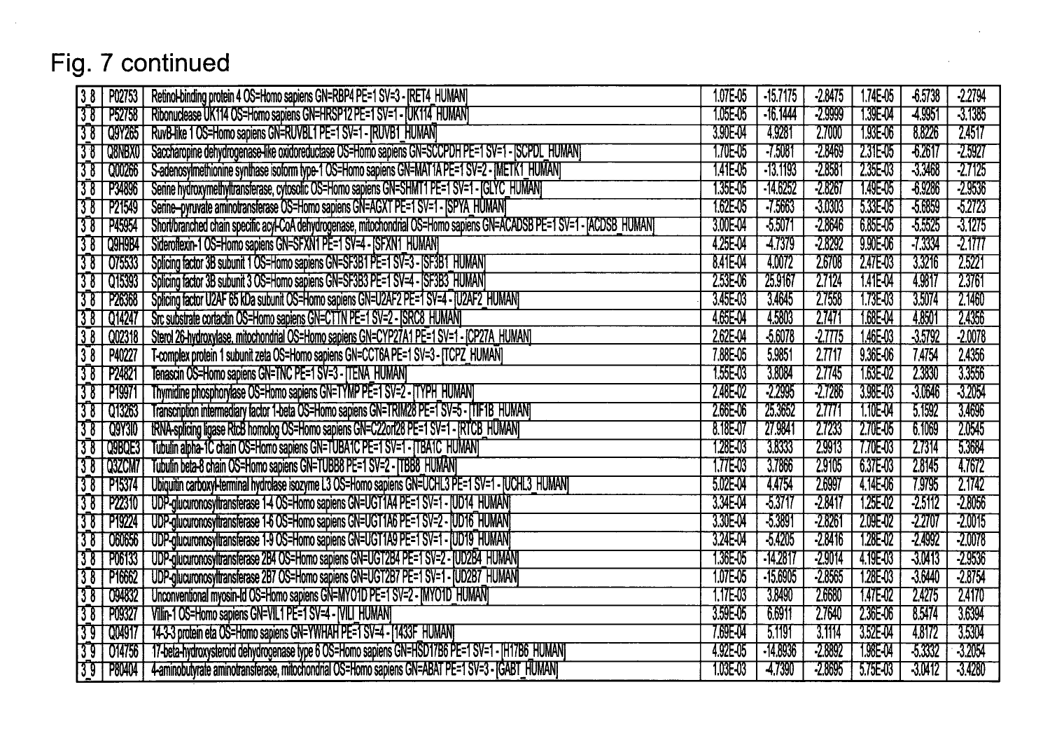

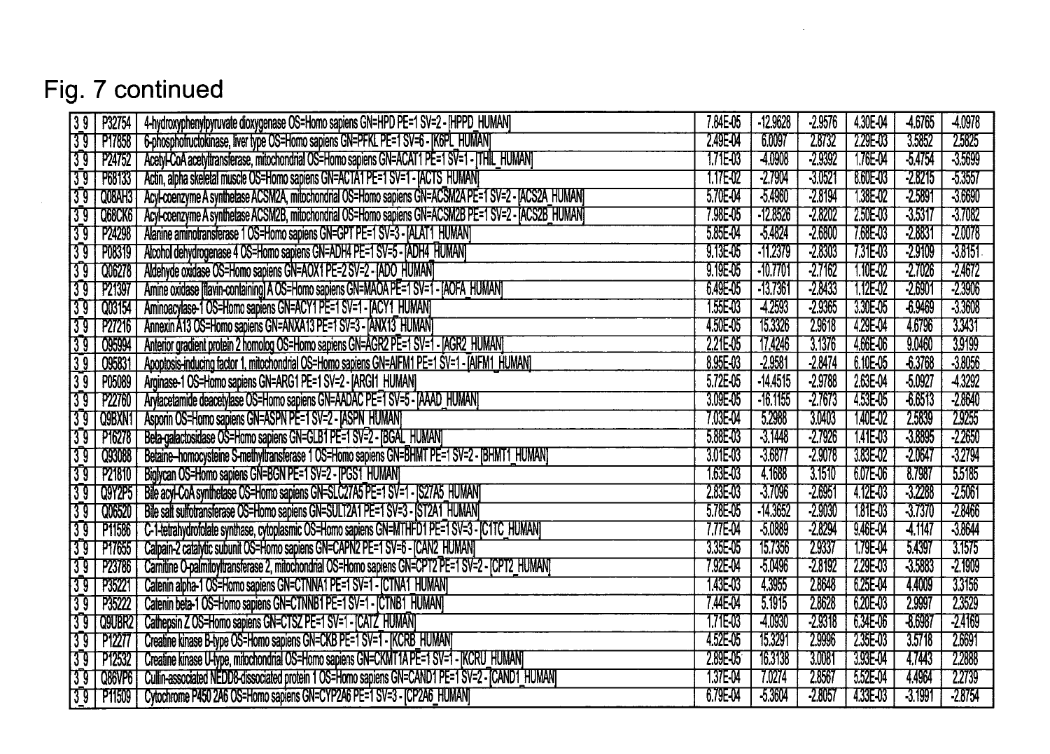

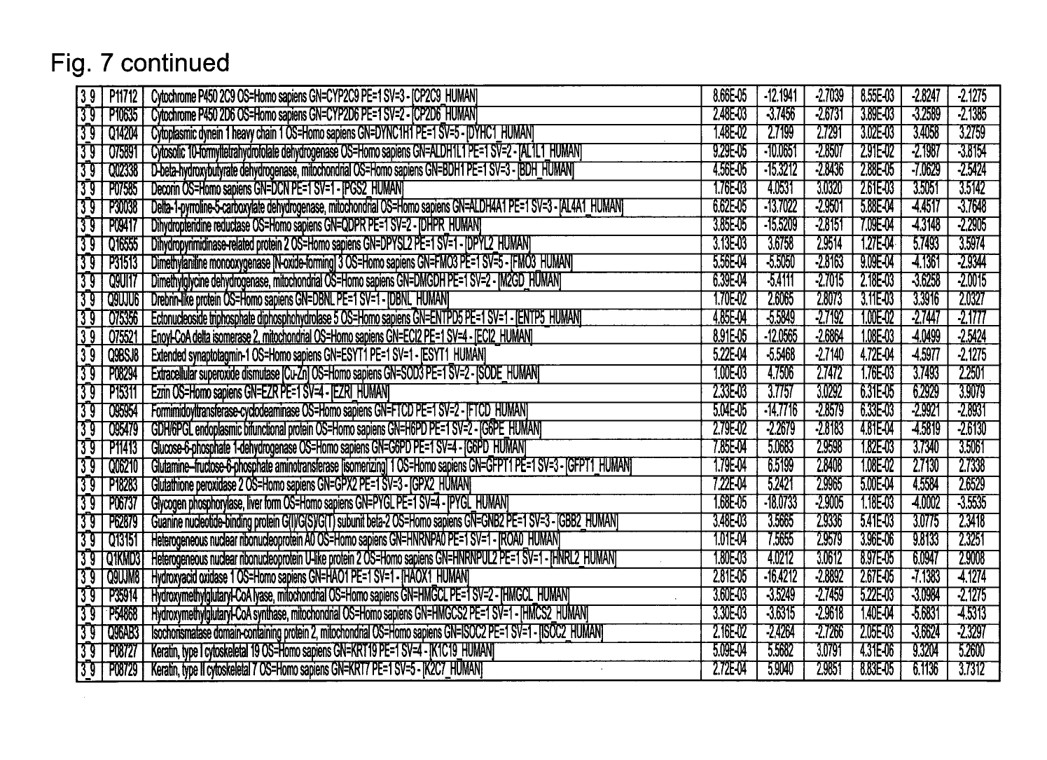

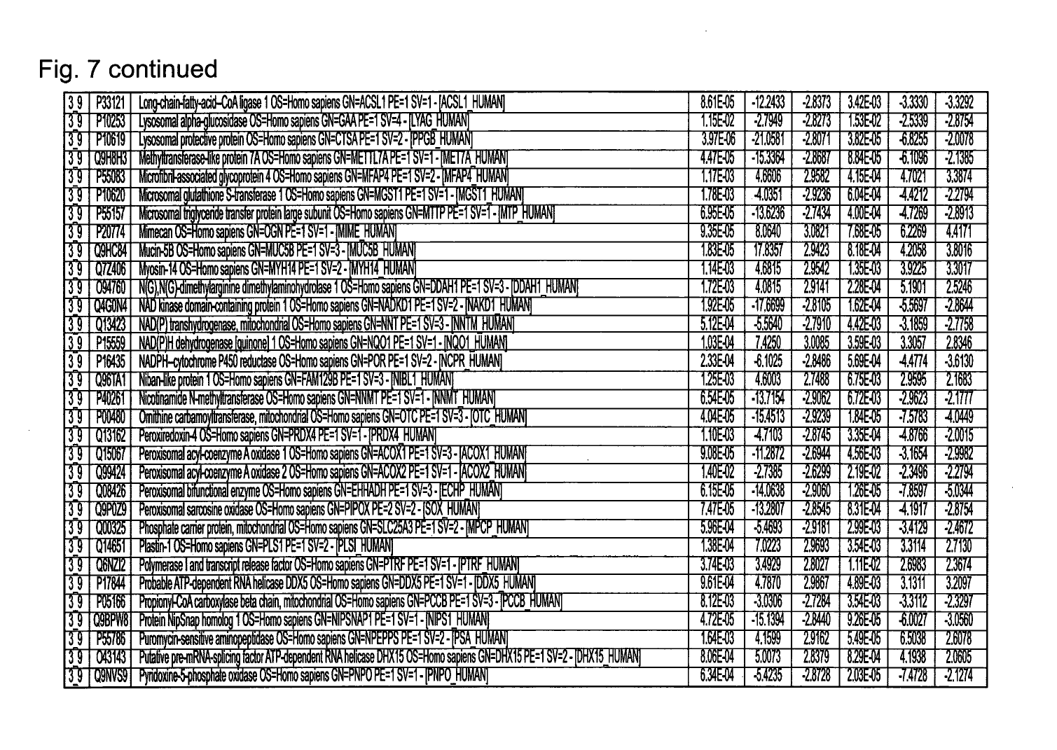

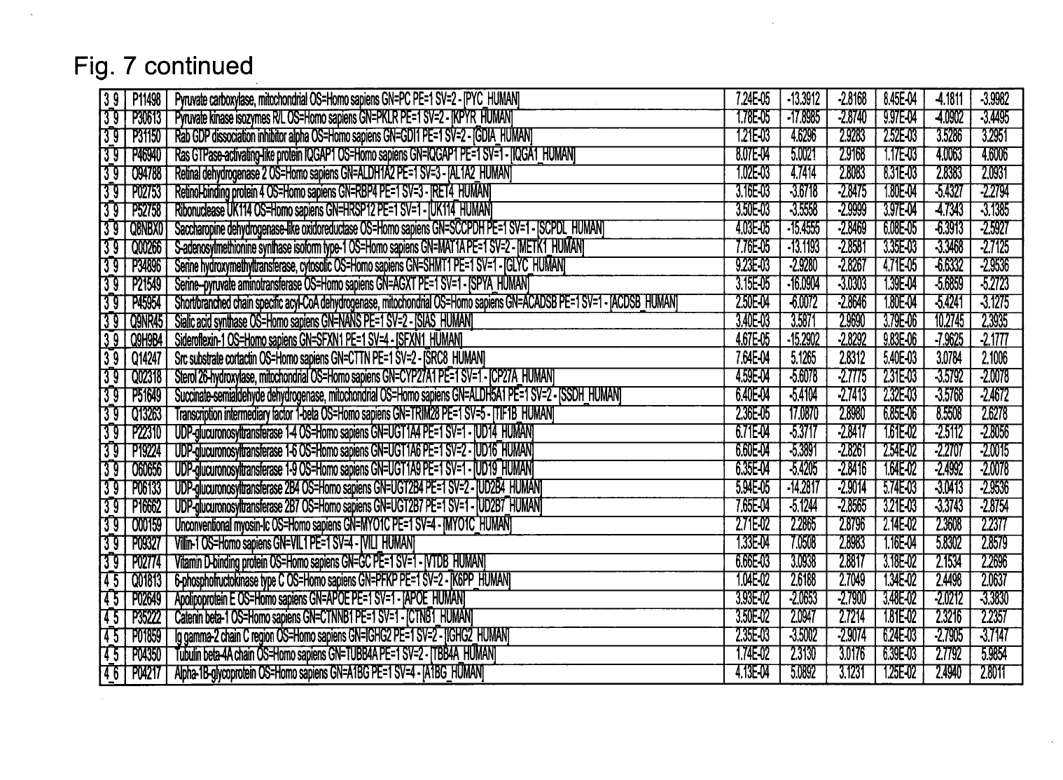

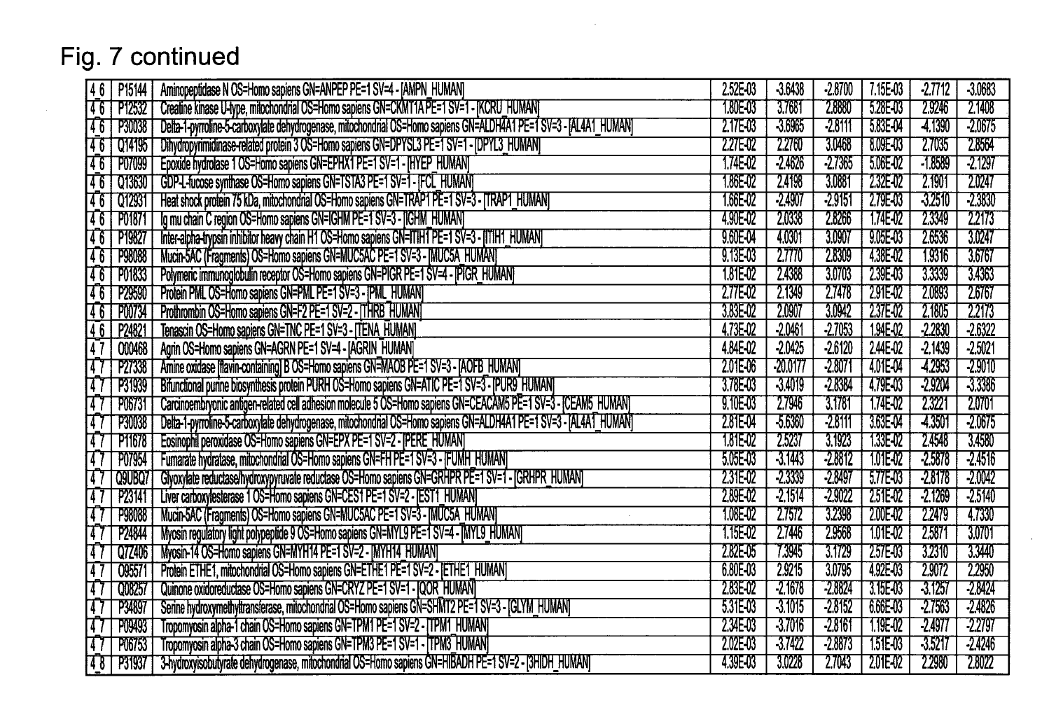

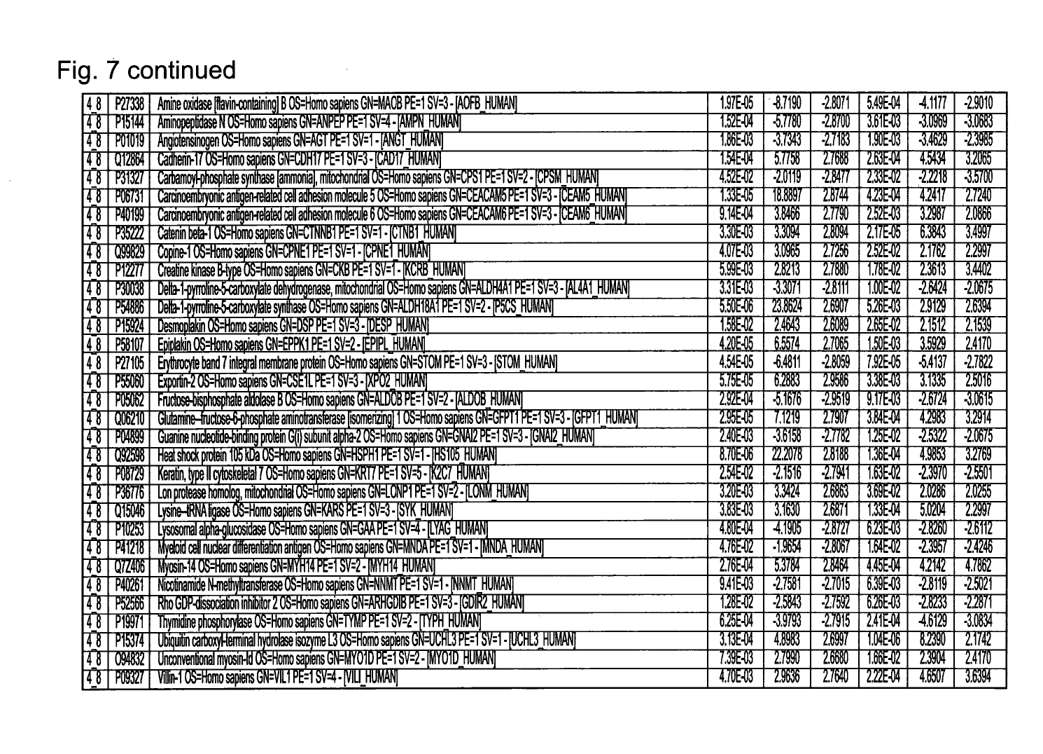

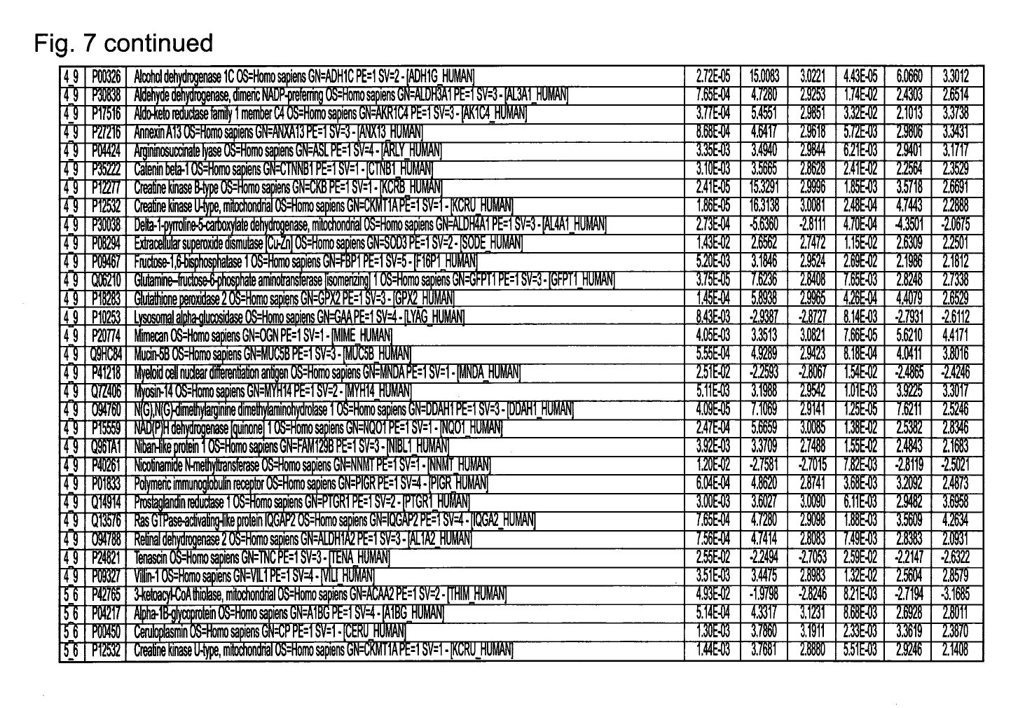

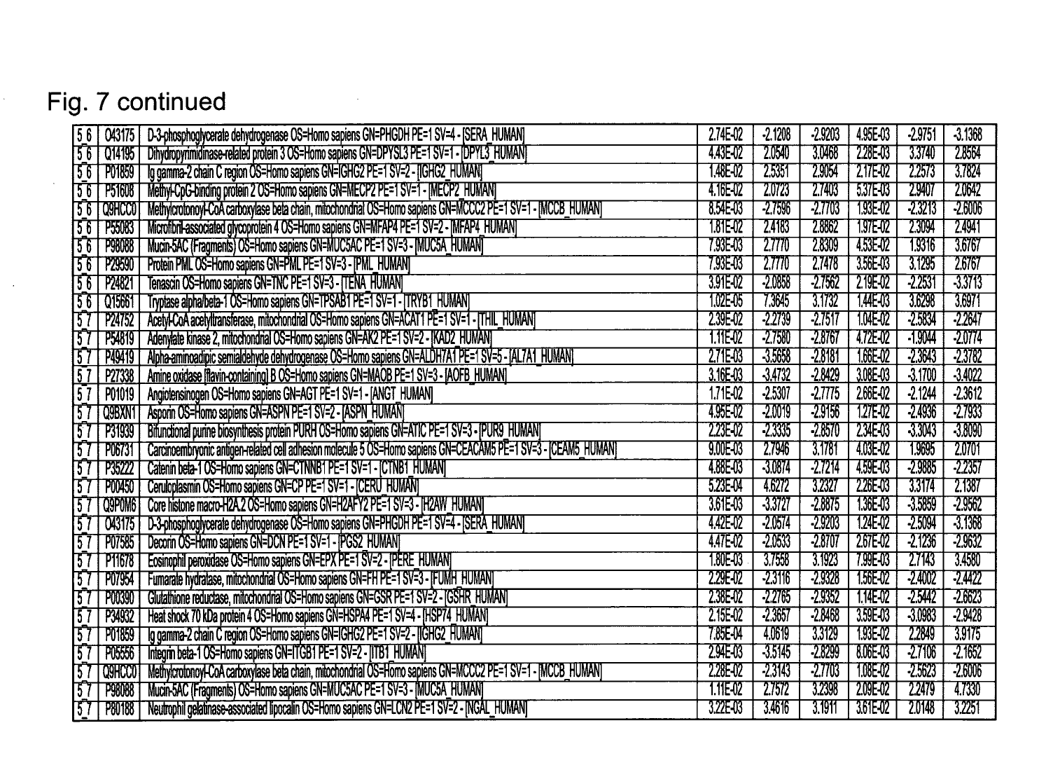

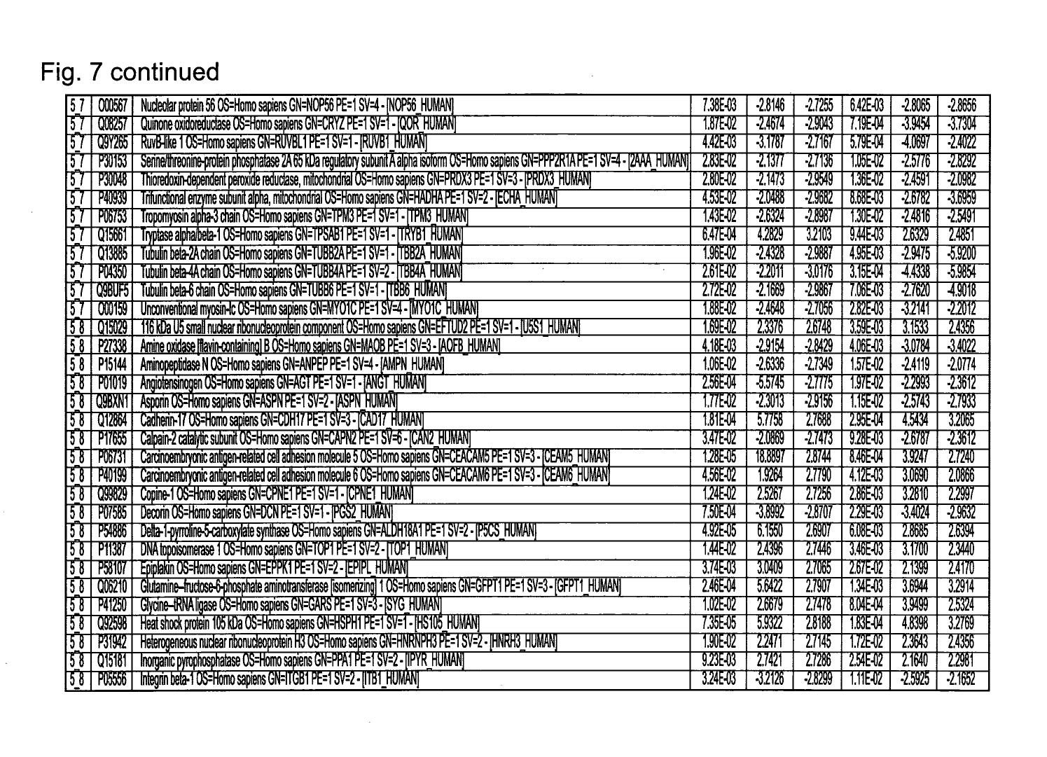

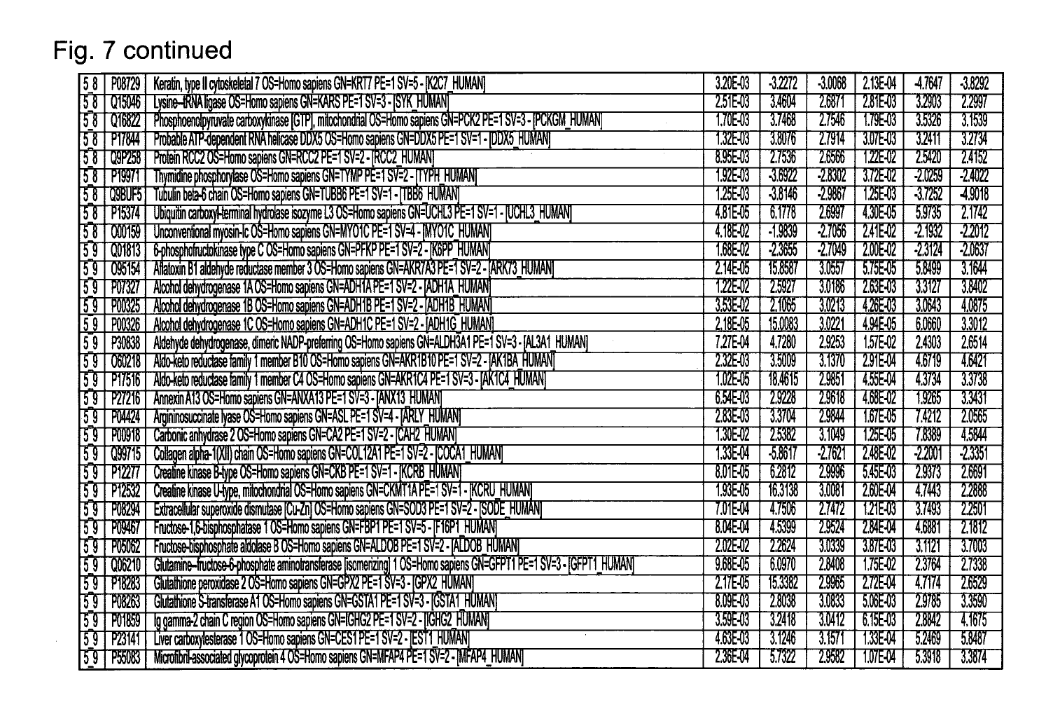

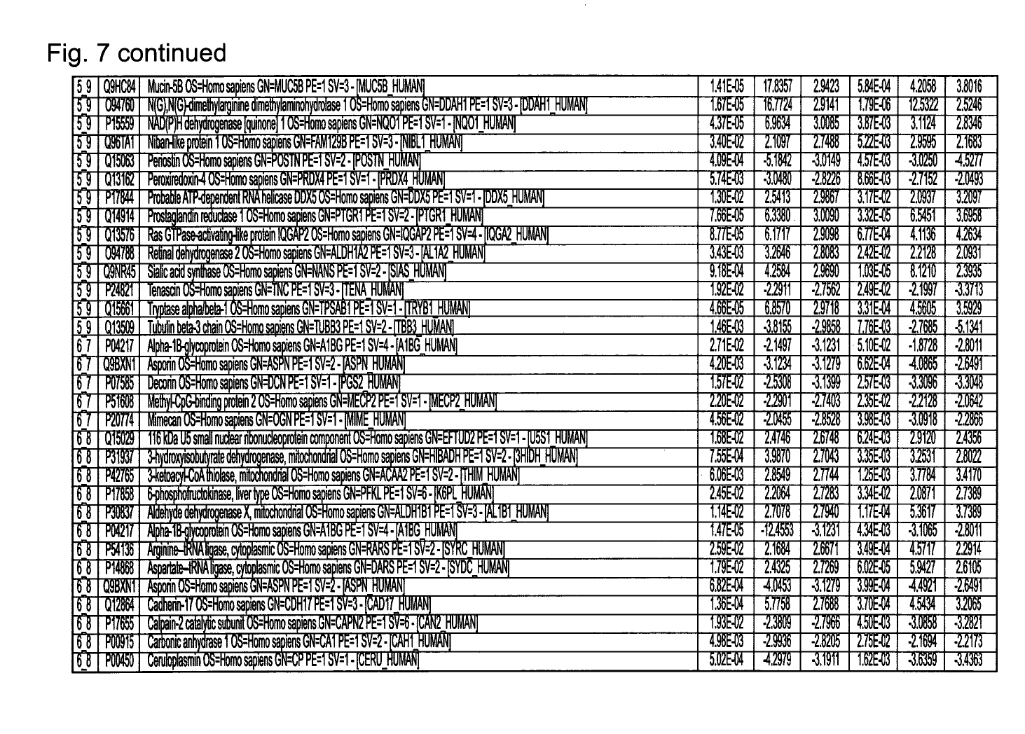

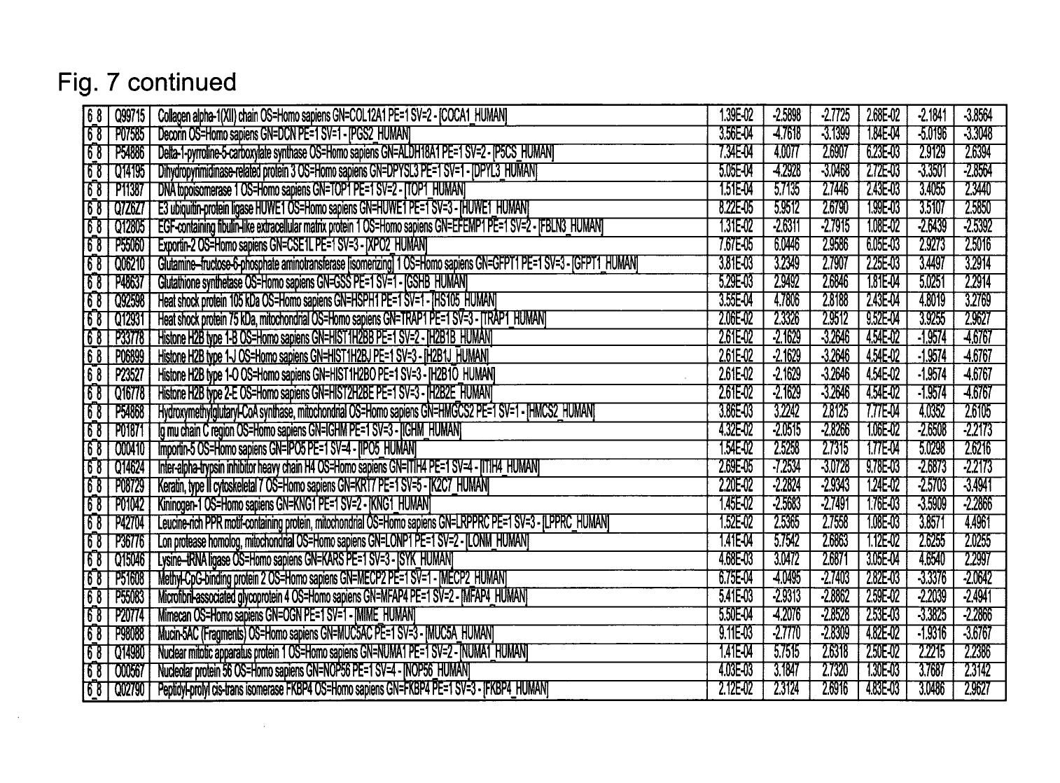

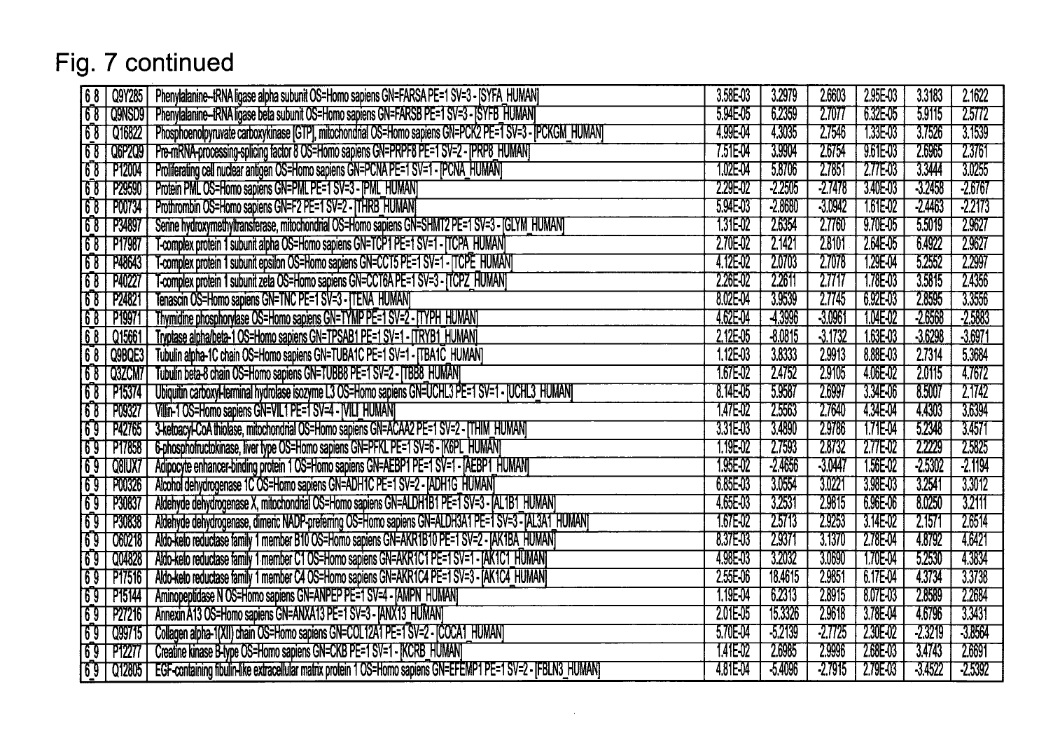

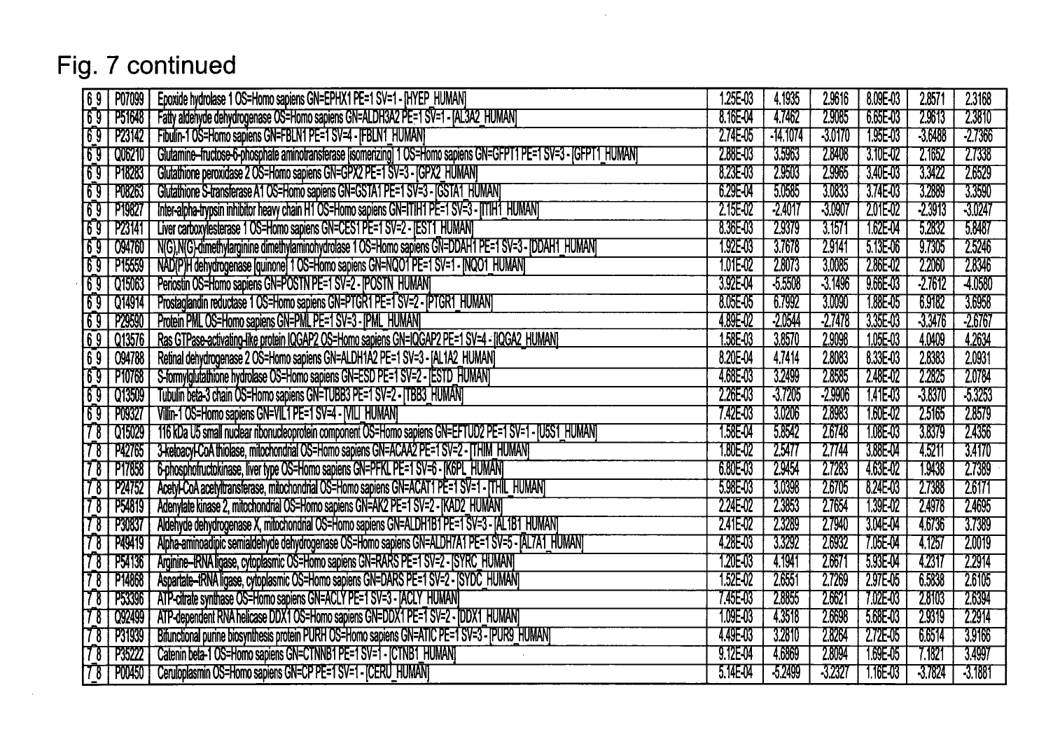

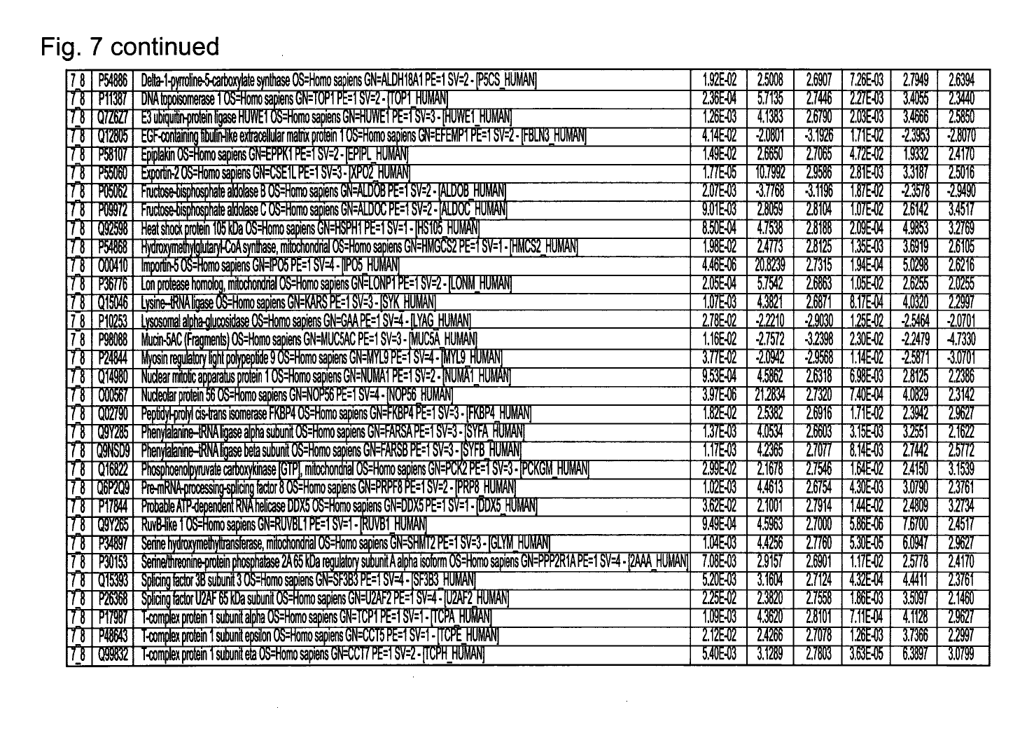

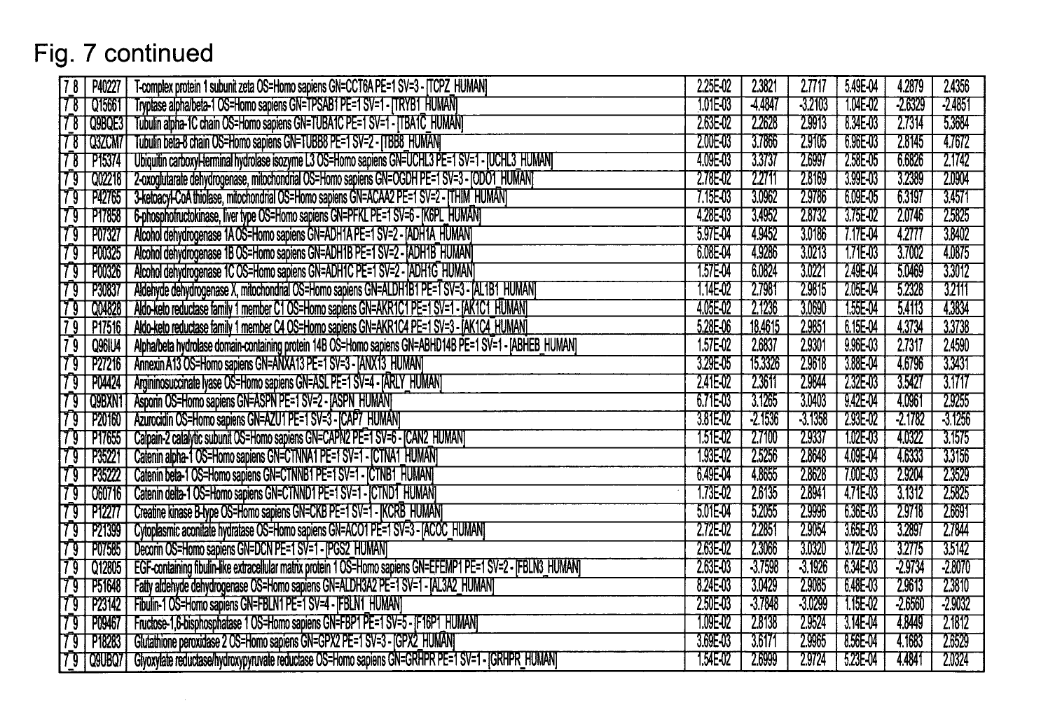

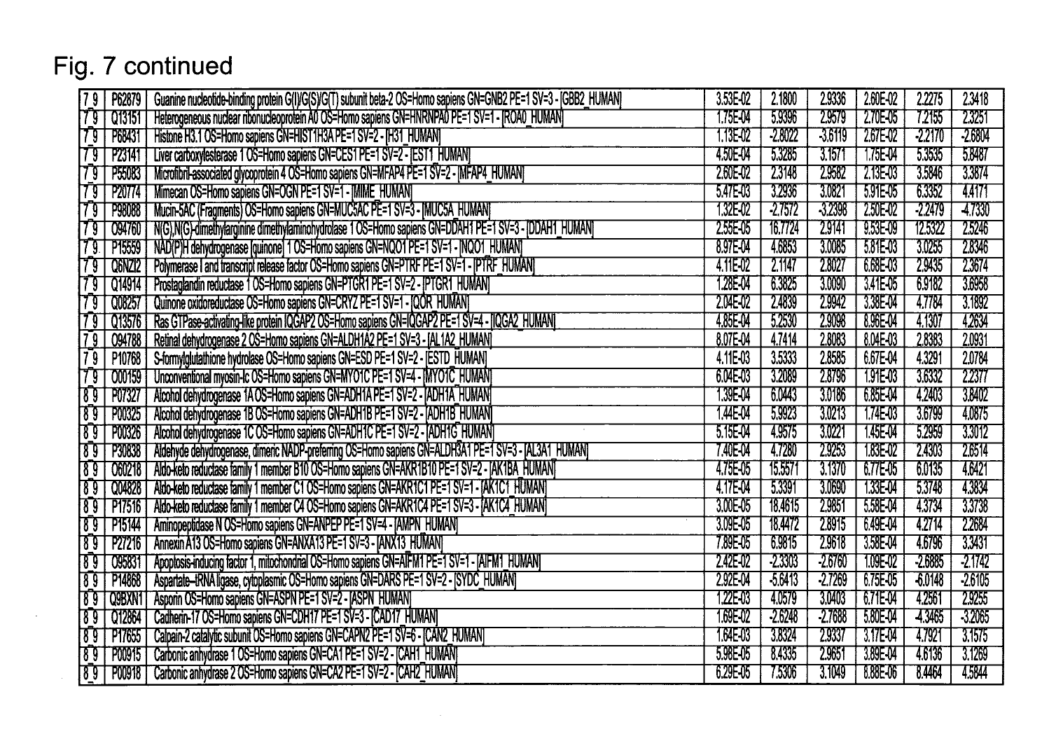

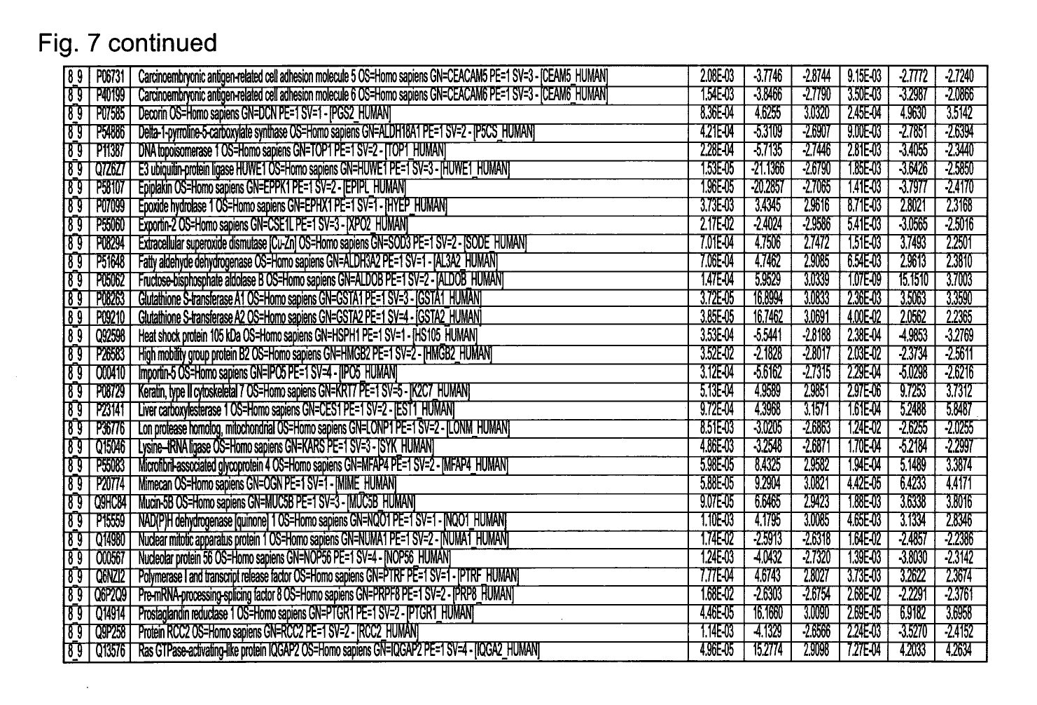

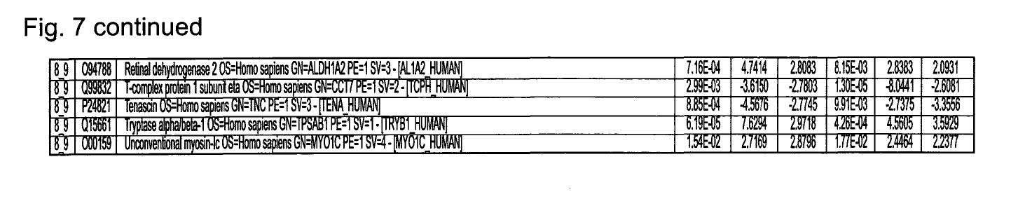

Protein expression levels for marker proteins shown in Tables 2 to 10 were determined using label free LC-MS/MS quantification based on spectral counting (shared and unique peptides) which is well known in the art. All marker proteins showing statistically significant differences in mean spectral counts between two tissue types are display in Tables 2-10. We have also used an alternate method of data analysis based on the area under the curve (AUC) of the MS1 peak of the three most intense peptides for each protein. All marker proteins in Table 11 (FIG. 7) marker proteins (467 marker proteins) were found to be significantly regulated in at least one of the tissue comparisons that were common to both quantification methods (spectral counting and AUC of both shared and unique peptides). The Table contains tissue type comparison (Tissue type number versus tissue type number), uniprot ID, and protein names along with P-values, t-scores and log₂ Fold-change values for both quantitative methods.

In Tables 2 to 10 the significantly modulated marker proteins were filtered by the stringent q-values (section A), then the less stringent p-values (section B). In Table 11 (FIG. 7) the marker proteins were filtered on p-values and fold change (combined). In summary, Tables 2 to 10 only considered spectral counts for quantification, whereas Table 11 considered spectral counts and area under the curve.

Table 2 provides protein markers for use in distinguishing normal hepatocytes from hepatocellular carcinoma cells (HCC).

TABLE 2
Proteins differentiating normal hepatocytes from hepatocellular carcinoma*

| | | | | | | |
|---|---|---|---|---|---|---|
| | | | | | Effect | Magnitude |
| | | | Effect | Magnitude | Size | of |
| | | | Size | of Effect | (Mean | Mean dif |
| Protein | Name | P Value | (g) | Size (g) | Dif) | (Mean Dif) |

PART A:

| | | | | | | |
|---|---|---|---|---|---|---|
| P39060* | Collagen alpha-1(XVIII) | 2.28E−07 | −5.26 | 5.26 | −6.71 | 6.71 |
| | chain/Name = COL18A1 | | | | | |
| Q9H2A2 | Aldehyde dehydrogenase | 2.27E−05 | 3.39 | 3.39 | 7.71 | 7.71 |
| | family 8 member A1/ | | | | | |
| | Name = ALDH8A1; | | | | | |
| | Synonyms = ALDH12 | | | | | |
| P10632 | Cytochrome P4502C8/ | 3.25E−05 | 3.25 | 3.25 | 10.71 | 10.71 |
| | Name = CYP2C8 | | | | | |
| P00367 | Glutamate dehydrogenase 1, | 3.26E−05 | 3.52 | 3.52 | 34.86 | 34.86 |
| | mitochondrial/ | | | | | |
| | Name = GLUD1; | | | | | |
| | Synonyms = GLUD | | | | | |
| P07355* | Annexin A2/Name = ANXA2; | 3.36E−05 | −3.7 | 3.7 | −10.57 | 10.57 |
| | Synonyms = ANX2, | | | | | |
| | ANX2L4, CAL1H, LPC2D | | | | | |
| Q6IB77 | Glycine N-acyltransferase/ | 4.20E−05 | 3.14 | 3.14 | 10.29 | 10.29 |
| | Name = GLYAT; | | | | | |
| | Synonyms = ACGNAT, CAT, | | | | | |
| | GAT | | | | | |
| P02751* | Fibronectin/Name = FN1; | 1.58E−04 | −3.61 | 3.61 | −19.43 | 19.43 |
| | Synonyms = FN | | | | | |
| Q02338 | D-beta-hydroxybutyrate | 1.83E−04 | 2.68 | 2.68 | 7.86 | 7.86 |
| | dehydrogenase, mitochondrial/ | | | | | |
| | Name = BDH1; | | | | | |
| | Synonyms = BDH | | | | | |
| P05091 | Aldehyde dehydrogenase, | 2.02E−04 | 2.65 | 2.65 | 27.86 | 27.86 |
| | mitochondrial/ | | | | | |
| | Name = ALDH2; | | | | | |
| | Synonyms = ALDM | | | | | |
| P45954 | Short/branched chain specific | 2.95E−04 | 2.59 | 2.59 | 8.71 | 8.71 |
| | acyl-CoA dehydrogenase, | | | | | |
| | mitochondrial/ | | | | | |
| | Name = ACADSB | | | | | |
| Q9Y617 | Phosphoserine | 3.68E−04 | 3.41 | 3.41 | 5.43 | 5.43 |
| | aminotransferase/ | | | | | |
| | Name = PSAT1; | | | | | |
| | Synonyms = PSA | | | | | |
| P30084 | Enoyl-CoA hydratase, | 3.85E−04 | 2.48 | 2.48 | 12.57 | 12.57 |
| | mitochondrial/Name = ECHS1 | | | | | |
| O75452 | Retinol dehydrogenase 16/ | 4.23E−04 | 2.41 | 2.41 | 8.71 | 8.71 |
| | Name = RDH16; | | | | | |
| | Synonyms = RODH4 | | | | | |
| P16219 | Short-chain specific | 4.84E−04 | 2.61 | 2.61 | 12.14 | 12.14 |
| | acyl-CoA dehydrogenase, | | | | | |
| | mitochondrial/ | | | | | |
| | Name = ACADS | | | | | |
| P21695 | Glycerol-3-phosphate | 5.29E−04 | 2.36 | 2.36 | 6.71 | 6.71 |
| | dehydrogenase [NAD+], | | | | | |
| | cytoplasmic/Name = GPD1 | | | | | |
| P09467 | Fructose-1,6-bisphosphatase 1/ | 6.24E−04 | 2.4 | 2.4 | 12.43 | 12.43 |
| | Name = FBP1; | | | | | |
| | Synonyms = FBP | | | | | |
| P00439 | Phenylalanine-4-hydroxylase/ | 6.34E−04 | 2.32 | 2.32 | 4.71 | 4.71 |
| | Name = PAH | | | | | |
| O95954 | Formimidoyltransferase- | 7.19E−04 | 2.34 | 2.34 | 23.57 | 23.57 |
| | cyclodeaminase/Name = FTCD | | | | | |
| P34913 | Epoxide hydrolase 2/ | 8.34E−04 | 2.37 | 2.37 | 8 | 8 |
| | Name = EPHX2 | | | | | |
| P01876* | Ig alpha-1 chain C region/ | 1.03E−03 | −2.16 | 2.16 | −6.57 | 6.57 |
| | Name = IGHA1 | | | | | |

PART B:

| | | | | | | |
|---|---|---|---|---|---|---|
| P49189 | 4-trimethylaminobutyraldehyde | 1.23E−03 | 2.21 | 2.21 | 3 | 3 |
| | dehydrogenase/ | | | | | |
| | Name = ALDH9A1; | | | | | |
| | Synonyms = ALDH4, ALDH7, | | | | | |
| | ALDH9 | | | | | |
| P06737 | Glycogen phosphorylase, | 1.30E−03 | 2.1 | 2.1 | 11 | 11 |
| | liver form/Name = PYGL | | | | | |
| P62807 | Histone H2B type 1-C/E/F/G/I/ | 1.35E−03 | −2.08 | 2.08 | −10.57 | 10.57 |
| | Name = HIST1H2BC; | | | | | |
| | Synonyms = H2BFL | | | | | |
| P58876 | Histone H2B type 1-D/ | 1.35E−03 | −2.08 | 2.08 | −10.57 | 10.57 |
| | Name = HIST1H2BD; | | | | | |
| | Synonyms = H2BFB, HIRIP2 | | | | | |
| Q93079 | Histone H2B type 1-H/ | 1.35E−03 | −2.08 | 2.08 | −10.57 | 10.57 |
| | Name = HIST1H2BH; | | | | | |
| | Synonyms = H2BFJ | | | | | |
| O60814 | Histone H2B type 1-K/ | 1.35E−03 | −2.08 | 2.08 | −10.57 | 10.57 |
| | Name = HIST1H2BK; | | | | | |
| | Synonyms = H2BFT, HIRIP1 | | | | | |
| Q99880 | Histone H2B type 1-L/ | 1.35E−03 | −2.08 | 2.08 | −10.57 | 10.57 |
| | Name = HIST1H2BL; | | | | | |
| | Synonyms = H2BFC | | | | | |
| Q99879 | Histone H2B type 1-M/ | 1.35E−03 | −2.08 | 2.08 | −10.57 | 10.57 |
| | Name = HIST1H2BM; | | | | | |
| | Synonyms = H2BFE | | | | | |
| Q99877 | Histone H2B type 1-N/ | 1.35E−03 | −2.08 | 2.08 | −10.57 | 10.57 |
| | Name = HIST1H2BN; | | | | | |
| | Synonyms = H2BFD | | | | | |
| Q5QNW6 | Histone H2B type 2-F/ | 1.35E−03 | −2.08 | 2.08 | −10.57 | 10.57 |
| | Name = HIST2H2BF | | | | | |
| P57053 | Histone H2B type F-S/ | 1.35E−03 | −2.08 | 2.08 | −10.57 | 10.57 |
| | Name = H2BFS | | | | | |
| P16401 | Histone H1.5/ | 1.39E−03 | −2.29 | 2.29 | −4.86 | 4.86 |
| | Name = HIST1H1B; | | | | | |
| | Synonyms = H1F5 | | | | | |
| P27824 | Calnexin/Name = CANX | 1.56E−03 | −2.04 | 2.04 | −6.43 | 6.43 |
| Q02928 | Cytochrome P4504A11/ | 1.69E−03 | 2.02 | 2.02 | 5.43 | 5.43 |
| | Name = CYP4A11; | | | | | |
| | Synonyms = CYP4A2 | | | | | |
| Q9UL12 | Sarcosine dehydrogenase, | 1.85E−03 | 2.01 | 2.01 | 10 | 10 |
| | mitochondrial/ | | | | | |
| | Name = SARDH; | | | | | |
| | Synonyms = DMGDHL1 | | | | | |
| Q07020 | 60S ribosomal protein L18/ | 1.97E−03 | −2.03 | 2.03 | −3 | 3 |
| | Name = RPL18 | | | | | |
| P62269 | 40S ribosomalprotein S18/ | 2.26E−03 | −1.93 | 1.93 | −5.71 | 5.71 |
| | Name = RPS18; | | | | | |
| | Synonyms = D6S218E | | | | | |
| Q68CK6 | Acyl-coenzyme A synthetase | 2.74E−03 | 1.92 | 1.92 | 14.71 | 14.71 |
| | ACSM2B, mitochondrial/ | | | | | |
| | Name = ACSM2B; | | | | | |
| | Synonyms = ACSM2; | | | | | |
| | ORFNames = HYST1046 | | | | | |
| P26599 | Polypyrimidine tract-binding | 2.75E−03 | −2.01 | 2.01 | −4 | 4 |
| | protein 1/Name = PTBP1; | | | | | |
| | Synonyms = PTB | | | | | |
| Q04837 | Single-stranded DNA-binding | 2.93E−03 | −2.41 | 2.41 | −2.29 | 2.29 |
| | protein, mitochondrial/ | | | | | |
| | Name = SSBP1; | | | | | |
| | Synonyms = SSBP | | | | | |
| P35573 | Glycogen debranching enzyme/ | 3.20E−03 | 2.13 | 2.13 | 12.43 | 12.43 |
| | Name = AGL; | | | | | |
| | Synonyms = GDE | | | | | |
| Q16836 | Hydroxyacyl-coenzyme A | 3.20E−03 | 1.84 | 1.84 | 4.57 | 4.57 |
| | dehydrogenase, mitochondrial/ | | | | | |
| | Name = HADH; | | | | | |
| | Synonyms = HAD, HADHSC, | | | | | |
| | SCHAD | | | | | |
| P01834 | Ig kappa chain C region/ | 3.49E−03 | −1.93 | 1.93 | −10 | 10 |
| | Name = IGKC | | | | | |
| Q08AH3 | Acyl-coenzyme A synthetase | 3.66E−03 | 1.83 | 1.83 | 15.14 | 15.14 |
| | ACSM2A, mitochondrial/ | | | | | |
| | Name = ACSM2A; | | | | | |
| | Synonyms = ACSM2, MACS2 | | | | | |
| P19105 | Myosin regulatory light | 3.66E−03 | −1.8 | 1.8 | −2.14 | 2.14 |
| | chain 12A/Name = MYL12A; | | | | | |
| | Synonyms = MLCB, MRLC3, | | | | | |
| | RLC | | | | | |
| O14950 | Myosin regulatory light | 3.66E−03 | −1.8 | 1.8 | −2.14 | 2.14 |
| | chain 12B/Name = MYL12B; | | | | | |
| | Synonyms = MRLC2, | | | | | |
| | MYLC2B | | | | | |
| P02649 | Apolipoprotein E/ | 3.66E−03 | −1.81 | 1.81 | −7.43 | 7.43 |
| | Name = APOE | | | | | |
| Q9Y2P5 | Bile acyl-CoA synthetase/ | 3.95E−03 | 1.78 | 1.78 | 11 | 11 |
| | Name = SLC27A5; | | | | | |
| | Synonyms = ACSB, | | | | | |
| | ACSVL6, FACVL3, FATP5 | | | | | |
| P37802 | Transgelin-2/ | 4.12E−03 | −2.04 | 2.04 | −5.29 | 5.29 |
| | Name = TAGLN2; | | | | | |
| | Synonyms = KIAA0120; | | | | | |
| | ORFNames = CDABP0035 | | | | | |
| Q86XE5 | Probable | 4.84E−03 | 1.81 | 1.81 | 2 | 2 |
| | 4-hydroxy-2-oxoglutarate | | | | | |
| | aldolase, mitochondrial/ | | | | | |
| | Name = HOGA1; | | | | | |
| | Synonyms = C10orf65, | | | | | |
| | DHDPSL | | | | | |
| O43772 | Mitochondrial | 4.92E−03 | 1.75 | 1.75 | 4.86 | 4.86 |
| | carnitine/acylcarnitine carrier | | | | | |
| | protein/Name = SLC25A20; | | | | | |
| | Synonyms = CAC, CACT | | | | | |
| Q16134 | Electron transfer | 5.09E−03 | 1.72 | 1.72 | 4.86 | 4.86 |
| | flavoprotein-ubiquinone | | | | | |
| | oxidoreductase, mitochondrial/ | | | | | |
| | Name = ETFDH | | | | | |
| Q7Z5P4 | 17-beta-hydroxysteroid | 5.15E−03 | 1.71 | 1.71 | 6.57 | 6.57 |
| | dehydrogenase 13/ | | | | | |
| | Name = HSD17B13; | | | | | |
| | Synonyms = SCDR9; | | | | | |
| | ORFNames = HMFN0376, | | | | | |
| | UNQ497/PRO1014 | | | | | |
| P09651 | Heterogeneous nuclear | 5.18E−03 | −1.77 | 1.77 | −3.14 | 3.14 |
| | ribonucleoprotein A1/ | | | | | |
| | Name = HNRNPA1; | | | | | |
| | Synonyms = HNRPA1 | | | | | |
| Q3LXA3 | Bifunctional ATP-dependent | 5.25E−03 | 1.71 | 1.71 | 19.43 | 19.43 |
| | dihydroxyacetone kinase/ | | | | | |
| | FAD-AMP lyase (cyclizing)/ | | | | | |
| | Name = DAK | | | | | |
| Q02878 | 60S ribosomal protein L6/ | 5.39E−03 | −1.72 | 1.72 | −4.29 | 4.29 |
| | Name = RPL6; | | | | | |
| | Synonyms = TXREB1 | | | | | |
| P98160 | Basement membrane-specific | 5.49E−03 | −1.93 | 1.93 | −11 | 11 |
| | heparan sulfate proteoglycan | | | | | |
| | core protein/Name = HSPG2 | | | | | |
| P84103 | Serine/arginine-rich splicing | 5.59E−03 | −2.11 | 2.11 | −2.14 | 2.14 |
| | factor 3/Name = SRSF3; | | | | | |
| | Synonyms = SFRS3, SRP20 | | | | | |
| P05177 | Cytochrome P4501A2/ | 5.93E−03 | 1.78 | 1.78 | 6.29 | 6.29 |
| | Name = CYP1A2 | | | | | |
| P62847 | 40S ribosomal protein S24/ | 5.97E−03 | −1.72 | 1.72 | −2.29 | 2.29 |
| | Name = RPS24 | | | | | |
| Q16851 | UTP--glucose-1-phosphate | 6.19E−03 | 1.82 | 1.82 | 17 | 17 |
| | uridylyltransferase/ | | | | | |
| | Name = UGP2; | | | | | |
| | Synonyms = UGP1 | | | | | |
| Q9UQ80 | Proliferation-associated | 6.43E−03 | −1.65 | 1.65 | −2.86 | 2.86 |
| | protein 2G4/Name = PA2G4; | | | | | |
| | Synonyms = EBP1 | | | | | |
| P62937 | Peptidyl-prolyl cis-trans | 6.77E−03 | −1.71 | 1.71 | −7.57 | 7.57 |
| | isomerase A/Name = PPIA; | | | | | |
| | Synonyms = CYPA | | | | | |
| P08238 | Hea shock protein HSP | 6.79E−03 | −1.72 | 1.72 | −8.57 | 8.57 |
| | 90-beta/Name = HSP90AB1; | | | | | |
| | Synonyms = HSP90B, | | | | | |
| | HSPC2, HSPCB | | | | | |
| Q9U117 | Dimethylglycine | 6.89E−03 | 1.64 | 1.64 | 7 | 7 |
| | dehydrogenase, mitochondrial/ | | | | | |
| | Name = DMGDH | | | | | |
| O60218 | Aldo-keto reductase family 1 | 6.95E−03 | −1.97 | 1.97 | −14.43 | 14.43 |
| | member B10/ | | | | | |
| | Name = AKR1B10; | | | | | |
| | Synonyms = AKR1B11 | | | | | |
| P00918 | Carbonic anhydrase 2/ | 7.02E−03 | 1.65 | 1.65 | 4.86 | 4.86 |
| | Name = CA2 | | | | | |
| P36578 | 60S ribosomal protein L4/ | 7.35E−03 | −1.68 | 1.68 | −3.71 | 3.71 |
| | Name = RPL4; | | | | | |
| | Synonyms = RPL1 | | | | | |
| P00915 | Carbonic anhydrase 1/ | 7.43E−03 | 1.61 | 1.61 | 5.14 | 5.14 |
| | Name = CA1 | | | | | |
| P84077 | ADP-ribosylation factor 1/ | 7.87E−03 | −1.6 | 1.6 | −3.86 | 3.86 |
| | Name = ARF1 | | | | | |
| P61204 | ADP-ribosylation factor 3/ | 7.87E−03 | −1.6 | 1.6 | −3.86 | 3.86 |
| | Name = ARF3 | | | | | |
| P51857 | 3-oxo-5-beta-steroid | 8.04E−03 | 1.95 | 1.95 | 6.29 | 6.29 |
| | 4-dehydrogenase/ | | | | | |
| | Name = AKR1D1; | | | | | |
| | Synonyms = SRD5B1 | | | | | |
| Q86UE4 | Protein LYRIC/ | 8.40E−03 | −1.58 | 1.58 | −2 | 2 |
| | Name = MTDH; | | | | | |
| | Synonyms = AEG1, LYRIC | | | | | |
| P63104 | 14-3-3 protein zeta/delta/ | 8.55E−03 | −1.73 | 1.73 | −5.71 | 5.71 |
| | Name = YWHAZ | | | | | |
| P02730 | Band 3 anion transport | 8.62E−03 | 1.92 | 1.92 | 3.71 | 3.71 |
| | protein/Name = SLC4A1; | | | | | |
| | Synonyms = AE1, DI, EPB3 | | | | | |
| Q9UBR1 | Beta-ureidopropionase/ | 8.63E−03 | 1.61 | 1.61 | 7.43 | 7.43 |
| | Name = UPB1; | | | | | |
| | Synonyms = BUP1 | | | | | |
| P08319 | Alcohol dehydrogenase 4/ | 8.79E−03 | 1.57 | 1.57 | 37.86 | 37.86 |
| | Name = ADH4 | | | | | |
| P00491 | Purin nucleoside | 9.05E−03 | 1.56 | 1.56 | 4.29 | 4.29 |
| | phosphorylase/Name = PNP; | | | | | |
| | Synonyms = NP | | | | | |
| P61978 | Heterogeneous nuclear | 9.14E−03 | −1.68 | 1.68 | −5.57 | 5.57 |
| | ribonucleoprotein K/ | | | | | |
| | Name = HNRNPK; | | | | | |
| | Synonyms = HNRPK | | | | | |
| P34896 | Serine | 9.87E−03 | 1.62 | 1.62 | 9 | 9 |
| | hydroxymethyltransferase, | | | | | |
| | cytosolic/Name = SHMT1 | | | | | |
| Q12905 | Interleukin enhancer-binding | 9.90E−03 | −1.54 | 1.54 | −2.29 | 2.29 |
| | factor 2/Name = ILF2; | | | | | |
| | Synonyms = NF45; | | | | | |
| | ORFNames = PRO3063 | | | | | |

*Bold type indicates increased relative expression in hepatocellular carcinoma compared to normal hepatocytes.

Table 2 provides information as to whether the marker proteins are relatively over-expressed (identified in bold) or under-expressed in HCC versus normal hepatocytes. Accordingly, by determining the presence, absence or change in expression levels of a plurality of these marker proteins and comparing these changes with a reference of known expression levels, one is able to determine whether the cells under test are HCC or normal hepatocytes. The plurality of marker proteins may be selected from Table 2 as a whole, or preferably from Part A which lists those marker proteins showing a higher statistically significant difference between the two cell types.

Table 3 provides information as to whether the marker proteins are relatively over-expressed (identified in bold) or under-expressed in peripheral cholangiocarcinoma versus normal cholangiocytes. Accordingly, by determining the presence, absence or change in expression levels of a plurality of these marker proteins and comparing these changes with a reference of known expression levels, one is able to determine whether the cells under test are peripheral cholangiocarcinoma or normal cholangiocytes.

TABLE 3
Proteins differentiating peripheral cholangiocarcinoma from normal
cholangiocytes*

| | | | | | | |
|---|---|---|---|---|---|---|
| | | | | Magnitude | Effect | |
| | | | Effect | of | Size | Magnitude |
| | | | Size | Effect | (Mean | of Mean dif |
| Protein | Name | P Value | (g) | Size (g) | Dif) | (Mean Dif) |

PART A:

| | | | | | | |
|---|---|---|---|---|---|---|
| P04424 | Argininosuccinate lyase/ | 5.71E−05 | −3.6 | 3.6 | −6.57 | 6.57 |
| | Name = ASL | | | | | |
| O95994 | Anterior gradient protein 2 | 8.34E−05 | −3.5 | 3.5 | −10.55 | 10.55 |
| | homolog/Name = AGR2; | | | | | |
| | Synonyms = AG2; | | | | | |
| | ORFNames = UNQ515/PRO1030 | | | | | |
| P07327 | Alcohol dehydrogenase 1A/ | 9.97E−05 | −5.06 | 5.06 | −13.24 | 13.24 |
| | Name = ADH1A; | | | | | |
| | Synonyms = ADH1 | | | | | |
| O94760 | N(G),N(G)-dimethylarginine | 1.17E−04 | −6.1 | 6.1 | −5.17 | 5.17 |
| | dimethylaminohydrolase 1/ | | | | | |
| | Name = DDAH1; | | | | | |
| | Synonyms = DDAH | | | | | |
| Q9NR45 | Sialic acid synthase/ | 1.38E−04 | −3.09 | 3.09 | −3.43 | 3.43 |
| | Name = NANS; Synonyms = SAS | | | | | |
| P56470 | Galectin-4/Name = LGALS4 | 1.76E−04 | −3.02 | 3.02 | −14.74 | 14.74 |
| P00352 | Retinal dehydrogenase 1/ | 2.43E−04 | −2.83 | 2.83 | −27.55 | 27.55 |
| | Name = ALDH1A1; | | | | | |
| | Synonyms = ALDC, ALDH1, | | | | | |
| | PUMB1 | | | | | |
| P08263 | Glutathione S-transferase A1/ | 3.42E−04 | −2.9 | 2.9 | −8.14 | 8.14 |
| | Name = GSTA1 | | | | | |
| O95154 | Aflatoxin B1 aldehyde reductase | 3.52E−04 | −4.84 | 4.84 | −8 | 8 |
| | member 3/Name = AKR7A3; | | | | | |
| | Synonyms = AFAR2 | | | | | |
| P09211 | Glutathione S-transferase P/ | 4.23E−04 | −2.53 | 2.53 | −22.02 | 22.02 |
| | Name = GSTP1; | | | | | |
| | Synonyms = FAEES3, GST3 | | | | | |
| P00918 | Carbonic anhydrase 2/ | 6.13E−04 | −3.84 | 3.84 | −18.71 | 18.71 |
| | Name = CA2 | | | | | |
| Q13228 | Selenium-binding protein 1/ | 6.18E−04 | −2.81 | 2.81 | −9.81 | 9.81 |
| | Name = SELENBP1; | | | | | |
| | Synonyms = SBP | | | | | |
| P20774 | Mimecan/Name = OGN; | 6.39E−04 | −3.32 | 3.32 | −18.76 | 18.76 |
| | Synonyms = OIF, SLRR3A | | | | | |
| O75489 | NADH dehydrogenase | 7.45E−04 | −2.4 | 2.4 | −2.43 | 2.43 |
| | [ubiquinone] iron-sulfur protein | | | | | |
| | 3, mitochondrial/ | | | | | |
| | Name = NDUFS3 | | | | | |
| Q14914 | Prostaglandin reductase 1/ | 7.50E−04 | −3.54 | 3.54 | −9.57 | 9.57 |
| | Name = PTGR1; | | | | | |
| | Synonyms = LTB4DH | | | | | |
| P21333* | Filamin-A/Name = FLNA; | 9.79E−04 | 2.3 | 2.3 | 37.69 | 37.69 |
| | Synonyms = FLN, FLN1 | | | | | |
| P00325 | Alcohol dehydrogenase 1B/ | 1.03E−03 | −2.4 | 2.4 | −14.33 | 14.33 |
| | Name = ADH1B; | | | | | |
| | Synonyms = ADH2 | | | | | |

PART B:

| | | | | | | |
|---|---|---|---|---|---|---|
| P00326 | Alcohol dehydrogenase 1C/ | 1.33E−03 | −3.63 | 3.63 | −10 | 10 |
| | Name = ADH1C; | | | | | |
| | Synonyms = ADH3 | | | | | |
| P09525 | Annexin A4/Name = ANXA4; | 1.63E−03 | −2.44 | 2.44 | −34.64 | 34.64 |
| | Synonyms = ANX4 | | | | | |
| P55083 | Microfibril-associated | 1.82E−03 | −2.74 | 2.74 | −8.67 | 8.67 |
| | glycoprotein 4/Name = MFAP4 | | | | | |
| Q14376 | UDP-glucose 4-epimerase/ | 2.25E−03 | −2.14 | 2.14 | −3.12 | 3.12 |
| | Name = GALE | | | | | |
| P09467 | Fructose-1,6-bisphosphatase 1/ | 2.32E−03 | −3.21 | 3.21 | −4.33 | 4.33 |
| | Name = FBP1; Synonyms = FBP | | | | | |
| P13611 | Versican core protein/ | 3.00E−03 | 2.21 | 2.21 | 13.55 | 13.55 |
| | Name = VCAN; | | | | | |
| | Synonyms = CSPG2 | | | | | |
| P21810 | Biglycan/Name = BGN; | 3.04E−03 | −2.02 | 2.02 | −17.88 | 17.88 |
| | Synonyms = SLRR1A | | | | | |
| P18283 | Glutathione peroxidase 2/ | 3.21E−03 | −2.98 | 2.98 | −4.67 | 4.67 |
| | Name = GPX2 | | | | | |
| P23141 | Liver carboxylesterase 1/ | 3.26E−03 | −2.88 | 2.88 | −42.02 | 42.02 |
| | Name = CES1; | | | | | |
| | Synonyms = CES2, SES1 | | | | | |
| O60218 | Aldo-keto reductase family 1 | 3.53E−03 | −2.38 | 2.38 | −18.83 | 18.83 |
| | member B10/Name = AKR1B10; | | | | | |
| | Synonyms = AKR1B11 | | | | | |
| P12277 | Creatine kinase B-type/ | 3.53E−03 | −2.11 | 2.11 | −5.29 | 5.29 |
| | Name = CKB; Synonyms = CKBB | | | | | |
| P42330 | Aldo-keto reductase family 1 | 4.39E−03 | −2.11 | 2.11 | −13.24 | 13.24 |
| | member C3/Name = AKR1C3; | | | | | |
| | Synonyms = DDH1, HSD17B5, | | | | | |
| | KIAA0119, PGFS | | | | | |
| Q16762 | Thiosulfate sulfurtransferase/ | 4.70E−03 | −1.81 | 1.81 | −7.33 | 7.33 |
| | Name = TST | | | | | |
| P12532 | Creatine kinase U-type, | 4.72E−03 | −2.72 | 2.72 | −3.83 | 3.83 |
| | mitochondrial/Name = CKMT1A; | | | | | |
| | Synonyms = CKMT | | | | | |
| P01622 | Ig kappa chain V-III region Ti/ | 4.82E−03 | −2.07 | 2.07 | −2.21 | 2.21 |
| P04206 | Ig kappa chain V-III region GOL/ | 4.82E−03 | −2.07 | 2.07 | −2.21 | 2.21 |
| P13797 | Plastin-3/Name = PLS3 | 5.39E−03 | 1.76 | 1.76 | 8.38 | 8.38 |
| O60664 | Perilipin-3/Name = PLIN3; | 5.48E−03 | 1.75 | 1.75 | 4.26 | 4.26 |
| | Synonyms = M6PRBP1, TIP47 | | | | | |
| P08311 | Cathepsin G/Name = CTSG | 5.54E−03 | −1.82 | 1.82 | −4.19 | 4.19 |
| P17516 | Aldo-keto reductase family 1 | 6.54E−03 | −2.52 | 2.52 | −8.83 | 8.83 |
| | member C4/Name = AKR1C4; | | | | | |
| | Synonyms = CHDR | | | | | |
| P08238 | Heat shock protein HSP 90-beta/ | 7.56E−03 | 1.71 | 1.71 | 8.76 | 8.76 |
| | Name = HSP90AB1; | | | | | |
| | Synonyms = HSP90B, HSPC2, | | | | | |
| | HSPCB | | | | | |
| P18206 | Vinculin/Name = VCL | 8.06E−03 | 1.94 | 1.94 | 5.86 | 5.86 |
| Q13576 | Ras GTPase-activating-like | 8.14E−03 | −2.37 | 2.37 | −14.38 | 14.38 |
| | protein IQGAP2/ | | | | | |
| | Name = IQGAP2 | | | | | |
| Q13509 | Tubulin beta-3 chain/ | 9.15E−03 | 1.8 | 1.8 | 28.57 | 28.57 |
| | Name = TUBB3; | | | | | |
| | Synonyms = TUBB4 | | | | | |
| P02751 | Fibronectin/Name = FN1; | 9.73E−03 | 1.62 | 1.62 | 36.02 | 36.02 |
| | Synonyms = FN | | | | | |

*Bold type indicates increased relative expression in peripheral cholangiocarcinoma compared to normal cholangiocytes

Table 4 provides information as to whether the marker proteins are relatively over-expressed (identified in bold) or under-expressed in hilar cholangiocarcinoma versus normal cholangiocytes. Accordingly, by determining the presence, absence or change in expression levels of a plurality of these marker proteins and comparing these changes with a reference of known expression levels, one is able to determine whether the cells under test are hilar cholangiocarcinoma or normal cholangiocytes.

TABLE 4
Proteins differentiating hilar cholangiocarcinoma from normal
cholangiocytes*

| | | | | | | |
|---|---|---|---|---|---|---|
| | | | | | Effect | Magnitude |
| | | | Effect | Magnitude | Size | of Mean |
| | | | Size | of Effect | (Mean | dif (Mean |
| Protein | Name | P Value | (g) | Size (g) | Dif) | Dif) |

PART A:

| | | | | | | |
|---|---|---|---|---|---|---|
| P05062 | Fructose-bisphosphate aldolase | 2.36E−05 | −3.85 | 3.85 | −9.9 | 9.9 |
| | B/Name = ALDOB; | | | | | |
| | Synonyms = ALDB | | | | | |
| P49411 | Elongation factor Tu, | 5.80E−05 | −3.23 | 3.23 | −5.62 | 5.62 |
| | mitochondrial/Name = TUFM | | | | | |
| P04424 | Argininosuccinate lyase/ | 7.38E−05 | −3.22 | 3.22 | −6.57 | 6.57 |
| | Name = ASL | | | | | |
| P30837 | Aldehyde dehydrogenase X, | 7.57E−05 | −3.11 | 3.11 | −6.93 | 6.93 |
| | mitochondrial/ | | | | | |
| | Name = ALDH1B1; | | | | | |
| | Synonyms = ALDH5, ALDHX | | | | | |
| O94760 | N(G),N(G)-dimethylarginine | 7.84E−05 | −3.13 | 3.13 | −4.17 | 4.17 |
| | dimethylaminohydrolase 1/ | | | | | |
| | Name = DDAH1; | | | | | |
| | Synonyms = DDAH | | | | | |
| P42765 | 3-ketoacyl-CoA thiolase, | 3.45E−04 | −2.71 | 2.71 | −7.48 | 7.48 |
| | mitochondrial/Name = ACAA2 | | | | | |
| P09525 | Annexin A4/Name = ANXA4; | 3.75E−04 | −2.76 | 2.76 | −43.5 | 43.5 |
| | Synonyms = ANX4 | | | | | |
| P07327 | Alcohol dehydrogenase 1A/ | 4.00E−04 | −2.54 | 2.54 | −10.67 | 10.67 |
| | Name = ADH1A; | | | | | |
| | Synonyms = ADH1 | | | | | |
| P08263 | Glutathione S-transferase A1/ | 5.04E−04 | −3.5 | 3.5 | −8.29 | 8.29 |
| | Name = GSTA1 | | | | | |
| P13797* | Plastin-3/Name = PLS3 | 5.11E−04 | 2.68 | 2.68 | 8.52 | 8.52 |
| P00325 | Alcohol dehydrogenase 1B/ | 8.05E−04 | −2.84 | 2.84 | −14.76 | 14.76 |
| | Name = ADH1B; | | | | | |
| | Synonyms = ADH2 | | | | | |
| Q14914 | Prostaglandin reductase 1/ | 9.44E−04 | −3.92 | 3.92 | −10 | 10 |
| | Name = PTGR1; | | | | | |
| | Synonyms = LTB4DH | | | | | |

PART B:

| | | | | | | |
|---|---|---|---|---|---|---|
| P00352 | Retinal dehydrogenase 1/ | 1.29E−03 | −2.38 | 2.38 | −21.26 | 21.26 |
| | Name = ALDH1A1; | | | | | |
| | Synonyms = ALDC, ALDH1, | | | | | |
| | PUMB1 | | | | | |
| P42330 | Aldo-keto reductase family 1 | 1.31E−03 | −2.89 | 2.89 | −16.81 | 16.81 |
| | member C3/Name = AKR1C3; | | | | | |
| | Synonyms = DDH1, HSD17B5, | | | | | |
| | KIAA0119, PGFS | | | | | |
| Q96KP4 | Cytosolic non-specific | 1.61E−03 | −2.17 | 2.17 | −10.48 | 10.48 |
| | dipeptidase/Name = CNDP2; | | | | | |
| | Synonyms = CN2, CPGL, PEPA | | | | | |
| P06396 | Gelsolin/Name = GSN | 1.80E−03 | 2.09 | 2.09 | 7.62 | 7.62 |
| O95154 | Aflatoxin B1 aldehyde reductase | 1.83E−03 | −2.07 | 2.07 | −6 | 6 |
| | member 3/Name = AKR7A3; | | | | | |
| | Synonyms = AFAR2 | | | | | |
| O75489 | NADH dehydrogenase | 1.98E−03 | −2.14 | 2.14 | −2.57 | 2.57 |
| | [ubiquinone] iron-sulfur protein | | | | | |
| | 3, mitochondrial/ | | | | | |
| | Name = NDUFS3 | | | | | |
| P20774 | Mimecan/Name = OGN; | 2.05E−03 | −2.53 | 2.53 | −14.9 | 14.9 |
| | Synonyms = OIF, SLRR3A | | | | | |
| Q14195 | Dihydropyrimidinase-related | 2.74E−03 | 2.33 | 2.33 | 6.29 | 6.29 |
| | protein 3/Name = DPYSL3; | | | | | |
| | Synonyms = CRMP4, DRP3, | | | | | |
| | ULIP, ULIP1 | | | | | |
| P25787 | Proteasome subunit alpha | 2.97E−03 | −1.94 | 1.94 | −3.38 | 3.38 |
| | type-2/Name = PSMA2; | | | | | |
| | Synonyms = HC3, PSC3 | | | | | |
| O60218 | Aldo-keto reductase family 1 | 2.98E−03 | −2.55 | 2.55 | −19.69 | 19.69 |
| | member B10/Name = AKR1B10; | | | | | |
| | Synonyms = AKR1B11 | | | | | |
| P12277 | Creatine kinase B-type/ | 3.15E−03 | −2.74 | 2.74 | −5.71 | 5.71 |
| | Name = CKB; Synonyms = CKBB | | | | | |
| P02545 | Prelamin-A/C/Name = LMNA; | 3.23E−03 | 1.95 | 1.95 | 10.24 | 10.24 |
| | Synonyms = LMN1 | | | | | |
| P23141 | Liver carboxylesterase 1/ | 3.36E−03 | −2.92 | 2.92 | −42.17 | 42.17 |
| | Name = CES1; | | | | | |
| | Synonyms = CES2, SES1 | | | | | |
| Q13228 | Selenium-binding protein 1/ | 3.37E−03 | −1.96 | 1.96 | −7.95 | 7.95 |
| | Name = SELENBP1; | | | | | |
| | Synonyms = SBP | | | | | |
| P27216 | Annexin A13/Name = ANXA13; | 3.90E−03 | −2.85 | 2.85 | −8.33 | 8.33 |
| | Synonyms = ANX13 | | | | | |
| Q16762 | Thiosulfate sulfurtransferase/ | 3.92E−03 | −1.84 | 1.84 | −8.76 | 8.76 |
| | Name = TST | | | | | |
| P31930 | Cytochrome b-c1 complex | 3.94E−03 | −1.97 | 1.97 | −2.33 | 2.33 |
| | subunit 1, mitochondrial/ | | | | | |
| | Name = UQCRC1 | | | | | |
| P00918 | Carbonic anhydrase 2/ | 4.75E−03 | −1.79 | 1.79 | −15 | 15 |
| | Name = CA2 | | | | | |
| Q08257 | Quinone oxidoreductase/ | 4.78E−03 | −1.8 | 1.8 | −4.93 | 4.93 |
| | Name = CRYZ | | | | | |
| P56470 | Galectin-4/Name = LGALS4 | 5.53E−03 | −1.79 | 1.79 | −9.45 | 9.45 |
| P18283 | Glutathione peroxidase 2/ | 5.82E−03 | −1.82 | 1.82 | −3.95 | 3.95 |
| | Name = GPX2 | | | | | |
| P00738 | Haptoglobin/Name = HP | 6.33E−03 | 1.73 | 1.73 | 7.64 | 7.64 |
| P17516 | Aldo-keto reductase family 1 | 6.54E−03 | −2.52 | 2.52 | −8.83 | 8.83 |
| | member C4/Name = AKR1C4; | | | | | |
| | Synonyms = CHDR | | | | | |
| P23142 | Fibulin-1/Name = FBLN1; | 6.64E−03 | 1.93 | 1.93 | 8.14 | 8.14 |
| | ORFNames = PP213 | | | | | |
| P08670 | Vimentin/Name = VIM | 7.13E−03 | 1.68 | 1.68 | 15.83 | 15.83 |
| Q9Y3Z3 | SAM domain and HD | 7.45E−03 | 1.66 | 1.66 | 2.5 | 2.5 |
| | domain-containing protein 1/ | | | | | |
| | Name = SAMHD1; | | | | | |
| | Synonyms = MOP5 | | | | | |
| Q14376 | UDP-glucose 4-epimerase/ | 7.68E−03 | −1.75 | 1.75 | −2.55 | 2.55 |
| | Name = GALE | | | | | |
| P09467 | Fructose-1,6-bisphosphatase 1/ | 8.04E−03 | −1.73 | 1.73 | −3.19 | 3.19 |
| | Name = FBP1; Synonyms = FBP | | | | | |
| Q12805 | EGF-containing fibulin-like | 8.24E−03 | 1.84 | 1.84 | 5.71 | 5.71 |
| | extracellular matrix protein 1/ | | | | | |
| | Name = EFEMP1; | | | | | |
| | Synonyms = FBLN3, FBNL | | | | | |
| P12429 | Annexin A3/Name = ANXA3; | 8.25E−03 | 1.74 | 1.74 | 5.81 | 5.81 |
| | Synonyms = ANX3 | | | | | |
| P60842 | Eukaryotic initiation factor 4A-I/ | 8.54E−03 | −1.62 | 1.62 | −5.95 | 5.95 |
| | Name = EIF4A1; | | | | | |
| | Synonyms = DDX2A, EIF4A | | | | | |
| Q13509 | Tubulin beta-3 chain/ | 8.81E−03 | 1.82 | 1.82 | 27.86 | 27.86 |
| | Name = TUBB3; | | | | | |
| | Synonyms = TUBB4 | | | | | |
| Q9UBR2 | Cathepsin Z/Name = CTSZ | 8.92E−03 | 1.66 | 1.66 | 2.79 | 2.79 |
| Q13576 | Ras GTPase-activating-like | 8.98E−03 | −2.28 | 2.28 | −13.95 | 13.95 |
| | protein IQGAP2/ | | | | | |
| | Name = IQGAP2 | | | | | |
| Q99536 | Synaptic vesicle membrane | 9.04E−03 | 1.61 | 1.61 | 3.29 | 3.29 |
| | protein VAT-1 homolog/ | | | | | |
| | Name = VAT1 | | | | | |
| P13611 | Versican core protein/ | 9.26E−03 | 1.78 | 1.78 | 17.4 | 17.4 |
| | Name = VCAN; | | | | | |
| | Synonyms = CSPG2 | | | | | |

*Bold type indicates increased relative expression in hilar cholangiocarcinoma compared to normal cholangiocytes

Table 5 provides information as to whether the marker proteins are relatively over-expressed (identified in bold) or under-expressed in peripheral carcinoma versus hepatocellular carcinoma. Accordingly, by determining the presence, absence or change in expression levels of a plurality of these marker proteins and comparing these changes with a reference of known expression levels, one is able to determine whether the cells under test are hepatocellular carcinoma or peripheral carcinoma.

TABLE 5
Proteins differentiating hepatocellular carcinoma from peripheral
cholangiocarcinoma*

| | | | | | | |
|---|---|---|---|---|---|---|
| | | | | | | Magnitude |
| | | | | | | of |
| | | | | | | Mean dif |
| | | | Effect | Magnitude of | Effect Size | (Mean |
| Protein | Name | P Value | Size (g) | Effect Size (g) | (Mean Dif) | Dif) |

PART A:

| | | | | | | |
|---|---|---|---|---|---|---|
| P18206* | Vinculin/Name = VCL | 1.65E−08 | −7.07 | 7.07 | −13.14 | 13.14 |
| P15311* | Ezrin/Name = EZR; | 8.38E−07 | −5.26 | 5.26 | −11.14 | 11.14 |
| | Synonyms = VIL2 | | | | | |
| P51659 | Peroxisomal multifunctional | 1.09E−06 | 5.92 | 5.92 | 40.43 | 40.43 |
| | enzyme type 2/ | | | | | |
| | Name = HSD17B4; | | | | | |
| | Synonyms = EDH17B4 | | | | | |
| P00352 | Retinal dehydrogenase 1/ | 1.55E−05 | 3.61 | 3.61 | 39.57 | 39.57 |
| | Name = ALDH1A1; | | | | | |
| | Synonyms = ALDC, ALDH1, | | | | | |
| | PUMB1 | | | | | |
| Q8NBX0 | Probable saccharopine | 1.60E−05 | 3.47 | 3.47 | 5.71 | 5.71 |
| | dehydrogenase/ | | | | | |
| | Name = SCCPDH; | | | | | |
| | ORFNames = CGI-49 | | | | | |
| P04040 | Catalase/Name = CAT | 2.67E−05 | 3.8 | 3.8 | 37 | 37 |
| P07148 | Fatty acid-binding protein, liver/ | 3.16E−05 | 3.4 | 3.4 | 20.29 | 20.29 |
| | Name = FABP1; | | | | | |
| | Synonyms = FABPL | | | | | |
| P33121 | Long-chain-fatty-acid--CoA | 3.20E−05 | 5.55 | 5.55 | 11.57 | 11.57 |
| | ligase 1/Name = ACSL1; | | | | | |
| | Synonyms = FACL1, FACL2, | | | | | |
| | LACS, LACS1, LACS2 | | | | | |
| P63104* | 14-3-3 protein zeta/delta/ | 3.33E−05 | −3.22 | 3.22 | −13.29 | 13.29 |
| | Name = YWHAZ | | | | | |
| P14618* | Pyruvate kinase isozymes | 3.76E−05 | −4.42 | 4.42 | −39.86 | 39.86 |
| | M1/M2/Name = PKM2; | | | | | |
| | Synonyms = OIP3, PK2, PK3, | | | | | |
| | PKM | | | | | |
| P09525* | Annexin A4/Name = ANXA4; | 4.58E−05 | −3.8 | 3.8 | −33.14 | 33.14 |
| | Synonyms = ANX4 | | | | | |
| P31949* | Protein S100-A11/ | 4.81E−05 | −5.17 | 5.17 | −3.71 | 3.71 |
| | Name = S100A11; | | | | | |
| | Synonyms = MLN70, S100C | | | | | |
| O95831 | Apoptosis-inducing factor 1, | 5.22E−05 | 3.15 | 3.15 | 9.43 | 9.43 |
| | mitochondrial/Name = AIFM1; | | | | | |
| | Synonyms = AIF, PDCD8 | | | | | |
| P04075* | Fructose-bisphosphate | 6.82E−05 | −3.58 | 3.58 | −25 | 25 |
| | aldolase A/Name = ALDOA; | | | | | |
| | Synonyms = ALDA | | | | | |
| P00558* | Phosphoglycerate kinase 1/ | 7.65E−05 | −3.1 | 3.1 | −19.43 | 19.43 |
| | Name = PGK1; | | | | | |
| | Synonyms = PGKA; | | | | | |
| | ORFNames = MIG10, | | | | | |
| | OK/SW-cl.110 | | | | | |
| P46940* | Ras GTPase-activating-like | 7.86E−05 | −4.74 | 4.74 | −14.29 | 14.29 |
| | protein IQGAP1/ | | | | | |
| | Name = IQGAP1; | | | | | |
| | Synonyms = KIAA0051 | | | | | |
| P34897 | Serine | 8.07E−05 | 2.95 | 2.95 | 6.86 | 6.86 |
| | hydroxymethyltransferase, | | | | | |
| | mitochondrial/Name = SHMT2 | | | | | |
| P10620 | Microsomal glutathione | 8.41E−05 | 3.47 | 3.47 | 4.57 | 4.57 |
| | S-transferase 1/ | | | | | |
| | Name = MGST1; | | | | | |
| | Synonyms = GST12, MGST | | | | | |
| Q15019* | Septin-2/Name = SEPT2; | 8.46E−05 | −4.68 | 4.68 | −5 | 5 |
| | Synonyms = DIFF6, | | | | | |
| | KIAA0158, NEDD5 | | | | | |
| O14756 | 17-beta-hydroxysteroid | 8.58E−05 | 4.67 | 4.67 | 11.29 | 11.29 |
| | dehydrogenase type 6/ | | | | | |
| | Name = HSD17B6; | | | | | |
| | Synonyms = RODH | | | | | |
| P07099 | Epoxide hydrolase 1/ | 8.96E−05 | 2.99 | 2.99 | 38.14 | 38.14 |
| | Name = EPHX1; | | | | | |
| | Synonyms = EPHX, EPOX | | | | | |
| Q02338 | D-beta-hydroxybutyrate | 9.25E−05 | 4.61 | 4.61 | 8.86 | 8.86 |
| | dehydrogenase, mitochondrial/ | | | | | |
| | Name = BDH1; | | | | | |
| | Synonyms = BDH | | | | | |
| P45954 | Short/branched chain specific | 9.70E−05 | 4.57 | 4.57 | 9.29 | 9.29 |
| | acyl-CoA dehydrogenase, | | | | | |
| | mitochondrial/ | | | | | |
| | Name = ACADSB | | | | | |
| Q16822 | Phosphoenolpyruvate | 1.03E−04 | 4.29 | 4.29 | 30.86 | 30.86 |
| | carboxykinase [GTP], | | | | | |
| | mitochondrial/Name = PCK2; | | | | | |
| | Synonyms = PEPCK2 | | | | | |
| P08238* | Heat shock protein HSP | 1.09E−04 | −2.97 | 2.97 | −12.71 | 12.71 |
| | 90-beta/Name = HSP90AB1; | | | | | |
| | Synonyms = HSP90B, | | | | | |
| | HSPC2, HSPCB | | | | | |
| P50454* | Serpin H1/ | 1.10E−04 | −3.45 | 3.45 | −8.29 | 8.29 |
| | Name = SERPINH1; | | | | | |
| | Synonyms = CBP1, CBP2, | | | | | |
| | HSP47, SERPINH2; | | | | | |
| | ORFNames = PIG14 | | | | | |
| P05062 | Fructose-bisphosphate | 1.16E−04 | 3.47 | 3.47 | 50.71 | 50.71 |
| | aldolase B/Name = ALDOB; | | | | | |
| | Synonyms = ALDB | | | | | |
| P05091 | Aldehyde dehydrogenase, | 1.17E−04 | 2.86 | 2.86 | 28.86 | 28.86 |
| | mitochondrial/Name = ALDH2; | | | | | |
| | Synonyms = ALDM | | | | | |
| P13010* | X-ray repair | 1.20E−04 | −2.91 | 2.91 | −8.29 | 8.29 |
| | cross-complementing | | | | | |
| | protein 5/Name = XRCC5; | | | | | |
| | Synonyms = G22P2 | | | | | |
| P51649 | Succinate-semialdehyde | 1.28E−04 | 2.77 | 2.77 | 7.71 | 7.71 |
| | dehydrogenase, mitochondrial/ | | | | | |
| | Name = ALDH5A1; | | | | | |
| | Synonyms = SSADH | | | | | |
| P27348* | 14-3-3 protein theta/ | 1.30E−04 | −2.99 | 2.99 | −11.71 | 11.71 |
| | Name = YWHAQ | | | | | |
| P02649 | Apolipoprotein E/ | 1.32E−04 | 2.93 | 2.93 | 13.86 | 13.86 |
| | Name = APOE | | | | | |
| P09467 | Fructose-1,6-bisphosphatase | 1.44E−04 | 4.26 | 4.26 | 12.29 | 12.29 |
| | 1/Name = FBP1; | | | | | |
| | Synonyms = FBP | | | | | |
| P21333* | Filamin-A/Name = FLNA; | 1.57E−04 | −3.96 | 3.96 | −55.43 | 55.43 |
| | Synonyms = FLN, FLN1 | | | | | |
| P08133 | Annexin A6/Name = ANXA6; | 1.57E−04 | 2.72 | 2.72 | 23.14 | 23.14 |
| | Synonyms = ANX6 | | | | | |
| Q9UJS0 | Calcium-binding mitochondrial | 1.62E−04 | 2.73 | 2.73 | 9.71 | 9.71 |
| | carrier protein Aralar2/ | | | | | |
| | Name = SLC25A13; | | | | | |
| | Synonyms = ARALAR2 | | | | | |
| P61158* | Actin-related protein 3/ | 1.66E−04 | −2.74 | 2.74 | −6.29 | 6.29 |
| | Name = ACTR3; | | | | | |
| | Synonyms = ARP3 | | | | | |
| P55157 | Microsomal triglyceride | 1.76E−04 | 4.11 | 4.11 | 7.57 | 7.57 |
| | transfer protein large subunit/ | | | | | |
| | Name = MTTP; | | | | | |
| | Synonyms = MTP | | | | | |
| P24752 | Acetyl-CoA acetyltransferase, | 1.97E−04 | 3.2 | 3.2 | 18 | 18 |
| | mitochondrial/Name = ACAT1; | | | | | |
| | Synonyms = ACAT, MAT | | | | | |
| P52758 | Ribonuclease UK114/ | 1.99E−04 | 3.62 | 3.62 | 8.29 | 8.29 |
| | Name = HRSP12; | | | | | |
| | Synonyms = PSP | | | | | |
| P16930 | Fumarylacetoacetase/ | 2.11E−04 | 2.99 | 2.99 | 11.14 | 11.14 |
| | Name = FAH | | | | | |
| P00505 | Aspartate aminotransferase, | 2.16E−04 | 2.87 | 2.87 | 8.43 | 8.43 |
| | mitochondrial/Name = GOT2 | | | | | |
| P12429* | Annexin A3/Name = ANXA3; | 2.25E−04 | −3.14 | 3.14 | −4.57 | 4.57 |
| | Synonyms = ANX3 | | | | | |
| P68032* | Actin, alpha cardiac muscle | 2.30E−04 | −2.62 | 2.62 | −55.86 | 55.86 |
| | 1/Name = ACTC1; | | | | | |
| | Synonyms = ACTC | | | | | |
| P00367 | Glutamate dehydrogenase 1, | 2.42E−04 | 2.61 | 2.61 | 18 | 18 |
| | mitochondrial/Name = GLUD1; | | | | | |
| | Synonyms = GLUD | | | | | |
| P30038 | Delta-1-pyrroline-5-carboxylate | 2.42E−04 | 3.04 | 3.04 | 16.43 | 16.43 |
| | dehydrogenase, | | | | | |
| | mitochondrial/ | | | | | |
| | Name = ALDH4A1; | | | | | |
| | Synonyms = ALDH4, P5CDH | | | | | |
| Q12905* | Interleukin | 2.58E−04 | −2.58 | 2.58 | −3.86 | 3.86 |
| | enhancer-binding factor 2/ | | | | | |
| | Name = ILF2; | | | | | |
| | Synonyms = NF45; | | | | | |
| | ORFNames = PRO3063 | | | | | |
| P21810* | Biglycan/Name = BGN; | 2.80E−04 | −3.62 | 3.62 | −21.57 | 21.57 |
| | Synonyms = SLRR1A | | | | | |
| P78417 | Glutathione S-transferase | 2.87E−04 | 2.92 | 2.92 | 5.43 | 5.43 |
| | omega-1/Name = GSTO1; | | | | | |
| | Synonyms = GSTTLP28 | | | | | |
| Q14032 | Bile acid-CoA:amino acid | 3.13E−04 | 3.7 | 3.7 | 6.43 | 6.43 |
| | N-acyltransferase/ | | | | | |
| | Name = BAAT | | | | | |
| Q9H8H3 | Methyltransferase-like protein | 3.14E−04 | 2.6 | 2.6 | 3.86 | 3.86 |
| | 7A/Name = METTL7A; | | | | | |
| | ORFNames = PRO0066, | | | | | |
| | UNQ1902/PRO4348 | | | | | |
| Q9UJM8 | Hydroxyacid oxidase 1/ | 3.16E−04 | 3.69 | 3.69 | 20 | 20 |
| | Name = HAO1; | | | | | |
| | Synonyms = GOX1, HAOX1 | | | | | |
| P12956* | X-ray repair | 3.17E−04 | −2.71 | 2.71 | −10.57 | 10.57 |
| | cross-complementing | | | | | |
| | protein 6/Name = XRCC6; | | | | | |
| | Synonyms = G22P1 | | | | | |
| O00264 | Membrane-associated | 3.42E−04 | 3.21 | 3.21 | 10.71 | 10.71 |
| | progesterone receptor | | | | | |
| | component 1/ | | | | | |
| | Name = PGRMC1; | | | | | |
| | Synonyms = HPR6.6, PGRMC | | | | | |
| P67936* | Tropomyosin alpha-4 chain/ | 3.48E−04 | −2.88 | 2.88 | −9.43 | 9.43 |
| | Name = TPM4 | | | | | |
| P11498 | Pyruvate carboxylase, | 3.68E−04 | 3.54 | 3.54 | 20.14 | 20.14 |
| | mitochondrial/Name = PC | | | | | |
| Q9HDC9 | Adipocyte plasma | 3.72E−04 | 2.45 | 2.45 | 5.14 | 5.14 |
| | membrane-associated protein/ | | | | | |
| | Name = APMAP; | | | | | |
| | Synonyms = C20orf3; | | | | | |
| | ORFNames = UNQ1869/PRO4305 | | | | | |
| P36871 | Phosphoglucomutase-1/ | 3.85E−04 | 2.48 | 2.48 | 17.71 | 17.71 |
| | Name = PGM1 | | | | | |
| Q93099 | Homogentisate | 4.18E−04 | 2.64 | 2.64 | 12.14 | 12.14 |
| | 1,2-dioxygenase/ | | | | | |
| | Name = HGD; Synonyms = HGO | | | | | |
| Q969Z3 | MOSC domain-containing | 4.42E−04 | 3.47 | 3.47 | 6.14 | 6.14 |
| | protein 2, mitochondrial/ | | | | | |
| | Name = MOSC2 | | | | | |
| P30084 | Enoyl-CoA hydratase, | 4.53E−04 | 2.43 | 2.43 | 9.86 | 9.86 |
| | mitochondrial/Name = ECHS1 | | | | | |
| Q9Y2Q3 | Glutathione S-transferase | 4.71E−04 | 2.46 | 2.46 | 10.57 | 10.57 |
| | kappa 1/Name = GSTK1; | | | | | |
| | ORFNames = HDCMD47P | | | | | |
| Q16762 | Thiosulfate sulfurtransferase/ | 4.86E−04 | 2.54 | 2.54 | 14.29 | 14.29 |
| | Name = TST | | | | | |
| P00480 | Ornithine | 4.87E−04 | 3.41 | 3.41 | 14.71 | 14.71 |
| | carbamoyltransferase, | | | | | |
| | mitochondrial/Name = OTC | | | | | |
| O60664* | Perilipin-3/Name = PLIN3; | 5.02E−04 | −3.39 | 3.39 | −6.43 | 6.43 |
| | Synonyms = M6PRBP1, TIP47 | | | | | |
| Q9P0Z9 | Peroxisomal sarcosine | 5.12E−04 | 3.38 | 3.38 | 13.14 | 13.14 |
| | oxidase/Name = PIPOX; | | | | | |
| | Synonyms = LPIPOX, PSO | | | | | |
| Q4G0N4 | NAD kinase | 5.57E−04 | 3.33 | 3.33 | 8.57 | 8.57 |
| | domain-containing protein 1/ | | | | | |
| | Name = NADKD1; | | | | | |
| | Synonyms = C5orf33 | | | | | |
| Q06520 | Bile salt sulfotransferase/ | 5.74E−04 | 3.31 | 3.31 | 14.43 | 14.43 |
| | Name = SULT2A1; | | | | | |
| | Synonyms = HST, STD | | | | | |
| P06396* | Gelsolin/Name = GSN | 6.42E−04 | −2.49 | 2.49 | −8.43 | 8.43 |
| P31327 | Carbamoyl-phosphate | 6.50E−04 | 3 | 3 | 162.57 | 162.57 |
| | synthase [ammonia], | | | | | |
| | mitochondrial/Name = CPS1 | | | | | |
| P04424 | Argininosuccinate lyase/ | 6.65E−04 | 3.05 | 3.05 | 18 | 18 |
| | Name = ASL | | | | | |
| P11310 | Medium-chain specific | 6.71E−04 | 2.65 | 2.65 | 7.43 | 7.43 |
| | acyl-CoA dehydrogenase, | | | | | |
| | mitochondrial/Name = ACADM | | | | | |
| Q9BPW8 | Protein NipSnap homolog 1/ | 7.94E−04 | 2.28 | 2.28 | 6.71 | 6.71 |
| | Name = NIPSNAP1 | | | | | |
| P23141 | Liver carboxylesterase 1/ | 7.99E−04 | 3.05 | 3.05 | 57.14 | 57.14 |
| | Name = CES1; | | | | | |
| | Synonyms = CES2, SES1 | | | | | |
| P06737 | Glycogen phosphorylase, liver | 8.18E−04 | 3.1 | 3.1 | 12.29 | 12.29 |
| | form/Name = PYGL | | | | | |
| P50995* | Annexin A11/ | 8.64E−04 | −2.47 | 2.47 | −6.57 | 6.57 |
| | Name = ANXA11; | | | | | |
| | Synonyms = ANX11 | | | | | |
| Q03154 | Aminoacylase-1/ | 9.00E−04 | 3.04 | 3.04 | 16.71 | 16.71 |
| | Name = ACY1 | | | | | |
| P42765 | 3-ketoacyl-CoA thiolase, | 1.01E−03 | 2.26 | 2.26 | 18.71 | 18.71 |
| | mitochondrial/Name = ACAA2 | | | | | |

PART B:

| | | | | | | |
|---|---|---|---|---|---|---|
| O43399 | Tumor protein D54/ | 1.06E−03 | −2.34 | 2.34 | −3.57 | 3.57 |
| | Name = TPD52L2 | | | | | |
| P07954 | Fumarate hydratase, | 1.12E−03 | 2.38 | 2.38 | 6.43 | 6.43 |
| | mitochondrial/Name = FH | | | | | |
| P80404 | 4-aminobutyrate | 1.16E−03 | 2.83 | 2.83 | 24 | 24 |
| | aminotransferase, | | | | | |
| | mitochondrial/Name = ABAT; | | | | | |
| | Synonyms = GABAT | | | | | |
| P21549 | Serine--pyruvate | 1.18E−03 | 2.73 | 2.73 | 66.43 | 66.43 |
| | aminotransferase/ | | | | | |
| | Name = AGXT; | | | | | |
| | Synonyms = AGT1, SPAT | | | | | |
| P34913 | Epoxide hydrolase 2/ | 1.23E−03 | 2.87 | 2.87 | 5 | 5 |
| | Name = EPHX2 | | | | | |
| P04350 | Tubulin beta-4 chain/ | 1.25E−03 | −2.85 | 2.85 | −50.43 | 50.43 |
| | Name = TUBB4; | | | | | |
| | Synonyms = TUBB5 | | | | | |
| O95479 | GDH/6PGL endoplasmic | 1.27E−03 | 2.65 | 2.65 | 6.29 | 6.29 |
| | bifunctional protein/ | | | | | |
| | Name = H6PD; | | | | | |
| | Synonyms = GDH | | | | | |
| P00167 | Cytochrome b5/ | 1.29E−03 | 2.76 | 2.76 | 10.86 | 10.86 |
| | Name = CYB5A; | | | | | |
| | Synonyms = CYB5 | | | | | |
| Q08426 | Peroxisomal bifunctional | 1.29E−03 | 2.8 | 2.8 | 32.86 | 32.86 |
| | enzyme/Name = EHHADH; | | | | | |
| | Synonyms = ECHD | | | | | |
| P61981 | 14-3-3 protein gamma/ | 1.33E−03 | −2.15 | 2.15 | −4.57 | 4.57 |
| | Name = YWHAG | | | | | |
| P05089 | Arginase-1/Name = ARG1 | 1.35E−03 | 2.81 | 2.81 | 22.14 | 22.14 |
| P05455 | Lupus La protein/Name = SSB | 1.36E−03 | −2.09 | 2.09 | −4.57 | 4.57 |
| P51884 | Lumican/Name = LUM; | 1.37E−03 | −2.46 | 2.46 | −12.71 | 12.71 |
| | Synonyms = LDC, SLRR2D | | | | | |
| P21399 | Cytoplasmic aconitate | 1.45E−03 | 2.52 | 2.52 | 13.14 | 13.14 |
| | hydratase/Name = ACO1; | | | | | |
| | Synonyms = IREB1 | | | | | |
| Q15436 | Protein transport protein | 1.47E−03 | 2.17 | 2.17 | 4.43 | 4.43 |
| | Sec23A/Name = SEC23A | | | | | |
| P60660 | Myosin light polypeptide 6/ | 1.50E−03 | −2.06 | 2.06 | −5.29 | 5.29 |
| | Name = MYL6 | | | | | |
| P54868 | Hydroxymethylglutaryl-CoA | 1.51E−03 | 2.66 | 2.66 | 29.57 | 29.57 |
| | synthase, mitochondrial/ | | | | | |
| | Name = HMGCS2 | | | | | |
| Q9NVS9 | Pyridoxine-5′-phosphate | 1.52E−03 | 2.75 | 2.75 | 3.57 | 3.57 |
| | oxidase/Name = PNPO | | | | | |
| P23528 | Cofilin-1/Name = CFL1; | 1.67E−03 | −2.02 | 2.02 | −3.86 | 3.86 |
| | Synonyms = CFL | | | | | |
| Q6NVY1 | 3-hydroxyisobutyryl-CoA | 1.74E−03 | 2.04 | 2.04 | 2.71 | 2.71 |
| | hydrolase, mitochondrial/ | | | | | |
| | Name = HIBCH | | | | | |
| P28845 | Corticosteroid | 1.78E−03 | 2.67 | 2.67 | 4.86 | 4.86 |
| | 11-beta-dehydrogenase | | | | | |
| | isozyme 1/Name = HSD11B1; | | | | | |
| | Synonyms = HSD11, HSD11L | | | | | |
| P30086 | Phosphatidylethanolamine-binding | 1.87E−03 | 2.13 | 2.13 | 12.57 | 12.57 |
| | protein 1/Name = PEBP1; | | | | | |
| | Synonyms = PBP, PEBP | | | | | |
| Q04828 | Aldo-keto reductase family 1 | 1.96E−03 | 2.16 | 2.16 | 20.14 | 20.14 |
| | member C1/Name = AKR1C1; | | | | | |
| | Synonyms = DDH, DDH1 | | | | | |
| O43707 | Alpha-actinin-4/ | 1.98E−03 | −2.12 | 2.12 | −18.14 | 18.14 |
| | Name = ACTN4 | | | | | |
| Q9Y490 | Talin-1/Name = TLN1; | 2.03E−03 | −2.09 | 2.09 | −11.86 | 11.86 |
| | Synonyms = KIAA1027, TLN | | | | | |
| Q02252 | Methylmalonate-semialdehyde | 2.03E−03 | 2.41 | 2.41 | 26.57 | 26.57 |
| | dehydrogenase [acylating], | | | | | |
| | mitochondrial/ | | | | | |
| | Name = ALDH6A1; | | | | | |
| | Synonyms = MMSDH | | | | | |
| P09211 | Glutathione S-transferase P/ | 2.12E−03 | −2.51 | 2.51 | −17.14 | 17.14 |
| | Name = GSTP1; | | | | | |
| | Synonyms = FAEES3, GST3 | | | | | |
| P32754 | 4-hydroxyphenylpyruvate | 2.17E−03 | 2.56 | 2.56 | 19.86 | 19.86 |
| | dioxygenase/Name = HPD; | | | | | |
| | Synonyms = PPD | | | | | |
| P07384 | Calpain-1 catalytic subunit/ | 2.20E−03 | −1.99 | 1.99 | −8.14 | 8.14 |
| | Name = CAPN1; | | | | | |
| | Synonyms = CANPL1; | | | | | |
| | ORFNames = PIG30 | | | | | |
| Q9H2A2 | Aldehyde dehydrogenase | 2.23E−03 | 2.55 | 2.55 | 4.43 | 4.43 |
| | family 8 member A1/ | | | | | |
| | Name = ALDH8A1; | | | | | |
| | Synonyms = ALDH12 | | | | | |
| P30043 | Flavin reductase (NADPH)/ | 2.27E−03 | 2.18 | 2.18 | 6 | 6 |
| | Name = BLVRB; | | | | | |
| | Synonyms = FLR | | | | | |
| Q68CK6 | Acyl-coenzyme A synthetase | 2.31E−03 | 2.53 | 2.53 | 15.57 | 15.57 |
| | ACSM2B, mitochondrial/ | | | | | |
| | Name = ACSM2B; | | | | | |
| | Synonyms = ACSM2; | | | | | |
| | ORFNames = HYST1046 | | | | | |
| P13611 | Versican core protein/ | 2.34E−03 | −2.5 | 2.5 | −14.43 | 14.43 |
| | Name = VCAN; | | | | | |
| | Synonyms = CSPG2 | | | | | |
| P07237 | Protein disulfide-isomerase/ | 2.40E−03 | 2 | 2 | 11.14 | 11.14 |
| | Name = P4HB; | | | | | |
| | Synonyms = ERBA2L, PDI, | | | | | |
| | PDIA1, PO4DB | | | | | |
| P22307 | Non-specific lipid-transfer | 2.42E−03 | 2.46 | 2.46 | 11.14 | 11.14 |
| | protein/Name = SCP2 | | | | | |
| Q9Y265 | RuvB-like 1/Name = RUVBL1; | 2.47E−03 | −2.5 | 2.5 | −4.71 | 4.71 |
| | Synonyms = INO80H, NMP238, | | | | | |
| | TIP49, TIP49A | | | | | |
| P49419 | Alpha-aminoadipic | 2.47E−03 | 1.93 | 1.93 | 13 | 13 |
| | semialdehyde dehydrogenase/ | | | | | |
| | Name = ALDH7A1; | | | | | |
| | Synonyms = ATQ1 | | | | | |
| P62258 | 14-3-3 protein epsilon/ | 2.56E−03 | −1.9 | 1.9 | −6.86 | 6.86 |
| | Name = YWHAE | | | | | |
| P19105 | Myosin regulatory light chain | 2.59E−03 | −1.94 | 1.94 | −2.86 | 2.86 |
| | 12A/Name = MYL12A; | | | | | |
| | Synonyms = MLCB, MRLC3, | | | | | |
| | RLC | | | | | |
| O14950 | Myosin regulatory light chain | 2.59E−03 | −1.94 | 1.94 | −2.86 | 2.86 |
| | 12B/Name = MYL12B; | | | | | |
| | Synonyms = MRLC2, MYLC2B | | | | | |
| O75367 | Core histone macro-H2A.1/ | 2.59E−03 | −1.97 | 1.97 | −9 | 9 |
| | Name = H2AFY; | | | | | |
| | Synonyms = MACROH2A1 | | | | | |
| P11216 | Glycogen phosphorylase, | 2.60E−03 | −1.89 | 1.89 | −6.57 | 6.57 |
| | brain form/Name = PYGB | | | | | |
| P16152 | Carbonyl reductase [NADPH] | 2.61E−03 | 2.26 | 2.26 | 17 | 17 |
| | 1/Name = CBR1; | | | | | |
| | Synonyms = CBR, CRN | | | | | |
| Q7Z4W1 | L-xylulose reductase/ | 2.69E−03 | 2.42 | 2.42 | 26.29 | 26.29 |
| | Name = DCXR | | | | | |
| P07355 | Annexin A2/Name = ANXA2; | 2.85E−03 | −2.38 | 2.38 | −25.43 | 25.43 |
| | Synonyms = ANX2, ANX2L4, | | | | | |
| | CAL1H, LPC2D | | | | | |
| P78527 | DNA-dependent protein | 2.86E−03 | −2.11 | 2.11 | −13.71 | 13.71 |
| | kinase catalytic subunit/ | | | | | |
| | Name = PRKDC; | | | | | |
| | Synonyms = HYRC, HYRC1 | | | | | |
| Q15393 | Splicing factor 3B subunit 3/ | 2.91E−03 | −2.42 | 2.42 | −3.29 | 3.29 |
| | Name = SF3B3; | | | | | |
| | Synonyms = KIAA0017, | | | | | |
| | SAP130 | | | | | |
| Q04917 | 14-3-3 protein eta/ | 2.97E−03 | −1.99 | 1.99 | −6.86 | 6.86 |
| | Name = YWHAH; | | | | | |
| | Synonyms = YWHA1 | | | | | |
| P38117 | Electron transfer flavoprotein | 2.97E−03 | 2.01 | 2.01 | 8.43 | 8.43 |
| | subunit beta/Name = ETFB; | | | | | |
| | ORFNames = FP585 | | | | | |
| P08729 | Keratin, type II cytoskeletal 7/ | 3.10E−03 | −2.39 | 2.39 | −12 | 12 |
| | Name = KRT7; Synonyms = SCL | | | | | |
| Q14914 | Prostaglandin reductase 1/ | 3.21E−03 | 2.29 | 2.29 | 10.71 | 10.71 |
| | Name = PTGR1; | | | | | |
| | Synonyms = LTB4DH | | | | | |
| P17516 | Aldo-keto reductase family 1 | 3.24E−03 | 2.36 | 2.36 | 11.43 | 11.43 |
| | member C4/Name = AKR1C4; | | | | | |
| | Synonyms = CHDR | | | | | |
| P63261 | Actin, cytoplasmic 2/ | 3.35E−03 | −2.22 | 2.22 | −101.57 | 101.57 |
| | Name = ACTG1; | | | | | |
| | Synonyms = ACTB, ACTG | | | | | |
| P16435 | NADPH--cytochrome P450 | 3.46E−03 | 2.28 | 2.28 | 12.86 | 12.86 |
| | reductase/Name = POR; | | | | | |
| | Synonyms = CYPOR | | | | | |
| Q15067 | Peroxisomal acyl-coenzyme A | 3.54E−03 | 2.32 | 2.32 | 11 | 11 |
| | oxidase 1/Name = ACOX1; | | | | | |
| | Synonyms = ACOX | | | | | |
| P35914 | Hydroxymethylglutaryl-CoA | 3.55E−03 | 2.32 | 2.32 | 4.71 | 4.71 |
| | lyase, mitochondrial/ | | | | | |
| | Name = HMGCL | | | | | |
| P30046 | D-dopachrome decarboxylase/ | 3.63E−03 | 1.95 | 1.95 | 4.86 | 4.86 |
| | Name = DDT | | | | | |
| P30613 | Pyruvate kinase isozymes R/L/ | 3.63E−03 | 2.31 | 2.31 | 11.14 | 11.14 |
| | Name = PKLR; | | | | | |
| | Synonyms = PK1, PKL | | | | | |
| Q07960 | Rho GTPase-activating | 3.67E−03 | −2.07 | 2.07 | −3.43 | 3.43 |
| | protein 1/Name = ARHGAP1; | | | | | |
| | Synonyms = CDC42GAP, | | | | | |
| | RHOGAP1 | | | | | |
| Q6YN16 | Hydroxysteroid | 3.67E−03 | 2.07 | 2.07 | 3.43 | 3.43 |
| | dehydrogenase-like protein 2/ | | | | | |
| | Name = HSDL2; | | | | | |
| | Synonyms = C9orf99 | | | | | |
| Q08AH3 | Acyl-coenzyme A synthetase | 3.69E−03 | 2.3 | 2.3 | 15.14 | 15.14 |
| | ACSM2A, mitochondrial/ | | | | | |
| | Name = ACSM2A; | | | | | |
| | Synonyms = ACSM2, MACS2 | | | | | |
| Q9UL12 | Sarcosine dehydrogenase, | 3.70E−03 | 2.3 | 2.3 | 7.29 | 7.29 |
| | mitochondrial/ | | | | | |
| | Name = SARDH; | | | | | |
| | Synonyms = DMGDHL1 | | | | | |
| O00299 | Chloride intracellular channel | 3.76E−03 | −2 | 2 | −7.71 | 7.71 |
| | protein 1/Name = CLIC1; | | | | | |
| | Synonyms = G6, NCC27 | | | | | |
| Q13838 | Spliceosome RNA helicase | 3.79E−03 | −1.85 | 1.85 | −4.43 | 4.43 |
| | DDX39B/Name = DDX39B; | | | | | |
| | Synonyms = BAT1, UAP56 | | | | | |
| P09110 | 3-ketoacyl-CoA thiolase, | 3.81E−03 | 2 | 2 | 14.71 | 14.71 |
| | peroxisomal/Name = ACAA1; | | | | | |
| | Synonyms = ACAA, PTHIO | | | | | |
| P52907 | F-actin-capping protein | 3.82E−03 | −1.96 | 1.96 | −3.29 | 3.29 |
| | subunit alpha-1/ | | | | | |
| | Name = CAPZA1 | | | | | |
| P34896 | Serine | 3.85E−03 | 2.07 | 2.07 | 10.71 | 10.71 |
| | hydroxymethyltransferase, | | | | | |
| | cytosolic/Name = SHMT1 | | | | | |
| P00491 | Purine nucleoside | 3.87E−03 | −1.84 | 1.84 | −5.86 | 5.86 |
| | phosphorylase/Name = PNP; | | | | | |
| | Synonyms = NP | | | | | |
| Q9P0M6 | Core histone macro-H2A.2/ | 4.02E−03 | −2.26 | 2.26 | −6 | 6 |
| | Name = H2AFY2; | | | | | |
| | Synonyms = MACROH2A2 | | | | | |
| Q01995 | Transgelin/Name = TAGLN; | 4.04E−03 | −2.16 | 2.16 | −9.86 | 9.86 |
| | Synonyms = SM22, WS3-10 | | | | | |
| Q16851 | UTP--glucose-1-phosphate | 4.09E−03 | 2.09 | 2.09 | 18.57 | 18.57 |
| | uridylyltransferase/ | | | | | |
| | Name = UGP2; | | | | | |
| | Synonyms = UGP1 | | | | | |
| P05090 | Apolipoprotein D/ | 4.10E−03 | 2.25 | 2.25 | 2.57 | 2.57 |
| | Name = APOD | | | | | |
| P22033 | Methylmalonyl-CoA mutase, | 4.20E−03 | 2.24 | 2.24 | 2.71 | 2.71 |
| | mitochondrial/Name = MUT | | | | | |
| P31939 | Bifunctional purine | 4.22E−03 | −2.01 | 2.01 | −11.43 | 11.43 |
| | biosynthesis protein PURH/ | | | | | |
| | Name = ATIC; | | | | | |
| | Synonyms = PURH; | | | | | |
| | ORFNames = OK/SW-cl.86 | | | | | |
| Q08380 | Galectin-3-binding protein/ | 4.25E−03 | −1.98 | 1.98 | −7.71 | 7.71 |
| | Name = LGALS3BP; | | | | | |
| | Synonyms = M2BP | | | | | |
| P07437 | Tubulin beta chain/ | 4.34E−03 | −1.76 | 1.76 | −24.71 | 24.71 |
| | Name = TUBB; | | | | | |
| | Synonyms = TUBB5; | | | | | |
| | ORFNames = OK/SW-cl.56 | | | | | |
| P07585 | Decorin/Name = DCN; | 4.35E−03 | −2.22 | 2.22 | −8 | 8 |
| | Synonyms = SLRR1B | | | | | |
| O75533 | Splicing factor 3B subunit 1/ | 4.39E−03 | −2.22 | 2.22 | −3.71 | 3.71 |
| | Name = SF3B1; | | | | | |
| | Synonyms = SAP155 | | | | | |
| P22310 | UDP-glucuronosyltransferase | 4.46E−03 | 2.21 | 2.21 | 8.29 | 8.29 |
| | 1-4/Name = UGT1A4; | | | | | |
| | Synonyms = GNT1, UGT1 | | | | | |
| P07195 | L-lactate dehydrogenase B | 4.47E−03 | −1.88 | 1.88 | −9.43 | 9.43 |
| | chain/Name = LDHB | | | | | |
| P24298 | Alanine aminotransferase 1/ | 4.50E−03 | 2.21 | 2.21 | 8 | 8 |
| | Name = GPT; | | | | | |
| | Synonyms = AAT1, GPT1 | | | | | |
| Q9H9B4 | Sideroflexin-1/Name = SFXN1 | 4.52E−03 | 1.84 | 1.84 | 4.14 | 4.14 |
| P14923 | Junction plakoglobin/ | 4.73E−03 | −2.01 | 2.01 | −7.57 | 7.57 |
| | Name = JUP; | | | | | |
| | Synonyms = CTNNG, DP3 | | | | | |
| P11586 | C-1-tetrahydrofolate synthase, | 4.73E−03 | 2.1 | 2.1 | 17.57 | 17.57 |
| | cytoplasmic/Name = MTHFD1; | | | | | |
| | Synonyms = MTHFC, MTHFD | | | | | |
| P28838 | Cytosol aminopeptidase/ | 4.91E−03 | 1.86 | 1.86 | 9.29 | 9.29 |
| | Name = LAP3; | | | | | |
| | Synonyms = LAPEP, PEPS | | | | | |
| P21291 | Cysteine and glycine-rich | 4.96E−03 | −1.79 | 1.79 | −3.29 | 3.29 |
| | protein 1/Name = CSRP1; | | | | | |
| | Synonyms = CSRP, CYRP | | | | | |
| P00441 | Superoxide dismutase [Cu—Zn]/ | 4.99E−03 | 2.03 | 2.03 | 7.43 | 7.43 |
| | Name = SOD1 | | | | | |
| Q9Y6C9 | Mitochondrial carrier homolog | 5.15E−03 | 1.79 | 1.79 | 6 | 6 |
| | 2/Name = MTCH2; | | | | | |
| | Synonyms = MIMP; | | | | | |
| | ORFNames = HSPC032 | | | | | |
| P23284 | Peptidyl-prolyl cis-trans | 5.21E−03 | 1.71 | 1.71 | 4.14 | 4.14 |
| | isomerase B/Name = PPIB; | | | | | |
| | Synonyms = CYPB | | | | | |
| O60701 | UDP-glucose | 5.24E−03 | 1.96 | 1.96 | 6.86 | 6.86 |
| | 6-dehydrogenase/ | | | | | |
| | Name = UGDH | | | | | |
| P16422 | Epithelial cell adhesion | 5.30E−03 | −2.13 | 2.13 | −2.86 | 2.86 |
| | molecule/Name = EPCAM; | | | | | |
| | Synonyms = GA733-2, M1S2, | | | | | |
| | M4S1, MIC18, TACSTD1, | | | | | |
| | TROP1 | | | | | |
| P07327 | Alcohol dehydrogenase 1A/ | 5.37E−03 | 2.12 | 2.12 | 38.86 | 38.86 |
| | Name = ADH1A; | | | | | |
| | Synonyms = ADH1 | | | | | |
| Q9UIJ7 | GTP:AMP | 5.41E−03 | 1.98 | 1.98 | 4 | 4 |
| | phosphotransferase, | | | | | |
| | mitochondrial/Name = AK3; | | | | | |
| | Synonyms = AK3L1, AK6, | | | | | |
| | AKL3L | | | | | |
| P61160 | Actin-related protein 2/ | 5.48E−03 | −1.86 | 1.86 | −5.14 | 5.14 |
| | Name = ACTR2; | | | | | |
| | Synonyms = ARP2 | | | | | |
| P06703 | Protein S100-A6/ | 5.49E−03 | −1.94 | 1.94 | −3.43 | 3.43 |
| | Name = S100A6; | | | | | |
| | Synonyms = CACY | | | | | |
| Q96HR9 | Receptor | 5.56E−03 | 2.11 | 2.11 | 3.43 | 3.43 |
| | expression-enhancing protein | | | | | |
| | 6/Name = REEP6; | | | | | |
| | Synonyms = C19orf32, DP1L1 | | | | | |
| O75521 | Enoyl-CoA delta isomerase 2, | 5.59E−03 | 2.11 | 2.11 | 6.43 | 6.43 |
| | mitochondrial/Name = ECI2; | | | | | |
| | Synonyms = DRS1, HCA88, | | | | | |
| | PECI | | | | | |
| P13639 | Elongation factor 2/ | 5.61E−03 | −1.79 | 1.79 | −12.43 | 12.43 |
| | Name = EEF2; Synonyms = EF2 | | | | | |
| P13804 | Electron transfer flavoprotein | 5.64E−03 | 1.87 | 1.87 | 8 | 8 |
| | subunit alpha, mitochondrial/ | | | | | |
| | Name = ETFA | | | | | |
| Q16698 | 2,4-dienoyl-CoA reductase, | 5.68E−03 | 1.85 | 1.85 | 9 | 9 |
| | mitochondrial/ | | | | | |
| | Name = DECR1; | | | | | |
| | Synonyms = DECR | | | | | |
| O95154 | Aflatoxin B1 aldehyde | 5.77E−03 | 2.09 | 2.09 | 8.14 | 8.14 |
| | reductase member 3/ | | | | | |
| | Name = AKR7A3; | | | | | |
| | Synonyms = AFAR2 | | | | | |
| O14773 | Tripeptidyl-peptidase 1/ | 5.93E−03 | 1.69 | 1.69 | 7.29 | 7.29 |
| | Name = TPP1; | | | | | |
| | Synonyms = CLN2; | | | | | |
| | ORFNames = GIG1, | | | | | |
| | UNQ267/PRO304 | | | | | |
| P19338 | Nucleolin/Name = NCL | 6.02E−03 | −1.7 | 1.7 | −7.71 | 7.71 |
| P07900 | Heat shock protein HSP | 6.08E−03 | −1.89 | 1.89 | −19.71 | 19.71 |
| | 90-alpha/Name = HSP90AA1; | | | | | |
| | Synonyms = HSP90A, HSPC1, | | | | | |
| | HSPCA | | | | | |
| Q9BWD1 | Acetyl-CoA acetyltransferase, | 6.15E−03 | 1.99 | 1.99 | 5.57 | 5.57 |
| | cytosolic/Name = ACAT2; | | | | | |
| | Synonyms = ACTL | | | | | |
| Q07507 | Dermatopontin/Name = DPT | 6.20E−03 | −1.98 | 1.98 | −5.14 | 5.14 |
| Q9Y2P5 | Bile acyl-CoA synthetase/ | 6.22E−03 | 2.06 | 2.06 | 9.14 | 9.14 |
| | Name = SLC27A5; | | | | | |
| | Synonyms = ACSB, ACSVL6, | | | | | |
| | FACVL3, FATP5 | | | | | |
| P17174 | Aspartate aminotransferase, | 6.37E−03 | 1.8 | 1.8 | 23.14 | 23.14 |
| | cytoplasmic/Name = GOT1 | | | | | |
| P08727 | Keratin, type I cytoskeletal 19/ | 6.49E−03 | −2.04 | 2.04 | −27.29 | 27.29 |
| | Name = KRT19 | | | | | |
| P51888 | Prolargin/Name = PRELP; | 6.58E−03 | −2 | 2 | −25.29 | 25.29 |
| | Synonyms = SLRR2A | | | | | |
| Q9UBQ7 | Glyoxylate | 6.71E−03 | 1.99 | 1.99 | 13.57 | 13.57 |
| | reductase/hydroxypyruvate | | | | | |
| | reductase/Name = GRHPR; | | | | | |
| | Synonyms = GLXR; | | | | | |
| | ORFNames = MSTP035 | | | | | |
| P34932 | Heat shock 70 kDa protein 4/ | 6.91E−03 | −1.63 | 1.63 | −3.71 | 3.71 |
| | Name = HSPA4; | | | | | |
| | Synonyms = APG2 | | | | | |
| Q15149 | Plectin/Name = PLEC; | 6.91E−03 | −1.95 | 1.95 | −14 | 14 |
| | Synonyms = PLEC1 | | | | | |
| P00403 | Cytochrome c oxidase subunit | 6.92E−03 | 1.65 | 1.65 | 2.71 | 2.71 |
| | 2/Name = MT-CO2; | | | | | |
| | Synonyms = COII, COXII, | | | | | |
| | MTCO2 | | | | | |
| Q15274 | Nicotinate-nucleotide | 7.04E−03 | 2.01 | 2.01 | 3.86 | 3.86 |
| | pyrophosphorylase | | | | | |
| | [carboxylating]/Name = QPRT | | | | | |
| Q14117 | Dihydropyrimidinase/ | 7.23E−03 | 1.99 | 1.99 | 3.57 | 3.57 |
| | Name = DPYS | | | | | |
| P27695 | DNA-(apurinic or apyrimidinic | 7.51E−03 | −1.64 | 1.64 | −4.14 | 4.14 |
| | site) lyase/Name = APEX1; | | | | | |
| | Synonyms = APE, APE1, | | | | | |
| | APEX, APX, HAP1, REF1 | | | | | |
| P51858 | Hepatoma-derived growth | 7.54E−03 | −1.7 | 1.7 | −5.86 | 5.86 |
| | factor/Name = HDGF; | | | | | |
| | Synonyms = HMG1L2 | | | | | |
| Q13228 | Selenium-binding protein 1/ | 7.54E−03 | 1.89 | 1.89 | 16.57 | 16.57 |
| | Name = SELENBP1; | | | | | |
| | Synonyms = SBP | | | | | |
| P46783 | 40S ribosomal protein S10/ | 7.58E−03 | 1.63 | 1.63 | 3 | 3 |
| | Name = RPS10 | | | | | |
| Q00796 | Sorbitol dehydrogenase/ | 7.61E−03 | 1.97 | 1.97 | 12.86 | 12.86 |
| | Name = SORD | | | | | |
| P00325 | Alcohol dehydrogenase 1B/ | 7.68E−03 | 1.91 | 1.91 | 37.57 | 37.57 |
| | Name = ADH1B; | | | | | |
| | Synonyms = ADH2 | | | | | |
| P01024 | Complement C3/Name = C3; | 7.77E−03 | −1.77 | 1.77 | −23.71 | 23.71 |
| | Synonyms = CPAMD1 | | | | | |
| Q93088 | Betaine--homocysteine | 7.81E−03 | 1.96 | 1.96 | 16.29 | 16.29 |
| | S-methyltransferase 1/ | | | | | |
| | Name = BHMT | | | | | |
| P17655 | Calpain-2 catalytic subunit/ | 8.05E−03 | −1.95 | 1.95 | −5.29 | 5.29 |
| | Name = CAPN2; | | | | | |
| | Synonyms = CANPL2 | | | | | |
| Q13724 | Mannosyl-oligosaccharide | 8.22E−03 | 1.68 | 1.68 | 1.86 | 1.86 |
| | glucosidase/Name = MOGS; | | | | | |
| | Synonyms = GCS1 | | | | | |
| O75489 | NADH dehydrogenase | 8.27E−03 | 1.62 | 1.62 | 2.43 | 2.43 |
| | [ubiquinone] iron-sulfur protein | | | | | |
| | 3, mitochondrial/ | | | | | |
| | Name = NDUFS3 | | | | | |
| P50226 | Sulfotransferase 1A2/ | 8.35E−03 | 1.73 | 1.73 | 4.14 | 4.14 |
| | Name = SULT1A2; | | | | | |
| | Synonyms = STP2 | | | | | |
| P02790 | Hemopexin/Name = HPX | 8.37E−03 | −1.63 | 1.63 | −5.14 | 5.14 |
| Q02318 | Sterol 26-hydroxylase, | 8.51E−03 | 1.92 | 1.92 | 4.43 | 4.43 |
| | mitochondrial/ | | | | | |
| | Name = CYP27A1; | | | | | |
| | Synonyms = CYP27 | | | | | |
| P30041 | Peroxiredoxin-6/ | 8.74E−03 | 1.58 | 1.58 | 9.86 | 9.86 |
| | Name = PRDX6; | | | | | |
| | Synonyms = AOP2, KIAA0106 | | | | | |
| P00966 | Argininosuccinate synthase/ | 8.90E−03 | 1.9 | 1.9 | 16.71 | 16.71 |
| | Name = ASS1; Synonyms = ASS | | | | | |
| P02768 | Serum albumin/Name = ALB | 9.02E−03 | −1.68 | 1.68 | −69.14 | 69.14 |
| P04632 | Calpain small subunit 1/ | 9.08E−03 | −1.84 | 1.84 | −5.57 | 5.57 |
| | Name = CAPNS1; | | | | | |
| | Synonyms = CAPN4, CAPNS | | | | | |
| Q13509 | Tubulin beta-3 chain/ | 9.15E−03 | −1.89 | 1.89 | −28.57 | 28.57 |
| | Name = TUBB3; | | | | | |
| | Synonyms = TUBB4 | | | | | |
| Q96FW1 | Ubiquitin thioesterase OTUB1/ | 9.17E−03 | −1.55 | 1.55 | −2.43 | 2.43 |
| | Name = OTUB1; | | | | | |
| | Synonyms = OTB1, OTU1; | | | | | |
| | ORFNames = HSPC263 | | | | | |
| Q96QK1 | Vacuolar protein | 9.26E−03 | −1.67 | 1.67 | −5 | 5 |
| | sorting-associated protein 35/ | | | | | |
| | Name = VPS35; | | | | | |
| | Synonyms = MEM3; | | | | | |
| | ORFNames = TCCCTA00141 | | | | | |
| O00515 | Ladinin-1/Name = LAD1; | 9.35E−03 | −1.88 | 1.88 | −8.29 | 8.29 |
| | Synonyms = LAD | | | | | |
| Q9Y678 | Coatomer subunit gamma/ | 9.47E−03 | −1.88 | 1.88 | −2.57 | 2.57 |
| | Name = COPG; | | | | | |
| | Synonyms = COPG1 | | | | | |
| Q99424 | Peroxisomal acyl-coenzyme A | 9.52E−03 | 1.88 | 1.88 | 9.57 | 9.57 |
| | oxidase 2/Name = ACOX2 | | | | | |
| Q7Z6Z7 | E3 ubiquitin-protein ligase | 9.60E−03 | −1.87 | 1.87 | −2 | 2 |
| | HUWE1/Name = HUWE1; | | | | | |
| | Synonyms = KIAA0312, | | | | | |
| | KIAA1578, UREB1; | | | | | |
| | ORFNames = HSPC272 | | | | | |
| Q9BUF5 | Tubulin beta-6 chain/ | 9.62E−03 | −1.87 | 1.87 | −21 | 21 |
| | Name = TUBB6 | | | | | |
| P02774 | Vitamin D-binding protein/ | 9.72E−03 | −1.7 | 1.7 | −3.29 | 3.29 |
| | Name = GC | | | | | |
| P09417 | Dihydropteridine reductase/ | 9.74E−03 | 1.87 | 1.87 | 5.57 | 5.57 |
| | Name = QDPR; | | | | | |
| | Synonyms = DHPR | | | | | |
| P50225 | Sulfotransferase 1A1/ | 9.79E−03 | 1.69 | 1.69 | 4.29 | 4.29 |
| | Name = SULT1A1; | | | | | |
| | Synonyms = STP, STP1; | | | | | |
| | ORFNames = OK/SW-cl.88 | | | | | |
| Q9NUI1 | Peroxisomal 2,4-dienoyl-CoA | 9.84E−03 | 1.86 | 1.86 | 4.71 | 4.71 |
| | reductase/Name = DECR2; | | | | | |
| | Synonyms = PDCR | | | | | |
| P31947 | 14-3-3 protein sigma/ | 9.95E−03 | −1.86 | 1.86 | −9.14 | 9.14 |
| | Name = SFN; | | | | | |
| | Synonyms = HME1 | | | | | |

*Bold type indicates increased relative expression in peripheral cholangiocarcinoma compared to hepatocellular carcinoma

Table 6 provides information as to whether the marker proteins are relatively over-expressed (identified in bold) or under-expressed in normal cholangiocytes versus normal hepatocytes. Accordingly, by determining the presence, absence or change in expression levels of a plurality of these marker proteins and comparing these changes with a reference of known expression levels, one is able to determine whether the cells under test are normal cholangiocytes or normal hepatocytes.

TABLE 6
Proteins differentiating normal hepatocytes from normal cholangiocytes*

| | | | | | | |
|---|---|---|---|---|---|---|
| | | | | | Effect | |
| | | | | Magnitude of | Size | Magnitude of |
| | | | Effect | Effect Size | (Mean | Mean dif |
| Protein | Name | P Value | Size (g) | (g) | Dif) | (Mean Dif) |

PART A:

| | | | | | | |
|---|---|---|---|---|---|---|
| P07099 | Epoxide hydrolase 1/ | 1.21E−08 | 9.4 | 9.4 | 38.02 | 38.02 |
| | Name = EPHX1; | | | | | |
| | Synonyms = EPHX, | | | | | |
| | EPOX | | | | | |
| P22307 | Non-specific | 3.92E−08 | 7.85 | 7.85 | 14.14 | 14.14 |
| | lipid-transfer protein/ | | | | | |
| | Name = SCP2 | | | | | |
| Q969Z3 | MOSC | 9.34E−08 | 6.25 | 6.25 | 6.1 | 6.1 |
| | domain-containing | | | | | |
| | protein 2, | | | | | |
| | mitochondrial/ | | | | | |
| | Name = MOSC2 | | | | | |
| Q16851 | UTP--glucose-1-phosphate | 1.06E−07 | 9.18 | 9.18 | 38.5 | 38.5 |
| | uridylyltransferase/ | | | | | |
| | Name = UGP2; | | | | | |
| | Synonyms = UGP1 | | | | | |
| Q16822 | Phosphoenolpyruvate | 2.74E−07 | 7.02 | 7.02 | 39.67 | 39.67 |
| | carboxykinase [GTP], | | | | | |
| | mitochondrial/ | | | | | |
| | Name = PCK2; | | | | | |
| | Synonyms = PEPCK2 | | | | | |
| P05091 | Aldehyde | 3.03E−07 | 5.98 | 5.98 | 49.64 | 49.64 |
| | dehydrogenase, | | | | | |
| | mitochondrial/ | | | | | |
| | Name = ALDH2; | | | | | |
| | Synonyms = ALDM | | | | | |
| P30740* | Leukocyte elastase | 3.97E−07 | −6.1 | 6.1 | −9.9 | 9.9 |
| | inhibitor/ | | | | | |
| | Name = SERPINB1; | | | | | |
| | Synonyms = ELANH2, | | | | | |
| | MNEI, PI2 | | | | | |
| Q02252 | Methylmalonate-semialdehyde | 4.15E−07 | 8.09 | 8.09 | 32.24 | 32.24 |
| | dehydrogenase | | | | | |
| | [acylating], | | | | | |
| | mitochondrial/ | | | | | |
| | Name = ALDH6A1; | | | | | |
| | Synonyms = MMSDH | | | | | |
| Q4G0N4 | NAD kinase | 5.85E−07 | 10.45 | 10.45 | 12.14 | 12.14 |
| | domain-containing | | | | | |
| | protein 1/ | | | | | |
| | Name = NADKD1; | | | | | |
| | Synonyms = C5orf33 | | | | | |
| P16435 | NADPH--cytochrome | 1.42E−06 | 4.81 | 4.81 | 7.79 | 7.79 |
| | P450 reductase/ | | | | | |
| | Name = POR; | | | | | |
| | Synonyms = CYPOR | | | | | |
| P34896 | Serine | 1.90E−06 | 7.21 | 7.21 | 20.24 | 20.24 |
| | hydroxymethyltransferase, | | | | | |
| | cytosolic/ | | | | | |
| | Name = SHMT1 | | | | | |
| P50440 | Glycine | 2.14E−06 | 5.62 | 5.62 | 21.98 | 21.98 |
| | amidinotransferase, | | | | | |
| | mitochondrial/ | | | | | |
| | Name = GATM; | | | | | |
| | Synonyms = AGAT | | | | | |
| Q96QK1* | Vacuolar protein | 2.15E−06 | −5.61 | 5.61 | −5.38 | 5.38 |
| | sorting-associated | | | | | |
| | protein 35/ | | | | | |
| | Name = VPS35; | | | | | |
| | Synonyms = MEM3; | | | | | |
| | ORFNames = TCCCTA00141 | | | | | |
| P16930 | Fumarylacetoacetase/ | 2.34E−06 | 6.04 | 6.04 | 17.38 | 17.38 |
| | Name = FAH | | | | | |
| P30084 | Enoyl-CoA hydratase, | 2.90E−06 | 4.97 | 4.97 | 22.81 | 22.81 |
| | mitochondrial/ | | | | | |
| | Name = ECHS1 | | | | | |
| P27338 | Amine oxidase | 3.06E−06 | 4.79 | 4.79 | 12.45 | 12.45 |
| | [flavin-containing] B/ | | | | | |
| | Name = MAOB | | | | | |
| Q9H2A2 | Aldehyde | 3.20E−06 | 7.83 | 7.83 | 12.14 | 12.14 |
| | dehydrogenase family | | | | | |
| | 8 member A1/ | | | | | |
| | Name = ALDH8A1; | | | | | |
| | Synonyms = ALDH12 | | | | | |
| Q9UHD8* | Septin-9/ | 3.32E−06 | −5.38 | 5.38 | −5.21 | 5.21 |
| | Name = SEPT9; | | | | | |
| | Synonyms = KIAA0991, | | | | | |
| | MSF | | | | | |
| P00367 | Glutamate | 3.36E−06 | 5.78 | 5.78 | 53.21 | 53.21 |
| | dehydrogenase 1, | | | | | |
| | mitochondrial/ | | | | | |
| | Name = GLUD1; | | | | | |
| | Synonyms = GLUD | | | | | |
| O43175 | D-3-phosphoglycerate | 3.48E−06 | 4.35 | 4.35 | 8.43 | 8.43 |
| | dehydrogenase/ | | | | | |
| | Name = PHGDH; | | | | | |
| | Synonyms = PGDH3 | | | | | |
| O95831 | Apoptosis-inducing | 3.83E−06 | 4.5 | 4.5 | 13.74 | 13.74 |
| | factor 1, mitochondrial/ | | | | | |
| | Name = AIFM1; | | | | | |
| | Synonyms = AIF, | | | | | |
| | PDCD8 | | | | | |
| P22760 | Arylacetamide | 3.95E−06 | 7.56 | 7.56 | 8.29 | 8.29 |
| | deacetylase/ | | | | | |
| | Name = AADAC; | | | | | |
| | Synonyms = DAC | | | | | |
| P51659 | Peroxisomal | 4.43E−06 | 4.58 | 4.58 | 26.17 | 26.17 |
| | multifunctional enzyme | | | | | |
| | type 2/ | | | | | |
| | Name = HSD17B4; | | | | | |
| | Synonyms = EDH17B4 | | | | | |
| Q02338 | D-beta-hydroxybutyrate | 5.31E−06 | 7.19 | 7.19 | 16.71 | 16.71 |
| | dehydrogenase, | | | | | |
| | mitochondrial/ | | | | | |
| | Name = BDH1; | | | | | |
| | Synonyms = BDH | | | | | |
| P45954 | Short/branched chain | 5.84E−06 | 6.12 | 6.12 | 17.67 | 17.67 |
| | specific acyl-CoA | | | | | |
| | dehydrogenase, | | | | | |
| | mitochondrial/ | | | | | |
| | Name = ACADSB | | | | | |
| Q01995* | Transgelin/ | 6.29E−06 | −8.08 | 8.08 | −13.71 | 13.71 |
| | Name = TAGLN; | | | | | |
| | Synonyms = SM22, | | | | | |
| | WS3-10 | | | | | |
| P21399 | Cytoplasmic aconitate | 6.70E−06 | 4.62 | 4.62 | 12.4 | 12.4 |
| | hydratase/ | | | | | |
| | Name = ACO1; | | | | | |
| | Synonyms = IREB1 | | | | | |
| O14756 | 17-beta-hydroxysteroid | 6.81E−06 | 6.89 | 6.89 | 14.43 | 14.43 |
| | dehydrogenase type 6/ | | | | | |
| | Name = HSD17B6; | | | | | |
| | Synonyms = RODH | | | | | |
| P21397 | Amine oxidase | 8.06E−06 | 6.69 | 6.69 | 7.43 | 7.43 |
| | [flavin-containing] A/ | | | | | |
| | Name = MAOA | | | | | |
| P35914 | Hydroxymethylglutaryl- | 8.14E−06 | 4.03 | 4.03 | 6.79 | 6.79 |
| | CoA lyase, | | | | | |
| | mitochondrial/ | | | | | |
| | Name = HMGCL | | | | | |
| Q16836 | Hydroxyacyl-coenzyme | 8.65E−06 | 4.51 | 4.51 | 8.62 | 8.62 |
| | A dehydrogenase, | | | | | |
| | mitochondrial/ | | | | | |
| | Name = HADH; | | | | | |
| | Synonyms = HAD, | | | | | |
| | HADHSC, SCHAD | | | | | |
| Q06520 | Bile salt | 8.73E−06 | 6.6 | 6.6 | 15.86 | 15.86 |
| | sulfotransferase/ | | | | | |
| | Name = SULT2A1; | | | | | |
| | Synonyms = HST, STD | | | | | |
| Q9Y6C9 | Mitochondrial carrier | 8.80E−06 | 6.59 | 6.59 | 8 | 8 |
| | homolog 2/ | | | | | |
| | Name = MTCH2; | | | | | |
| | Synonyms = MIMP; | | | | | |
| | ORFNames = HSPC032 | | | | | |
| P30038 | Delta-1-pyrroline-5-carboxylate | 9.19E−06 | 6.54 | 6.54 | 22.43 | 22.43 |
| | dehydrogenase, | | | | | |
| | mitochondrial/ | | | | | |
| | Name = ALDH4A1; | | | | | |
| | Synonyms = ALDH4, | | | | | |
| | P5CDH | | | | | |
| P06737 | Glycogen | 9.88E−06 | 6.46 | 6.46 | 23.29 | 23.29 |
| | phosphorylase, liver | | | | | |
| | form/Name = PYGL | | | | | |
| P50995* | Annexin A11/ | 1.11E−05 | −9.87 | 9.87 | −8.67 | 8.67 |
| | Name = ANXA11; | | | | | |
| | Synonyms = ANX11 | | | | | |
| Q07960* | Rho | 1.15E−05 | −9.79 | 9.79 | −3.67 | 3.67 |
| | GTPase-activating | | | | | |
| | protein 1/ | | | | | |
| | Name = ARHGAP1; | | | | | |
| | Synonyms = CDC42GAP, | | | | | |
| | RHOGAP1 | | | | | |
| Q68CK6 | Acyl-coenzyme A | 1.17E−05 | 6.28 | 6.28 | 30.29 | 30.29 |
| | synthetase ACSM2B, | | | | | |
| | mitochondrial/ | | | | | |
| | Name = ACSM2B; | | | | | |
| | Synonyms = ACSM2; | | | | | |
| | ORFNames = HYST1046 | | | | | |
| Q9Y2Q3 | Glutathione | 1.22E−05 | 3.82 | 3.82 | 6.93 | 6.93 |
| | S-transferase kappa 1/ | | | | | |
| | Name = GSTK1; | | | | | |
| | ORFNames = HDCMD47P | | | | | |
| O95954 | Formimidoyltransferase- | 1.27E−05 | 6.19 | 6.19 | 37.43 | 37.43 |
| | cyclodeaminase/ | | | | | |
| | Name = FTCD | | | | | |
| P17174 | Aspartate | 1.39E−05 | 4.69 | 4.69 | 19.57 | 19.57 |
| | aminotransferase, | | | | | |
| | cytoplasmic/ | | | | | |
| | Name = GOT1 | | | | | |
| P08670* | Vimentin/Name = VIM | 1.42E−05 | −3.89 | 3.89 | −33.31 | 33.31 |
| P21912 | Succinate | 1.44E−05 | 6.06 | 6.06 | 5.14 | 5.14 |
| | dehydrogenase | | | | | |
| | [ubiquinone] iron-sulfur | | | | | |
| | subunit, mitochondrial/ | | | | | |
| | Name = SDHB; | | | | | |
| | Synonyms = SDH, | | | | | |
| | SDH1 | | | | | |
| P51649 | Succinate-semialdehyde | 1.46E−05 | 4.81 | 4.81 | 9.95 | 9.95 |
| | dehydrogenase, | | | | | |
| | mitochondrial/ | | | | | |
| | Name = ALDH5A1; | | | | | |
| | Synonyms = SSADH | | | | | |
| Q9UJS0 | Calcium-binding | 1.52E−05 | 3.88 | 3.88 | 12.31 | 12.31 |
| | mitochondrial carrier | | | | | |
| | protein Aralar2/ | | | | | |
| | Name = SLC25A13; | | | | | |
| | Synonyms = ARALAR2 | | | | | |
| Q7Z4W1 | L-xylulose reductase/ | 1.60E−05 | 5.42 | 5.42 | 30.26 | 30.26 |
| | Name = DCXR | | | | | |
| P10632 | Cytochrome P450 2C8/ | 1.62E−05 | 5.94 | 5.94 | 13.57 | 13.57 |
| | Name = CYP2C8 | | | | | |
| P00480 | Ornithine | 1.68E−05 | 5.9 | 5.9 | 15.71 | 15.71 |
| | carbamoyltransferase, | | | | | |
| | mitochondrial/ | | | | | |
| | Name = OTC | | | | | |
| P08758* | Annexin A5/ | 1.70E−05 | −5.87 | 5.87 | −18.48 | 18.48 |
| | Name = ANXA5; | | | | | |
| | Synonyms = ANX5, | | | | | |
| | ENX2, PP4 | | | | | |
| P30041 | Peroxiredoxin-6/ | 1.72E−05 | 4.32 | 4.32 | 11.26 | 11.26 |
| | Name = PRDX6; | | | | | |
| | Synonyms = AOP2, | | | | | |
| | KIAA0106 | | | | | |
| P49189 | 4-trimethylaminobutyraldehyde | 1.87E−05 | 3.76 | 3.76 | 6.1 | 6.1 |
| | dehydrogenase/ | | | | | |
| | Name = ALDH9A1; | | | | | |
| | Synonyms = ALDH4, | | | | | |
| | ALDH7, ALDH9 | | | | | |
| P49419 | Alpha-aminoadipic | 1.89E−05 | 4.84 | 4.84 | 18.69 | 18.69 |
| | semialdehyde | | | | | |
| | dehydrogenase/ | | | | | |
| | Name = ALDH7A1; | | | | | |
| | Synonyms = ATQ1 | | | | | |
| Q08AH3 | Acyl-coenzyme A | 1.99E−05 | 5.73 | 5.73 | 30.29 | 30.29 |
| | synthetase ACSM2A, | | | | | |
| | mitochondrial/ | | | | | |
| | Name = ACSM2A; | | | | | |
| | Synonyms = ACSM2, | | | | | |
| | MACS2 | | | | | |
| P32754 | 4-hydroxyphenylpyruvate | 1.99E−05 | 5.73 | 5.73 | 18.71 | 18.71 |
| | dioxygenase/ | | | | | |
| | Name = HPD; | | | | | |
| | Synonyms = PPD | | | | | |
| Q6IB77 | Glycine | 2.03E−05 | 5.71 | 5.71 | 13.57 | 13.57 |
| | N-acyltransferase/ | | | | | |
| | Name = GLYAT; | | | | | |
| | Synonyms = ACGNAT, | | | | | |
| | CAT, GAT | | | | | |
| Q13838* | Spliceosome RNA | 2.36E−05 | −3.75 | 3.75 | −7.12 | 7.12 |
| | helicase DDX39B/ | | | | | |
| | Name = DDX39B; | | | | | |
| | Synonyms = BAT1, | | | | | |
| | UAP56 | | | | | |
| P00439 | Phenylalanine-4-hydroxylase/ | 2.40E−05 | 3.54 | 3.54 | 5.88 | 5.88 |
| | Name = PAH | | | | | |
| P28838 | Cytosol | 2.51E−05 | 3.63 | 3.63 | 13.05 | 13.05 |
| | aminopeptidase/ | | | | | |
| | Name = LAP3; | | | | | |
| | Synonyms = LAPEP, | | | | | |
| | PEPS | | | | | |
| P09211* | Glutathione | 2.59E−05 | −7.63 | 7.63 | −39.45 | 39.45 |
| | S-transferase P/ | | | | | |
| | Name = GSTP1; | | | | | |
| | Synonyms = FAEES3, | | | | | |
| | GST3 | | | | | |
| P50053 | Ketohexokinase/ | 2.65E−05 | 5.45 | 5.45 | 5 | 5 |
| | Name = KHK | | | | | |
| O14979* | Heterogeneous | 2.78E−05 | −8.18 | 8.18 | −5.83 | 5.83 |
| | nuclear | | | | | |
| | ribonucleoprotein | | | | | |
| | D-like/ | | | | | |
| | Name = HNRPDL; | | | | | |
| | Synonyms = JKTBP | | | | | |
| P37802* | Transgelin-2/ | 2.84E−05 | −4.52 | 4.52 | −7.26 | 7.26 |
| | Name = TAGLN2; | | | | | |
| | Synonyms = KIAA0120; | | | | | |
| | ORFNames = CDABP0035 | | | | | |
| Q9UBQ7 | Glyoxylate | 2.99E−05 | 4.68 | 4.68 | 20.74 | 20.74 |
| | reductase/hydroxypyruvate | | | | | |
| | reductase/ | | | | | |
| | Name = GRHPR; | | | | | |
| | Synonyms = GLXR; | | | | | |
| | ORFNames = MSTP035 | | | | | |
| P09467 | Fructose-1,6-bisphosphatase | 2.99E−05 | 4.33 | 4.33 | 20.38 | 20.38 |
| | 1/ | | | | | |
| | Name = FBP1; | | | | | |
| | Synonyms = FBP | | | | | |
| P60660* | Myosin light | 3.13E−05 | −3.8 | 3.8 | −6.83 | 6.83 |
| | polypeptide 6/ | | | | | |
| | Name = MYL6 | | | | | |
| P06396* | Gelsolin/Name = GSN | 3.33E−05 | −5.01 | 5.01 | −10.38 | 10.38 |
| P08729* | Keratin, type II | 3.41E−05 | −7.85 | 7.85 | −12.17 | 12.17 |
| | cytoskeletal 7/ | | | | | |
| | Name = KRT7; | | | | | |
| | Synonyms = SCL | | | | | |
| P07954 | Fumarate hydratase, | 3.43E−05 | 3.46 | 3.46 | 10.88 | 10.88 |
| | mitochondrial/ | | | | | |
| | Name = FH | | | | | |
| O75452 | Retinol dehydrogenase | 3.53E−05 | 5.19 | 5.19 | 13.43 | 13.43 |
| | 16/Name = RDH16; | | | | | |
| | Synonyms = RODH4 | | | | | |
| P08107* | Heat shock 70 kDa | 3.76E−05 | −4.78 | 4.78 | −22.98 | 22.98 |
| | protein 1A/1B/ | | | | | |
| | Name = HSPA1A; | | | | | |
| | Synonyms = HSPA1 | | | | | |
| P80404 | 4-aminobutyrate | 3.85E−05 | 5.02 | 5.02 | 38.81 | 38.81 |
| | aminotransferase, | | | | | |
| | mitochondrial/ | | | | | |
| | Name = ABAT; | | | | | |
| | Synonyms = GABAT | | | | | |
| P00167 | Cytochrome b5/ | 4.00E−05 | 3.42 | 3.42 | 9.81 | 9.81 |
| | Name = CYB5A; | | | | | |
| | Synonyms = CYB5 | | | | | |
| P05181 | Cytochrome P450 2E1/ | 4.19E−05 | 5.04 | 5.04 | 8 | 8 |
| | Name = CYP2E1; | | | | | |
| | Synonyms = CYP2E | | | | | |
| O00299* | Chloride intracellular | 4.23E−05 | −4.64 | 4.64 | −11.76 | 11.76 |
| | channel protein 1/ | | | | | |
| | Name = CLIC1; | | | | | |
| | Synonyms = G6, | | | | | |
| | NCC27 | | | | | |
| Q9Y2S2 | Lambda-crystallin | 4.36E−05 | 5 | 5 | 6.43 | 6.43 |
| | homolog/ | | | | | |
| | Name = CRYL1; | | | | | |
| | Synonyms = CRY | | | | | |
| P31943* | Heterogeneous | 4.37E−05 | −3.33 | 3.33 | −7.24 | 7.24 |
| | nuclear | | | | | |
| | ribonucleoprotein H/ | | | | | |
| | Name = HNRNPH1; | | | | | |
| | Synonyms = HNRPH, | | | | | |
| | HNRPH1 | | | | | |
| P08684 | Cytochrome P450 3A4/ | 4.65E−05 | 4.95 | 4.95 | 12 | 12 |
| | Name = CYP3A4; | | | | | |
| | Synonyms = CYP3A3 | | | | | |
| Q05707* | Collagen alpha-1(XIV) | 4.98E−05 | −7.27 | 7.27 | −19.33 | 19.33 |
| | chain/ | | | | | |
| | Name = COL14A1; | | | | | |
| | Synonyms = UND | | | | | |
| P05089 | Arginase-1/ | 5.04E−05 | 4.88 | 4.88 | 28.14 | 28.14 |
| | Name = ARG1 | | | | | |
| P05455* | Lupus La protein/ | 5.30E−05 | −3.69 | 3.69 | −4.43 | 4.43 |
| | Name = SSB | | | | | |
| P30086 | Phosphatidylethanolamine- | 5.38E−05 | 3.67 | 3.67 | 17.29 | 17.29 |
| | binding protein 1/ | | | | | |
| | Name = PEBP1; | | | | | |
| | Synonyms = PBP, | | | | | |
| | PEBP | | | | | |
| Q14032 | Bile acid-CoA:amino | 6.14E−05 | 4.71 | 4.71 | 6.43 | 6.43 |
| | acid N-acyltransferase/ | | | | | |
| | Name = BAAT | | | | | |
| P68366* | Tubulin alpha-4A | 6.27E−05 | −3.82 | 3.82 | −40 | 40 |
| | chain/ | | | | | |
| | Name = TUBA4A; | | | | | |
| | Synonyms = TUBA1 | | | | | |
| Q08426 | Peroxisomal | 6.30E−05 | 4.69 | 4.69 | 41.29 | 41.29 |
| | bifunctional enzyme/ | | | | | |
| | Name = EHHADH; | | | | | |
| | Synonyms = ECHD | | | | | |
| Q8NBX0 | Probable saccharopine | 6.32E−05 | 4.69 | 4.69 | 5.86 | 5.86 |
| | dehydrogenase/ | | | | | |
| | Name = SCCPDH; | | | | | |
| | ORFNames = CGI-49 | | | | | |
| Q03154 | Aminoacylase-1/ | 6.39E−05 | 4.68 | 4.68 | 16.71 | 16.71 |
| | Name = ACY1 | | | | | |
| Q08211* | ATP-dependent RNA | 6.61E−05 | −5.12 | 5.12 | −10.1 | 10.1 |
| | helicase A/ | | | | | |
| | Name = DHX9; | | | | | |
| | Synonyms = DDX9, | | | | | |
| | LKP, NDH2 | | | | | |
| O43390* | Heterogeneous | 6.75E−05 | −3.34 | 3.34 | −6.38 | 6.38 |
| | nuclear | | | | | |
| | ribonucleoprotein R/ | | | | | |
| | Name = HNRNPR; | | | | | |
| | Synonyms = HNRPR | | | | | |
| P19105* | Myosin regulatory | 7.33E−05 | −3.86 | 3.86 | −5.64 | 5.64 |
| | light chain 12A/ | | | | | |
| | Name = MYL12A; | | | | | |
| | Synonyms = MLCB, | | | | | |
| | MRLC3, RLC | | | | | |
| O14950* | Myosin regulatory | 7.33E−05 | −3.86 | 3.86 | −5.64 | 5.64 |
| | light chain 12B/ | | | | | |
| | Name = MYL12B; | | | | | |
| | Synonyms = MRLC2, | | | | | |
| | MYLC2B | | | | | |
| Q99623 | Prohibitin-2/ | 7.56E−05 | 3.18 | 3.18 | 5.14 | 5.14 |
| | Name = PHB2; | | | | | |
| | Synonyms = BAP, REA | | | | | |
| P26440 | Isovaleryl-CoA | 7.76E−05 | 3.12 | 3.12 | 4.83 | 4.83 |
| | dehydrogenase, | | | | | |
| | mitochondrial/ | | | | | |
| | Name = IVD | | | | | |
| P61978* | Heterogeneous | 8.02E−05 | −4.28 | 4.28 | −12.88 | 12.88 |
| | nuclear | | | | | |
| | ribonucleoprotein K/ | | | | | |
| | Name = HNRNPK; | | | | | |
| | Synonyms = HNRPK | | | | | |
| P67936* | Tropomyosin alpha-4 | 8.10E−05 | −4.86 | 4.86 | −7.21 | 7.21 |
| | chain/Name = TPM4 | | | | | |
| P21810* | Biglycan/ | 8.14E−05 | −6.13 | 6.13 | −39.6 | 39.6 |
| | Name = BGN; | | | | | |
| | Synonyms = SLRR1A | | | | | |
| P04424 | Argininosuccinate | 8.16E−05 | 3.42 | 3.42 | 12.43 | 12.43 |
| | lyase/Name = ASL | | | | | |
| P24752 | Acetyl-CoA | 8.38E−05 | 4.3 | 4.3 | 34.83 | 34.83 |
| | acetyltransferase, | | | | | |
| | mitochondrial/ | | | | | |
| | Name = ACAT1; | | | | | |
| | Synonyms = ACAT, | | | | | |
| | MAT | | | | | |
| O00748 | Cocaine esterase/ | 8.46E−05 | 3.33 | 3.33 | 7.36 | 7.36 |
| | Name = CES2; | | | | | |
| | Synonyms = ICE | | | | | |
| Q9Y2P5 | Bile acyl-CoA | 8.48E−05 | 4.45 | 4.45 | 20.14 | 20.14 |
| | synthetase/ | | | | | |
| | Name = SLC27A5; | | | | | |
| | Synonyms = ACSB, | | | | | |
| | ACSVL6, FACVL3, | | | | | |
| | FATP5 | | | | | |
| P31040 | Succinate | 8.68E−05 | 3.1 | 3.1 | 8.88 | 8.88 |
| | dehydrogenase | | | | | |
| | [ubiquinone] | | | | | |
| | flavoprotein subunit, | | | | | |
| | mitochondrial/ | | | | | |
| | Name = SDHA; | | | | | |
| | Synonyms = SDH2, | | | | | |
| | SDHF | | | | | |
| P11586 | C-1-tetrahydrofolate | 8.73E−05 | 4.43 | 4.43 | 26 | 26 |
| | synthase, cytoplasmic/ | | | | | |
| | Name = MTHFD1; | | | | | |
| | Synonyms = MTHFC, | | | | | |
| | MTHFD | | | | | |
| P36871 | Phosphoglucomutase- | 8.74E−05 | 3.43 | 3.43 | 26.21 | 26.21 |
| | 1/Name = PGM1 | | | | | |
| Q9H8H3 | Methyltransferase-like | 8.77E−05 | 4.42 | 4.42 | 5.14 | 5.14 |
| | protein 7A/ | | | | | |
| | Name = METTL7A; | | | | | |
| | ORFNames = PRO0066, | | | | | |
| | UNQ1902/PRO4348 | | | | | |
| Q9NVS9 | Pyridoxine-5′-phosphate | 9.05E−05 | 3.66 | 3.66 | 3.69 | 3.69 |
| | oxidase/ | | | | | |
| | Name = PNPO | | | | | |
| P21695 | Glycerol-3-phosphate | 9.12E−05 | 4.39 | 4.39 | 8.71 | 8.71 |
| | dehydrogenase | | | | | |
| | [NAD+], cytoplasmic/ | | | | | |
| | Name = GPD1 | | | | | |
| Q9UI17 | Dimethylglycine | 9.49E−05 | 4.36 | 4.36 | 12.29 | 12.29 |
| | dehydrogenase, | | | | | |
| | mitochondrial/ | | | | | |
| | Name = DMGDH | | | | | |
| Q9UL12 | Sarcosine | 1.06E−04 | 4.28 | 4.28 | 17.29 | 17.29 |
| | dehydrogenase, | | | | | |
| | mitochondrial/ | | | | | |
| | Name = SARDH; | | | | | |
| | Synonyms = DMGDHL1 | | | | | |
| P34913 | Epoxide hydrolase 2/ | 1.07E−04 | 4.27 | 4.27 | 13 | 13 |
| | Name = EPHX2 | | | | | |
| O00571* | ATP-dependent RNA | 1.11E−04 | −3.66 | 3.66 | −4.93 | 4.93 |
| | helicase DDX3X/ | | | | | |
| | Name = DDX3X; | | | | | |
| | Synonyms = DBX, | | | | | |
| | DDX3 | | | | | |
| Q9UIJ7 | GTP:AMP | 1.17E−04 | 3.16 | 3.16 | 5.45 | 5.45 |
| | phosphotransferase, | | | | | |
| | mitochondrial/ | | | | | |
| | Name = AK3; | | | | | |
| | Synonyms = AK3L1, | | | | | |
| | AK6, AKL3L | | | | | |
| P40926 | Malate | 1.21E−04 | 2.97 | 2.97 | 7.29 | 7.29 |
| | dehydrogenase, | | | | | |
| | mitochondrial/ | | | | | |
| | Name = MDH2 | | | | | |
| P13010* | X-ray repair | 1.25E−04 | −4.51 | 4.51 | −8.6 | 8.6 |
| | cross-complementing | | | | | |
| | protein 5/ | | | | | |
| | Name = XRCC5; | | | | | |
| | Synonyms = G22P2 | | | | | |
| P09525* | Annexin A4/ | 1.30E−04 | −5.9 | 5.9 | −68.21 | 68.21 |
| | Name = ANXA4; | | | | | |
| | Synonyms = ANX4 | | | | | |
| P08727* | Keratin, type I | 1.31E−04 | −5.96 | 5.96 | −33 | 33 |
| | cytoskeletal 19/ | | | | | |
| | Name = KRT19 | | | | | |
| Q86WU2 | Probable D-lactate | 1.32E−04 | 4.12 | 4.12 | 2.57 | 2.57 |
| | dehydrogenase, | | | | | |
| | mitochondrial/ | | | | | |
| | Name = LDHD | | | | | |
| Q01105* | Protein SET/ | 1.33E−04 | −2.98 | 2.98 | −3.95 | 3.95 |
| | Name = SET | | | | | |
| Q99714 | 3-hydroxyacyl-CoA | 1.33E−04 | 3.03 | 3.03 | 9.55 | 9.55 |
| | dehydrogenase type-2/ | | | | | |
| | Name = HSD17B10; | | | | | |
| | Synonyms = ERAB, | | | | | |
| | HADH2, MRPP2, | | | | | |
| | SCHAD, XH98G2 | | | | | |
| Q9NQR4 | Omega-amidase NIT2/ | 1.34E−04 | 3.01 | 3.01 | 6.31 | 6.31 |
| | Name = NIT2; | | | | | |
| | ORFNames = CUA002 | | | | | |
| Q9NR45* | Sialic acid synthase/ | 1.38E−04 | −3.09 | 3.09 | −3.43 | 3.43 |
| | Name = NANS; | | | | | |
| | Synonyms = SAS | | | | | |
| O75521 | Enoyl-CoA delta | 1.41E−04 | 4.07 | 4.07 | 7.43 | 7.43 |
| | isomerase 2, | | | | | |
| | mitochondrial/ | | | | | |
| | Name = ECI2; | | | | | |
| | Synonyms = DRS1, | | | | | |
| | HCA88, PECI | | | | | |
| Q00839* | Heterogeneous | 1.43E−04 | −4.08 | 4.08 | −11.9 | 11.9 |
| | nuclear | | | | | |
| | ribonucleoprotein U/ | | | | | |
| | Name = HNRNPU; | | | | | |
| | Synonyms = HNRPU, | | | | | |
| | SAFA, U21.1 | | | | | |
| P12111* | Collagen alpha-3(VI) | 1.55E−04 | −3.89 | 3.89 | −57.57 | 57.57 |
| | chain/ | | | | | |
| | Name = COL6A3 | | | | | |
| Q13263* | Transcription | 1.58E−04 | −5.72 | 5.72 | −5.67 | 5.67 |
| | intermediary factor | | | | | |
| | 1-beta/ | | | | | |
| | Name = TRIM28; | | | | | |
| | Synonyms = KAP1, | | | | | |
| | RNF96, TIF1B | | | | | |
| P21549 | Serine--pyruvate | 1.64E−04 | 3.96 | 3.96 | 68.43 | 68.43 |
| | aminotransferase/ | | | | | |
| | Name = AGXT; | | | | | |
| | Synonyms = AGT1, | | | | | |
| | SPAT | | | | | |
| O60506* | Heterogeneous | 1.70E−04 | −2.89 | 2.89 | −5.83 | 5.83 |
| | nuclear | | | | | |
| | ribonucleoprotein Q/ | | | | | |
| | Name = SYNCRIP; | | | | | |
| | Synonyms = HNRPQ, | | | | | |
| | NSAP1 | | | | | |
| P24298 | Alanine | 1.70E−04 | 3.93 | 3.93 | 13 | 13 |
| | aminotransferase 1/ | | | | | |
| | Name = GPT; | | | | | |
| | Synonyms = AAT1, | | | | | |
| | GPT1 | | | | | |
| P46952 | 3-hydroxyanthranilate | 1.71E−04 | 3.93 | 3.93 | 8.14 | 8.14 |
| | 3,4-dioxygenase/ | | | | | |
| | Name = HAAO | | | | | |
| P43243* | Matrin-3/ | 1.80E−04 | −2.85 | 2.85 | −4.69 | 4.69 |
| | Name = MATR3; | | | | | |
| | Synonyms = KIAA0723 | | | | | |
| O95994* | Anterior gradient | 1.83E−04 | −5.55 | 5.55 | −12.83 | 12.83 |
| | protein 2 homolog/ | | | | | |
| | Name = AGR2; | | | | | |
| | Synonyms = AG2; | | | | | |
| | ORFNames = UNQ515/ | | | | | |
| | PRO1030 | | | | | |
| P25325 | 3-mercaptopyruvate | 1.89E−04 | 3.74 | 3.74 | 12.05 | 12.05 |
| | sulfurtransferase/ | | | | | |
| | Name = MPST; | | | | | |
| | Synonyms = TST2 | | | | | |
| P40121* | Macrophage-capping | 1.91E−04 | −5.5 | 5.5 | −5.5 | 5.5 |
| | protein/ | | | | | |
| | Name = CAPG; | | | | | |
| | Synonyms = AFCP, | | | | | |
| | MCP | | | | | |
| P15311* | Ezrin/Name = EZR; | 1.93E−04 | −5.49 | 5.49 | −13.5 | 13.5 |
| | Synonyms = VIL2 | | | | | |
| P08133 | Annexin A6/ | 1.94E−04 | 2.96 | 2.96 | 15.69 | 15.69 |
| | Name = ANXA6; | | | | | |
| | Synonyms = ANX6 | | | | | |
| Q6YN16 | Hydroxysteroid | 2.02E−04 | 2.8 | 2.8 | 4.43 | 4.43 |
| | dehydrogenase-like | | | | | |
| | protein 2/ | | | | | |
| | Name = HSDL2; | | | | | |
| | Synonyms = C9orf99 | | | | | |
| P62258* | 14-3-3 protein epsilon/ | 2.02E−04 | −2.96 | 2.96 | −9.52 | 9.52 |
| | Name = YWHAE | | | | | |
| Q16698 | 2,4-dienoyl-CoA | 2.15E−04 | 3.08 | 3.08 | 13.31 | 13.31 |
| | reductase, | | | | | |
| | mitochondrial/ | | | | | |
| | Name = DECR1; | | | | | |
| | Synonyms = DECR | | | | | |
| P05062 | Fructose-bisphosphate | 2.19E−04 | 3.73 | 3.73 | 71.67 | 71.67 |
| | aldolase B/ | | | | | |
| | Name = ALDOB; | | | | | |
| | Synonyms = ALDB | | | | | |
| Q04917* | 14-3-3 protein eta/ | 2.20E−04 | −3.17 | 3.17 | −10.83 | 10.83 |
| | Name = YWHAH; | | | | | |
| | Synonyms = YWHA1 | | | | | |
| P53007 | Tricarboxylate | 2.24E−04 | 3.35 | 3.35 | 4.57 | 4.57 |
| | transport protein, | | | | | |
| | mitochondrial/ | | | | | |
| | Name = SLC25A1; | | | | | |
| | Synonyms = SLC20A3 | | | | | |
| P30613 | Pyruvate kinase | 2.25E−04 | 3.74 | 3.74 | 14.43 | 14.43 |
| | isozymes R/L/ | | | | | |
| | Name = PKLR; | | | | | |
| | Synonyms = PK1, PKL | | | | | |
| Q16555* | Dihydropyrimidinase- | 2.26E−04 | −4.73 | 4.73 | −10.88 | 10.88 |
| | related protein 2/ | | | | | |
| | Name = DPYSL2; | | | | | |
| | Synonyms = CRMP2, | | | | | |
| | ULIP2 | | | | | |
| P11712 | Cytochrome P450 2C9/ | 2.29E−04 | 3.73 | 3.73 | 17 | 17 |
| | Name = CYP2C9; | | | | | |
| | Synonyms = CYP2C10 | | | | | |
| Q00266 | S-adenosylmethionine | 2.33E−04 | 3.72 | 3.72 | 10.14 | 10.14 |
| | synthase isoform | | | | | |
| | type-1/ | | | | | |
| | Name = MAT1A; | | | | | |
| | Synonyms = AMS1, | | | | | |
| | MATA1 | | | | | |
| Q9BPW8 | Protein NipSnap | 2.38E−04 | 3.7 | 3.7 | 7.86 | 7.86 |
| | homolog 1/ | | | | | |
| | Name = NIPSNAP1 | | | | | |
| P31930 | Cytochrome b-c1 | 2.38E−04 | 2.73 | 2.73 | 4.52 | 4.52 |
| | complex subunit 1, | | | | | |
| | mitochondrial/ | | | | | |
| | Name = UQCRC1 | | | | | |
| Q14117 | Dihydropyrimidinase/ | 2.41E−04 | 3.69 | 3.69 | 6.71 | 6.71 |
| | Name = DPYS | | | | | |
| O75356 | Ectonucleoside | 2.43E−04 | 3.69 | 3.69 | 4.43 | 4.43 |
| | triphosphate | | | | | |
| | diphosphohydrolase 5/ | | | | | |
| | Name = ENTPD5; | | | | | |
| | Synonyms = CD39L4, | | | | | |
| | PCPH | | | | | |
| P35520 | Cystathionine | 2.43E−04 | 3.69 | 3.69 | 4.43 | 4.43 |
| | beta-synthase/ | | | | | |
| | Name = CBS | | | | | |
| P30039 | Phenazine | 2.60E−04 | 3.64 | 3.64 | 9.43 | 9.43 |
| | biosynthesis-like | | | | | |
| | domain-containing | | | | | |
| | protein/Name = PBLD; | | | | | |
| | Synonyms = MAWBP | | | | | |
| Q93088 | Betaine--homocysteine | 2.62E−04 | 3.64 | 3.64 | 29.86 | 29.86 |
| | S-methyltransferase 1/ | | | | | |
| | Name = BHMT | | | | | |
| Q3LXA3 | Bifunctional | 2.66E−04 | 3.47 | 3.47 | 31.83 | 31.83 |
| | ATP-dependent | | | | | |
| | dihydroxyacetone | | | | | |
| | kinase/FAD-AMP lyase | | | | | |
| | (cyclizing)/ | | | | | |
| | Name = DAK | | | | | |
| Q9HDC9 | Adipocyte plasma | 2.90E−04 | 2.65 | 2.65 | 4.26 | 4.26 |
| | membrane-associated | | | | | |
| | protein/ | | | | | |
| | Name = APMAP; | | | | | |
| | Synonyms = C20orf3; | | | | | |
| | ORFNames = UNQ1869/ | | | | | |
| | PRO4305 | | | | | |
| P78417 | Glutathione | 3.10E−04 | 2.8 | 2.8 | 8.17 | 8.17 |
| | S-transferase omega-1/ | | | | | |
| | Name = GSTO1; | | | | | |
| | Synonyms = GSTTLP28 | | | | | |
| O75367* | Core histone | 3.15E−04 | −4.24 | 4.24 | −11.52 | 11.52 |
| | macro-H2A.1/ | | | | | |
| | Name = H2AFY; | | | | | |
| | Synonyms = MACROH2A1 | | | | | |
| P26599* | Polypyrimidine | 3.31E−04 | −3.17 | 3.17 | −10.24 | 10.24 |
| | tract-binding protein | | | | | |
| | 1/Name = PTBP1; | | | | | |
| | Synonyms = PTB | | | | | |
| P16662 | UDP-glucuronosyltransferase | 3.34E−04 | 3.21 | 3.21 | 13.93 | 13.93 |
| | 2B7/ | | | | | |
| | Name = UGT2B7; | | | | | |
| | Synonyms = UGTB2B9 | | | | | |
| P56470* | Galectin-4/ | 3.40E−04 | −2.63 | 2.63 | −18.6 | 18.6 |
| | Name = LGALS4 | | | | | |
| Q9P0Z9 | Peroxisomal sarcosine | 3.41E−04 | 3.47 | 3.47 | 12.29 | 12.29 |
| | oxidase/ | | | | | |
| | Name = PIPOX; | | | | | |
| | Synonyms = LPIPOX, | | | | | |
| | PSO | | | | | |
| P00966 | Argininosuccinate | 3.44E−04 | 2.93 | 2.93 | 15.98 | 15.98 |
| | synthase/ | | | | | |
| | Name = ASS1; | | | | | |
| | Synonyms = ASS | | | | | |
| P68371* | Tubulin beta-2C | 3.46E−04 | −2.61 | 2.61 | −42.24 | 42.24 |
| | chain/ | | | | | |
| | Name = TUBB2C | | | | | |
| P16219 | Short-chain specific | 3.46E−04 | 3.16 | 3.16 | 13.83 | 13.83 |
| | acyl-CoA | | | | | |
| | dehydrogenase, | | | | | |
| | mitochondrial/ | | | | | |
| | Name = ACADS | | | | | |
| P33121 | Long-chain-fatty-acid-- | 3.46E−04 | 3.46 | 3.46 | 22.29 | 22.29 |
| | CoA ligase 1/ | | | | | |
| | Name = ACSL1; | | | | | |
| | Synonyms = FACL1, | | | | | |
| | FACL2, LACS, LACS1, | | | | | |
| | LACS2 | | | | | |
| P09110 | 3-ketoacyl-CoA | 3.48E−04 | 3.4 | 3.4 | 33.38 | 33.38 |
| | thiolase, peroxisomal/ | | | | | |
| | Name = ACAA1; | | | | | |
| | Synonyms = ACAA, | | | | | |
| | PTHIO | | | | | |
| Q02928 | Cytochrome P450 | 3.52E−04 | 3.45 | 3.45 | 7.14 | 7.14 |
| | 4A11/ | | | | | |
| | Name = CYP4A11; | | | | | |
| | Synonyms = CYP4A2 | | | | | |
| O75891 | Aldehyde | 3.59E−04 | 3.43 | 3.43 | 32 | 32 |
| | dehydrogenase family | | | | | |
| | 1 member L1/ | | | | | |
| | Name = ALDH1L1; | | | | | |
| | Synonyms = FTHFD | | | | | |
| P31327 | Carbamoyl-phosphate | 3.68E−04 | 3.4 | 3.4 | 228.5 | 228.5 |
| | synthase [ammonia], | | | | | |
| | mitochondrial/ | | | | | |
| | Name = CPS1 | | | | | |
| P11509 | Cytochrome P450 2A6/ | 3.73E−04 | 3.41 | 3.41 | 22 | 22 |
| | Name = CYP2A6; | | | | | |
| | Synonyms = CYP2A3 | | | | | |
| P34897 | Serine | 3.80E−04 | 2.84 | 2.84 | 9.95 | 9.95 |
| | hydroxymethyltransferase, | | | | | |
| | mitochondrial/ | | | | | |
| | Name = SHMT2 | | | | | |
| Q9H9B4 | Sideroflexin-1/ | 3.86E−04 | 3.39 | 3.39 | 7.43 | 7.43 |
| | Name = SFXN1 | | | | | |
| Q9Y617 | Phosphoserine | 3.86E−04 | 3.39 | 3.39 | 5.57 | 5.57 |
| | aminotransferase/ | | | | | |
| | Name = PSAT1; | | | | | |
| | Synonyms = PSA | | | | | |
| Q9UBX3 | Mitochondrial | 3.86E−04 | 3.39 | 3.39 | 3.71 | 3.71 |
| | dicarboxylate carrier/ | | | | | |
| | Name = SLC25A10; | | | | | |
| | Synonyms = DIC | | | | | |
| P00441 | Superoxide dismutase | 4.11E−04 | 2.97 | 2.97 | 10.07 | 10.07 |
| | [Cu—Zn]/Name = SOD1 | | | | | |
| Q16775 | Hydroxyacylglutathione | 4.35E−04 | 3.32 | 3.32 | 4.14 | 4.14 |
| | hydrolase, | | | | | |
| | mitochondrial/ | | | | | |
| | Name = HAGH; | | | | | |
| | Synonyms = GLO2, | | | | | |
| | HAGH1 | | | | | |
| Q9Y3I0* | tRNA-splicing ligase | 4.35E−04 | −4.63 | 4.63 | −3 | 3 |
| | RtcB homolog/ | | | | | |
| | Name = C22orf28; | | | | | |
| | ORFNames = HSPC117 | | | | | |
| Q15067 | Peroxisomal | 4.40E−04 | 3.31 | 3.31 | 13.29 | 13.29 |
| | acyl-coenzyme A | | | | | |
| | oxidase 1/ | | | | | |
| | Name = ACOX1; | | | | | |
| | Synonyms = ACOX | | | | | |
| O00264 | Membrane-associated | 4.40E−04 | 2.52 | 2.52 | 7.57 | 7.57 |
| | progesterone receptor | | | | | |
| | component 1/ | | | | | |
| | Name = PGRMC1; | | | | | |
| | Synonyms = HPR6.6, | | | | | |
| | PGRMC | | | | | |
| P04083* | Annexin A1/ | 4.62E−04 | −3.05 | 3.05 | −9.62 | 9.62 |
| | Name = ANXA1; | | | | | |
| | Synonyms = ANX1, | | | | | |
| | LPC1 | | | | | |
| P08319 | Alcohol | 4.68E−04 | 3.27 | 3.27 | 62 | 62 |
| | dehydrogenase 4/ | | | | | |
| | Name = ADH4 | | | | | |
| P22314* | Ubiquitin-like | 4.70E−04 | −2.74 | 2.74 | −10.45 | 10.45 |
| | modifier-activating | | | | | |
| | enzyme 1/ | | | | | |
| | Name = UBA1; | | | | | |
| | Synonyms = A1S9T, | | | | | |
| | UBE1 | | | | | |
| P07195* | L-lactate | 4.77E−04 | −2.58 | 2.58 | −7.24 | 7.24 |
| | dehydrogenase B | | | | | |
| | chain/Name = LDHB | | | | | |
| P43155 | Carnitine | 4.78E−04 | 3.26 | 3.26 | 3.14 | 3.14 |
| | O-acetyltransferase/ | | | | | |
| | Name = CRAT; | | | | | |
| | Synonyms = CAT1 | | | | | |
| P12956* | X-ray repair | 4.80E−04 | −3.94 | 3.94 | −10.98 | 10.98 |
| | cross-complementing | | | | | |
| | protein 6/ | | | | | |
| | Name = XRCC6; | | | | | |
| | Synonyms = G22P1 | | | | | |
| Q16134 | Electron transfer | 4.85E−04 | 3.25 | 3.25 | 7.43 | 7.43 |
| | flavoprotein-ubiquinone | | | | | |
| | oxidoreductase, | | | | | |
| | mitochondrial/ | | | | | |
| | Name = ETFDH | | | | | |
| Q96PK6* | RNA-binding protein | 4.93E−04 | −4.5 | 4.5 | −2.67 | 2.67 |
| | 14/Name = RBM14; | | | | | |
| | Synonyms = SIP | | | | | |
| P30046 | D-dopachrome | 4.99E−04 | 2.54 | 2.54 | 6.67 | 6.67 |
| | decarboxylase/ | | | | | |
| | Name = DDT | | | | | |
| P09417 | Dihydropteridine | 5.12E−04 | 3.22 | 3.22 | 9.14 | 9.14 |
| | reductase/ | | | | | |
| | Name = QDPR; | | | | | |
| | Synonyms = DHPR | | | | | |
| P55786* | Puromycin-sensitive | 5.70E−04 | −3.16 | 3.16 | −4.93 | 4.93 |
| | aminopeptidase/ | | | | | |
| | Name = NPEPPS; | | | | | |
| | Synonyms = PSA | | | | | |
| P07384* | Calpain-1 catalytic | 5.70E−04 | −4.07 | 4.07 | −14.43 | 14.43 |
| | subunit/ | | | | | |
| | Name = CAPN1; | | | | | |
| | Synonyms = CANPL1; | | | | | |
| | ORFNames = PIG30 | | | | | |
| P04040 | Catalase/Name = CAT | 5.88E−04 | 3.06 | 3.06 | 50.98 | 50.98 |
| P60842* | Eukaryotic initiation | 6.15E−04 | −2.67 | 2.67 | −7.1 | 7.1 |
| | factor 4A-I/ | | | | | |
| | Name = EIF4A1; | | | | | |
| | Synonyms = DDX2A, | | | | | |
| | EIF4A | | | | | |
| P11498 | Pyruvate carboxylase, | 6.17E−04 | 3.11 | 3.11 | 31.71 | 31.71 |
| | mitochondrial/ | | | | | |
| | Name = PC | | | | | |
| P23528* | Cofilin-1/ | 6.28E−04 | −2.4 | 2.4 | −4.81 | 4.81 |
| | Name = CFL1; | | | | | |
| | Synonyms = CFL | | | | | |
| P00558* | Phosphoglycerate | 6.29E−04 | −2.61 | 2.61 | −13.74 | 13.74 |
| | kinase 1/ | | | | | |
| | Name = PGK1; | | | | | |
| | Synonyms = PGKA; | | | | | |
| | ORFNames = MIG10, | | | | | |
| | OK/SW-cl.110 | | | | | |
| P07327 | Alcohol | 6.32E−04 | 2.98 | 2.98 | 52.05 | 52.05 |
| | dehydrogenase 1A/ | | | | | |
| | Name = ADH1A; | | | | | |
| | Synonyms = ADH1 | | | | | |
| Q9BTZ2 | Dehydrogenase/reductase | 6.39E−04 | 3.09 | 3.09 | 3.86 | 3.86 |
| | SDR family | | | | | |
| | member 4/ | | | | | |
| | Name = DHRS4; | | | | | |
| | ORFNames = UNQ851/ | | | | | |
| | PRO1800 | | | | | |
| P04632* | Calpain small subunit | 6.42E−04 | −3.26 | 3.26 | −7.29 | 7.29 |
| | 1/Name = CAPNS1; | | | | | |
| | Synonyms = CAPN4, | | | | | |
| | CAPNS | | | | | |
| P78329 | Leukotriene-B(4) | 6.50E−04 | 3.08 | 3.08 | 4.43 | 4.43 |
| | omega-hydroxylase 1/ | | | | | |
| | Name = CYP4F2 | | | | | |
| P06748* | Nucleophosmin/ | 6.64E−04 | −2.89 | 2.89 | −5.83 | 5.83 |
| | Name = NPM1; | | | | | |
| | Synonyms = NPM | | | | | |
| Q13151* | Heterogeneous | 7.14E−04 | −4.16 | 4.16 | −4.83 | 4.83 |
| | nuclear | | | | | |
| | ribonucleoprotein A0/ | | | | | |
| | Name = HNRNPA0; | | | | | |
| | Synonyms = HNRPA0 | | | | | |
| Q7Z5P4 | 17-beta-hydroxysteroid | 7.45E−04 | 3 | 3 | 8.57 | 8.57 |
| | dehydrogenase 13/ | | | | | |
| | Name = HSD17B13; | | | | | |
| | Synonyms = SCDR9; | | | | | |
| | ORFNames = HMFN0376, | | | | | |
| | UNQ497/PRO1014 | | | | | |
| O95479 | GDH/6PGL | 7.49E−04 | 2.68 | 2.68 | 5.95 | 5.95 |
| | endoplasmic | | | | | |
| | bifunctional protein/ | | | | | |
| | Name = H6PD; | | | | | |
| | Synonyms = GDH | | | | | |
| P50991* | T-complex protein 1 | 7.55E−04 | −3.16 | 3.16 | −4.21 | 4.21 |
| | subunit delta/ | | | | | |
| | Name = CCT4; | | | | | |
| | Synonyms = CCTD, | | | | | |
| | SRB | | | | | |
| Q9UBR2 | Cathepsin Z/ | 7.56E−04 | 2.42 | 2.42 | 3.36 | 3.36 |
| | Name = CTSZ | | | | | |
| Q15019* | Septin-2/ | 7.71E−04 | −4.09 | 4.09 | −4.5 | 4.5 |
| | Name = SEPT2; | | | | | |
| | Synonyms = DIFF6, | | | | | |
| | KIAA0158, NEDD5 | | | | | |
| P24539 | ATP synthase subunit | 7.76E−04 | 2.37 | 2.37 | 2.95 | 2.95 |
| | b, mitochondrial/ | | | | | |
| | Name = ATP5F1 | | | | | |
| Q96199 | Succinyl-CoA ligase | 8.21E−04 | 2.56 | 2.56 | 6.14 | 6.14 |
| | [GDP-forming] subunit | | | | | |
| | beta, mitochondrial/ | | | | | |
| | Name = SUCLG2 | | | | | |
| P48735 | Isocitrate | 8.39E−04 | 2.49 | 2.49 | 16.29 | 16.29 |
| | dehydrogenase | | | | | |
| | [NADP], mitochondrial/ | | | | | |
| | Name = IDH2 | | | | | |
| P54868 | Hydroxymethylglutaryl- | 8.44E−04 | 2.92 | 2.92 | 59.1 | 59.1 |
| | CoA synthase, | | | | | |
| | mitochondrial/ | | | | | |
| | Name = HMGCS2 | | | | | |
| P20774* | Mimecan/ | 8.69E−04 | −3.99 | 3.99 | −20.33 | 20.33 |
| | Name = OGN; | | | | | |
| | Synonyms = OIF, | | | | | |
| | SLRR3A | | | | | |
| P54578* | Ubiquitin | 8.82E−04 | −3.97 | 3.97 | −2.83 | 2.83 |
| | carboxyl-terminal | | | | | |
| | hydrolase 14/ | | | | | |
| | Name = USP14; | | | | | |
| | Synonyms = TGT | | | | | |
| P51888* | Prolargin/ | 9.02E−04 | −3.64 | 3.64 | −30.1 | 30.1 |
| | Name = PRELP; | | | | | |
| | Synonyms = SLRR2A | | | | | |
| P09960* | Leukotriene A-4 | 9.14E−04 | −2.52 | 2.52 | −4 | 4 |
| | hydrolase/ | | | | | |
| | Name = LTA4H; | | | | | |
| | Synonyms = LTA4 | | | | | |
| P52758 | Ribonuclease UK114/ | 9.24E−04 | 2.64 | 2.64 | 6.52 | 6.52 |
| | Name = HRSP12; | | | | | |
| | Synonyms = PSP | | | | | |
| P27348* | 14-3-3 protein theta/ | 9.53E−04 | −2.97 | 2.97 | −7.31 | 7.31 |
| | Name = YWHAQ | | | | | |
| Q96913 | Glycine | 9.65E−04 | 2.86 | 2.86 | 1.71 | 1.71 |
| | N-acyltransferase-like | | | | | |
| | protein 1/ | | | | | |
| | Name = GLYATL1; | | | | | |
| | Synonyms = GNAT | | | | | |
| Q9UBR1 | Beta-ureidopropionase/ | 9.87E−04 | 2.84 | 2.84 | 11.43 | 11.43 |
| | Name = UPB1; | | | | | |
| | Synonyms = BUP1 | | | | | |
| Q93099 | Homogentisate | 1.01E−03 | 2.83 | 2.83 | 9 | 9 |
| | 1,2-dioxygenase/ | | | | | |
| | Name = HGD; | | | | | |
| | Synonyms = HGO | | | | | |
| O43772 | Mitochondrial | 1.03E−03 | 2.82 | 2.82 | 6.57 | 6.57 |
| | carnitine/acylcarnitine | | | | | |
| | carrier protein/ | | | | | |
| | Name = SLC25A20; | | | | | |
| | Synonyms = CAC, | | | | | |
| | CACT | | | | | |

PART B:

| | | | | | | |
|---|---|---|---|---|---|---|
| Q99424 | Peroxisomal | 1.05E−03 | 2.81 | 2.81 | 17.43 | 17.43 |
| | acyl-coenzyme A | | | | | |
| | oxidase 2/ | | | | | |
| | Name = ACOX2 | | | | | |
| P19338 | Nucleolin/Name = NCL | 1.05E−03 | −2.73 | 2.73 | −6.88 | 6.88 |
| P07585 | Decorin/Name = DCN; | 1.06E−03 | −3.82 | 3.82 | −9.17 | 9.17 |
| | Synonyms = SLRR1B | | | | | |
| P09651 | Heterogeneous | 1.08E−03 | −3.49 | 3.49 | −13.93 | 13.93 |
| | nuclear | | | | | |
| | ribonucleoprotein A1/ | | | | | |
| | Name = HNRNPA1; | | | | | |
| | Synonyms = HNRPA1 | | | | | |
| P55072 | Transitional | 1.08E−03 | −2.46 | 2.46 | −13.43 | 13.43 |
| | endoplasmic reticulum | | | | | |
| | ATPase/Name = VCP | | | | | |
| P12429 | Annexin A3/ | 1.09E−03 | −3.8 | 3.8 | −3.33 | 3.33 |
| | Name = ANXA3; | | | | | |
| | Synonyms = ANX3 | | | | | |
| O43143 | Putative | 1.09E−03 | −3.8 | 3.8 | −3.33 | 3.33 |
| | pre-mRNA-splicing | | | | | |
| | factor ATP-dependent | | | | | |
| | RNA helicase DHX15/ | | | | | |
| | Name = DHX15; | | | | | |
| | Synonyms = DBP1, | | | | | |
| | DDX15 | | | | | |
| P31513 | Dimethylaniline | 1.11E−03 | 2.78 | 2.78 | 17 | 17 |
| | monooxygenase | | | | | |
| | [N-oxide-forming] 3/ | | | | | |
| | Name = FMO3 | | | | | |
| P04075 | Fructose-bisphosphate | 1.11E−03 | −3.19 | 3.19 | −18.79 | 18.79 |
| | aldolase A/ | | | | | |
| | Name = ALDOA; | | | | | |
| | Synonyms = ALDA | | | | | |
| P42765 | 3-ketoacyl-CoA | 1.12E−03 | 2.71 | 2.71 | 44.1 | 44.1 |
| | thiolase, mitochondrial/ | | | | | |
| | Name = ACAA2 | | | | | |
| P09327 | Villin-1/Name = VIL1; | 1.12E−03 | −3.77 | 3.77 | −7.67 | 7.67 |
| | Synonyms = VIL | | | | | |
| Q15185 | Prostaglandin E | 1.14E−03 | −2.48 | 2.48 | −2.38 | 2.38 |
| | synthase 3/ | | | | | |
| | Name = PTGES3; | | | | | |
| | Synonyms = P23, TEBP | | | | | |
| P13716 | Delta-aminolevulinic | 1.16E−03 | 2.76 | 2.76 | 10.14 | 10.14 |
| | acid dehydratase/ | | | | | |
| | Name = ALAD | | | | | |
| Q13765 | Nascent | 1.21E−03 | −2.32 | 2.32 | −2.57 | 2.57 |
| | polypeptide-associated | | | | | |
| | complex subunit alpha/ | | | | | |
| | Name = NACA; | | | | | |
| | ORFNames = HSD48 | | | | | |
| Q96AB3 | Isochorismatase | 1.22E−03 | 2.44 | 2.44 | 5.07 | 5.07 |
| | domain-containing | | | | | |
| | protein 2, | | | | | |
| | mitochondrial/ | | | | | |
| | Name = ISOC2 | | | | | |
| P07437 | Tubulin beta chain/ | 1.23E−03 | −2.88 | 2.88 | −28.74 | 28.74 |
| | Name = TUBB; | | | | | |
| | Synonyms = TUBB5; | | | | | |
| | ORFNames = OK/SW-cl. | | | | | |
| | 56 | | | | | |
| Q92506 | Estradiol | 1.28E−03 | 2.7 | 2.7 | 3.14 | 3.14 |
| | 17-beta-dehydrogenase | | | | | |
| | 8/Name = HSD17B8; | | | | | |
| | Synonyms = FABGL, | | | | | |
| | HKE6, RING2 | | | | | |
| P13804 | Electron transfer | 1.35E−03 | 2.29 | 2.29 | 12.43 | 12.43 |
| | flavoprotein subunit | | | | | |
| | alpha, mitochondrial/ | | | | | |
| | Name = ETFA | | | | | |
| P08238 | Heat shock protein | 1.36E−03 | −2.17 | 2.17 | −12.52 | 12.52 |
| | HSP 90-beta/ | | | | | |
| | Name = HSP90AB1; | | | | | |
| | Synonyms = HSP90B, | | | | | |
| | HSPC2, HSPCB | | | | | |
| P27144 | Adenylate kinase | 1.37E−03 | 2.67 | 2.67 | 6 | 6 |
| | isoenzyme 4, | | | | | |
| | mitochondrial/ | | | | | |
| | Name = AK4; | | | | | |
| | Synonyms = AK3, | | | | | |
| | AK3L1 | | | | | |
| P42330 | Aldo-keto reductase | 1.38E−03 | −2.35 | 2.35 | −16.24 | 16.24 |
| | family 1 member C3/ | | | | | |
| | Name = AKR1C3; | | | | | |
| | Synonyms = DDH1, | | | | | |
| | HSD17B5, KIAA0119, | | | | | |
| | PGFS | | | | | |
| Q86YB7 | Enoyl-CoA hydratase | 1.39E−03 | 2.66 | 2.66 | 5.57 | 5.57 |
| | domain-containing | | | | | |
| | protein 2, | | | | | |
| | mitochondrial/ | | | | | |
| | Name = ECHDC2 | | | | | |
| Q00610 | Clathrin heavy chain 1/ | 1.40E−03 | −2.4 | 2.4 | −7.4 | 7.4 |
| | Name = CLTC; | | | | | |
| | Synonyms = CLH17, | | | | | |
| | CLTCL2, KIAA0034 | | | | | |
| P62807 | Histone H2B type | 1.43E−03 | −3.01 | 3.01 | −28.57 | 28.57 |
| | 1-C/E/F/G/I/ | | | | | |
| | Name = HIST1H2BC; | | | | | |
| | Synonyms = H2BFL | | | | | |
| P58876 | Histone H2B type 1-D/ | 1.43E−03 | −3.01 | 3.01 | −28.57 | 28.57 |
| | Name = HIST1H2BD; | | | | | |
| | Synonyms = H2BFB, | | | | | |
| | HIRIP2 | | | | | |
| Q93079 | Histone H2B type 1-H/ | 1.43E−03 | −3.01 | 3.01 | −28.57 | 28.57 |
| | Name = HIST1H2BH; | | | | | |
| | Synonyms = H2BFJ | | | | | |
| O60814 | Histone H2B type 1-K/ | 1.43E−03 | −3.01 | 3.01 | −28.57 | 28.57 |
| | Name = HIST1H2BK; | | | | | |
| | Synonyms = H2BFT, | | | | | |
| | HIRIP1 | | | | | |
| Q99880 | Histone H2B type 1-L/ | 1.43E−03 | −3.01 | 3.01 | −28.57 | 28.57 |
| | Name = HIST1H2BL; | | | | | |
| | Synonyms = H2BFC | | | | | |
| Q99879 | Histone H2B type 1-M/ | 1.43E−03 | −3.01 | 3.01 | −28.57 | 28.57 |
| | Name = HIST1H2BM; | | | | | |
| | Synonyms = H2BFE | | | | | |
| Q99877 | Histone H2B type 1-N/ | 1.43E−03 | −3.01 | 3.01 | −28.57 | 28.57 |
| | Name = HIST1H2BN; | | | | | |
| | Synonyms = H2BFD | | | | | |
| Q5QNW6 | Histone H2B type 2-F/ | 1.43E−03 | −3.01 | 3.01 | −28.57 | 28.57 |
| | Name = HIST2H2BF | | | | | |
| P57053 | Histone H2B type F—S/ | 1.43E−03 | −3.01 | 3.01 | −28.57 | 28.57 |
| | Name = H2BFS | | | | | |
| P68032 | Actin, alpha cardiac | 1.46E−03 | −2.43 | 2.43 | −35.74 | 35.74 |
| | muscle 1/ | | | | | |
| | Name = ACTC1; | | | | | |
| | Synonyms = ACTC | | | | | |
| P35573 | Glycogen debranching | 1.50E−03 | 2.62 | 2.62 | 15.14 | 15.14 |
| | enzyme/Name = AGL; | | | | | |
| | Synonyms = GDE | | | | | |
| Q96HR9 | Receptor | 1.57E−03 | 2.6 | 2.6 | 3.71 | 3.71 |
| | expression-enhancing | | | | | |
| | protein 6/ | | | | | |
| | Name = REEP6; | | | | | |
| | Synonyms = C19orf32, | | | | | |
| | DP1L1 | | | | | |
| P62753 | 40S ribosomal protein | 1.58E−03 | −2.15 | 2.15 | −2.81 | 2.81 |
| | S6/Name = RPS6; | | | | | |
| | ORFNames = OK/SW-cl.2 | | | | | |
| P00325 | Alcohol | 1.60E−03 | 2.45 | 2.45 | 53.24 | 53.24 |
| | dehydrogenase 1B/ | | | | | |
| | Name = ADH1B; | | | | | |
| | Synonyms = ADH2 | | | | | |
| Q08257 | Quinone | 1.63E−03 | 2.1 | 2.1 | 6.21 | 6.21 |
| | oxidoreductase/ | | | | | |
| | Name = CRYZ | | | | | |
| Q00796 | Sorbitol | 1.66E−03 | 2.48 | 2.48 | 16.05 | 16.05 |
| | dehydrogenase/ | | | | | |
| | Name = SORD | | | | | |
| P14618 | Pyruvate kinase | 1.66E−03 | −3.37 | 3.37 | −27.81 | 27.81 |
| | isozymes M1/M2/ | | | | | |
| | Name = PKM2; | | | | | |
| | Synonyms = OIP3, PK2, | | | | | |
| | PK3, PKM | | | | | |
| Q9BY49 | Peroxisomal | 1.66E−03 | 2.57 | 2.57 | 5 | 5 |
| | trans-2-enoyl-CoA | | | | | |
| | reductase/ | | | | | |
| | Name = PECR; | | | | | |
| | ORFNames = PRO1004 | | | | | |
| P16152 | Carbonyl reductase | 1.70E−03 | 3.2 | 3.2 | 8.24 | 8.24 |
| | [NADPH] 1/ | | | | | |
| | Name = CBR1; | | | | | |
| | Synonyms = CBR, CRN | | | | | |
| Q15274 | Nicotinate-nucleotide | 1.77E−03 | 2.54 | 2.54 | 6.57 | 6.57 |
| | pyrophosphorylase | | | | | |
| | [carboxylating]/ | | | | | |
| | Name = QPRT | | | | | |
| Q13011 | Delta(3,5)-Delta(2,4)-dienoyl- | 1.79E−03 | 2.08 | 2.08 | 8.33 | 8.33 |
| | CoA isomerase, | | | | | |
| | mitochondrial/ | | | | | |
| | Name = ECH1 | | | | | |
| P55083 | Microfibril-associated | 1.82E−03 | −3.39 | 3.39 | −9.67 | 9.67 |
| | glycoprotein 4/ | | | | | |
| | Name = MFAP4 | | | | | |
| Q96DG6 | Carboxymethylenebutenolidase | 1.86E−03 | 2.51 | 2.51 | 4.71 | 4.71 |
| | homolog/ | | | | | |
| | Name = CMBL | | | | | |
| Q14749 | Glycine | 1.88E−03 | 2.51 | 2.51 | 7 | 7 |
| | N-methyltransferase/ | | | | | |
| | Name = GNMT | | | | | |
| P29401 | Transketolase/ | 1.91E−03 | −2.66 | 2.66 | −13.76 | 13.76 |
| | Name = TKT | | | | | |
| Q1KMD3 | Heterogeneous | 1.92E−03 | −3.35 | 3.35 | −5.83 | 5.83 |
| | nuclear | | | | | |
| | ribonucleoprotein | | | | | |
| | U-like protein 2/ | | | | | |
| | Name = HNRNPUL2; | | | | | |
| | Synonyms = HNRPUL2 | | | | | |
| P11216 | Glycogen | 1.92E−03 | −3.35 | 3.35 | −9.67 | 9.67 |
| | phosphorylase, brain | | | | | |
| | form/Name = PYGB | | | | | |
| P35237 | Serpin B6/ | 1.95E−03 | −3.34 | 3.34 | −4.17 | 4.17 |
| | Name = SERPINB6; | | | | | |
| | Synonyms = PI6, PTI | | | | | |
| P13929 | Beta-enolase/ | 1.96E−03 | 2.49 | 2.49 | 10.57 | 10.57 |
| | Name = ENO3 | | | | | |
| P52272 | Heterogeneous | 2.06E−03 | −2.48 | 2.48 | −12.19 | 12.19 |
| | nuclear | | | | | |
| | ribonucleoprotein M/ | | | | | |
| | Name = HNRNPM; | | | | | |
| | Synonyms = HNRPM, | | | | | |
| | NAGR1 | | | | | |
| Q96CX2 | BTB/POZ | 2.06E−03 | −3.3 | 3.3 | −4 | 4 |
| | domain-containing | | | | | |
| | protein KCTD12/ | | | | | |
| | Name = KCTD12; | | | | | |
| | Synonyms = C13orf2, | | | | | |
| | KIAA1778, PFET1 | | | | | |
| P02649 | Apolipoprotein E/ | 2.07E−03 | 2.13 | 2.13 | 7.6 | 7.6 |
| | Name = APOE | | | | | |
| P45974 | Ubiquitin | 2.13E−03 | −3.27 | 3.27 | −3 | 3 |
| | carboxyl-terminal | | | | | |
| | hydrolase 5/ | | | | | |
| | Name = USP5; | | | | | |
| | Synonyms = ISOT | | | | | |
| P63104 | 14-3-3 protein | 2.26E−03 | −2.92 | 2.92 | −13.14 | 13.14 |
| | zeta/delta/ | | | | | |
| | Name = YWHAZ | | | | | |
| P55084 | Trifunctional enzyme | 2.33E−03 | 2.11 | 2.11 | 15.31 | 15.31 |
| | subunit beta, | | | | | |
| | mitochondrial/ | | | | | |
| | Name = HADHB; | | | | | |
| | ORFNames = MSTP029 | | | | | |
| P07900 | Heat shock protein | 2.33E−03 | −2.22 | 2.22 | −18.76 | 18.76 |
| | HSP 90-alpha/ | | | | | |
| | Name = HSP90AA1; | | | | | |
| | Synonyms = HSP90A, | | | | | |
| | HSPC1, HSPCA | | | | | |
| P30042 | ES1 protein homolog, | 2.33E−03 | 2.4 | 2.4 | 2.43 | 2.43 |
| | mitochondrial/ | | | | | |
| | Name = C21orf33; | | | | | |
| | Synonyms = HES1, | | | | | |
| | KNPI | | | | | |
| P08311 | Cathepsin G/ | 2.38E−03 | −2.23 | 2.23 | −4.62 | 4.62 |
| | Name = CTSG | | | | | |
| Q15149 | Plectin/Name = PLEC; | 2.45E−03 | −3.12 | 3.12 | −16.21 | 16.21 |
| | Synonyms = PLEC1 | | | | | |
| P07858 | Cathepsin B/ | 2.46E−03 | 2.13 | 2.13 | 8.14 | 8.14 |
| | Name = CTSB; | | | | | |
| | Synonyms = CPSB | | | | | |
| P54819 | Adenylate kinase 2, | 2.57E−03 | 2.03 | 2.03 | 5.81 | 5.81 |
| | mitochondrial/ | | | | | |
| | Name = AK2; | | | | | |
| | Synonyms = ADK2 | | | | | |
| Q14651 | Plastin-1/ | 2.58E−03 | −3.13 | 3.13 | −8 | 8 |
| | Name = PLS1 | | | | | |
| P01860 | Ig gamma-3 chain C | 2.61E−03 | −2.28 | 2.28 | −18.93 | 18.93 |
| | region/Name = IGHG3 | | | | | |
| P50454 | Serpin H1/ | 2.62E−03 | −3.12 | 3.12 | −6.67 | 6.67 |
| | Name = SERPINH1; | | | | | |
| | Synonyms = CBP1, | | | | | |
| | CBP2, HSP47, | | | | | |
| | SERPINH2; | | | | | |
| | ORFNames = PIG14 | | | | | |
| Q12905 | Interleukin | 2.69E−03 | −2.59 | 2.59 | −6.21 | 6.21 |
| | enhancer-binding | | | | | |
| | factor 2/Name = ILF2; | | | | | |
| | Synonyms = NF45; | | | | | |
| | ORFNames = PRO3063 | | | | | |
| Q14974 | Importin subunit beta-1/ | 2.78E−03 | −2.16 | 2.16 | −3.45 | 3.45 |
| | Name = KPNB1; | | | | | |
| | Synonyms = NTF97 | | | | | |
| Q9ULC5 | Long-chain-fatty-acid-- | 2.81E−03 | 2.32 | 2.32 | 4.71 | 4.71 |
| | CoA ligase 5/ | | | | | |
| | Name = ACSL5; | | | | | |
| | Synonyms = ACS5, | | | | | |
| | FACL5; | | | | | |
| | ORFNames = UNQ633/ | | | | | |
| | PRO1250 | | | | | |
| P05177 | Cytochrome P450 1A2/ | 2.81E−03 | 2.32 | 2.32 | 7.57 | 7.57 |
| | Name = CYP1A2 | | | | | |
| P00505 | Aspartate | 2.88E−03 | 2.23 | 2.23 | 14.43 | 14.43 |
| | aminotransferase, | | | | | |
| | mitochondrial/ | | | | | |
| | Name = GOT2 | | | | | |
| P25705 | ATP synthase subunit | 2.90E−03 | 2 | 2 | 13.52 | 13.52 |
| | alpha, mitochondrial/ | | | | | |
| | Name = ATP5A1; | | | | | |
| | Synonyms = ATP5A, | | | | | |
| | ATP5AL2, ATPM | | | | | |
| Q86XE5 | Probable | 2.93E−03 | 2.3 | 2.3 | 2.29 | 2.29 |
| | 4-hydroxy-2-oxoglutarate | | | | | |
| | aldolase, | | | | | |
| | mitochondrial/ | | | | | |
| | Name = HOGA1; | | | | | |
| | Synonyms = C10orf65, | | | | | |
| | DHDPSL | | | | | |
| Q15717 | ELAV-like protein 1/ | 2.95E−03 | −1.95 | 1.95 | −3.26 | 3.26 |
| | Name = ELAVL1; | | | | | |
| | Synonyms = HUR | | | | | |
| O60218 | Aldo-keto reductase | 2.98E−03 | −2.93 | 2.93 | −20.98 | 20.98 |
| | family 1 member B10/ | | | | | |
| | Name = AKR1B10; | | | | | |
| | Synonyms = AKR1B11 | | | | | |
| P31937 | 3-hydroxyisobutyrate | 2.99E−03 | 1.96 | 1.96 | 6.02 | 6.02 |
| | dehydrogenase, | | | | | |
| | mitochondrial/ | | | | | |
| | Name = HIBADH | | | | | |
| Q15493 | Regucalcin/ | 3.00E−03 | 2.28 | 2.28 | 5.43 | 5.43 |
| | Name = RGN; | | | | | |
| | Synonyms = SMP30 | | | | | |
| Q06278 | Aldehyde oxidase/ | 3.06E−03 | 2.27 | 2.27 | 27 | 27 |
| | Name = AOX1; | | | | | |
| | Synonyms = AO | | | | | |
| P31150 | Rab GDP dissociation | 3.07E−03 | −3.01 | 3.01 | −9.33 | 9.33 |
| | inhibitor alpha/ | | | | | |
| | Name = GDI1; | | | | | |
| | Synonyms = GDIL, | | | | | |
| | OPHN2, RABGDIA, | | | | | |
| | XAP4 | | | | | |
| P18206 | Vinculin/Name = VCL | 3.11E−03 | −2.28 | 2.28 | −7 | 7 |
| P12277 | Creatine kinase B-type/ | 3.11E−03 | −3 | 3 | −6 | 6 |
| | Name = CKB; | | | | | |
| | Synonyms = CKBB | | | | | |
| P11310 | Medium-chain specific | 3.19E−03 | 2.1 | 2.1 | 10.33 | 10.33 |
| | acyl-CoA | | | | | |
| | dehydrogenase, | | | | | |
| | mitochondrial/ | | | | | |
| | Name = ACADM | | | | | |
| P84243 | Histone H3.3/ | 3.20E−03 | −1.93 | 1.93 | −4.26 | 4.26 |
| | Name = H3F3A; | | | | | |
| | Synonyms = H3.3A, | | | | | |
| | H3F3; | | | | | |
| | ORFNames = PP781 | | | | | |
| P18283 | Glutathione peroxidase | 3.21E−03 | −2.98 | 2.98 | −4.67 | 4.67 |
| | 2/Name = GPX2 | | | | | |
| Q02318 | Sterol 26-hydroxylase, | 3.22E−03 | 2.25 | 2.25 | 4 | 4 |
| | mitochondrial/ | | | | | |
| | Name = CYP27A1; | | | | | |
| | Synonyms = CYP27 | | | | | |
| P40925 | Malate | 3.25E−03 | 1.9 | 1.9 | 3.33 | 3.33 |
| | dehydrogenase, | | | | | |
| | cytoplasmic/ | | | | | |
| | Name = MDH1; | | | | | |
| | Synonyms = MDHA | | | | | |
| P05787 | Keratin, type II | 3.35E−03 | −1.93 | 1.93 | −23.17 | 23.17 |
| | cytoskeletal 8/ | | | | | |
| | Name = KRT8; | | | | | |
| | Synonyms = CYK8 | | | | | |
| Q9UJM8 | Hydroxyacid oxidase 1/ | 3.39E−03 | 2.23 | 2.23 | 21.71 | 21.71 |
| | Name = HAO1; | | | | | |
| | Synonyms = GOX1, | | | | | |
| | HAOX1 | | | | | |
| P17655 | Calpain-2 catalytic | 3.47E−03 | −2.93 | 2.93 | −8.67 | 8.67 |
| | subunit/ | | | | | |
| | Name = CAPN2; | | | | | |
| | Synonyms = CANPL2 | | | | | |
| P98160 | Basement | 3.48E−03 | −2.5 | 2.5 | −12.55 | 12.55 |
| | membrane-specific | | | | | |
| | heparan sulfate | | | | | |
| | proteoglycan core | | | | | |
| | protein/ | | | | | |
| | Name = HSPG2 | | | | | |
| P05166 | Propionyl-CoA | 3.49E−03 | 2.21 | 2.21 | 7.71 | 7.71 |
| | carboxylase beta | | | | | |
| | chain, mitochondrial/ | | | | | |
| | Name = PCCB | | | | | |
| P01620 | Ig kappa chain V-III | 3.52E−03 | −2.28 | 2.28 | −2.71 | 2.71 |
| | region SIE/ | | | | | |
| P01623 | Ig kappa chain V-III | 3.52E−03 | −2.28 | 2.28 | −2.71 | 2.71 |
| | region WOL/ | | | | | |
| Q14764 | Major vault protein/ | 3.57E−03 | −2.91 | 2.91 | −8 | 8 |
| | Name = MVP; | | | | | |
| | Synonyms = LRP | | | | | |
| P40939 | Trifunctional enzyme | 3.79E−03 | 1.85 | 1.85 | 18.24 | 18.24 |
| | subunit alpha, | | | | | |
| | mitochondrial/ | | | | | |
| | Name = HADHA; | | | | | |
| | Synonyms = HADH | | | | | |
| Q86VP6 | Cullin-associated | 3.82E−03 | −2.86 | 2.86 | −4.5 | 4.5 |
| | NEDD8-dissociated | | | | | |
| | protein 1/ | | | | | |
| | Name = CAND1; | | | | | |
| | Synonyms = KIAA0829, | | | | | |
| | TIP120, TIP120A | | | | | |
| P27216 | Annexin A13/ | 3.90E−03 | −2.85 | 2.85 | −8.33 | 8.33 |
| | Name = ANXA13; | | | | | |
| | Synonyms = ANX13 | | | | | |
| P23786 | Carnitine | 3.93E−03 | 2.16 | 2.16 | 10.29 | 10.29 |
| | O-palmitoyltransferase | | | | | |
| | 2, mitochondrial/ | | | | | |
| | Name = CPT2; | | | | | |
| | Synonyms = CPT1 | | | | | |
| Q14103 | Heterogeneous | 4.09E−03 | −2.06 | 2.06 | −3.5 | 3.5 |
| | nuclear | | | | | |
| | ribonucleoprotein D0/ | | | | | |
| | Name = HNRNPD; | | | | | |
| | Synonyms = AUF1, | | | | | |
| | HNRPD | | | | | |
| P38117 | Electron transfer | 4.26E−03 | 1.98 | 1.98 | 11.6 | 11.6 |
| | flavoprotein subunit | | | | | |
| | beta/Name = ETFB; | | | | | |
| | ORFNames = FP585 | | | | | |
| P01876 | Ig alpha-1 chain C | 4.48E−03 | −2.01 | 2.01 | −6.79 | 6.79 |
| | region/Name = IGHA1 | | | | | |
| Q07507 | Dermatopontin/ | 4.49E−03 | −2.76 | 2.76 | −7.83 | 7.83 |
| | Name = DPT | | | | | |
| P01834 | Ig kappa chain C | 4.68E−03 | −2.31 | 2.31 | −14.74 | 14.74 |
| | region/Name = IGKC | | | | | |
| P13639 | Elongation factor 2/ | 4.71E−03 | −2.32 | 2.32 | −12.57 | 12.57 |
| | Name = EEF2; | | | | | |
| | Synonyms = EF2 | | | | | |
| P00738 | Haptoglobin/ | 4.71E−03 | 1.8 | 1.8 | 7.21 | 7.21 |
| | Name = HP | | | | | |
| P12532 | Creatine kinase | 4.72E−03 | −2.72 | 2.72 | −3.83 | 3.83 |
| | U-type, mitochondrial/ | | | | | |
| | Name = CKMT1A; | | | | | |
| | Synonyms = CKMT | | | | | |
| Q14247 | Src substrate cortactin/ | 4.72E−03 | −2.72 | 2.72 | −3.83 | 3.83 |
| | Name = CTTN; | | | | | |
| | Synonyms = EMS1 | | | | | |
| P01622 | Ig kappa chain V-III | 4.82E−03 | −2.07 | 2.07 | −2.21 | 2.21 |
| | region Ti/ | | | | | |
| P04206 | Ig kappa chain V-III | 4.82E−03 | −2.07 | 2.07 | −2.21 | 2.21 |
| | region GOL/ | | | | | |
| P28845 | Corticosteroid | 4.98E−03 | 2.06 | 2.06 | 4 | 4 |
| | 11-beta-dehydrogenase | | | | | |
| | isozyme 1/ | | | | | |
| | Name = HSD11B1; | | | | | |
| | Synonyms = HSD11, | | | | | |
| | HSD11L | | | | | |
| P14314 | Glucosidase 2 subunit | 5.09E−03 | 2.04 | 2.04 | 3.33 | 3.33 |
| | beta/ | | | | | |
| | Name = PRKCSH; | | | | | |
| | Synonyms = G19P1 | | | | | |
| P68363 | Tubulin alpha-1B chain/ | 5.15E−03 | −2.45 | 2.45 | −27.93 | 27.93 |
| | Name = TUBA1B | | | | | |
| P04792 | Heat shock protein | 5.24E−03 | −2.37 | 2.37 | −7.45 | 7.45 |
| | beta-1/ | | | | | |
| | Name = HSPB1; | | | | | |
| | Synonyms = HSP27, | | | | | |
| | HSP28 | | | | | |
| P78527 | DNA-dependent | 5.25E−03 | −2.29 | 2.29 | −8.62 | 8.62 |
| | protein kinase catalytic | | | | | |
| | subunit/ | | | | | |
| | Name = PRKDC; | | | | | |
| | Synonyms = HYRC, | | | | | |
| | HYRC1 | | | | | |
| P14866 | Heterogeneous | 5.43E−03 | −2.06 | 2.06 | −6.19 | 6.19 |
| | nuclear | | | | | |
| | ribonucleoprotein L/ | | | | | |
| | Name = HNRNPL; | | | | | |
| | Synonyms = HNRPL; | | | | | |
| | ORFNames = P/OKcl.14 | | | | | |
| P27169 | Serum | 5.56E−03 | 2.01 | 2.01 | 3.43 | 3.43 |
| | paraoxonase/arylesterase | | | | | |
| | 1/Name = PON1; | | | | | |
| | Synonyms = PON | | | | | |
| P17844 | Probable | 5.58E−03 | −2.62 | 2.62 | −7 | 7 |
| | ATP-dependent RNA | | | | | |
| | helicase DDX5/ | | | | | |
| | Name = DDX5; | | | | | |
| | Synonyms = G17P1, | | | | | |
| | HELR, HLR1 | | | | | |
| P07355 | Annexin A2/ | 5.72E−03 | −2.49 | 2.49 | −30.07 | 30.07 |
| | Name = ANXA2; | | | | | |
| | Synonyms = ANX2, | | | | | |
| | ANX2L4, CAL1H, | | | | | |
| | LPC2D | | | | | |
| P09382 | Galectin-1/ | 5.94E−03 | −2 | 2 | −3.24 | 3.24 |
| | Name = LGALS1 | | | | | |
| P07148 | Fatty acid-binding | 6.00E−03 | 1.94 | 1.94 | 37.48 | 37.48 |
| | protein, liver/ | | | | | |
| | Name = FABP1; | | | | | |
| | Synonyms = FABPL | | | | | |
| Q9H4A4 | Aminopeptidase B/ | 6.02E−03 | −2.57 | 2.57 | −3.67 | 3.67 |
| | Name = RNPEP; | | | | | |
| | Synonyms = APB | | | | | |
| P05165 | Propionyl-CoA | 6.04E−03 | 1.97 | 1.97 | 6 | 6 |
| | carboxylase alpha | | | | | |
| | chain, mitochondrial/ | | | | | |
| | Name = PCCA | | | | | |
| Q15233 | Non-POU | 6.08E−03 | −1.79 | 1.79 | −4.64 | 4.64 |
| | domain-containing | | | | | |
| | octamer-binding | | | | | |
| | protein/ | | | | | |
| | Name = NONO; | | | | | |
| | Synonyms = NRB54 | | | | | |
| Q9BXN1 | Asporin/ | 6.39E−03 | −2.54 | 2.54 | −7.83 | 7.83 |
| | Name = ASPN; | | | | | |
| | Synonyms = PLAP1, | | | | | |
| | SLRR1C; | | | | | |
| | ORFNames = UNQ215/ | | | | | |
| | PRO241 | | | | | |
| P22392 | Nucleoside | 6.41E−03 | −1.7 | 1.7 | −3.79 | 3.79 |
| | diphosphate kinase B/ | | | | | |
| | Name = NME2; | | | | | |
| | Synonyms = NM23B | | | | | |
| P21333 | Filamin-A/ | 6.47E−03 | −2.08 | 2.08 | −18.31 | 18.31 |
| | Name = FLNA; | | | | | |
| | Synonyms = FLN, FLN1 | | | | | |
| P12694 | 2-oxoisovalerate | 6.53E−03 | 1.94 | 1.94 | 5.14 | 5.14 |
| | dehydrogenase | | | | | |
| | subunit alpha, | | | | | |
| | mitochondrial/ | | | | | |
| | Name = BCKDHA | | | | | |
| Q86TX2 | Acyl-coenzyme A | 6.55E−03 | 1.94 | 1.94 | 5.86 | 5.86 |
| | thioesterase 1/ | | | | | |
| | Name = ACOT1; | | | | | |
| | Synonyms = CTE1 | | | | | |
| Q14011 | Cold-inducible | 6.57E−03 | −2.52 | 2.52 | −2 | 2 |
| | RNA-binding protein/ | | | | | |
| | Name = CIRBP; | | | | | |
| | Synonyms = A18HNRNP, | | | | | |
| | CIRP | | | | | |
| Q9NUI1 | Peroxisomal | 6.57E−03 | 1.94 | 1.94 | 4.43 | 4.43 |
| | 2,4-dienoyl-CoA | | | | | |
| | reductase/ | | | | | |
| | Name = DECR2; | | | | | |
| | Synonyms = PDCR | | | | | |
| P09429 | High mobility group | 6.59E−03 | −1.75 | 1.75 | −5.21 | 5.21 |
| | protein B1/ | | | | | |
| | Name = HMGB1; | | | | | |
| | Synonyms = HMG1 | | | | | |
| Q9HCC0 | Methylcrotonoyl-CoA | 6.63E−03 | 1.83 | 1.83 | 7.12 | 7.12 |
| | carboxylase beta | | | | | |
| | chain, mitochondrial/ | | | | | |
| | Name = MCCC2; | | | | | |
| | Synonyms = MCCB | | | | | |
| P42126 | Enoyl-CoA delta | 6.78E−03 | 1.68 | 1.68 | 3.69 | 3.69 |
| | isomerase 1, | | | | | |
| | mitochondrial/ | | | | | |
| | Name = ECI1; | | | | | |
| | Synonyms = DCI | | | | | |
| P05026 | Sodium/potassium-transporting | 6.78E−03 | −2.5 | 2.5 | −5.33 | 5.33 |
| | ATPase | | | | | |
| | subunit beta-1/ | | | | | |
| | Name = ATP1B1; | | | | | |
| | Synonyms = ATP1B | | | | | |
| Q8N4T8 | Carbonyl reductase | 6.78E−03 | 1.92 | 1.92 | 2.29 | 2.29 |
| | family member 4/ | | | | | |
| | Name = CBR4 | | | | | |
| Q9Y5M8 | Signal recognition | 7.00E−03 | 1.68 | 1.68 | 1.67 | 1.67 |
| | particle receptor | | | | | |
| | subunit beta/ | | | | | |
| | Name = SRPRB; | | | | | |
| | ORFNames = PSEC0230 | | | | | |
| Q96GK7 | Fumarylacetoacetate | 7.04E−03 | 1.91 | 1.91 | 3.86 | 3.86 |
| | hydrolase | | | | | |
| | domain-containing | | | | | |
| | protein 2A/ | | | | | |
| | Name = FAHD2A; | | | | | |
| | ORFNames = CGI-105 | | | | | |
| P63261 | Actin, cytoplasmic 2/ | 7.15E−03 | −2.2 | 2.2 | −70.62 | 70.62 |
| | Name = ACTG1; | | | | | |
| | Synonyms = ACTB, | | | | | |
| | ACTG | | | | | |
| P61160 | Actin-related protein 2/ | 7.35E−03 | −1.86 | 1.86 | −3.64 | 3.64 |
| | Name = ACTR2; | | | | | |
| | Synonyms = ARP2 | | | | | |
| P00390 | Glutathione reductase, | 7.37E−03 | −2.45 | 2.45 | −3.67 | 3.67 |
| | mitochondrial/ | | | | | |
| | Name = GSR; | | | | | |
| | Synonyms = GLUR, | | | | | |
| | GRD1 | | | | | |
| P07737 | Profilin-1/ | 7.41E−03 | −1.95 | 1.95 | −5.57 | 5.57 |
| | Name = PFN1 | | | | | |
| P22033 | Methylmalonyl-CoA | 7.42E−03 | 1.89 | 1.89 | 4.71 | 4.71 |
| | mutase, mitochondrial/ | | | | | |
| | Name = MUT | | | | | |
| P61158 | Actin-related protein 3/ | 7.44E−03 | −2.1 | 2.1 | −6.07 | 6.07 |
| | Name = ACTR3; | | | | | |
| | Synonyms = ARP3 | | | | | |
| P31153 | S-adenosylmethionine | 7.49E−03 | −2.44 | 2.44 | −2.83 | 2.83 |
| | synthase isoform | | | | | |
| | type-2/ | | | | | |
| | Name = MAT2A; | | | | | |
| | Synonyms = AMS2, | | | | | |
| | MATA2 | | | | | |
| Q12906 | Interleukin | 7.56E−03 | −2.05 | 2.05 | −5.64 | 5.64 |
| | enhancer-binding | | | | | |
| | factor 3/Name = ILF3; | | | | | |
| | Synonyms = DRBF, | | | | | |
| | MPHOSPH4, NF90 | | | | | |
| P10620 | Microsomal glutathione | 7.60E−03 | 1.65 | 1.65 | 3.9 | 3.9 |
| | S-transferase 1/ | | | | | |
| | Name = MGST1; | | | | | |
| | Synonyms = GST12, | | | | | |
| | MGST | | | | | |
| Q15436 | Protein transport | 7.63E−03 | 2.06 | 2.06 | 2.93 | 2.93 |
| | protein Sec23A/ | | | | | |
| | Name = SEC23A | | | | | |
| P12109 | Collagen alpha-1(VI) | 7.80E−03 | −2.03 | 2.03 | −10.07 | 10.07 |
| | chain/Name = COL6A1 | | | | | |
| P01008 | Antithrombin-III/ | 7.88E−03 | −1.93 | 1.93 | −4.95 | 4.95 |
| | Name = SERPINC1; | | | | | |
| | Synonyms = AT3; | | | | | |
| | ORFNames = PRO0309 | | | | | |
| P51991 | Heterogeneous | 7.92E−03 | −2.08 | 2.08 | −8.57 | 8.57 |
| | nuclear | | | | | |
| | ribonucleoprotein A3/ | | | | | |
| | Name = HNRNPA3; | | | | | |
| | Synonyms = HNRPA3 | | | | | |
| O75874 | Isocitrate | 7.94E−03 | 1.65 | 1.65 | 10.67 | 10.67 |
| | dehydrogenase | | | | | |
| | [NADP] cytoplasmic/ | | | | | |
| | Name = IDH1; | | | | | |
| | Synonyms = PICD | | | | | |
| P51857 | 3-oxo-5-beta-steroid | 8.04E−03 | 1.85 | 1.85 | 6.29 | 6.29 |
| | 4-dehydrogenase/ | | | | | |
| | Name = AKR1D1; | | | | | |
| | Synonyms = SRD5B1 | | | | | |
| Q16181 | Septin-7/ | 8.17E−03 | −2.39 | 2.39 | −4.33 | 4.33 |
| | Name = SEPT7; | | | | | |
| | Synonyms = CDC10 | | | | | |
| P51570 | Galactokinase/ | 8.24E−03 | 1.84 | 1.84 | 5 | 5 |
| | Name = GALK1; | | | | | |
| | Synonyms = GALK | | | | | |
| P49326 | Dimethylaniline | 8.38E−03 | 1.84 | 1.84 | 7.43 | 7.43 |
| | monooxygenase | | | | | |
| | [N-oxide-forming] 5/ | | | | | |
| | Name = FMO5 | | | | | |
| P14550 | Alcohol | 8.58E−03 | 1.65 | 1.65 | 4.38 | 4.38 |
| | dehydrogenase | | | | | |
| | [NADP+]/ | | | | | |
| | Name = AKR1A1; | | | | | |
| | Synonyms = ALDR1, | | | | | |
| | ALR | | | | | |
| P06703 | Protein S100-A6/ | 8.63E−03 | −2.36 | 2.36 | −2.33 | 2.33 |
| | Name = S100A6; | | | | | |
| | Synonyms = CACY | | | | | |
| P31949 | Protein S100-A11/ | 8.63E−03 | −2.36 | 2.36 | −2.33 | 2.33 |
| | Name = S100A11; | | | | | |
| | Synonyms = MLN70, | | | | | |
| | S100C | | | | | |
| P61163 | Alpha-centractin/ | 8.63E−03 | −2.36 | 2.36 | −2.33 | 2.33 |
| | Name = ACTR1A; | | | | | |
| | Synonyms = CTRN1 | | | | | |
| Q96I15 | Selenocysteine lyase/ | 8.83E−03 | 1.81 | 1.81 | 3 | 3 |
| | Name = SCLY; | | | | | |
| | Synonyms = SCL | | | | | |
| P07910 | Heterogeneous | 8.95E−03 | −1.94 | 1.94 | −5.24 | 5.24 |
| | nuclear | | | | | |
| | ribonucleoproteins | | | | | |
| | C1/C2/ | | | | | |
| | Name = HNRNPC; | | | | | |
| | Synonyms = HNRPC | | | | | |
| Q02878 | 60S ribosomal protein | 9.27E−03 | −1.6 | 1.6 | −3.07 | 3.07 |
| | L6/Name = RPL6; | | | | | |
| | Synonyms = TXREB1 | | | | | |
| P30043 | Flavin reductase | 9.29E−03 | 1.65 | 1.65 | 5.19 | 5.19 |
| | (NADPH)/ | | | | | |
| | Name = BLVRB; | | | | | |
| | Synonyms = FLR | | | | | |
| P01011 | Alpha-1-antichymotrypsin/ | 9.34E−03 | −1.84 | 1.84 | −4.98 | 4.98 |
| | Name = SERPINA3; | | | | | |
| | Synonyms = AACT; | | | | | |
| | ORFNames = GIG24, | | | | | |
| | GIG25 | | | | | |
| P68133 | Actin, alpha skeletal | 9.54E−03 | 1.78 | 1.78 | 25.43 | 25.43 |
| | muscle/ | | | | | |
| | Name = ACTA1; | | | | | |
| | Synonyms = ACTA | | | | | |
| O43809 | Cleavage and | 9.56E−03 | −1.91 | 1.91 | −2.38 | 2.38 |
| | polyadenylation | | | | | |
| | specificity factor | | | | | |
| | subunit 5/ | | | | | |
| | Name = NUDT21; | | | | | |
| | Synonyms = CFIM25, | | | | | |
| | CPSF25, CPSF5 | | | | | |
| Q7Z406 | Myosin-14/ | 9.57E−03 | −2.3 | 2.3 | −8.33 | 8.33 |
| | Name = MYH14; | | | | | |
| | Synonyms = KIAA2034; | | | | | |
| | ORFNames = FP17425 | | | | | |
| P23381 | Tryptophanyl-tRNA | 9.94E−03 | −2.27 | 2.27 | −2.5 | 2.5 |
| | synthetase, | | | | | |
| | cytoplasmic/ | | | | | |
| | Name = WARS; | | | | | |
| | Synonyms = IFI53, | | | | | |
| | WRS | | | | | |

*Bold type indicates increased relative expression in normal cholangiocytes compared to normal hepatocytes

Table 7 provides information as to whether the marker proteins are relatively over-expressed (identified in bold) or under-expressed in hepatocellular carcinoma versus cholangiocarcinoma in post-TACE liver tumours. Accordingly, by determining the presence, absence or change in expression levels of a plurality of these marker proteins and comparing these changes with a reference of known expression levels, one is able to determine whether the cells under test are hepatocellular carcinoma versus cholangiocarcinoma in post-TACE liver tumours.

All the marker proteins in section A of Table 7 are proteins with q-values less than or equal to 0.05. Those marker proteins in bold text (and with −ve effect size (g)) were less abundant in the HCC regions of the post-TACE, relative to the CC regions of the post-TACE. All the marker proteins in Table 7B are marker proteins with p-values less than or equal to 0.05.

TABLE 7
Proteins differentiating between hepatocellular carcinoma and
cholangiocarcinoma in post-TACE liver tumors*

| | | | | | Effect | Magnitude |
|---|---|---|---|---|---|---|
| | | | | | Size | of Mean |
| | | | Effect | Magnitude of | (Mean | dif (Mean |
| Protein | Name | P Value | Size (g) | Effect Size (g) | Dif) | Dif) |
| PART A: | | | | | | |
| P68032* | Actin, alpha cardiac | 5.30E−06 | −5 | 5 | −36.86 | 36.86 |
| | muscle 1/Name = ACTC1; | | | | | |
| | Synonyms = ACTC | | | | | |
| P00491* | Purine nucleoside | 1.19E−05 | −3.7 | 3.7 | −6.71 | 6.71 |
| | phosphorylase/ | | | | | |
| | Name = PNP; | | | | | |
| | Synonyms = NP | | | | | |
| Q9UHD8* | Septin-9/Name = SEPT9; | 4.03E−05 | −3.18 | 3.18 | −5.43 | 5.43 |
| | Synonyms = KIAA0991, | | | | | |
| | MSF | | | | | |
| P50440 | Glycine amidinotransferase, | 4.05E−05 | 3.16 | 3.16 | 12.43 | 12.43 |
| | mitochondrial/ | | | | | |
| | Name = GATM; | | | | | |
| | Synonyms = AGAT | | | | | |
| Q04917* | 14-3-3 protein eta/ | 4.45E−05 | −3.11 | 3.11 | −10.71 | 10.71 |
| | Name = YWHAH; | | | | | |
| | Synonyms = YWHA1 | | | | | |
| Q03154 | Aminoacylase-1/ | 5.54E−05 | 4.05 | 4.05 | 11.29 | 11.29 |
| | Name = ACY1 | | | | | |
| Q02338 | D−beta-hydroxybutyrate | 8.10E−05 | 4.72 | 4.72 | 5.71 | 5.71 |
| | dehydrogenase, | | | | | |
| | mitochondrial/ | | | | | |
| | Name = BDH1; | | | | | |
| | Synonyms = BDH | | | | | |
| Q14974* | Importin subunit beta-1/ | 1.01E−04 | −3.14 | 3.14 | −4.43 | 4.43 |
| | Name = KPNB1; | | | | | |
| | Synonyms = NTF97 | | | | | |
| Q9UBR2 | Cathepsin Z/Name = CTSZ | 1.85E−04 | 2.69 | 2.69 | 3.57 | 3.57 |
| P00167 | Cytochrome b5/ | 1.96E−04 | 2.99 | 2.99 | 5.86 | 5.86 |
| | Name = CYB5A; | | | | | |
| | Synonyms = CYB5 | | | | | |
| Q08426 | Peroxisomal bifunctional | 1.97E−04 | 3.56 | 3.56 | 28 | 28 |
| | enzyme/Name = EHHADH; | | | | | |
| | Synonyms = ECHD | | | | | |
| P07099 | Epoxide hydrolase 1/ | 2.05E−04 | 2.64 | 2.64 | 30.14 | 30.14 |
| | Name = EPHX1; | | | | | |
| | Synonyms = EPHX, EPOX | | | | | |
| P17174 | Aspartate aminotransferase, | 2.08E−04 | 2.9 | 2.9 | 18.71 | 18.71 |
| | cytoplasmic/Name = GOT1 | | | | | |
| O95831 | Apoptosis-inducing factor 1, | 2.36E−04 | 2.89 | 2.89 | 9.71 | 9.71 |
| | mitochondrial/ | | | | | |
| | Name = AIFM1; | | | | | |
| | Synonyms = AIF, PDCD8 | | | | | |
| P34896 | Serine | 2.50E−04 | 2.57 | 2.57 | 6 | 6 |
| | hydroxymethyltransferase, | | | | | |
| | cytosolic/Name = SHMT1 | | | | | |
| P00480 | Ornithine | 2.86E−04 | 3.76 | 3.76 | 14.29 | 14.29 |
| | carbamoyltransferase, | | | | | |
| | mitochondrial/Name = OTC | | | | | |
| P08133 | Annexin A6/Name = ANXA6; | 3.22E−04 | 2.64 | 2.64 | 14.71 | 14.71 |
| | Synonyms = ANX6 | | | | | |
| Q93099 | Homogentisate | 4.38E−04 | 3.22 | 3.22 | 12.14 | 12.14 |
| | 1,2-dioxygenase/ | | | | | |
| | Name = HGD; | | | | | |
| | Synonyms = HGO | | | | | |
| P16930 | Fumarylacetoacetase/ | 4.52E−04 | 2.54 | 2.54 | 6.29 | 6.29 |
| | Name = FAH | | | | | |
| P78417 | Glutathione S-transferase | 4.68E−04 | 2.52 | 2.52 | 4.43 | 4.43 |
| | omega-1/Name = GSTO1; | | | | | |
| | Synonyms = GSTTLP28 | | | | | |
| Q00839* | Heterogeneous nuclear | 5.13E−04 | −2.36 | 2.36 | −9.14 | 9.14 |
| | ribonucleoprotein U/ | | | | | |
| | Name = HNRNPU; | | | | | |
| | Synonyms = HNRNPU, SAFA, | | | | | |
| | U21.1 | | | | | |
| Q16762 | Thiosulfate sulfurtransferase/ | 5.46E−04 | 2.69 | 2.69 | 16 | 16 |
| | Name = TST | | | | | |
| P13010* | X-ray repair | 5.80E−04 | −2.36 | 2.36 | −6 | 6 |
| | cross-complementing | | | | | |
| | protein 5/Name = XRCC5; | | | | | |
| | Synonyms = G22P2 | | | | | |
| P07737* | Profilin-1/Name = PFN1 | 6.04E−04 | −2.52 | 2.52 | −6.14 | 6.14 |
| Q4G0N4 | NAD kinase | 6.39E−04 | 2.36 | 2.36 | 6.86 | 6.86 |
| | domain-containing protein 1/ | | | | | |
| | Name = NADKD1; | | | | | |
| | Synonyms = C5orf33 | | | | | |
| P04632* | Calpain small subunit 1/ | 6.49E−04 | −2.39 | 2.39 | −3.71 | 3.71 |
| | Name = CAPNS1; | | | | | |
| | Synonyms = CAPN4, | | | | | |
| | CAPNS | | | | | |
| P52907* | F-actin-capping protein | 6.74E−04 | −2.41 | 2.41 | −4.29 | 4.29 |
| | subunit alpha-1/ | | | | | |
| | Name = CAPZA1 | | | | | |
| P04040 | Catalase/Name = CAT | 7.26E−04 | 2.5 | 2.5 | 25.29 | 25.29 |
| P52565* | Rho GDP-dissociation | 7.34E−04 | −2.28 | 2.28 | −3.57 | 3.57 |
| | inhibitor 1/ | | | | | |
| | Name = ARHGDIA; | | | | | |
| | Synonyms = GDIA1 | | | | | |
| Q9Y2Q3 | Glutathione S-transferase | 8.67E−04 | 2.38 | 2.38 | 9 | 9 |
| | kappa 1/Name = GSTK1; | | | | | |
| | ORFNames = HDCMD47P | | | | | |
| O15144* | Actin-related protein 2/3 | 8.69E−04 | −2.59 | 2.59 | −5.57 | 5.57 |
| | complex subunit 2/ | | | | | |
| | Name = ARPC2; | | | | | |
| | Synonyms = ARC34; | | | | | |
| | ORFNames = PRO2446 | | | | | |
| Q9Y6C9 | Mitochondrial carrier | 8.73E−04 | 2.53 | 2.53 | 4.43 | 4.43 |
| | homolog 2/Name = MTCH2; | | | | | |
| | Synonyms = MIMP; | | | | | |
| | ORFNames = HSPC032 | | | | | |
| Q9UJM8 | Hydroxyacid oxidase 1/ | 8.77E−04 | 3.06 | 3.06 | 17.43 | 17.43 |
| | Name = HAO1; | | | | | |
| | Synonyms = GOX1, HAOX1 | | | | | |
| P63261* | Actin, cytoplasmic 2/ | 8.89E−04 | −2.74 | 2.74 | −93.86 | 93.86 |
| | Name = ACTG1; | | | | | |
| | Synonyms = ACTB, ACTG | | | | | |
| P23141 | Liver carboxylesterase 1/ | 9.00E−04 | 2.48 | 2.48 | 42.29 | 42.29 |
| | Name = CES1; | | | | | |
| | Synonyms = CES2, SES1 | | | | | |
| PART B: | | | | | | |
| P12956 | X-ray repair | 1.06E−03 | −2.41 | 2.41 | −6.86 | 6.86 |
| | cross-complementing protein | | | | | |
| | 6/Name = XRCC6; | | | | | |
| | Synonyms = G22P1 | | | | | |
| P09651 | Heterogeneous nuclear | 1.08E−03 | −2.23 | 2.23 | −8.43 | 8.43 |
| | ribonucleoprotein A1/ | | | | | |
| | Name = HNRNPA1; | | | | | |
| | Synonyms = HNRPA1 | | | | | |
| Q8NBX0 | Probable saccharopine | 1.09E−03 | 2.15 | 2.15 | 5.57 | 5.57 |
| | dehydrogenase/ | | | | | |
| | Name = SCCPDH; | | | | | |
| | ORFNames = CGI-49 | | | | | |
| P14618 | Pyruvate kinase isozymes | 1.14E−03 | −2.7 | 2.7 | −29 | 29 |
| | M1/M2/Name = PKM2; | | | | | |
| | Synonyms = OIP3, PK2, PK3, | | | | | |
| | PKM | | | | | |
| Q9H8H3 | Methyltransferase-like | 1.16E−03 | 2.9 | 2.9 | 4 | 4 |
| | protein 7A/ | | | | | |
| | Name = METTL7A; | | | | | |
| | ORFNames = PRO0066, | | | | | |
| | UNQ1902/PRO4348 | | | | | |
| P30086 | Phosphatidylethanolamine- | 1.27E−03 | 2.16 | 2.16 | 9.14 | 9.14 |
| | binding protein 1/ | | | | | |
| | Name = PEBP1; | | | | | |
| | Synonyms = PBP, PEBP | | | | | |
| P54868 | Hydroxymethylglutaryl-CoA | 1.35E−03 | 2.58 | 2.58 | 18.86 | 18.86 |
| | synthase, mitochondrial/ | | | | | |
| | Name = HMGCS2 | | | | | |
| P13804 | Electron transfer flavoprotein | 1.37E−03 | 2.4 | 2.4 | 9.43 | 9.43 |
| | subunit alpha, mitochondrial/ | | | | | |
| | Name = ETFA | | | | | |
| P38117 | Electron transfer flavoprotein | 1.39E−03 | 2.28 | 2.28 | 9.86 | 9.86 |
| | subunit beta/Name = ETFB; | | | | | |
| | ORFNames = FP585 | | | | | |
| P10620 | Microsomal glutathione | 1.43E−03 | 2.06 | 2.06 | 4.29 | 4.29 |
| | S-transferase 1/ | | | | | |
| | Name = MGST1; | | | | | |
| | Synonyms = GST12, MGST | | | | | |
| Q13228 | Selenium-binding protein 1/ | 1.44E−03 | 2.14 | 2.14 | 10.57 | 10.57 |
| | Name = SELENBP1; | | | | | |
| | Synonyms = SBP | | | | | |
| O75521 | Enoyl-CoA delta isomerase | 1.45E−03 | 2.13 | 2.13 | 4.29 | 4.29 |
| | 2, mitochondrial/ | | | | | |
| | Name = ECI2; | | | | | |
| | Synonyms = DRS1, HCA88, | | | | | |
| | PECI | | | | | |
| P30084 | Enoyl-CoA hydratase, | 1.49E−03 | 2.09 | 2.09 | 12 | 12 |
| | mitochondrial/ | | | | | |
| | Name = ECHS1 | | | | | |
| O00299 | Chloride intracellular channel | 1.50E−03 | −2.19 | 2.19 | −5.71 | 5.71 |
| | protein 1/Name = CLIC1; | | | | | |
| | Synonyms = G6, NCC27 | | | | | |
| O14756 | 17-beta-hydroxysteroid | 1.55E−03 | 2.48 | 2.48 | 9.57 | 9.57 |
| | dehydrogenase type 6/ | | | | | |
| | Name = HSD17B6; | | | | | |
| | Synonyms = RODH | | | | | |
| Q9P2E9 | Ribosome-binding protein 1/ | 1.59E−03 | 2.35 | 2.35 | 18 | 18 |
| | Name = RRBP1; | | | | | |
| | Synonyms = KIAA1398 | | | | | |
| P40121 | Macrophage-capping protein/ | 1.65E−03 | −2.06 | 2.06 | −6.71 | 6.71 |
| | Name = CAPG; | | | | | |
| | Synonyms = AFCP, MCP | | | | | |
| Q16698 | 2,4-dienoyl-CoA reductase, | 1.66E−03 | 2.24 | 2.24 | 10.57 | 10.57 |
| | mitochondrial/ | | | | | |
| | Name = DECR1; | | | | | |
| | Synonyms = DECR | | | | | |
| P21549 | Serine--pyruvate | 1.70E−03 | 2.36 | 2.36 | 38 | 38 |
| | aminotransferase/ | | | | | |
| | Name = AGXT; | | | | | |
| | Synonyms = AGT1, SPAT | | | | | |
| Q9NVS9 | Pyridoxine-5′-phosphate | 1.80E−03 | 2.05 | 2.05 | 2.57 | 2.57 |
| | oxidase/Name = PNPO | | | | | |
| P61978 | Heterogeneous nuclear | 1.93E−03 | −2.01 | 2.01 | −7.29 | 7.29 |
| | ribonucleoprotein K/ | | | | | |
| | Name = HNRNPK; | | | | | |
| | Synonyms = HNRPK | | | | | |
| P09525 | Annexin A4/Name = ANXA4; | 1.97E−03 | −2.26 | 2.26 | −20.43 | 20.43 |
| | Synonyms = ANX4 | | | | | |
| P02753 | Retinol-binding protein 4/ | 2.01E−03 | 2.6 | 2.6 | 3.71 | 3.71 |
| | Name = RBP4; | | | | | |
| | ORFNames = PRO2222 | | | | | |
| P32754 | 4-hydroxyphenylpyruvate | 2.09E−03 | 2.58 | 2.58 | 19.86 | 19.86 |
| | dioxygenase/Name = HPD; | | | | | |
| | Synonyms = PPD | | | | | |
| P00367 | Glutamate dehydrogenase 1, | 2.10E−03 | 2.05 | 2.05 | 22.29 | 22.29 |
| | mitochondrial/ | | | | | |
| | Name = GLUD1; | | | | | |
| | Synonyms = GLUD | | | | | |
| Q3LXA3 | Bifunctional ATP-dependent | 2.12E−03 | 2.26 | 2.26 | 12.71 | 12.71 |
| | dihydroxyacetone | | | | | |
| | kinase/FAD-AMP lyase | | | | | |
| | (cyclizing)/Name = DAK | | | | | |
| P61158 | Actin-related protein 3/ | 2.26E−03 | −2.29 | 2.29 | −8.71 | 8.71 |
| | Name = ACTR3; | | | | | |
| | Synonyms = ARP3 | | | | | |
| O43776 | Asparaginyl-tRNA | 2.27E−03 | −1.94 | 1.94 | −2.71 | 2.71 |
| | synthetase, cytoplasmic/ | | | | | |
| | Name = NARS | | | | | |
| P31947 | 14-3-3 protein sigma/ | 2.36E−03 | −2.15 | 2.15 | −10.29 | 10.29 |
| | Name = SFN; | | | | | |
| | Synonyms = HME1 | | | | | |
| P27348 | 14-3-3 protein theta/ | 2.45E−03 | −2.13 | 2.13 | −8.43 | 8.43 |
| | Name = YWHAQ | | | | | |
| O95479 | GDH/6PGL endoplasmic | 2.45E−03 | 2.01 | 2.01 | 4.57 | 4.57 |
| | bifunctional protein/ | | | | | |
| | Name = H6PD; | | | | | |
| | Synonyms = GDH | | | | | |
| P05062 | Fructose-bisphosphate | 2.49E−03 | 2.01 | 2.01 | 29.43 | 29.43 |
| | aldolase B/Name = ALDOB; | | | | | |
| | Synonyms = ALDB | | | | | |
| Q9H9B4 | Sideroflexin-1/ | 2.59E−03 | 1.9 | 1.9 | 3.29 | 3.29 |
| | Name = SFXN1 | | | | | |
| Q16836 | Hydroxyacyl-coenzyme A | 2.59E−03 | 2.25 | 2.25 | 4.43 | 4.43 |
| | dehydrogenase, | | | | | |
| | mitochondrial/ | | | | | |
| | Name = HADH; | | | | | |
| | Synonyms = HAD, HADHSC, | | | | | |
| | SCHAD | | | | | |
| P51858 | Hepatoma-derived growth | 2.60E−03 | −1.91 | 1.91 | −6.14 | 6.14 |
| | factor/Name = HDGF; | | | | | |
| | Synonyms = HMG1L2 | | | | | |
| O14745 | Na(+)/H(+) exchange | 2.63E−03 | −2.01 | 2.01 | −3.43 | 3.43 |
| | regulatory cofactor NHE-RF1/ | | | | | |
| | Name = SLC9A3R1; | | | | | |
| | Synonyms = NHERF, | | | | | |
| | NHERF1 | | | | | |
| P07437 | Tubulin beta chain/ | 2.66E−03 | −1.94 | 1.94 | −28.86 | 28.86 |
| | Name = TUBB; | | | | | |
| | Synonyms = TUBB5; | | | | | |
| | ORFNames = OK/SW-cl.56 | | | | | |
| Q9UJSO | Calcium-binding | 2.69E−03 | 1.89 | 1.89 | 9.14 | 9.14 |
| | mitochondrial carrier protein | | | | | |
| | Aralar2/Name = SLC25A13; | | | | | |
| | Synonyms = ARALAR2 | | | | | |
| P45954 | Short/branched chain | 2.71E−03 | 1.92 | 1.92 | 6.29 | 6.29 |
| | specific acyl-CoA | | | | | |
| | dehydrogenase, | | | | | |
| | mitochondrial/ | | | | | |
| | Name = ACADSB | | | | | |
| P49419 | Alpha-aminoadipic | 2.75E−03 | 1.88 | 1.88 | 7 | 7 |
| | semialdehyde | | | | | |
| | dehydrogenase/ | | | | | |
| | Name = ALDH7A1; | | | | | |
| | Synonyms = ATQ1 | | | | | |
| O00264 | Membrane-associated | 2.80E−03 | 2.07 | 2.07 | 7 | 7 |
| | progesterone receptor | | | | | |
| | component 1/ | | | | | |
| | Name = PGRMC1; | | | | | |
| | Synonyms = HPR6.6, | | | | | |
| | PGRMC | | | | | |
| P05089 | Arginase-1/Name = ARG1 | 2.82E−03 | 2.43 | 2.43 | 18.43 | 18.43 |
| P28288 | ATP-binding cassette | 2.82E−03 | 2.43 | 2.43 | 3 | 3 |
| | sub-family D member 3/ | | | | | |
| | Name = ABCD3; | | | | | |
| | Synonyms = PMP70, PXMP1 | | | | | |
| P04424 | Argininosuccinate lyase/ | 2.84E−03 | 2.01 | 2.01 | 14.14 | 14.14 |
| | Name = ASL | | | | | |
| P22307 | Non-specific lipid-transfer | 2.91E−03 | 2.21 | 2.21 | 6.29 | 6.29 |
| | protein/Name = SCP2 | | | | | |
| P60660 | Myosin light polypeptide 6/ | 3.20E−03 | −1.84 | 1.84 | −4 | 4 |
| | Name = MYL6 | | | | | |
| P07954 | Fumarate hydratase, | 3.20E−03 | 2.12 | 2.12 | 6.57 | 6.57 |
| | mitochondrial/Name = FH | | | | | |
| P09417 | Dihydropteridine reductase/ | 3.32E−03 | 2.35 | 2.35 | 4.57 | 4.57 |
| | Name = QDPR; | | | | | |
| | Synonyms = DHPR | | | | | |
| Q02252 | Methylmalonate-semialdehyde | 3.40E−03 | 2.29 | 2.29 | 15.43 | 15.43 |
| | dehydrogenase | | | | | |
| | [acylating], mitochondrial/ | | | | | |
| | Name = ALDH6A1; | | | | | |
| | Synonyms = MMSDH | | | | | |
| P51659 | Peroxisomal multifunctional | 3.85E−03 | 2.15 | 2.15 | 31.86 | 31.86 |
| | enzyme type 2 / | | | | | |
| | Name = HSD17B4; | | | | | |
| | Synonyms = EDH17B4 | | | | | |
| O00571 | ATP-dependent RNA | 3.93E−03 | −1.88 | 1.88 | −3.86 | 3.86 |
| | helicase DDX3X/ | | | | | |
| | Name = DDX3X; | | | | | |
| | Synonyms = DBX, DDX3 | | | | | |
| Q9BPW8 | Protein NipSnap homolog 1/ | 3.94E−03 | 1.96 | 1.96 | 5.57 | 5.57 |
| | Name = NIPSNAP1 | | | | | |
| P06703 | Protein S100-A6/ | 3.94E−03 | −1.95 | 1.95 | −2.57 | 2.57 |
| | Name = S100A6; | | | | | |
| | Synonyms = CACY | | | | | |
| P00403 | Cytochrome c oxidase | 4.03E−03 | 1.78 | 1.78 | 3 | 3 |
| | subunit 2/Name = MT-CO2; | | | | | |
| | Synonyms = COII, COXII, | | | | | |
| | MTCO2 | | | | | |
| P22760 | Arylacetamide deacetylase/ | 4.06E−03 | 2.26 | 2.26 | 5.86 | 5.86 |
| | Name = AADAC; | | | | | |
| | Synonyms = DAC | | | | | |
| P51991 | Heterogeneous nuclear | 4.11E−03 | −1.81 | 1.81 | −5.14 | 5.14 |
| | ribonucleoprotein A3/ | | | | | |
| | Name = HNRNPA3; | | | | | |
| | Synonyms = HNRPA3 | | | | | |
| P55084 | Trifunctional enzyme subunit | 4.13E−03 | 1.77 | 1.77 | 7.43 | 7.43 |
| | beta, mitochondrial/ | | | | | |
| | Name = HADHB; | | | | | |
| | ORFNames = MSTP029 | | | | | |
| Q12905 | Interleukin enhancer-binding | 4.15E−03 | −1.93 | 1.93 | −5.43 | 5.43 |
| | factor 2/Name = ILF2; | | | | | |
| | Synonyms = NF45; | | | | | |
| | ORFNames = PRO3063 | | | | | |
| Q68CK6 | Acyl-coenzyme A synthetase | 4.16E−03 | 2.25 | 2.25 | 14.71 | 14.71 |
| | ACSM2B, mitochondrial/ | | | | | |
| | Name = ACSM2B; | | | | | |
| | Synonyms = ACSM2; | | | | | |
| | ORFNames = HYST1046 | | | | | |
| P27338 | Amine oxidase | 4.32E−03 | 1.79 | 1.79 | 5.43 | 5.43 |
| | [flavin-containing] B/ | | | | | |
| | Name = MAOB | | | | | |
| P47756 | F-actin-capping protein | 4.35E−03 | −1.85 | 1.85 | −2.71 | 2.71 |
| | subunit beta/Name = CAPZB | | | | | |
| P68371 | Tubulin beta-2C chain/ | 4.44E−03 | −1.75 | 1.75 | −26.71 | 26.71 |
| | Name = TUBB2C | | | | | |
| P55157 | Microsomal triglyceride | 4.60E−03 | 2.2 | 2.2 | 7.71 | 7.71 |
| | transfer protein large subunit/ | | | | | |
| | Name = MTTP; | | | | | |
| | Synonyms = MTP | | | | | |
| P16435 | NADPH--cytochrome P450 | 4.65E−03 | 1.98 | 1.98 | 9.86 | 9.86 |
| | reductase/Name = POR; | | | | | |
| | Synonyms = CYPOR | | | | | |
| P07148 | Fatty acid-binding protein, | 4.78E−03 | 1.92 | 1.92 | 16.43 | 16.43 |
| | liver/Name = FABP1; | | | | | |
| | Synonyms = FABPL | | | | | |
| P30153 | Serine/threonine-protein | 4.81E−03 | −1.73 | 1.73 | −3 | 3 |
| | phosphatase 2A 65 kDa | | | | | |
| | regulatory subunit A alpha | | | | | |
| | isoform/Name = PPP2R1A | | | | | |
| Q16822 | Phosphoenolpyruvate | 4.83E−03 | 1.97 | 1.97 | 16.57 | 16.57 |
| | carboxykinase [GTP], | | | | | |
| | mitochondrial/ | | | | | |
| | Name = PCK2; | | | | | |
| | Synonyms = PEPCK2 | | | | | |
| O43707 | Alpha-actinin-4/ | 4.86E−03 | −1.72 | 1.72 | −15 | 15 |
| | Name = ACTN4 | | | | | |
| Q13838 | Spliceosome RNA helicase | 5.28E−03 | −1.74 | 1.74 | −5.29 | 5.29 |
| | DDX39B/Name = DDX39B; | | | | | |
| | Synonyms = BAT1, UAP56 | | | | | |
| O95154 | Aflatoxin B1 aldehyde | 5.39E−03 | 1.88 | 1.88 | 5.43 | 5.43 |
| | reductase member 3/ | | | | | |
| | Name = AKR7A3; | | | | | |
| | Synonyms = AFAR2 | | | | | |
| P24752 | Acetyl-CoA | 5.49E−03 | 1.93 | 1.93 | 14.86 | 14.86 |
| | acetyltransferase, | | | | | |
| | mitochondrial/ | | | | | |
| | Name = ACAT1; | | | | | |
| | Synonyms = ACAT, MAT | | | | | |
| P11498 | Pyruvate carboxylase, | 5.55E−03 | 2 | 2 | 16.29 | 16.29 |
| | mitochondrial/Name = PC | | | | | |
| Q9UI17 | Dimethylglycine | 5.56E−03 | 2.11 | 2.11 | 3.43 | 3.43 |
| | dehydrogenase, | | | | | |
| | mitochondrial/ | | | | | |
| | Name = DMGDH | | | | | |
| P11216 | Glycogen phosphorylase, | 5.62E−03 | −1.68 | 1.68 | −4.86 | 4.86 |
| | brain form/Name = PYGB | | | | | |
| P50995 | Annexin A11/ | 5.63E−03 | −1.73 | 1.73 | −3.71 | 3.71 |
| | Name = ANXA11; | | | | | |
| | Synonyms = ANX11 | | | | | |
| Q9NUI1 | Peroxisomal | 5.67E−03 | 1.73 | 1.73 | 3.57 | 3.57 |
| | 2,4-dienoyl-CoA reductase/ | | | | | |
| | Name = DECR2; | | | | | |
| | Synonyms = PDCR | | | | | |
| Q99613 | Eukaryotic translation | 5.80E−03 | −1.68 | 1.68 | −2.71 | 2.71 |
| | initiation factor 3 subunit C/ | | | | | |
| | Name = EIF3C; | | | | | |
| | Synonyms = EIF3S8 | | | | | |
| Q15365 | Poly(rC)-binding protein 1/ | 6.00E−03 | −1.74 | 1.74 | −5.29 | 5.29 |
| | Name = PCBP1 | | | | | |
| Q00796 | Sorbitol dehydrogenase/ | 6.09E−03 | 1.83 | 1.83 | 5.29 | 5.29 |
| | Name = SORD | | | | | |
| P18754 | Regulator of chromosome | 6.15E−03 | −1.79 | 1.79 | −2.29 | 2.29 |
| | condensation/ | | | | | |
| | Name = RCC1; | | | | | |
| | Synonyms = CHC1 | | | | | |
| Q86VP6 | Cullin-associated | 6.15E−03 | −1.94 | 1.94 | −4 | 4 |
| | NEDD8-dissociated protein 1/ | | | | | |
| | Name = CAND1; | | | | | |
| | Synonyms = KIAA0829, | | | | | |
| | TIP120, TIP120A | | | | | |
| P37802 | Transgelin-2/ | 6.24E−03 | −1.79 | 1.79 | −8.86 | 8.86 |
| | Name = TAGLN2; | | | | | |
| | Synonyms = KIAA0120; | | | | | |
| | ORFNames = CDABP0035 | | | | | |
| P04075 | Fructose-bisphosphate | 6.27E−03 | −1.95 | 1.95 | −23.57 | 23.57 |
| | aldolase A/Name = ALDOA; | | | | | |
| | Synonyms = ALDA | | | | | |
| P13667 | Protein disulfide-isomerase | 6.30E−03 | 1.73 | 1.73 | 13 | 13 |
| | A4/Name = PDIA4; | | | | | |
| | Synonyms = ERP70, ERP72 | | | | | |
| P26599 | Polypyrimidine tract-binding | 6.69E−03 | −1.66 | 1.66 | −5.57 | 5.57 |
| | protein 1/Name = PTBP1; | | | | | |
| | Synonyms = PTB | | | | | |
| P09467 | Fructose-1,6-bisphosphatase 1/ | 6.84E−03 | 1.86 | 1.86 | 9.71 | 9.71 |
| | Name = FBP1; | | | | | |
| | Synonyms = FBP | | | | | |
| P68363 | Tubulin alpha-1B chain/ | 6.88E−03 | −1.67 | 1.67 | −17.71 | 17.71 |
| | Name = TUBA1B | | | | | |
| P05455 | Lupus La protein/ | 6.91E−03 | −1.71 | 1.71 | −4 | 4 |
| | Name = SSB | | | | | |
| P09382 | Galectin-1/Name = LGALS1 | 7.15E−03 | −1.62 | 1.62 | −3.29 | 3.29 |
| P10619 | Lysosomal protective protein/ | 7.30E−03 | 1.62 | 1.62 | 2.57 | 2.57 |
| | Name = CTSA; | | | | | |
| | Synonyms = PPGB | | | | | |
| Q9Y265 | RuvB-like 1/ | 7.46E−03 | −1.98 | 1.98 | −2.57 | 2.57 |
| | Name = RUVBL1; | | | | | |
| | Synonyms = INO80H, | | | | | |
| | NMP238, TIP49, TIP49A | | | | | |
| P31939 | Bifunctional purine | 7.52E−03 | −1.9 | 1.9 | −10 | 10 |
| | biosynthesis protein PURH/ | | | | | |
| | Name = ATIC; | | | | | |
| | Synonyms = PURH; | | | | | |
| | ORFNames = OK/SW-cl.86 | | | | | |
| P52272 | Heterogeneous nuclear | 7.52E−03 | −1.63 | 1.63 | −7.57 | 7.57 |
| | ribonucleoprotein M/ | | | | | |
| | Name = HNRNPM; | | | | | |
| | Synonyms = HNRPM, | | | | | |
| | NAGR1 | | | | | |
| P00441 | Superoxide dismutase | 7.76E−03 | 1.63 | 1.63 | 2.86 | 2.86 |
| | [Cu—Zn]/Name = SOD1 | | | | | |
| Q9NTK5 | Obg-like ATPase 1/ | 7.85E−03 | −1.64 | 1.64 | −1.71 | 1.71 |
| | Name = OLA1; | | | | | |
| | Synonyms = GTPBP9; | | | | | |
| | ORFNames = PTD004, | | | | | |
| | PRO2455 | | | | | |
| P13797 | Plastin-3/Name = PLS3 | 7.99E−03 | −1.62 | 1.62 | −5.71 | 5.71 |
| P30038 | Delta-1-pyrroline-5-carboxylate | 8.31E−03 | 1.88 | 1.88 | 13.43 | 13.43 |
| | dehydrogenase, | | | | | |
| | mitochondrial/ | | | | | |
| | Name = ALDH4A1; | | | | | |
| | Synonyms = ALDH4, P5CDH | | | | | |
| Q01518 | Adenylyl cyclase-associated | 8.41E−03 | −1.72 | 1.72 | −7 | 7 |
| | protein 1/Name = CAP1; | | | | | |
| | Synonyms = CAP | | | | | |
| P11586 | C-1-tetrahydrofolate | 8.47E−03 | 1.87 | 1.87 | 9.43 | 9.43 |
| | synthase, cytoplasmic/ | | | | | |
| | Name = MTHFD1; | | | | | |
| | Synonyms = MTHFC, MTHFD | | | | | |
| P22314 | Ubiquitin-like | 8.69E−03 | −1.63 | 1.63 | −8.14 | 8.14 |
| | modifier-activating enzyme 1/ | | | | | |
| | Name = UBA1; | | | | | |
| | Synonyms = A1S9T, UBE1 | | | | | |
| Q07954 | Prolow-density lipoprotein | 8.71E−03 | 1.68 | 1.68 | 2.14 | 2.14 |
| | receptor-related protein 1/ | | | | | |
| | Name = LRP1; | | | | | |
| | Synonyms = A2MR, APR | | | | | |
| P52758 | Ribonuclease UK114/ | 8.71E−03 | 1.72 | 1.72 | 4.14 | 4.14 |
| | Name = HRSP12; | | | | | |
| | Synonyms = PSP | | | | | |
| Q9P0Z9 | Peroxisomal sarcosine | 8.79E−03 | 1.73 | 1.73 | 7.71 | 7.71 |
| | oxidase/Name = PIPOX; | | | | | |
| | Synonyms = LPIPOX, PSO | | | | | |
| P31930 | Cytochrome b-c1 complex | 8.86E−03 | 1.7 | 1.7 | 6.57 | 6.57 |
| | subunit 1, mitochondrial/ | | | | | |
| | Name = UQCRC1 | | | | | |
| P31513 | Dimethylaniline | 8.91E−03 | 1.68 | 1.68 | 6 | 6 |
| | monooxygenase | | | | | |
| | [N-oxide-forming] 3/ | | | | | |
| | Name = FMO3 | | | | | |
| Q15233 | Non-POU domain-containing | 9.27E−03 | −1.55 | 1.55 | −4.14 | 4.14 |
| | octamer-binding protein/ | | | | | |
| | Name = NONO; | | | | | |
| | Synonyms = NRB54 | | | | | |
| P07858 | Cathepsin B/Name = CTSB; | 9.46E−03 | 1.55 | 1.55 | 4.71 | 4.71 |
| | Synonyms = CPSB | | | | | |
| P23528 | Cofilin-1/Name = CFL1; | 9.69E−03 | −1.66 | 1.66 | −4.14 | 4.14 |
| | Synonyms = CFL | | | | | |
| O00515 | Ladinin-1/Name = LAD1; | 9.73E−03 | −1.87 | 1.87 | −4.29 | 4.29 |
| | Synonyms = LAD | | | | | |

*Bold type indicates increased relative expression in cholangiocarcinoma compared to hepatocellular carcinoma post-TACE

Table 8 provides information as to whether the marker proteins are relatively over-expressed (identified in bold) or under-expressed in peripheral cholangiocarcinoma versus metastatic colorectal cancer in post-TACE liver tumours. Accordingly, by determining the presence, absence or change in expression levels of a plurality of these marker proteins and comparing these changes with a reference of known expression levels, one is able to determine whether the cells under test are peripheral cholangiocarcinoma or metastatic colorectal cancer.

TABLE 8
Proteins differentiating peripheral cholangiocarcinoma from metastatic
colorectal cancer*

| | | | | | Effect | |
|---|---|---|---|---|---|---|
| | | | | Magnitude of | Size | Magnitude of |
| | | | Effect | Effect Size | (Mean | Mean dif |
| Protein | Name | P Value | Size (g) | (g) | Dif) | (Mean Dif) |
| PART A: | | | | | | |
| P17987* | T-complex protein 1 | 9.41E−05 | −2.98 | 2.98 | −5.29 | 5.29 |
| | subunit alpha/ | | | | | |
| | Name = TCP1; | | | | | |
| | Synonyms = CCT1, CCTA | | | | | |
| Q8NFW8* | N-acylneuraminate | 1.05E−04 | −2.91 | 2.91 | −2.71 | 2.71 |
| | cytidylyltransferase/ | | | | | |
| | Name = CMAS | | | | | |
| P15374* | Ubiquitin | 2.53E−04 | −2.61 | 2.61 | −3.57 | 3.57 |
| | carboxyl-terminal | | | | | |
| | hydrolase isozyme L3/ | | | | | |
| | Name = UCHL3 | | | | | |
| P09525 | Annexin A4/ | 2.77E−04 | 2.92 | 2.92 | 25.71 | 25.71 |
| | Name = ANXA4; | | | | | |
| | Synonyms = ANX4 | | | | | |
| Q99829* | Copine-1/Name = CPNE1; | 6.96E−04 | −2.32 | 2.32 | −3.29 | 3.29 |
| | Synonyms = CPN1 | | | | | |
| P08758 | Annexin A5/ | 8.30E−04 | 2.69 | 2.69 | 14.71 | 14.71 |
| | Name = ANXA5; | | | | | |
| | Synonyms = ANX5, ENX2, | | | | | |
| | PP4 | | | | | |
| P48643* | T-complex protein 1 | 9.45E−04 | −2.27 | 2.27 | −3.86 | 3.86 |
| | subunit epsilon/ | | | | | |
| | Name = CCT5; | | | | | |
| | Synonyms = CCTE, | | | | | |
| | KIAA0098 | | | | | |
| PART B: | | | | | | |
| P50990 | T-complex protein 1 subunit | 1.52E−03 | −2.25 | 2.25 | −6.43 | 6.43 |
| | theta/Name = CCT8; | | | | | |
| | Synonyms = C21orf112, | | | | | |
| | CCTQ, KIAA0002 | | | | | |
| P23526 | Adenosylhomocysteinase/ | 1.55E−03 | −2.07 | 2.07 | −5.43 | 5.43 |
| | Name = AHCY; | | | | | |
| | Synonyms = SAHH | | | | | |
| Q14444 | Caprin-1/ | 1.55E−03 | −2.21 | 2.21 | −1.86 | 1.86 |
| | Name = CAPRIN1; | | | | | |
| | Synonyms = GPIAP1, | | | | | |
| | GPIP137, M11S1, RNG105 | | | | | |
| Q8NE71 | ATP-binding cassette | 1.57E−03 | −2.73 | 2.73 | −1.86 | 1.86 |
| | sub-family F member 1/ | | | | | |
| | Name = ABCF1; | | | | | |
| | Synonyms = ABC50 | | | | | |
| O60547 | GDP-mannose 4,6 | 1.61E−03 | −2.72 | 2.72 | −2.29 | 2.29 |
| | dehydratase/ | | | | | |
| | Name = GMDS | | | | | |
| P56470 | Galectin-4/Name = LGALS4 | 1.65E−03 | −2.17 | 2.17 | −8 | 8 |
| P09429 | High mobility group protein | 1.72E−03 | −2.04 | 2.04 | −7 | 7 |
| | B1/Name = HMGB1; | | | | | |
| | Synonyms = HMG1 | | | | | |
| P50991 | T-complex protein 1 subunit | 1.76E−03 | −2.01 | 2.01 | −4.57 | 4.57 |
| | delta/Name = CCT4; | | | | | |
| | Synonyms = CCTD, SRB | | | | | |
| P12532 | Creatine kinase U-type, | 1.87E−03 | −2.64 | 2.64 | −3.43 | 3.43 |
| | mitochondrial/ | | | | | |
| | Name = CKMT1A; | | | | | |
| | Synonyms = CKMT | | | | | |
| P06731 | Carcinoembryonic | 1.98E−03 | −2.35 | 2.35 | −8.43 | 8.43 |
| | antigen-related cell | | | | | |
| | adhesion molecule 5/ | | | | | |
| | Name = CEACAM5; | | | | | |
| | Synonyms = CEA | | | | | |
| P26038 | Moesin/Name = MSN | 2.09E−03 | 2.11 | 2.11 | 10.14 | 10.14 |
| O95994 | Anterior gradient protein 2 | 2.12E−03 | −1.96 | 1.96 | −5.43 | 5.43 |
| | homolog/Name = AGR2; | | | | | |
| | Synonyms = AG2; | | | | | |
| | ORFNames = UNQ515/PRO | | | | | |
| | 1030 | | | | | |
| Q9NR45 | Sialic acid synthase/ | 2.37E−03 | −1.94 | 1.94 | −2.29 | 2.29 |
| | Name = NANS; | | | | | |
| | Synonyms = SAS | | | | | |
| Q9H0W9 | Ester hydrolase C11orf54/ | 2.93E−03 | 2.41 | 2.41 | 2.29 | 2.29 |
| | Name = C11orf54; | | | | | |
| | ORFNames = LP4947, | | | | | |
| | PTD012 | | | | | |
| Q92598 | Heat shock protein 105 kDa/ | 2.98E−03 | −2.35 | 2.35 | −9.14 | 9.14 |
| | Name = HSPH1; | | | | | |
| | Synonyms = HSP105, | | | | | |
| | HSP110, KIAA0201 | | | | | |
| Q9UKM9 | RNA-binding protein Raly/ | 3.02E−03 | −1.86 | 1.86 | −4.14 | 4.14 |
| | Name = RALY; | | | | | |
| | Synonyms = HNRPCL2, | | | | | |
| | P542 | | | | | |
| P08729 | Keratin, type II cytoskeletal | 3.10E−03 | 2.39 | 2.39 | 12 | 12 |
| | 7/Name = KRT7; | | | | | |
| | Synonyms = SCL | | | | | |
| Q14498 | RNA-binding protein 39/ | 3.30E−03 | −1.88 | 1.88 | −1.86 | 1.86 |
| | Name = RBM39; | | | | | |
| | Synonyms = HCC1, RNPC2 | | | | | |
| O76021 | Ribosomal L1 | 3.30−E03 | −1.88 | 1.88 | −1.86 | 1.86 |
| | domain-containing protein 1/ | | | | | |
| | Name = RSL1D1; | | | | | |
| | Synonyms = CATX11, CSIG, | | | | | |
| | PBK1; ORFNames = L12 | | | | | |
| Q08J23 | tRNA | 3.30E−03 | −1.88 | 1.88 | −1.86 | 1.86 |
| | (cytosine(34)-C(5))- | | | | | |
| | methyltransferase/ | | | | | |
| | Name = NSUN2; | | | | | |
| | Synonyms = SAKI, TRM4 | | | | | |
| Q06210 | Glucosamine--fructose-6-p | 3.42E−03 | −2.02 | 2.02 | −8.29 | 8.29 |
| | hosphate aminotransferase | | | | | |
| | [isomerizing] 1/ | | | | | |
| | Name = GFPT1; | | | | | |
| | Synonyms = GFAT, GFPT | | | | | |
| Q12864 | Cadherin-17/ | 3.45E−03 | −2.33 | 2.33 | −6.14 | 6.14 |
| | Name = CDH17 | | | | | |
| P01024 | Complement C3/ | 3.53E−03 | 2.04 | 2.04 | 27.43 | 27.43 |
| | Name = C3; | | | | | |
| | Synonyms = CPAMD1 | | | | | |
| Q96C19 | EF-hand domain-containing | 3.76E−03 | −2.29 | 2.29 | −3 | 3 |
| | protein D2/Name = EFHD2; | | | | | |
| | Synonyms = SWS1 | | | | | |
| Q9H0D6 | 5′-3′ exoribonuclease 2/ | 3.94E−03 | −1.81 | 1.81 | −1.71 | 1.71 |
| | Name = XRN2 | | | | | |
| P21333 | Filamin-A/Name = FLNA; | 3.97E−03 | 1.79 | 1.79 | 32.29 | 32.29 |
| | Synonyms = FLN, FLN1 | | | | | |
| Q9Y6E2 | Basic leucine zipper and | 3.98E−03 | −1.9 | 1.9 | −2.29 | 2.29 |
| | W2 domain-containing | | | | | |
| | protein 2/Name = BZW2; | | | | | |
| | ORFNames = HSPC028, | | | | | |
| | MSTP017 | | | | | |
| Q86V81 | THO complex subunit 4/ | 4.10E−03 | −1.77 | 1.77 | −1.43 | 1.43 |
| | Name = THOC4; | | | | | |
| | Synonyms = ALY, BEF | | | | | |
| P34897 | Serine | 4.31E−03 | −1.76 | 1.76 | −4.29 | 4.29 |
| | hydroxymethyltransferase, | | | | | |
| | mitochondrial/ | | | | | |
| | Name = SHMT2 | | | | | |
| Q16822 | Phosphoenolpyruvate | 4.43E−03 | −1.87 | 1.87 | −4.71 | 4.71 |
| | carboxykinase [GTP], | | | | | |
| | mitochondrial/ | | | | | |
| | Name = PCK2; | | | | | |
| | Synonyms = PEPCK2 | | | | | |
| Q92820 | Gamma-glutamyl hydrolase/ | 5.13E−03 | −2.15 | 2.15 | −3.71 | 3.71 |
| | Name = GGH | | | | | |
| P21810 | Biglycan/Name = BGN; | 5.20E−03 | 1.71 | 1.71 | 13.43 | 13.43 |
| | Synonyms = SLRR1A | | | | | |
| P05556 | Integrin beta-1/ | 5.34E−03 | 1.81 | 1.81 | 5 | 5 |
| | Name = ITGB1; | | | | | |
| | Synonyms = FNRB, MDF2, | | | | | |
| | MSK12 | | | | | |
| P07585 | Decorin/Name = DCN; | 5.72E−03 | 1.88 | 1.88 | 7.29 | 7.29 |
| | Synonyms = SLRR1B | | | | | |
| Q6YN16 | Hydroxysteroid | 5.79E−03 | −1.79 | 1.79 | −2.14 | 2.14 |
| | dehydrogenase-like protein | | | | | |
| | 2/Name = HSDL2; | | | | | |
| | Synonyms = C9orf99 | | | | | |
| P16401 | Histone H1.5/ | 5.98E−03 | −1.67 | 1.67 | −5.86 | 5.86 |
| | Name = HIST1H1B; | | | | | |
| | Synonyms = H1F5 | | | | | |
| P18206 | Vinculin/Name = 32VCL | 6.58E−03 | 1.8 | 1.8 | 6.43 | 6.43 |
| P30837 | Aldehyde dehydrogenase | 6.65E−03 | −1.66 | 1.66 | −7.71 | 7.71 |
| | X, mitochondrial/ | | | | | |
| | Name = ALDH1B1; | | | | | |
| | Synonyms = ALDH5, | | | | | |
| | ALDHX | | | | | |
| P14618 | Pyruvate kinase isozymes | 7.11E−03 | 1.67 | 1.67 | 17.14 | 17.14 |
| | M1/M2/Name = PKM2; | | | | | |
| | Synonyms = OIP3, PK2, | | | | | |
| | PK3, PKM | | | | | |
| Q13148 | TAR DNA-binding protein | 7.69E−03 | −1.6 | 1.6 | −2.14 | 2.14 |
| | 43/Name = TARDBP; | | | | | |
| | Synonyms = TDP43 | | | | | |
| O43488 | Aflatoxin B1 aldehyde | 8.24E−03 | −1.94 | 1.94 | −1.43 | 1.43 |
| | reductase member 2/ | | | | | |
| | Name = AKR7A2; | | | | | |
| | Synonyms = AFAR, AFAR1, | | | | | |
| | AKR7 | | | | | |
| P35659 | Protein DEK/Name = DEK | 8.93E−03 | −1.72 | 1.72 | −3.29 | 3.29 |
| P11940 | Polyadenylate-binding | 9.11E−03 | −1.6 | 1.6 | −4.86 | 4.86 |
| | protein 1/Name = PABPC1; | | | | | |
| | Synonyms = PAB1, PABP1, | | | | | |
| | PABPC2 | | | | | |
| P56537 | Eukaryotic translation | 9.52E−03 | −1.65 | 1.65 | −4.29 | 4.29 |
| | initiation factor 6/ | | | | | |
| | Name = EIF6; | | | | | |
| | Synonyms = EIF3A, | | | | | |
| | ITGB4BP; | | | | | |
| | ORFNames = OK/SW-cl.27 | | | | | |
| Q9BUF5 | Tubulin beta-6 chain/ | 9.62E−03 | 1.87 | 1.87 | 21 | 21 |
| | Name = TUBB6 | | | | | |

*Bold type indicates increased relative expression in metastatic colorectal cancer compared to peripheral cholangiocarcinoma

Table 9 provides information as to whether the marker proteins are relatively over-expressed or under-expressed in hilar cholangiocarcinoma versus HCC plus primary sclerosis cholangitis in post-TACE liver tumours. Accordingly, by determining the presence, absence or change in expression levels of a plurality of these marker proteins and comparing these changes with a reference of known expression levels, one is able to determine whether the cells under test are peripheral hilar cholangiocarcinoma or primary sclerosis cholangitis.

TABLE 9
Proteins differentiating hilar cholangiocarcinoma from hilar
cholangiocarcinoma with primary sclerosing cholangitis

| | | | | | Effect | |
|---|---|---|---|---|---|---|
| | | | | Magnitude | Size | Magnitude |
| | | | Effect | of Effect | (Mean | of Mean dif |
| Protein | Name | P Value | Size (g) | Size (g) | Dif) | (Mean Dif) |
| P08670 | Vimentin/Name = VIM | 2.45E−03 | 1.93 | 1.93 | 21.57 | 21.57 |
| Q9Y3Z3 | SAM domain and HD | 4.30E−03 | 1.79 | 1.79 | 2.57 | 2.57 |
| | domain-containing protein 1/ | | | | | |
| | Name = SAMHD1; | | | | | |
| | Synonyms = MOP5 | | | | | |
| Q07960 | Rho GTPase-activating protein | 4.43E−03 | 1.76 | 1.76 | 3.29 | 3.29 |
| | 1/Name = ARHGAP1; | | | | | |
| | Synonyms = CDC42GAP, | | | | | |
| | RHOGAP1 | | | | | |
| P13797 | Plastin-3/Name = PLS3 | 4.74E−03 | 1.76 | 1.76 | 6 | 6 |
| Q9BXN1 | Asporin/Name = ASPN; | 5.73E−03 | 1.92 | 1.92 | 5 | 5 |
| | Synonyms = PLAP1, SLRR1C; | | | | | |
| | ORFNames = UNQ215/PRO241 | | | | | |
| P06396 | Gelsolin/Name = GSN | 9.95E−03 | 1.53 | 1.53 | 7 | 7 |

Table 10 provides information as marker proteins that showed significant difference between hilar cholangiocarcinoma and colorectal metastasis.

TABLE 10

| | | | | | | |
|---|---|---|---|---|---|---|
| | | | | Effect | | |
| | | | Magnitude | Size | Magnitude | |
| | | Effect | of Effect | (Mean | of Mean dif | |
| Protein Name | P Value | Size (g) | Size (g) | Dif) | (Mean Dif) | Q Value |

Hilar cholangiocarcinoma > Colorectal metastasis (3 proteins)

| | | | | | | |
|---|---|---|---|---|---|---|
| P08758; Annexin A5/ | 1.11E−04 | 3.24 | 3.24 | 12.86 | 12.86 | 0.0108085 |
| Name = ANXA5; | | | | | | |
| Synonyms = ANX5, ENX2, PP4 | | | | | | |
| P21980; Protein-glutamine | 1.51E−04 | 2.73 | 2.73 | 8.71 | 8.71 | 0.0134021 |
| gamma-glutamyltransferase 2/ | | | | | | |
| Name = TGM2 | | | | | | |
| P05155; Plasma protease C1 | 7.78E−04 | 3.13 | 3.13 | 3.57 | 3.57 | 0.04078436 |
| inhibitor/Name = SERPING1; | | | | | | |
| Synonyms = 32C1IN, C1NH | | | | | | |

Hilar cholangiocarcinoma < Colorectal metastasis (27 proteins)

| | | | | | | |
|---|---|---|---|---|---|---|
| P17987; T-complex protein 1 | 3.21E−05 | −3.82 | 3.82 | −6.14 | 6.14 | 0.00485819 |
| subunit alpha/Name = TCP1; | | | | | | |
| Synonyms = CCT1, CCTA | | | | | | |
| P31939; Bifunctional purine | 4.25E−05 | −3.13 | 3.13 | −8.86 | 8.86 | 0.0056545 |
| biosynthesis protein PURH/ | | | | | | |
| Name = ATIC; | | | | | | |
| Synonyms = PURH; | | | | | | |
| ORFNames = OK/SW-c1.86 | | | | | | |
| P50990; T-complex protein 1 | 4.49E−05 | −3.29 | 3.29 | −7.43 | 7.43 | 0.00588578 |
| subunit theta/Name = CCT8; | | | | | | |
| Synonyms = C21orf112, CCTQ, | | | | | | |
| KIAA0002 | | | | | | |
| P34897; Serine | 7.58E−05 | −3.02 | 3.02 | −6.43 | 6.43 | 0.00856843 |
| hydroxymethyltransferase, | | | | | | |
| mitochondrial/Name = SHMT2 | | | | | | |
| Q99832; T-complex protein 1 | 8.76E−05 | −2.89 | 2.89 | −6.71 | 6.71 | 0.0091784 |
| subunit eta/Name = CCT7; | | | | | | |
| Synonyms = CCTH, NIP7-1 | | | | | | |
| P22102; Trifunctional purine | 1.25E−04 | −2.87 | 2.87 | −2.71 | 2.71 | 0.01173283 |
| biosynthetic protein | | | | | | |
| adenosine-3/Name = GART; | | | | | | |
| Synonyms = PGFT, PRGS | | | | | | |
| P54136; Arginyl-tRNA | 1.26E−04 | −2.91 | 2.91 | −3.71 | 3.71 | 0.01177183 |
| synthetase, cytoplasmic/ | | | | | | |
| Name = RARS | | | | | | |
| P23526; | 2.03E−04 | −2.64 | 2.64 | −5.71 | 5.71 | 0.0164932 |
| Adenosylhomocysteinase/ | | | | | | |
| Name = AHCY; | | | | | | |
| Synonyms = SAHH | | | | | | |
| P78371; T-complex protein 1 | 2.09E−04 | −2.71 | 2.71 | −9.29 | 9.29 | 0.01687596 |
| subunit beta/Name = CCT2; | | | | | | |
| Synonyms = 99D8.1, CCTB | | | | | | |
| P10809; 60 kDa heat shock | 2.47E−04 | −2.63 | 2.63 | −27.86 | 27.86 | 0.01851074 |
| protein, mitochondrial/ | | | | | | |
| Name = 2HSPD1; | | | | | | |
| Synonyms = HSP60 | | | | | | |
| P31948; | 2.83E−04 | −2.87 | 2.87 | −3.57 | 3.57 | 0.02052934 |
| Stress-induced-phosphoprotein | | | | | | |
| 1/Name = STIP1 | | | | | | |
| P15374; Ubiquitin | 2.94E−04 | −3.74 | 3.74 | −4 | 4 | 0.02113147 |
| carboxyl-terminal hydrolase | | | | | | |
| isozyme L3/Name = UCHL3 | | | | | | |
| P16422; Epithelial cell | 3.03E−04 | −2.53 | 2.53 | −2.86 | 2.86 | 0.02170325 |
| adhesion molecule/ | | | | | | |
| Name = EPCAM; | | | | | | |
| Synonyms = GA733-2, M1S2, | | | | | | |
| M4S1, MIC18, TACSTD1, | | | | | | |
| TROP1 | | | | | | |
| Q9NSD9; | 3.13E−04 | −3.07 | 3.07 | −4.86 | 4.86 | 0.02225712 |
| Phenylalanyl-tRNA synthetase | | | | | | |
| beta chain/Name = FARSB; | | | | | | |
| Synonyms = FARSLB, FRSB; | | | | | | |
| ORFNames = HSPC173 | | | | | | |
| P48643; T-complex protein 1 | 3.34E−04 | −2.61 | 2.61 | −4.43 | 4.43 | 0.023226731 |
| subunit epsilon/Name = CCT5; | | | | | | |
| Synonyms = CCTE, KIAA0098 | | | | | | |

Table 11 (FIG. 7) shows a list of marker proteins (467) with both unique and shared peptide sequences that were found to be significantly regulated in at least one of the tissue comparisons that were common to both quantification methods (spectral counting and area under the curve). Table 11 contains tissue type comparison (Tissue type number versus tissue type number), Uniprot ID, and protein names along with P-values, t-scores and log₂FoldChanges values for both quantitative methods. Positive Log 2 Fold changes refer to proteins which were up-regulated in the second tissue type compared to the first tissue type, while negative Log 2 Fold Changes refer to proteins which were down-regulated in the second tissue (e.g. In Tissue comparison 1_2, P51857 (AK1D1) gave a Log 2 fold change of −3.0581 for Area under the curve and −2.6145 for spectral counts. This means P51857 went down in Tissue 2 (HCC) compared to Tissue 1 (normal liver)).

Accordingly, the invention provides for the first time marker proteins which, by determining their relative expression by any appropriate means, can distinguish between different liver cell phenotypes. The disclosure herein provides details of how such expression levels may be determined and/or quantified, but may be employed and the method of quantification itself is not a limiting component of the invention.

The invention provides for a method of determining the cellular phenotype of a liver tissue sample said method comprising
(1) extracting marker proteins from said liver tissue sample;
(2) determining expression levels of a plurality of marker proteins in said sample, wherein said plurality of marker proteins are selected from a biomarker panel as represented by any one of Tables 2 to 10 or the relevant section of Table 11; optionally, repeating step (2) with a different plurality of marker proteins selected from any one of Tables 2 to 11;
(3) comparing said determined expression levels with reference expression levels for said plurality of marker proteins in known cellular phenotypes, thereby determining the cellular phenotype of the liver tissue sample.

The invention also provides for a method of identifying the cellular phenotype of a liver cell, said method comprising
(1) determining expression levels of a plurality of marker proteins in said liver cell;
(2) comparing said determined expression levels with reference set of expression levels for said plurality of marker proteins, said reference levels representing a particular cellular phenotype;
(3) identifying the cellular phenotype of the liver cell based on the comparison between the expression levels of the marker proteins in the liver cell and the reference expression levels;
wherein the plurality of marker proteins are selected from a biomarker panel as represented by any one of Tables 2 to 11.

Preferably the known cellular phenotypes comprise normal liver epithelium cells (hepatocytes), normal biliary epithelium cells (cholangiocytes), hepatocellular carcinoma cells, peripheral cholangiocellular carcinoma cells and hilar cholangiocellular carcinoma cells.

In all cases, the plurality of marker proteins may be selected from a biomarker panel as represented by the relevant Table as a whole or from Part A of the Table which contains those marker proteins showing the highest statistically significant difference. Alternatively the plurality of marker proteins may be selected from those shown to be over-expressed (identified in bold) or under-expressed as compared to the two cell types from either the whole Table or part A.

Alternatively, the plurality of marker proteins may be selected from Table 11 for the relevant tissue comparison.

In all cases, the plurality of protein may comprises 2, 3, 5, 10, 20, 30, 40, 50, 60, 70, 80, 90, 100, 120 or more protein markers provided in the respective Tables. With respect to Table 5, the plurality of marker proteins may comprise 2, 3, 5, 10, 20, 30, 40, 50, 60, 70, or more protein markers.

Alternatively, the method may comprises determining the presence or change in level of expression of at least 5%, 10%, 20%, 30%, 40%, 50%, 60%, 70%, 80%, 90% of the protein markers provided in any one of Tables 2 to 10.

In one embodiment, the method may determine the presence or change in level of expression of 100% of the protein markers provided in any one of Tables 2 to 10 or Table 11 or the relevant section of Table 11.

Preferably, the plurality of marker proteins are selected from any one of Collagen alpha 1 (XVIII) chain, Plastin-3, AKR1B10, Fibronectin, Beta 3 tubulin, Asporin, 14-3-3 protein eta or Dihydropyrimidinase-related protein 3 or combinations thereof, preferably the plurality of marker proteins comprises AKR1B10 and/or Beta 3 tubulin.

The liver tissue sample may be a biopsy sample taken from an individual suspect of having a liver tumor. Alternatively, the biopsy may be taken from an individual having previously received treatment for a liver tumor such as surgery, transplantation with or without transarterial chemoembolization.

The step of determining expression levels of said plurality of marker proteins includes determining the presence or absence of the marker proteins in said sample as well as the degree of change in expression levels. When compared to a reference or standard of known expression levels for the cellular phenotype, the presence, absence or change in degree of expression will be indicative of the cellular phenotype.

For this and all other aspects of the invention, the reference or standard protein expression levels may be determined from non-tumor liver tissue from the same subject. In this way, the difference in protein expression levels may be used to determine the cellular phenotype of the liver tumor. Alternatively, the reference levels may be a database comprising data representing expression levels for the marker proteins of interest as selected from any one or more of Tables 2 to 10 or the relevant section of Table 11. Ideally, the reference levels are provided by a liver tumor classification system, such as according to the present invention. The data representing expression levels may be a collection of data obtained from multiple liver samples and presented as an average or range. The data may relate to the levels of specific peptides each being unique to a protein of interest.

The biomarkers provided in Table 5 allow for the first time accurate and reliable distinction to be made between HCC and CC cells.

In particular the marker proteins selected from Table 5 or fragments thereof, or antibodies against said proteins or nucleic acids encoding said proteins or fragments thereof, can be used as a marker for the determination of cellular phenotype of a liver cell wherein said cellular phenotype is selected from HCC or CC.

Preferably, there is provided a method of determining the cellular phenotype of a liver tumor cell, said method comprising
(1) determining protein expression levels in said liver tumor cell of a plurality of marker proteins selected from Table 5;
(2) comparing said protein expression levels with a reference of expression levels for said plurality of marker proteins; and
(3) determining said cellular phenotype of said cell based on the comparison of expression levels of said plurality of marker proteins;
   wherein said cellular phenotype of said liver tumor cell is selected from HCC or CC.

Where the liver tumor cell is from a biopsy taken from an individual having previously received treatment for a liver tumor such as surgery, transplantation with transarterial chemoembolization, it may be preferably to determine the protein expression levels of a plurality of marker proteins selected from Table 7, in addition to, or instead of, those selected from Table 5.

Hence, in one embodiment, the method of determining the cellular phenotype of a liver tumor cell, comprises:
(1) determining protein expression levels in said liver tumor cell of a plurality of marker proteins selected from
   a) Table 5, or
   b) Table 7, or
   c) Table 5 and Table 7, or
   d) Table 5A or
   e) Table 7A, or
   f) Table 5A and Table 7A;
(2) comparing said protein expression levels with a reference of expression levels for said plurality of marker proteins; and
(3) determining said cellular phenotype of said cell based on the comparison of expression levels of said plurality of marker proteins;
   wherein said cellular phenotype of said liver tumor cell is selected from HCC or CC.

It will also be appreciated that the marker proteins determined herein may also be used as tumor antigens for the purpose of diagnostic and/or prognostic methods and/or for selecting or determining a treatment regimen for an individual based on determination of a cellular phenotype of the liver tumour cell. For example, the marker proteins or fragments thereof may be secreted or lost into the bloodstream as a result of cell death and may therefore be detected from blood, urine or saliva samples using standard techniques, e.g. antibodies. The detection of such protein markers (e.g. tumor antigens) will enable the clinician to determine whether the individual has liver cancer and the cellular classification of the tumor. Thus, it is envisaged that the same methods may be used to diagnose liver tumor at an early stage using samples, including but not exclusively, from blood, saliva or urine.

Hence, there is provided a method for the diagnosis or prognostic monitoring of a liver tumor in an individual, said method comprising
(a) determining the presence or level of expression of a plurality of marker proteins selected from a biomarker panel as represented by any one of Tables 2 to 10 or relevant section of Table 11, in a liver tumor cell obtained from said individual;
(b) identifying the cellular phenotype of the liver tumor cell; and
(c) selecting a diagnosis or prognosis based on the cellular phenotype of the liver tumor cell.

Furthermore, there is provided a method for determining a treatment regimen for an individual having a liver tumor, said method comprising
(a) determining the presence or level of expression of a plurality of marker proteins selected from a biomarker panel as represented by any one of Tables 2 to 10 or relevant section of Table 11, in a liver tumor cell obtained from said individual;
(b) identifying the cellular phenotype of the liver tumor cell; and
(c) selecting a treatment regimen based on the cellular phenotype of the liver tumor cell.

In particular, the methods according to the invention may be based on the determination of cellular phenotypes of the liver tumor cells based on the protein expression levels identified in respect of a plurality of protein markers provided in Table 5 and/or Table 7.

The method may comprise comparing the determined expression levels with a previously determined reference level for said plurality of marker proteins.

The reference level is preferably a pre-determined level, which may, for example be provided in the form of an accessible data record. The reference level is preferable representative of the expression levels of a number of the biomarkers identified in Table 5 and/or Table 7, each one being the derived mean and range of values obtained from known cellular phenotypes. It will be appreciated that in other embodiments the reference level may be representative of the expression levels of marker proteins selected from other biomarker panels represented by the Tables provided herein depending on the cellular phenotype under investigation.

The liver tumor cell is preferably from a liver tumor biopsy from the individual and more preferably the biomarker panel is represented by Table 5, more preferably Table 5, Part A.

Still further, it is preferred that the plurality of marker proteins selected from the biomarker panel of Table 11 section 2_5.

Where the biopsy is from a patient having previously been treated with transarterial chemoembolization (TACE), it is preferred that the plurality of marker proteins are selected from the biomarker panel of Table 7 or Table 7, Part A. More preferably, the plurality of marker proteins selected from Table 11 section 3_4.

For all methods provided herein, it is envisaged that a further step of determining expressions levels for a second set of marker proteins may be performed. The second set of marker proteins may be selected from the same biomarker panel as the first set, or may be selected from a different biomarker panel as represented by the Tables herein. For example, the method may include firstly determining the expression levels for a plurality of marker proteins selected from Table 5 or relevant section of Table 11, and then determining the expression levels of a plurality of marker proteins selected from Table 7 (or relevant section of Table 11). The expression levels for the first and second set of marker proteins may be measured sequentially or at the same time.

Determining the presence or change in expression level of the plurality of marker proteins may be achieved in many ways all of which are well within the capabilities of the skilled person.

The determination may involve direct quantification of marker protein levels, of nucleic acid encoding those marker proteins or it may involve indirect quantification, e.g. using an assay that provides a measure that is correlated with the amount of marker protein present.

Accordingly, determining the presence or level of expression of the plurality of marker proteins may comprise
(a) contacting the liver cell with a plurality of binding members, wherein each binding member selectively binds to one of said plurality of marker proteins or nucleic acid sequences encoding said marker proteins; and
(b) detecting and/or quantifying a complex formed by said specific binding members and marker proteins or nucleic acid sequences encoding said marker protein.

The binding member may be an antibody specific for a marker protein or a part thereof, or it may be a nucleic acid molecule which binds to a nucleic acid molecule representing the presence, increase or decrease of expression of a marker protein, e.g. an mRNA sequence.

The antibodies raised against specific marker proteins may be anti- to any biologically relevant state of the marker protein. Thus, for example, they can be raised against the unglycosylated form of a protein which exists in the body in a glycosylated form, against a precursor form of the protein, or a more mature form of the precursor protein, e.g. minus its signal sequence, or against a peptide carrying a relevant epitope of the marker protein. The detection and/or quantification may include preparing a standard curve using standards of known expression levels of the one or more marker proteins and comparing to the level of complex obtained in step (b) above.

A variety of methods may be suitable for determining the presence or changes in level of the plurality of marker proteins: by way of a non-limiting example, these include Western blot, ELISA (Enzyme-Linked Immunosorbent Assay), RIA (Radioimmunoassay), Competitive EIA (Competitive Enzyme Immunoassay), DAS-ELISA (Double Antibody Sandwich-ELISA), Liquid Immunoarray technology), immunocytochemical or immunohistochemical techniques, techniques based on the use of protein microarrays that include specific antibodies, “dipstick” assays, affinity chromatography techniques and liquid binding assays.

Antibodies may be obtained using techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992). Isolation of antibodies and/or antibody-producing cells from an animal may be accompanied by a step of sacrificing the animal. As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways that are well known in the art. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope. Humanised antibodies in which CDRs from a non-human source are grafted onto human framework regions, typically with the alteration of some of the framework amino acid residues, to provide antibodies which are less immunogenic than the parent non-human antibodies, are also included within the present invention.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2 188 638 A or EP 0 239 400 A. Cloning and expression of chimeric antibodies are described in EP 0 120 694 A and EP 0 125 023 A.

Preferred antibodies for use in accordance with the methods disclosed herein are isolated, in the sense of being free from contaminants such as antibodies able to bind other polypeptides and/or free of serum components. Monoclonal antibodies are preferred for some purposes, though polyclonal antibodies are within the scope of the present invention. For example, the primary monoclonal antibodies used herein were anti-AKR1B10 (clone 1A6; 1:500; Abcam, Cambridge, UK) and anti-tubulin beta 3 (clone TU20; 1:500; Abcam).

The binding of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. One favoured mode is by covalent linkage of each antibody with an individual fluorochrome, phosphor or laser exciting dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

The determination of over expression of the one or more (or plurality) of marker proteins according to the present invention may be carried out in many different ways well known to those skilled in the art that include, by way of example, determining the presence or amount of expression of said marker protein, or a fragment thereof, in the sample (tissue or blood) obtained from the individual, or determining the expression of the marker protein gene, for example by examining the marker protein mRNA levels expressed from the marker protein gene.

Preferably, the methods comprise detecting the expression levels of the marker proteins. Such detection may involve the step of contacting an antibody or antibody fragment capable of recognising said polypeptide, or fragment thereof, with said sample (tissue or blood).

The analysis may comprise a qualitative analysis, e.g. by monitoring the presence of the one or more marker proteins by microscopy, e.g. using immunohistochemical staining. Immunohistochemical analysis can be performed on either formalin-fixed, paraffin fixed samples or on frozen tissue samples. Examples of possible IHC methods which could be used to detect and quantify the one or more marker proteins are as described in the present invention.

In one aspect, the present invention provides for a method for diagnosing recurrent or primary liver tumor in a subject, the method comprising determining the presence or absence of one or more marker proteins selected from the group consisting of Collagen alpha 1 (XVIII) chain, Plastin-3, AKR1B10, Fibronectin, Beta 3 tubulin, Asporin, 14-3-3 protein eta, and Dihydropyrimidinase-related protein 3 in a sample. Preferably, the liver tumor is selected from the group consisting of hepatocellular carcinoma, peripheral cholangiocellular carcinoma or hilar cholangiocellular carcinoma cells.

In one embodiment of this aspect the marker protein is Beta 3 tubulin and/or AKR1B10, preferably Beta 3 tubulin.

In another embodiment, the sample is selected from any one of blood, plasma, serum, liver tissue, liver cells or combinations thereof, preferably the sample is liver tissue, optionally formalin-fixed paraffin-embedded liver tissue section.

In another embodiment, the determining the presence or absence of one or more marker proteins in the sample is performed by either Immunohistochemistry (IHC) or mass-spectrometry.

In one preferred embodiment, the method for diagnosing recurrent or primary liver tumor in a subject comprises determining the presence or absence of Beta 3 tubulin, and optionally, AKR1B10, in a sample, wherein the liver tumor is selected from the group consisting of hepatocellular carcinoma, peripheral cholangiocellular carcinoma or hilar cholangiocellular carcinoma cells and wherein the sample is liver tissue, optionally formalin-fixed paraffin-embedded liver tissue section and wherein determining the presence or absence of one or more marker proteins in the sample is performed by Immunohistochemistry (IHC).

More preferably, the method comprises determining Beta 3 tubulin with a primary antibody.

By way of further example a primary antibody that is capable of specifically binding to a marker protein, e.g. Beta 3 tubulin and/or AKR1B10 in a binding assay may be labelled with a detectable molecule such as, but not limited to, radioactive or fluorescent labels or to enzymes which utilise a chromogenic substrate. Examples of radiolabels of use in this technique are ³²P, ³H or ¹⁴C. Examples of fluorescent molecules of use in this technique are green fluorescent protein, Fluorescein IsoThioCyanate (FITC), Rhodamine IsoThioCyanate (TRICT) Cy3 and Cy5 Dyes. Examples of enzymes with chromagenic substrates of possible use in this technique are peroxidase, alkaline phosphatase or glucose oxidase.

Instead of detecting the signal from the primary antibody itself (as described above), a secondary antibody which binds to the primary antibody can be utilised.

The secondary antibody may be labelled with a suitable molecule for detection purposes examples of which are described above.

In an alternative method of detection the primary or secondary antibody may be labelled with a biotin molecule which can then be bound by a streptavidin or avidin linked enzyme with a suitable chromogenic substrate for detection.

Additional variations of the above techniques exist that will be apparent to someone skilled in the art.

In the context of this invention antibodies which could be used in such a technique could be generated by standard techniques involving immunisation of animals or could be generated in vitro by recombinant techniques. Antibodies could in this context be whole immunoglobulins or fragments of antibodies (Fab fragments) that correspond to the anti-idiotype. Such antibodies can be readily produced by the skilled person as discussed above.

The invention demonstrates the use of histological analysis to detect marker proteins and from this the cellular phenotype may be determined and the appropriate diagnosis and prognosis for the individual

In one embodiment, the method comprises the measurement of a plurality of marker proteins, preferably including tubulin beta 3 and/or AKR1B10 proteins in a liver tumour tissue section. The section may be a fresh-frozen section or formalin-fixed, paraffin embedded section such as is routine in the art of histology. Staining of sections may require a step of antigen retrieval prior to detection with a primary antibody specific for the target protein. Accordingly, the invention provides a method of determining the expression level of one or more marker proteins (preferably a plurality) using a binding member such as an antibody. Materials and methods relating to such assays are described in more detail below.

Alternatively, antibodies to the plurality of marker proteins may be detected in the blood or saliva of patients suspected of having liver cancer, using the marker proteins or fragments thereof as a detection agent.

In a further embodiment, the determination of the one or more (or plurality) of marker proteins in a sample from the individual may comprise the detection and quantification of autoantibodies. The marker protein or fragment thereof must be capable of specifically binding to such an autoantibody. Techniques such as ELISA may be used. An altered concentration of the plurality of marker proteins maybe identified by detecting the presence or altered levels of autoantibody thereto, compared to the level in a reference or control sample.

The level of autoantibody may be detected by Western blot (from 1D or 2D electrophoresis) against liver cell or liver tumor cells obtained from a biopsy or cell lines grown in vitro; or by ELISA, protein microarray or bead suspension array using purified marker proteins.

By way of example, detection of autoantibodies to marker proteins in different liver cell phenotypes can be carried out as follows. Recombinant marker proteins are expressed in baculovirus infected insect cells and used to coat the surface of microtitre plates. A blood or saliva sample, preferably a blood plasma sample and more preferably a blood serum sample is added to duplicate wells of each microtitre plate and incubated at 37° C. for 1 hour. Plates are aspirated and washed prior to the addition of a horse-radish peroxidase (HRP) labelled anti-human IgG antiserum and incubated for 1 hour at 37° C. Finally, binding of the antihuman antiserum is revealed by aspirating the plates, washing, and then adding tetra-methylbenzidine (TMB) which in the presence of HRP produces a coloured product the intensity of which is measured by reading the plates at 450 nm. An identical set of plates is tested with the exception that the second antibody is a HRP labelled anti-human IgM antiserum. The levels of IgG, IgE, IgA, IgD and/or IgM autoantibodies to each of the liver cell or liver tumor cell marker marker proteins is altered when compared to the levels found in reference standards or control samples.

In other embodiments, autoantibodies to the plurality of protein markers may be detected using the Western blotting approach using cells from the liver tumor sample, and then detecting the presence of antibodies specific for the protein markers that are present in the tumor.

It is contemplated within the invention to use (i) an antibody chip or array of chips, or a bead suspension array capable of detecting the plurality of marker proteins that interact with that antibody; or (ii) a protein chip or array of chips, or bead suspension array capable of detecting one or more autoantibodies that interact with the marker proteins; or (iii) a combination of both antibody arrays and protein arrays.

A further class of specific binding members contemplated herein in accordance with any aspect of the invention comprise aptamers (including nucleic acid aptamers and peptide aptamers). Advantageously, an aptamer directed to a protein marker may be provided by a technique known as SELEX (Systematic Evolution of Ligands by Exponential Enrichment), described in U.S. Pat. Nos. 5,475,096 and 5,270,163.

Alternatively, differential expression of nucleic acids encoding marker proteins may be used as a detection method. Expression of nucleic acids may be detected by methods known in the art, such as RT-PCR, Northern blotting or in situ hybridisation such as FISH.

Gene expression technologies such as reverse transcriptase—polymerase chain reaction (RT-PCR) can give accurate measurement of mRNA expression levels and the presence of the one or more marker proteins mRNA in a sample as opposed to its absence could also be used to provide the cellular phenotype classification. RT-PCR can be performed in a range of formats including quantitative versions and with sensitivities that enable the determination of mRNA levels in a single cell.

In one embodiment, the expression of the marker protein gene can be assessed by determining the presence or amount of marker protein mRNA in the sample and methods for doing this are well known to the skilled person. By way of example, they include determining the presence of marker protein mRNA in the sample (i) using a labelled probe capable of hybridising to the marker protein nucleic acid; and/or (ii) using PCR involving one or more primers based on a marker protein nucleic acid sequence to determine whether the marker protein transcript is present in a sample. The probe may also be immobilised as a sequence included in a microarray.

In accordance with these and other aspects of the invention, the plurality of marker proteins are selected from any one of Collagen alpha 1 (XVIII) chain, Plastin-3, AKR1B10, Fibronectin, Beta 3 tubulin, Asporin, 14-3-3 protein eta or Dihydropyrimidinase-related protein 3 or combinations thereof, preferably the plurality of marker proteins comprises AKR1B10 and/or Beta 3 tubulin.

In some embodiments, the determination of the presence or amount of the plurality of protein markers comprises measuring the presence or amount of mRNA derived from the cell under test. The presence or level of mRNA encoding the protein marker in the liver cells under examination will allow the cell to be classified according to its phenotype.

Techniques suitable for measuring the level of protein marker encoding mRNA are readily available to the skilled person and include “real-time” reverse transcriptase PCR or Northern blots. The method of measuring the level of a protein marker encoding mRNA may comprise using a plurality of primers or probes that are each independently directed to the sequence of one of the plurality of protein marker encoding genes or complement thereof. Each of the primers or probes may comprise a nucleotide sequence of at least 10, 15, 20, 25, 30 or 50 contiguous nucleotides that has at least 70%, 80%, 90%, 95%, 98%, 99% or 100% identity to a nucleotide sequence encoding the protein marker provided Table 5 (or any of Table 2 to 10 or relevant section of combined Table 11).

Preferably, the probes or primers according to the invention hybridise under stringent conditions to their specific protein marker encoding nucleic acid sequence.

The methods of the invention may comprise contacting the liver cell with a binding member as described above, but also includes contacting the binding member with cell lysate to increase contact directly or indirectly with the one or more of the marker proteins.

The binding members may be immobilised on a solid support. This may be in the form of an antibody array or a nucleic acid microarray. Arrays such as these are well known in the art. The solid support may be contacted with the cell lysate, thereby allowing the binding members to bind to the cell products representing the presence or amount of the one or more marker proteins.

In some embodiments, the binding member is an antibody or fragment thereof which is capable of binding to a marker protein or part thereof. In other embodiments, the binding member may be a nucleic acid molecule capable of binding (i.e. complementary to) the sequence of the nucleic acid to be detected.

The methods may further comprise contacting the solid support with a developing agent that is capable of binding to the one or more marker proteins, antibody or nucleic acid.

The developing agent may comprise a label and the method may comprise detecting the label to obtain a value representative of the presence or amount of the one or more marker proteins, antibody or nucleic acid in the cell, cell culture medium or cell lysate.

The label may be, for example, a radioactive label, a fluorophor, a phosphor, a laser dye, a chromogenic dye, a macromolecular colloidal particle, a latex bead which is coloured, magnetic or paramagnetic, an enzyme which catalyses a reaction producing a detectable result or the label is a tag.

The methods preferably comprise determining the presence or level of expression of a plurality of marker proteins or nucleic acids encoding said marker proteins in a single sample. For example, a plurality of binding members, each specific for one of a plurality of protein markers selected from Table 5 (or any one of Tables 2 to 10 or relevant section of combined Table 11), may be immobilised at predefined locations on the solid support. The number of binding members on the solid support may make up 10%, 20%, 30%, 40%, 50%, 60%, 70%, 80%, 90% or 100% of the total number of binding members on the support.

Additional methodologies to detect the one or more marker protein gene expression will be apparent to those skilled in the art.

In some embodiments, the determination of the presence or the level of expression of one or more of the marker proteins may be performed by mass spectrometry. Techniques suitable for measuring the level of a protein marker selected from Table 5 (or any other of Table 2 to 10 or relevant section of combined Table 11) include, but are not limited to techniques related to Selected Reaction Monitoring (SRM) and Multiple Reaction Monitoring (MRM) isotope dilution mass spectrometry including SILAC, AQUA (as disclosed in WO 03/016861, the entire content of which is specifically incorporated herein by reference) and TMTcalibrator (as disclosed in WO 2008/110581; the entire content of which is specifically incorporated herein by reference).

WO 2008/110581 discloses a method using isobaric mass tags to label separate aliquots of all marker proteins in a reference sample which can, after labelling, be mixed in quantitative ratios to deliver a standard calibration curve. A test sample is then labelled with a further independent member of the same set of isobaric mass tags and mixed with the calibration curve. This mixture is the subjected to tandem mass spectrometry and peptides derived from specific marker proteins can be identified and quantified based on the appearance of unique mass reported ions released from the isobaric mass tags in the MS/MS spectrum.

By way of a reference level, a known or predicted protein marker derived peptide may be created by trypsin, ArgC, AspN or Lys-C digestion of said protein marker. In some cases, when employing mass spectrometry based determination of protein markers, the methods of the invention comprises providing a calibration sample comprising at least two different aliquots comprising the protein marker and/or at least one protein marker derived peptide, each aliquot being of known quantity and wherein said biological sample and each of said aliquots are differentially labelled with one or more isobaric mass labels. Preferably, the isobaric mass labels each comprise a different mass spectrometrically distinct mass marker group.

Accordingly, the method of determining the cellular phenotype of a liver cell, wherein the method comprises determining the presence or expression level of one or more of the marker proteins selected from Table 5 (or from any one of Tables 2 to 10 or relevant section of combined Table 11 in a liver cell by Selected Reaction Monitoring using one or more determined transitions for known protein marker derived peptides; comparing the determined expression levels with reference set of expression levels previously determined to represent a particular cellular phenotype, e.g. HCC or CC; and determining or identifying the cellular phenotype based on changes in expression of said one or more, preferably plurality of marker proteins. The comparison step may include determining the amount of marker protein derived peptides from the liver cell with known amounts of corresponding synthetic peptides. The synthetic peptides are identical in sequence to the peptides obtained from the cell, but may be distinguished by a label such as a tag of a different mass or a heavy isotope.

More preferably, the determination and/or quantification is made by mass spectrometry.

One or more of these synthetic protein marker derived peptides with or without label form a further aspect of the present invention. These synthetic peptides may be provided in the form of a kit for the purpose of determining the cellular phenotype of a liver cell, in particular HCC or CC phenotype.

Other suitable methods for determining levels of protein expression include surface-enhanced laser desorption ionization-time of flight (SELDI-TOF) mass spectrometry; matrix assisted laser desorption ionization-time of flight (MALDI-TOF) mass spectrometry, including LS/MS/MS; electrospray ionization (ESI) mass spectrometry; as well as the preferred SRM and TMT-SRM. Each of these methods may be preceded by a step of marker protein enrichment by immunoprecipitation or affinity chromatography performed in column or batch mode. Any binding agent with the required specificity for the marker proteins may be employed in such enrichment including but not limited to polyclonal antibodies, monoclonal antibodies and aptamers.

Liquid chromatography-mass spectrometry (LC-MS/MS) based proteomics has proven to be superior over conventional biochemical methods at identifying and precisely quantifying thousands of marker proteins from complex samples including cultured cells (prokaryotes/eukaryotes), and tissue (Fresh Frozen/formalin fixed paraffin embedded), leading to the identification of novel biomarkers in an unbiased manner [7, 8, 9]. The present inventors have used laser microdissection (LMD) of specific formalin fixed tissue types thereby allowing regions of archival tumor material enriched for normal hepatocytes, normal cholangiocytes, and their respective transformed equivalents to be independently analysed by LC-MS proteomics. Spectral counting was used for relative quantification due to its good linear dynamic range (two to three orders of magnitude) and high quantitative proteome coverage [10, 11].

Thus, as detailed above, a differentially expressed protein which is a member of the plurality of protein markers described herein and illustrated in Tables 1A and Tables 2 to 11 may qualitatively have its expression activated or completely inactivated in first cellular phenotype versus a second cellular phenotype. Such a qualitatively regulated protein will exhibit an expression pattern within a given cell type which is detectable in one phenotype, e.g. HCC or CC, but not detectable in both. ‘Detectable’, as used herein, refers to a protein expression pattern, which is detectable using techniques described herein.

Alternatively, a differentially expressed protein which is a member of the plurality of marker proteins described herein may have its expression modulated, i.e. quantitatively increased or decreased, in a first cellular phenotype versus a second cellular phenotype. The degree to which expression differs between cellular phenotypes under comparison, e.g. HCC and CC, need only be large enough to be visualised via standard characterisation techniques, such as silver staining of 2D-electrophoretic gels. Other such standard characterisation techniques by which expression differences may be visualised are well known to those skilled in the art. These include successive chromatographic separations of fractions and comparisons of the peaks, capillary electrophoresis, separations using micro-channel networks, including on a micro-chip, SELDI analysis and qPST analysis.

Chromatographic separations can be carried out by high performance liquid chromatography as described in Pharmacia literature, the chromatogram being obtained in the form of a plot of absorbance of light at 280 nm against time of separation. The material giving incompletely resolved peaks is then re-chromatographed and so on.

Capillary electrophoresis is a technique described in many publications, for example in the literature “Total CE Solutions” supplied by Beckman with their P/ACE 5000 system. The technique depends on applying an electric potential across the sample contained in a small capillary tube. The tube has a charged surface, such as negatively charged silicate glass. Oppositely charged ions (in this instance, positive ions) are attracted to the surface and then migrate to the appropriate electrode of the same polarity as the surface (in this instance, the cathode). In this electroosmotic flow (EOF) of the sample, the positive ions move fastest, followed by uncharged material and negatively charged ions. Thus, marker proteins are separated essentially according to charge on them.

Micro-channel networks function somewhat like capillaries and can be formed by photoablation of a polymeric material. In this technique, a UV laser is used to generate high energy light pulses that are fired in bursts onto polymers having suitable UV absorption characteristics, for example polyethylene terephthalate or polycarbonate. The incident photons break chemical bonds with a confined space, leading to a rise in internal pressure, mini-explosions and ejection of the ablated material, leaving behind voids which form micro-channels. The micro-channel material achieves a separation based on EOF, as for capillary electrophoresis. It is adaptable to micro-chip form, each chip having its own sample injector, separation column and electrochemical detector: see J. S. Rossier et al., 1999, Electrophoresis 20: pages 727-731.

Surface enhanced laser desorption ionisation time of flight mass spectrometry (SELDI-TOF-MS) combined with ProteinChip technology can also provide a rapid and sensitive means of profiling marker proteins and is used as an alternative to 2D gel electrophoresis in a complementary fashion. The ProteinChip system consists of aluminium chips to which protein samples can be selectively bound on the surface chemistry of the chip (eg. anionic, cationic, hydrophobic, hydrophilic etc). Bound marker proteins are then co-crystallised with a molar excess of small energy-absorbing molecules. The chip is then analysed by short intense pulses of N2 320 nm UV laser with protein separation and detection being by time of flight mass spectrometry. Spectral profiles of each group within an experiment are compared and any peaks of interest can be further analysed using techniques as described below to establish the identity of the protein.

Isotopic or isobaric Tandem Mass Tags® (TMT®) (Thermo Scientific, Rockford, USA) technology may also be used to detect differentially expressed marker proteins which are members of a biomarker panel described herein. Briefly, the marker proteins in the samples for comparison are optionally digested, labelled with a stable isotope tag and quantified by mass spectrometry. In this way, expression of equivalent marker proteins in the different samples can be compared directly by comparing the intensities of their respective isotopic peaks or of reporter ions released from the TMT reagents during fragmentation in a tandem mass spectrometry experiment.

Differentially expressed marker proteins which are members of the plurality of protein markers described herein may be further described as target marker proteins and/or fingerprint marker proteins. ‘Fingerprint marker proteins’, as used herein, refer to a differentially expressed protein whose expression pattern may be utilised as part of a prognostic or diagnostic cellular phenotype evaluation. A fingerprint protein may also have characteristics of a target protein or a pathway protein. For example, the one or more marker proteins described herein may be used as liver tumor markers as well as determining the cellular phenotype of the liver cell. For example, it is contemplated that any of the markers provided in Tables 2 to 11, but at least tubulin beta 3 and/or AKR1B10 proteins may be used as markers for liver tumor. The detection of these proteins in blood may well provide a diagnostic tool for liver cancer. The marker proteins may be secreted or lost into the blood stream following cell death and may serve as circulating tumor antigens.

As described above, the invention provides a number of methods by which the one or marker proteins may be determined in a liver tissue sample, blood or saliva sample from an individual. The method comprises detecting the expression levels of the one or more (preferably plurality) of marker proteins selected from any one of Tables 2 to 10 or the relevant section of Table 11.

Unless context dictates otherwise, the descriptions and definitions of the features set out above are not limited to any particular aspect or embodiment of the invention and apply equally to all aspects and embodiments which are described. Thus, the features set out above are disclosed in all combinations and permutations.

### 2. KITS

One or more of the marker proteins selected From table 2 to 11 may be used as diagnostic marker for liver cancer in the methods described above and kits for use in carrying out these methods, in particular determining the cellular phenotype of a liver cell, preferably a liver tumor cell, in vitro, are encompassed herein.

Preferably, the kit allows the determination/identification of a cellular phenotype selected from normal liver epithelium cells (hepatocytes), normal biliary epithelium cells (cholangiocytes), hepatocellular carcinoma cells, peripheral cholangiocellular carcinoma cells and hilar cholangiocellular carcinoma cells.

More preferably, the kit allows the liver tumor cell to be identified as an HCC cell or a CC cell.

The kit allows the user to determine the presence or level of expression of a plurality of analytes selected from
a) a plurality of marker proteins or fragments thereof provided in Table 5 (or one of Tables 2 to 10 or relevant section of combined Table 11);
b) antibodies against said marker proteins and nucleic acid molecules encoding said marker proteins or fragments thereof, in a liver cell under test; the kit comprising:
   (a) a solid support having a plurality of binding members, each being independently specific for one of said plurality of analytes immobilised thereon;
   (b) a developing agent comprising a label; and, optionally
   (c) one or more components selected from the group consisting of washing solutions, diluents and buffers.

Suitable binding members have been described herein. In particular, for detection of a marker protein or fragment thereof, the binding member may be an antibody which is capable of binding to one or more of the marker proteins selected from Table 5 (or any one of Tables 2 to 10 or relevant section of combined Table 11), or a combination thereof.

Kits according for the invention may be used for diagnosing recurrent or primary liver tumor in a subject by comprising reagents for determining the presence or absence of one or more marker proteins selected from the group consisting of Collagen alpha 1 (XVIII) chain, Plastin-3, AKR1B10, Fibronectin, Beta 3 tubulin, Asporin, 14-3-3 protein eta, and Dihydropyrimidinase-related protein 3 in a sample. Preferably, the liver tumor is selected from the group consisting of hepatocellular carcinoma, peripheral cholangiocellular carcinoma or hilar cholangiocellular carcinoma cells.

In one embodiment, the marker protein is Beta 3 tubulin and/or AKR1B10, preferably Beta 3 tubulin.

In another embodiment, the kit comprises reagents suitable for preparing the sample, wherein the sample is selected from any one of blood, plasma, serum, liver tissue, liver cells or combinations thereof.

In yet another embodiment, the sample is liver tissue and the kit comprises reagents suitable for preparing liver tissue, optionally for preparing formalin-fixed paraffin-embedded liver tissue sections.

In another embodiment, the determining the presence or absence of one or more marker proteins in the sample is performed by either Immuno-hystochemistry.

In one preferred embodiment, the kit for diagnosing recurrent or primary liver tumor in a subject comprises reagents for determining the presence or absence of Beta 3 tubulin, and optionally, AKR1B10, in a sample, wherein the liver tumor is selected from the group consisting of hepatocellular carcinoma, peripheral cholangiocellular carcinoma or hilar cholangiocellular carcinoma cells and wherein the kit comprises reagents suitable for preparing liver tissue, optionally for preparing formalin-fixed paraffin-embedded liver tissue sections and wherein the kit is suitable for determining the presence or absence of one or more marker proteins in the sample by Immuno-hystochemistry (IHC).

More preferably, the kit comprises a primary antibody for Beta 3 tubulin.

As mentioned above, various methodologies are known in the art for determining the presence or amount of a marker protein, antibody or nucleic acid molecule in a sample. Various suitable assays are described below in more detail and each form embodiments of the invention.

The kit may additionally provide a standard or reference which provides a quantitative measure by which determination of an expression level of one or more marker proteins can be compared. The standard may indicate the levels of marker protein expression which indicate the cellular phenotype of the liver cell, e.g. HCC or CC

The kit may also comprise printed instructions for performing the method.

In one embodiment, the kit may be for performance of a mass spectrometry assay and may comprise a set of reference peptides (e.g. SRM peptides) in an assay compatible format wherein each peptide in the set is uniquely representative of each of the plurality of marker proteins provided in Table 5, (or any one of Tables 2 to 10 or relevant section of combined Table 11). Preferably two and more preferably three such unique peptides are used for each protein for which the kit is designed, and wherein each set of unique peptides are provided in known amounts which reflect the levels of such proteins in a standard preparation of said cell of known phenotype, e.g. HCC or CC cells. Optionally, the kit may also provide protocols and reagents for the isolation and extraction of proteins from a sample, a purified preparation of a proteolytic enzyme such as trypsin and a detailed protocol of the method including details of the precursor mass and specific transitions to be monitored. The peptides may be synthetic peptides and may comprise one or more heavy isotopes of carbon, nitrogen, oxygen and/or hydrogen.

Optionally, the kits of the present invention may also comprise appropriate cells, vessels, growth media and buffers.

The invention also includes the use of a plurality of binding members each capable of independently binding to one or more of a plurality of marker proteins or fragments thereof provided in Table 5, one or more antibodies against said marker proteins and one or more nucleic acid molecules encoding said marker proteins or fragments thereof, for the in vitro diagnosis or prognostic monitoring of an individual having or suspecting a liver tumor, or following treatment for a liver tumor.

The kit may comprise reagents for the detection of the plurality of protein markers in a liver tumor sample, wherein said plurality of protein markers are selected from Table 5 or part A of Table 5, or section 2_5 of Table 11.

A kit may comprise a plurality of primary antibodies, each antibody binding specifically to a different individual protein marker of the plurality of protein markers selected from Table 5 or section 2_5 of Table 11.

The antibodies may be immobilised on an assay plate, beads, microspheres or particles. Optionally, beads, microspheres or particles may be dyed, tagged or labelled.

A kit may further comprise one or more secondary antibodies which bind specifically bind to the primary antibodies. The secondary antibodies may be labelled, for example fluorescent labelled or tagged.

A kit may further comprise one or more detection reagents for detecting the presence of the tagged secondary antibodies.

Furthermore, the invention provides for a kit for classifying the cellular phenotype of a liver tumor cell or for determining a liver tumor in an individual in line with the methods described herein. Preferably, the kit comprises the reagents necessary for carrying out the determination of the presence or level of expression of one or more (preferably a plurality) of the marker proteins selected from one or more of Tables 2 to 11 on a sample (tissue or blood) and instructions for carrying out the test and interpreting the results. Preferred types of kit may comprise one or more of the following reagents:
(a) an antibody capable of recognising said one or more marker proteins or fragments thereof, for example for use in a binding assay such as an ELISA or in an immunohistochemical test. The antibody may be detected either by being directly labelled or through interaction with one or more other species, for example a labelled secondary antibody; and/or
(b) one or more primers based on the nucleic acid sequence of the one or more marker proteins, for example for detecting the presence and/or amount of the marker protein mRNA; and/or
(c) a probe based on the nucleic acid sequence of the one or more marker protein gene, for example for detecting the marker protein gene expression.

As for antibody reagents, the probes may conveniently be directly or indirectly labelled to enable them to be detected.

### 3. LIVER CELLULAR CLASSIFICATION SYSTEM

The invention also provide for a liver cellular classification system comprising a liver cellular classification apparatus and an information communication terminal apparatus, said liver cellular classification apparatus including a control component and a memory component, said apparatuses being communicatively connected to each other via a network;
(1) wherein the information communication terminal apparatus includes
(1a) a protein data sending unit that transmits the protein data derived from a liver tissue sample of a subject to the liver cellular classification apparatus;
(1b) a result-receiving unit that receives the result of the liver cellular classification of the subject transmitted from the liver cellular classification apparatus;
(2) wherein the liver cellular classification apparatus includes
(2a) a protein data-receiving unit that receives protein data derived from the liver tissue sample of the subject transmitted from the information communication terminal apparatus;
(2b) a data comparison unit which compares the data from the data-receiving unit with the data stored in the memory unit;
(2c) a classifier unit that determines the class (e.g. cellular phenotype) of the liver tissue of the subject, based on the results of the data comparison unit; and
(2d) a classification result-sending unit that transmits the classification result of the subject obtained by the classifier unit to the information communication terminal apparatus; and
wherein the memory unit contains protein expression level data of at least one (preferably a plurality) proteins selected from any one or more of Tables 2 to 10 or Table 11.

The data derived from the liver tissue sample of the subject is preferably expression level data such as that obtained from methods described herein e.g. LC-MS/MS and other proteomic approaches. The data may be derived just from the tissue being either normal tissue of tumor (or suspected tumor) tissue sample.

The protein data received by the data-receiving unit may be the actual protein levels, or it may be peptide levels from which the protein levels can be calculated. The peptide is unique to the at least one (preferably plurality) protein. In some embodiments it is preferable to use multiple, i.e. 2, 3, 4, or 5 peptides which are all unique to said protein. Where multiple peptides are used, data may be collated and optionally a median value used in the data comparison step.

The memory unit preferably includes data sets relating to protein expression levels representative of liver tissue or tumor sample. In a preferred embodiment, the protein expression levels are derived from actual peptide levels in the sample. This is particularly so if the data has been obtained using proteomic methods such as the LC-MS/MS method described herein. The data sets may provide a representative (e.g. average) level of protein expression levels found in liver tissue (normal or tumor) from a collection of data sets, e.g. as provided herein by Table 11. Alternatively, it may be preferable for the data sets to include a value representing a ratio of the protein expression level as compared to the protein expression level of a different cellular phenotype (e.g. HCC v peripheral CC) tissue obtained from the same source.

In this way, the system can compare the protein expression levels obtained from liver tissue samples (non-tumor or tumor) with protein expression levels representative of a particular liver cellular phenotype for the same protein and thereby classify the tissue by its cell type.

The system may further comprise the means to add the inputted data via the data sending unit to the stored data already held in the memory unit so that this new data can be included in the analysis performed by the determining unit. In this way the data representative of liver cellular phenotype (tumor or non-tumor) is constantly updated.

The liver tissue classification system may be connected to an apparatus for determining protein expression levels in a liver tissue (tumor or non-tumor) sample and feeding this data to the protein data sending unit.

Ideally the apparatus can process multiple samples using LC-MS/MS as described herein.

In accordance with this aspect of the invention, there is also provided a liver tissue (tumor or non-tumor) cellular classification program that makes an information processing apparatus including a control component and a memory component execute a method of determining and/or classifying the liver tissue of a subject, the method comprising:
(i) a comparing step of comparing data based on the protein expression levels of at least one (preferably a plurality) protein selected from Tables 2 to 11 obtained of a subject with the protein expression level data stored in the memory component; and
(ii) a classifying step for classifying the liver tissue cells of said subject, based on the comparison calculated at the comparing step; and wherein said tissue is classified into phenotypes including normal (hepatocytes, cholangiocytes), hepatocellular carcinoma, hepatocholangiocellular carcinoma (pre or post TACE therapy), peripheral cholangiocarcinoma, Hilar cholangiocarcinoma (with or without primary sclerosing cholangitis), or metastatic colo-rectal carcinoma.

In accordance with this aspect of the invention, there is also provided a computer-readable recording medium, comprising the liver tissue cellular classification program described above recorded thereon.

The data representing protein expression levels may be derived from peptide levels in the sample where said peptides are each unique to a particular protein selected from any one of Tables 2 to 11. It will be appreciated that peptides may be designed which will be unique for the protein from which they are derived, e.g. by proteolytic enzyme digestion such as trypsin, aspN, gluC and other such enzymes well known in the art.

In accordance with all aspects and embodiments of the invention the plurality of marker proteins are selected from any one of Collagen alpha 1 (XVIII) chain, Plastin-3, AKR1B10, Fibronectin, Beta 3 tubulin, Asporin, 14-3-3 protein eta or Dihydropyrimidinase-related protein 3 or combinations thereof, preferably the plurality of marker protein comprises AKR1B10 and/or Beta 3 tubulin.

### 4. EXAMPLES

Certain aspects and embodiments of the invention will now be illustrated by way of example and with reference to the figures and tables described above.

All documents mentioned in this specification are incorporated herein by reference in their entirety for all purposes.

4.1 Material and Methods

Liver Tissue

This study consists of 9 types of liver tissue taken from a total of 55 archived specimens: mixed HCC/CC after TACE (areas of HCC and areas of CC separately examined), non-treated HCC, normal liver parenchyma, normal bile duct, non-treated peripheral CC, non-treated hilar/perihilar CC, PSC-associated hilar CC, and metastatic colorectal cancer. All specimens were surgically resected or explanted livers from adult patients ranging from 27 to 80 years in age. Details of tissues in each group are as follows:
Mixed cancer after TACE (n=7): Before the treatment with TACE, all nodules were radiologically diagnosed as HCC according to European Association for the Study of the Liver (EASL) criteria of concordant imaging of nodular arterialized lesions with portal venous washout [16]. Presence of features suggestive of combined HCC and CC and in particular hypoarterialization was a criterion of exclusion for TACE. In explanted livers, cholangiocellular differentiation in addition to HCC components was histologically suggested. HCC and CC components were separately examined.
HCC (n=7): Well to moderately differentiated HCCs, which did not receive any treatment before transplantation, arranged in a trabecular or pseudoglandular pattern and developed in cirrhotic livers were examined. Etiologies of liver cirrhosis were viral hepatitis (n=3) and excessive alcoholic intake (n=4). One tumor was selected from each case when multiple tumors were present.
Normal liver tissue (n=7): Histologically unremarkable liver tissues without steatosis, inflammation, or fibrosis were selected from specimens that were surgically resected for metastatic cancers.
Normal bile duct (n=6): Histologically unremarkable hilar bile ducts are selected from liver specimens that were explanted for acute liver failure due to paracetamol overdose.
Peripheral CC (n=7): All cases showed well to moderately differentiated tubular adenocarcinoma against the background of fibrotic stroma. Hilar or perihilar tissue was not involved in any cases. The background liver was not cirrhotic in any cases, but two had early bridging fibrosis with moderate steatosis.
Hilar CC (n=7): All were ductal adenocarcinoma predominantly involving hilar/perihilar bile ducts. No patients had histories of chronic hepatobiliary diseases.
PSC-associated CC (n=7): Four cases were diagnosed to have CCs during treatment for PSC and underwent surgical resection, whereas three were incidentally found to have CC in explanted livers. All tumors were histologically adenocarcinoma, four associated with mucinous foci, which are sometimes seen in PSC-associated CC.
Metastatic colorectal cancer (n=7): All were typical intestinal type adenocarcinoma.

Tissue Sampling

Fresh liver specimens, which were surgically resected, were immediately received at our pathology laboratory. After macroscopic examination, samples were extensively taken, and were fixed in 10% formalin for at least 4 hours before being embedded in paraffin.

Microdissection of FFPE Tissue

10 μm thick sections were prepared from FFPE tissue blocks. After deparaffinization with xylene and alcohol, a target area of 1.5×10⁷ μm² (0.15 mm³) was selectively cut using the Laser Capture Microdissection System (LMD6500, Leica Microsystems, Wetzlar, Germany). Dissected tissues were directly immersed in 50 μL of Qproteome® FFPE Tissue Extraction Buffer (QIAGEN, Valencia, Calif.) and stored in −80° C. until protein extraction. Samples were prepared in batches e.g. week 1 (batch 1) the 1^{st} biological replicate of each tissue/tumor type were prepared and analysed. At week 2 (batch 2), the 2^{nd} biological replicates of each tissue/tumor type were prepared and analysed. This process was continued up until week 7 (batch 7) where the 7^{th} biological replicate of each tissue/tumor type were prepared and analysed. Samples were prepared and analysed in this way to ensure that differences in protein expression levels between different tissue types were due to biology/pathology of the sample, rather than any sample preparation variability.

Protein Extraction from FFPE Liver Tissue

Following storage at −80° C. samples were thawed on ice then homogenised, vortexed and centrifuged. Samples were transferred to 1.5 ml collection tubes and sealed with collection tube sealing clip, as provided in the Qproteome® kit. Samples were incubated on a heating block at 100° C. for 20 min, then for a further 2 hours at 80° C. with agitation at 750 rpm. After heating, the sample tubes were placed on ice for 1 min and the collection tube sealing clip removed. Each tube was centrifuged for 15 min at 14,000 g at 4° C. The supernatant were then transferred to a new siliconised collection tubes. The protein concentration of each sample were then determined using the Bradford protein assay and microplate luminometer.

1D Electrophoresis Gels

Stacking gels were constructed to comprise a 1 cm height 4% w/v polyacrylamide matrix on top of a 20% w/v polyacrylamide matrix. Protein samples and pre-stained molecular weight markers were each prepared in Sigma 2× Laemmli sample buffer (1:1) and run into the gels in Tris-glycine running buffer (Invitrogen, Loughborough, UK) for 20 min at 150 V, or until the protein sample and molecular weight markers were observed to concentrate at the 4-20% w/v gel interface. Each sample was loaded onto the gel at 100 μg/well. Following electrophoresis the gels were briefly stained with Imperial protein stain (Pierce, Ill., USA) then de-stained in water to visualize the proteins and to confirm their migration as a homogeneous population. The protein band visible at the 4-20% w/v gel interface was excised from each lane.

For separation gels we used the 1 mm thick 10 well Nu-PAGE 4 to 12% Bis-Tris gels from Invitrogen, Carlsbad, Calif., USA.

Reduction/Alkylation/Trypsin Digestion

Gel bands were chopped into small 1 mm³ pieces then destained and dehydrated with ACN. Proteins were subsequently reduced with 10 mM dithiothreitol in 25 mM ammonium bicarbonate at 56° C. for 1 h and alkylated with 55 mM iodoacetamide in 25 mM ammonium bicarbonate at room temperature for 45 min. Gel pieces were then washed, dried, rehydrated on ice for 10 min in 2 μg of sequence grade trypsin, reconstituted in 100 μL of 25 mM ammonium bicarbonate, then covered with an additional 20 μL ammonium bicarbonate solution, and incubated overnight at 37° C. The resulting proteolytic peptides were subjected to aqueous (30 μL ammonium bicarbonate, 20 min vortex) and two hydrophobic extractions (100 μL of 50% ACN, 5% formic acid, 20 min vortex, 10 min sonication). Samples were quickly vortexed and centrifuged then frozen to −80° C. The frozen sample were then concentrated under vacuum to ˜20 μL, then topped up with 0.1% Formic acid to 70 μL, gel particulates filtered out using 30000 MW filters (Millipore), and finally stored at −80° C. until used for LC-MS/MS.

Liquid Chromatography Mass Spectrometry (LC-MS/MS).

After freeze/thaw, 10 μL of each sample were injected onto a Thermo pre-column (EASY-Column, 2 cm, ID 100 μm, 5 μm C18-A1), using the Proxeon EASY-nLC II system (Thermo Fisher Scientific). Peptides were then resolved using an increasing gradient of 0.1% formic acid in acetonitirile (5 to 50% over 80 minute) through a Thermo analytical column (EASY-Column, 10 cm, ID 75 μm, 3 μm C18-A2) at a flow rate of 300 nL/min. Mass spectra were acquired on an LTQ-Orbitrap Velos (Thermo Fisher Scientific) throughout the chromatographic run (115 minutes), using 20×CID scans following each FTMS scan (2× pScans at 30000 resolving power @400 m/z). CID was carried out on 20 of the most intense ions from each FTMS scan then put on a dynamic exclusion list for 30 secs (20 ppm m/z window). AGC ion injection target for each FTMS scan were 1000000 (500 ms max injection time). AGC ion injection target for each MSA CID scan were 10000 (50 ms max ion injection time).

Data Pre-Processing

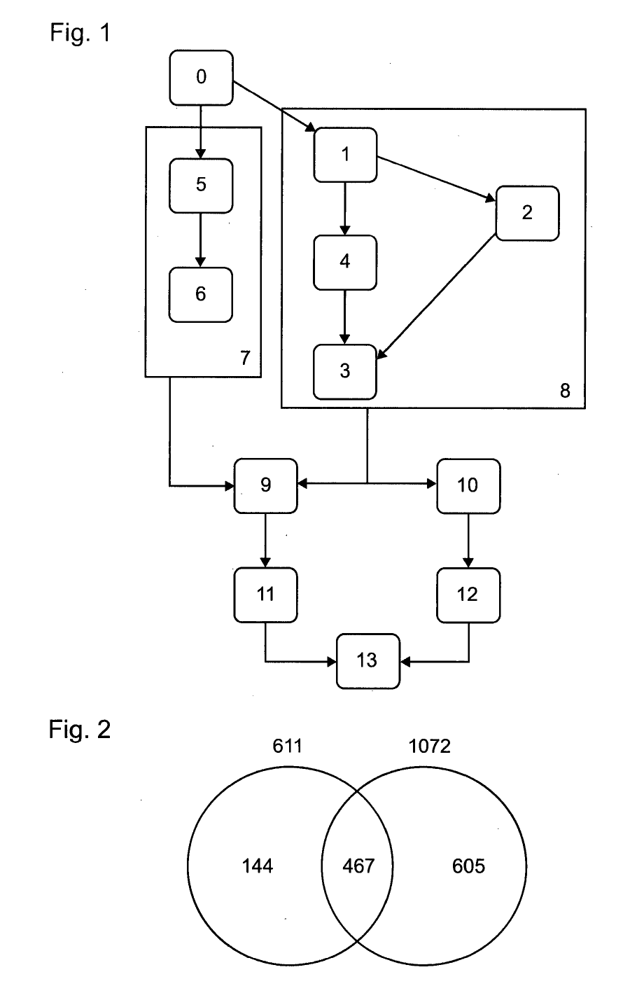

Peptide identification. Peak lists were extracted from Xcalibur Raw data file format using Proteome Discoverer 1.4 and searched using Mascot 2.2 and Sequest HT search engines. FIG. 1 illustrates the overall data analysis workflow used for peptide identification and quantification prior to the statistical analysis. The spectrum files node (0) was used to select the raw data files of interest. Spectrum selector (1) node was set to its default values (data was not smoothed, no signal to noise threshold) and no charge state filtering or de-isotoping took place. Both Mascot (4) and Sequest Nodes (2), were set up to search data against the UniProtKB/Swiss-Prot database (uniprot_sprot. fasta, downloaded from http://www.uniprot.org/downloads 20th February 2013), taxonomy Homo-sapiens (human). These nodes (2; 4) were programmed to search for tryptic peptides with up to 2 missed cleavages (C-Term K/R restrict P), with static modifications set as carbamidomethyl (C). Dynamic modifications were set to deamidation (N/Q), oxidation (M). Precursor mass tolerance was set to 10 ppm and fragment mass tolerance 0.5 Da for both data base search engines. Following peptide identification their q-values were calculated based on target-decoy approach with a 1% false discovery rate (FDR) and filtered in the Percolator node (3). The protein filter ‘Peptides per Protein’ option was set to 2. Both ‘Count only rank 1 peptides’ and ‘Count peptide only in top scored proteins’ were set as active. This ensured the highest stringency at the Protein level. Node 5 represents the ‘Event Detector’ which clusters isotopes of precursor ions in MS1 spectra that elute during the same retention time, whilst removing noise and spike signals from the spectra used for further processing. It is used in precursor ion quantification and peak area quantification. Node 6 represents the Precursor ions area detector node, calculating the area of each precursor ion. For accuracy it uses an average of the three most abundant peptides rather than the actual peptides to calculate the proteins area. The two data quantification methods that were implemented; Area under the Curve (AUC) (9) and Spectral Counting (10) generated a data matrix (11; 12) that was used for further statistical analysis. The final list (13) was obtained for marker proteins that were common to both quantitation methods and showed differential regulation.

Protein quantification. For each of the 62 tissue specimen data files (n=7 for 8 of the tissue types, n=6 for normal bile duct), Proteome Discover 1.4 was used to export the list of identified proteins to excel. For quantification purposes we utilized the node ‘The Precursor Ions Area Detector’ (FIG. 1 (6)) of Proteome Discoverer 1.4 which calculates the area under the curve (AUC) of each precursor ion using integration. For greater accuracy, it uses an average of the three most abundant peptides per protein rather than all peptides per protein to calculate the protein area. The number of PSMs for each protein in each sample was also used for quantification (spectral counting). Proteome Discoverer results were exported into an Excel sheet containing uniprot accession number, protein name, number of peptide spectrum matches and the protein area for each protein from each sample file. Both number of spectral counts and protein area estimates for each protein in each sample were used for further statistical validation.

Normalization: Both spectral counts and AUC for each protein from each sample were normalized to compensate for any artifact differences between samples such as unequal loading of protein onto the gel, variable in-gel digestion and peptide extraction and variable injection volume into the LC-MS/MS system.

The protein area estimates were log₁₀ transformed prior to normalization. Normalization was done using the following equation;

$$N = [ \frac{p}{\sum n / \tilde{x}} ]$$

Where
N is the normalized value for each protein in each sample
p is the un-normalized value for each protein in each sample
Σ n is the total number of PSMs or the total log₁₀ transformed protein area per LC-MS/MS analysis
\tilde{x} is the median value of Σ n of all the LC-MS/MS analyses

Statistical Analysis

Principal component analysis (PCA) was performed to investigate the multivariate datasets and identify outliers and groups/clusters nested within the datasets. Normalized protein values were used for PCA, which was performed using Simca v. 11, MKS Umetrics AB, Sweden [17].

Hierarchical clustering to build a class hierarchy for tissue types in relation to normalized protein values, alongside statistical analyses to observe differential regulation of proteins between tissue-types were both carried out in MATLAB: The MathWorks Inc., (R2012a) [18]. Two types of hierarchical clustering were performed to group the normalized protein abundances using agglomerative based clustering. In the first approach Pearson's correlation coefficients were obtained by comparing all normalized protein levels in all the samples (62) across all other samples (62), which resulted in a square data matrix consisting of 62×62 r2 Pearson's correlation coefficients. The second clustering was performed using ‘city-block’ distance metric (also known as the Manhattan distance) with un-weighted average distance (UPGMA) linkage to generate a hierarchical tree. The process clustered all data points first along all the columns (producing row-clustered data), and then along all the rows in the data matrix where rows corresponded to marker proteins and columns corresponded to the samples.

For Tables 2 to 10; the statistical analyses were run using R and the following R packages: q value and MBESS. For each group comparison and each protein an unrelated t-tests was computed to obtain the p value. Then q values (adjusted p values) were computed using a direct False Discovery Rate approach proposed by Storey (A direct approach to false discovery rates. Journal of the Royal Statistical Society, 2002, Series B, 64: 479-498). Hedges' g unbiased standardized effect size estimates were calculated, along with 95% confidence intervals for these estimates (Hedges, L. V. & Olkin, I. (1985). Statistical methods for meta-analysis. New York: Academic press). g<0.2 are regarded as very small differences, g=0.5 average differences, g>0.8 regarded as large differences. Unstandardized effect size estimates (i.e., mean difference) were calculated, along with 95% confidence intervals for these estimates.

For Table 11 (FIG. 7) each of the between group (tissue type) comparisons unpaired t-tests were performed for each protein to obtain significance level estimates (p-values) from the protein expression data matrix that consisted of normalized protein areas or normalized Spectral Counts (number of PSMs).

A protein was considered to be differentially modulated between the two tissue types when it had a p-value<0.05, log₂ fold ratio>2 or log₂ fold ratio<−2 (representing fourfold up- and down-regulation respectively). Volcano plots were created using these p-values and log₂ fold changes as described by Cui, X et al and Best, C. J. M et al [19-20].

Immunohistochemistry (IHC)

Four tissue types (normal liver (n=7), normal bile duct (n=6), HCC (n=7), and peripheral CC (n=7)) were used for validation IHC, as these are clinically most important to differentiate. One representative section selected from each case was used for immunostaining. Sections for IHC were taken from the same cases that were analysed by LC-MS. Immunostaining on FFPE specimens was performed using an autostainer Bond Max (Leica Microsystems, Wetzlar, Germany) The deparaffinized sections were heat-treated in a pH6.0 buffer for 10 mins. The primary monoclonal antibodies used were anti-AKR1B10 (clone 1A6; 1:500; Abcam, Cambridge, UK) and anti-tubulin beta 3 (clone TU20; 1:500; Abcam). Tissue type number key; (as used in Table 11 first column)

1)=Normal liver epithelium (Hepatocytes).
2)=Hepatocellular carcinoma. Combined hepato-cholangiocellular carcinoma after TACE therapy i.e.
3)=areas of hepatocellular differentiation, and
4)=areas of cholangiocellular differentiation.
5)=Peripheral (intrahepatic) cholangiocarcinoma.
6)=Hilar cholangiocarcinoma originated in patients without primary sclerosing cholangitis.
7)=Hilar cholangiocarcinoma originated in patients with primary sclerosing cholangitis.
8)=Metastatic colo-rectal carcinoma.
9)=Normal biliary epithelium (Cholangiocytes).

4.2 Results

Protein Markers Identification

In total 2864 proteins were identified using rank 1 peptides at 1% FDR at peptide level (2 rank 1 peptides per protein ID). Of the 2864 proteins 2628 (92%) had at least 1 unique peptide sequence and 2009 (70%) proteins had only unique peptide sequences. It was further observed that 236 (8%) proteins out of 2864 proteins had only shared peptide sequences. Of the 619 proteins with unique and shared peptides the inventors performed quantification using only unique peptides and compared this quantification to using the unique & shared peptides and found a correlation of 0.99 when comparing the fold change values from the two datasets (0.992 for spectral counting and 0.999 for area under the curve). Thus, as there appears to be no detrimental effect on accuracy using the shared and unique peptides, compared to only unique peptides, and due to the additional coverage gained by using shared and unique peptides, the inventors present results here obtained using shared and unique peptide sequences from both spectral counting and AUC forms of quantitation in the main text (Table 1, Table 11)

Protein Quantification and Hierarchical Clustering

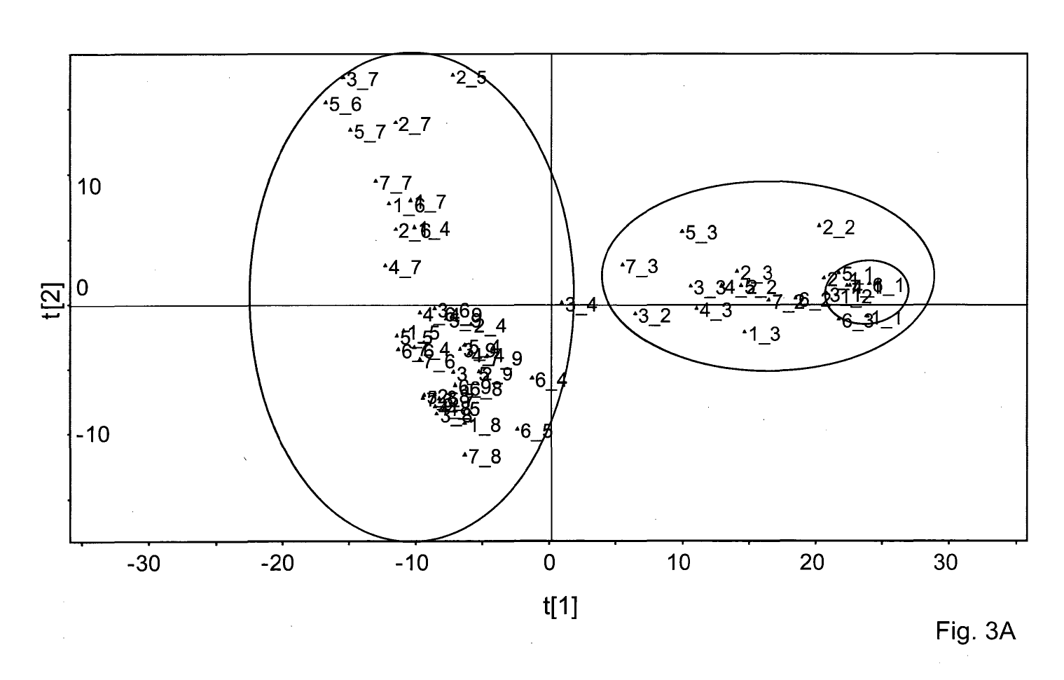

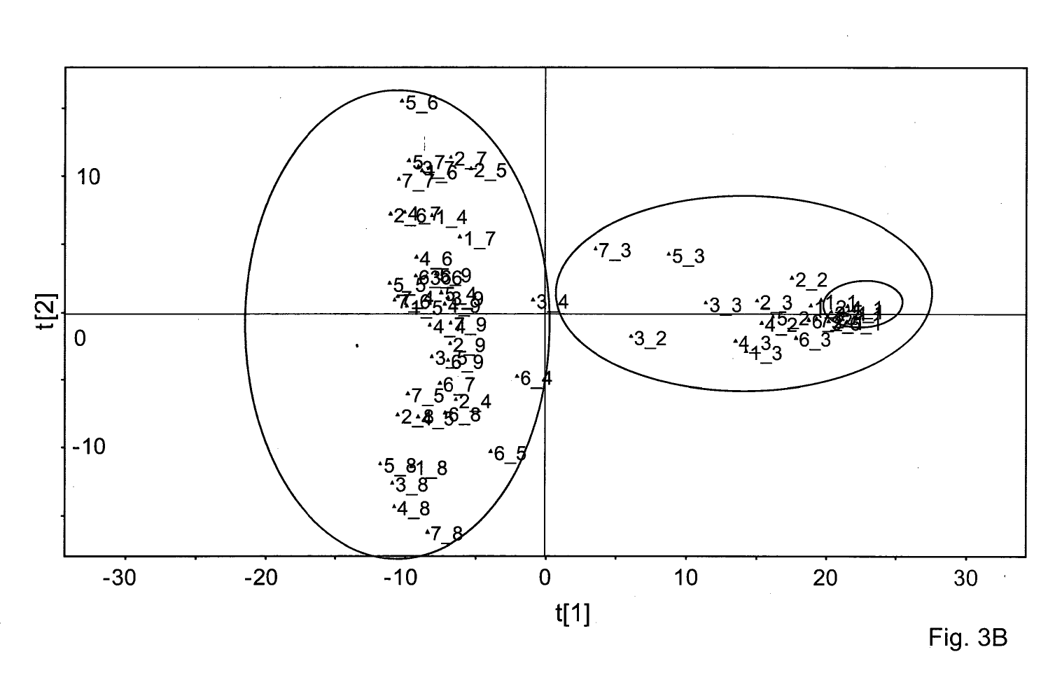

The inventors found 1072 proteins significantly regulated in at least one of the tissue type comparisons when using the area under the curve dataset, while 611 proteins were significantly regulated using the spectral counting dataset (in at least one of the tissue type comparisons). A total of 467 marker proteins were found to be significantly regulated in at least one of the tissue type comparisons, as observed in both quantification methods (e.g. common to both spectral counting (right) and AUC (left) in the Venn-Diagram in FIG. 2). PCA bi-plots using area under the curve (AUC) dataset (FIG. 3A) and spectral counting dataset (FIG. 3B) for these 467 common marker proteins shows clear separation between tissue types/groups that consists of cells that have common origin. Tissue types 1, 2 and 3 (left side of the plot, outer ellipse in FIGS. 3A and 3B) were all hepatocellular in origin and tissue types 4 to 9 (ellipse at right side of the plot in FIGS. 3A and 3B) belonged to glandular epithelium which clearly separated across two planes of the Bi-plot. Within each bi-plot it can also be seen that all cases of normal liver parenchyma (tissue type−1) are close to each other (left side of the plot, inner ellipse in FIGS. 3A and 3B).

Hierarchal clustering of the same 467 common marker proteins also supported the results obtained using PCA which clusters hepatocellular tissue types from glandular epithelium. Clustering of these 467 marker proteins based on Pearson's correlation coefficients and on protein data matrix using normalized protein area values clustered samples that originated from tissue types 1, 8 and 9 within single nested sub-groups (data not shown). Although using spectral counting as a data matrix produced similar results, it was found that area under curve data matrix produced better separation between groups when hierarchical clustering was performed. Table 1 illustrates the number of differentially modulated marker proteins that were common to both area under the curve and spectral counting datasets per tissue type comparison.

Difference in Protein Expression Profiling Among 9 Tissue Types

Post-TACE mixed cancer: Although HCC and CC components of post-TACE cancer are theoretically same in origin, these two areas showed significantly different protein markers' profiles as clearly demonstrated by PCA (FIG. 3) and hierarchal clustering (data not shown). In total 95 marker proteins were shown to be significantly modulated in the post-TACE HCC regions compared to the CC regions. Among the 95 marker proteins, 60 (63%) were overlapped with molecules that were identified in the comparison between normal liver parenchyma and bile duct (see below), in keeping with the hypothesis that post-TACE cancers can show bilineage differentiation. Seventy-eight marker proteins were found to be more abundant in HCC components, whereas 17 marker proteins were significantly up-regulated in CC areas. Two and five marker proteins showed significant difference between HCC components of post-TACE cancer and conventional HCC, and between CC components of post-TACE cancer and peripheral CC, respectively (Table 1). Names of those marker proteins are available in Table 11.

Normal liver parenchyma vs. normal bile duct: (See Table 6) Over 200 marker proteins were expressed at significantly different levels between normal liver and bile duct (Table 1 and Table 6). About a half of those marker proteins were liver enzymes, which were more abundantly present in normal liver parenchyma. In contrast, marker proteins that were more strongly expressed in normal bile ducts were diverse, including keratins 7 and 19, annexins, and galectins (Table 11).

Normal liver parenchyma vs. HCC: (See Table 2) Among 11 marker proteins that showed statistically significant difference between normal liver parenchyma and HCC, 5 marker proteins (14-3-3 protein eta, Aldo-keto reductase family 1 member B10 [AKR1B10], Heterogeneous nuclear ribonucleoprotein R, Histone H1.5, Keratin type II cytoskeletal 6B) appeared overexpressed in the cancer tissue. The remaining six, which were less abundant in HCC, were mostly liver enzymes supposed to represent mature hepatocyte functions. In accordance with the invention the one or more, or plurality of marker marker proteins may be selected from the group consisting of 14-3-3 protein eta, Aldo-keto reductase family 1 member B10 [AKR1B10], Heterogeneous nuclear ribonucleoprotein R, Histone H1.5, Keratin type II cytoskeletal 6B.

Normal bile duct vs. peripheral or hilar CC: (See Tables 3 and 4) Numbers of marker proteins that showed statistically significant difference between normal and neoplastic bile ducts are 37 for peripheral CC and 32 for hilar CC (Table 1, Table 11). Six and eight marker proteins were significantly overexpressed in peripheral and hilar CC, respectively. Among them, 3 marker proteins (Tubulin-beta 3 chain, Periostin, Collagen alpha-1(XII) chain) were up-regulated in both types of CC. Fourteen marker proteins were significantly less abundant in both types of CCs. In accordance with the invention the one or more, or plurality of marker proteins may be selected from the group consisting of Tubulin-beta 3 chain, Periostin, and Collagen alpha-1(XII) chain.

The one or more, or plurality of marker proteins may also be selected from argininosuccinate lyase, N9G), N9G)-dimethylarginine dimethylaminohydrolase 1A and 1B, Filamin-A and plastin-3.

HCCvs. peripheral CC: (See Table 5) One hundred and sixty-five marker proteins showed statistically significant differences between these two types of cancers, which develop in the liver parenchyma (Table 1, Table 4 and Table 11). Most marker proteins that were overexpressed in HCC were liver enzymes or mitochondrial marker proteins, whereas marker proteins that were up-regulated in peripheral CC were diverse in function including cell-cell adhesion, cell migration, and signal transduction. Multi-functional marker proteins such as annexins and S100-A11 were also more abundantly present in peripheral CC. Ninety-six marker proteins (58%) were overlapped with marker proteins that were identified in the comparison between normal liver parenchyma and normal bile duct.

Peripheral CC vs. hilar CC: These two types of CC are both adenocarcinoma of the biliary epithelium in origin. Interestingly, 14 showed significant differences between peripheral and hilar CC. For example, MUC5AC, a gastric type mucin, was significantly more abundant in hilar CC, while Tenascin was upregulated in peripheral CC. In accordance with the present invention, the protein markers may include MUC5AC and Tenascin.

PSC-associated CC vs. hilar CC: (Table 9) These two types of CC are histologically indistinguishable. But 5 marker proteins (Alpha-1B-glycoprotein, Asporin, Decorin, Methyl-CpG-binding protein 2, and Mimecan) were significantly different in abundance between PSC-associated and conventional hilar CC. All of these were more abundant in hilar CC unrelated to PSC. In accordance with the present invention, the one or more, or plurality of marker proteins may be selected from the group consisiting of Alpha-1B-glycoprotein, Asporin, Decorin, Methyl-CpG-binding protein 2, and Mimecan.

Peripheral or hilar CC vs. colorectal metastasis: (See Table 10) There were only 29 marker proteins expressed at significantly different levels in peripheral CC vs. colorectal metastasis and 63 marker proteins expressed at significantly different levels in hilar CC vs. colorectal metastasis (Table 1, Table 10, Table 11). Keratin 20, which is the most commonly used intestinal marker in routine pathological examination, did not reach statistical significance in this tissue comparison. Marker proteins that were significantly more abundant in CCs included annexins A4 and A5, protein-glutamine gamma-glutamyltransferase 2, and plasma protease C1 inhibitor.

AKR1B10 and Tubulin-beta 3 were investigated further by Volcano plots and Immuno-hystochemistry as a validation study. AKR1B10 was chosen as it is significantly upregulated in HCC than in normal liver or peripheral CC, suggesting that this may become a diagnostic marker specific to HCC. Tubulin-beta 3 was significantly up-regulated in peripheral CC than in either tissue type of normal liver, HCC, or normal bile duct, suggesting Tubulin-beta 3 to have a diagnostic value specific to peripheral CC. The inventors focused on four tissue types: normal liver parenchyma, HCC, normal bile duct, and peripheral CC, as they are clinically most important to differentiate.

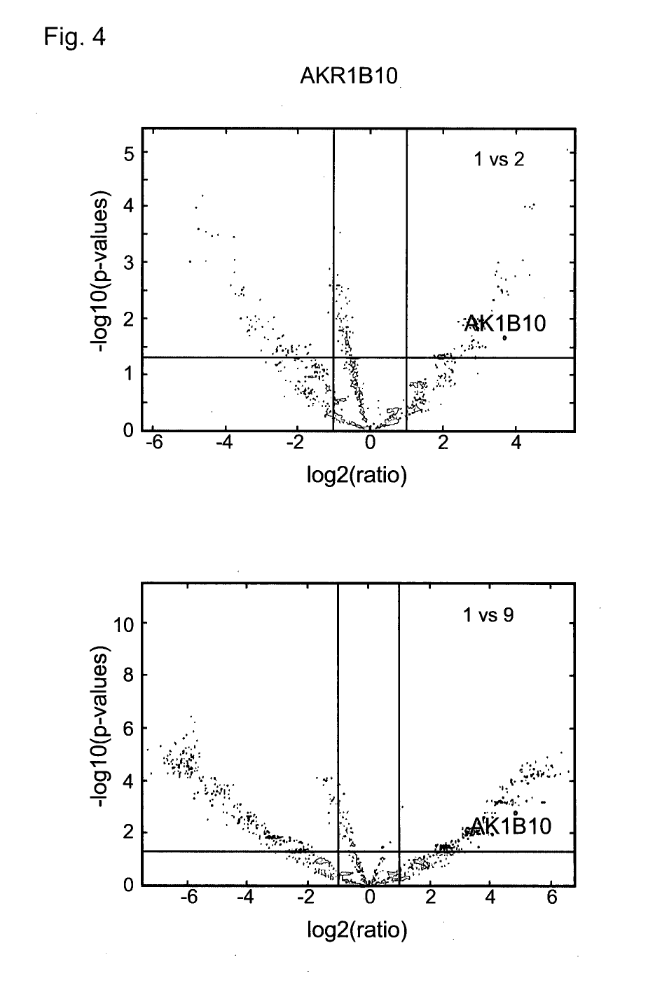

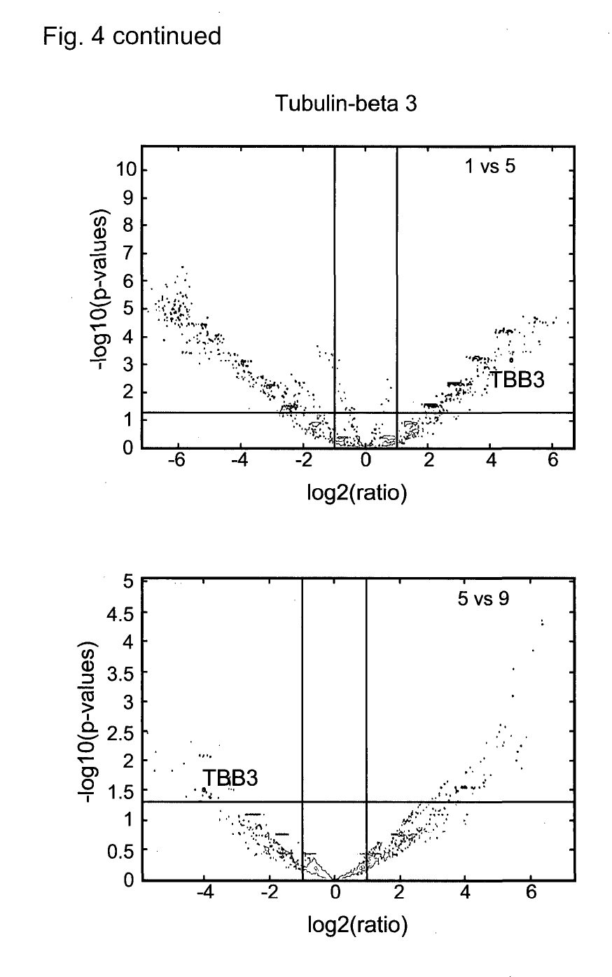

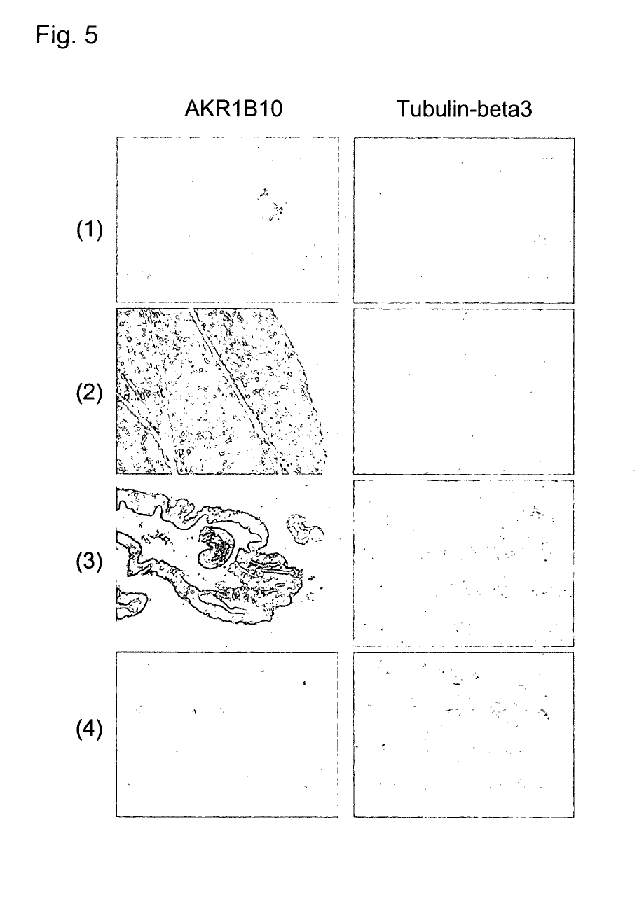

AKR1B10 (060218) is up-regulated inHCC (tissue type 2) (FIG. 4, upper panels) as statistically significant (p-value 2.83E-02 and log 2 fold change 2.95) when compared to normal liver parenchyma (tissue type 1) using AUC data matrix for quantitation. Spectral counting data also demonstrates statistically significant increase of AKR1B10 in HCC versus normal tissue (p-value 7.93 E-04 and log 2 fold change 3.87). AKR1B10 was surprisingly found to be up-regulated in normal bile duct (tissue type 9) when compared to normal liver parenchyma. On immunostaining, AKR1B10 was only focally expressed in normal liver, while this was more diffusely positive in HCC (FIG. 5). AKR1B10 was also moderately expressed in cirrhotic liver (background of HCC), suggesting this to be up-regulated at the early stage of multi-step hepato-carcinogenesis. AKR1B10 was diffusely positive in normal bile duct, and patchily positive in peripheral CC, in keeping with the proteomics results (Table 1).

Tubulin-beta 3 chain (Q13509) was found to be up-regulated in peripheral CC (tissue type 5) when compared to normal liver parenchyma or normal bile duct. Tubulin-beta 3 chain was surprisingly completely negative in normal liver, HCC, and normal bile duct, while it was diffusely expressed in 5 of 7 cases of peripheral CC (FIG. 5).

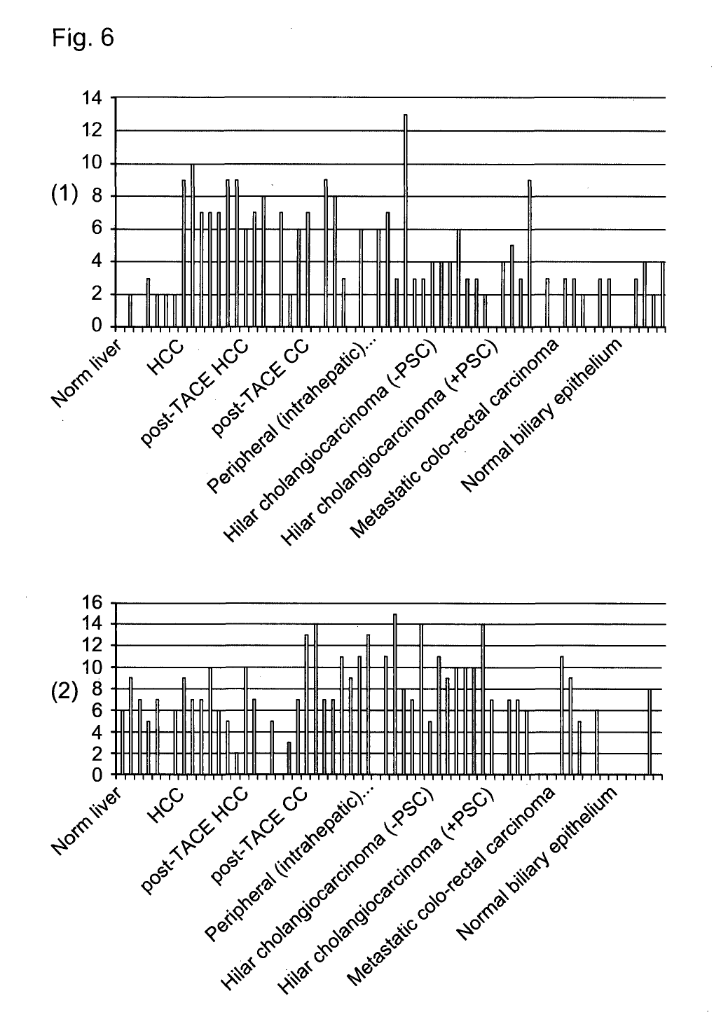

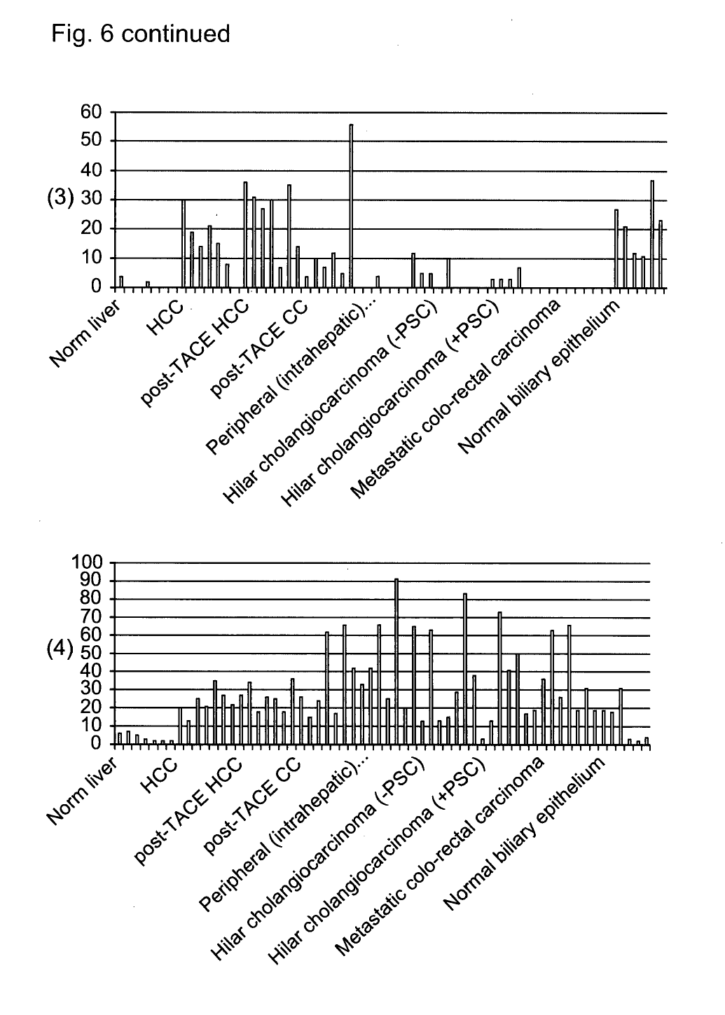

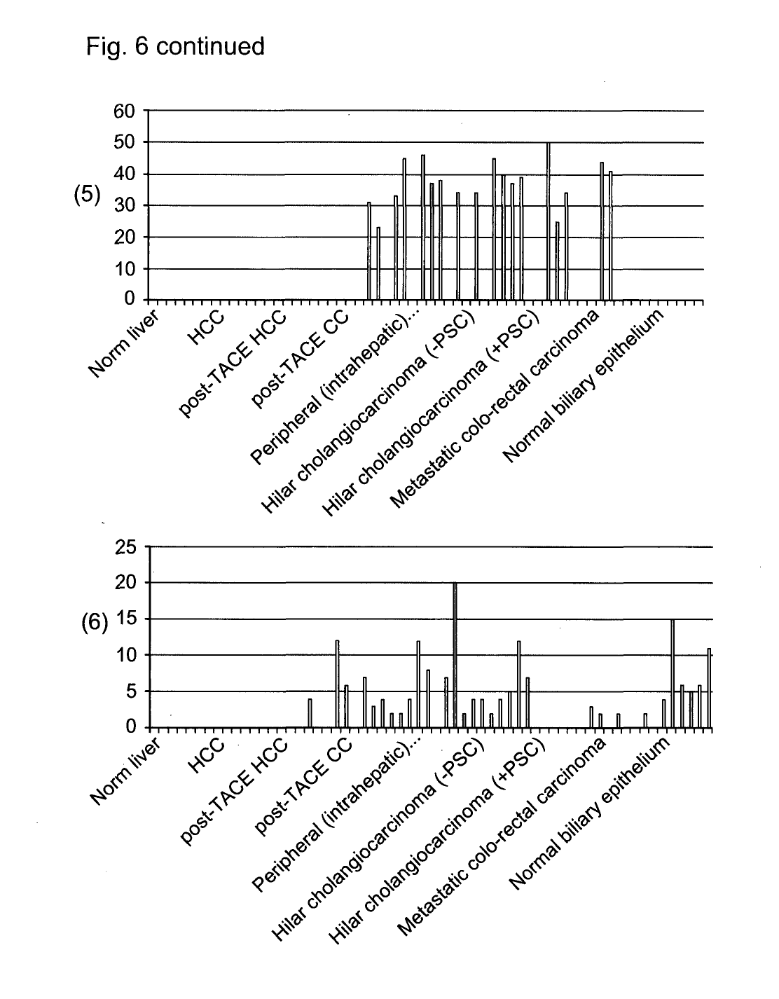

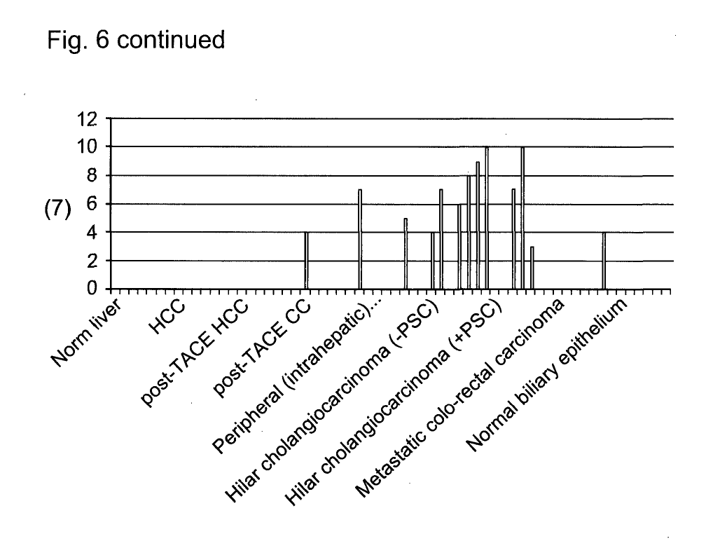

Finally, FIG. 6 shows the spectral counts obtained with respect to the expression of the marker proteins indicated therein in tissue types under study.

4.3 Discussion

The inventors have shown that the combination of laser microdissection and LC-MS/MS proteomics is a powerful approach which allows extensive profiling of protein expression in selected tumor sub-populations. This technique can be applied to FFPE histological archival material, a major advantage in the design of both prospective and retrospective tissue based studies. The identification of marker proteins already known to be specific to certain lineages (e.g. keratins 7 and 19 in biliary epithelium [21]), supports the robustness of the technique. The inventors have identified sets of marker proteins specific to well characterised hepato-biliary lineages and their neoplastic counterparts, and which could be used as biomarkers with diagnostic and prognostic potential, therapeutic targets or to understand the underlying carcinogenetic processes.

The identification of protein sets specific to the hepatocellular and cholangiocellular phenotype of post-TACE mixed tumors, and their similarity to their normal and typical neoplastic counterparts confirms that the differentiation process is truly divergent, despite a probable origin from a common progenitor. Of equal importance is the identification of marker proteins differentially expressed between normal and neoplastic hepatocytes and biliary epithelial cells, as these provide markers of malignant transformation or tumor differentiation; and between HCC and peripheral CC, which often overlap in both clinical presentation, and appearance on imaging and histology [22-23]. Of note alpha-fetoprotein (AFP), a marker commonly increased in the serum of patients with HCC [2], was not identified in any tissue type in this study. This is probably due to expression levels in tissue samples being below the LC-MS/MS detection threshold. Serum AFP levels are known to be elevated in about 75% of patients with HCC, but its expression in tissue is detectable in than 40% of patients even by IHC [24-25].

Interestingly one (14-3-3 protein eta) of the five marker proteins (14-3-3 protein eta; AK1BA; H15 and K2C6B) shown to be significantly over-expressed in HCC compared to normal liver parenchyma is known to play a role in mechanisms known to contribute to the cancer phenotype, as the abnormal expression of 14-3-3 protein eta has been reported in some human neoplasms [26-27]. Another two (Heterogeneous nuclear ribonucleoprotein R and Histone H1.5) are involved in gene transcription through chromatin remodeling, DNA methylation, and processing of precursor mRNA in the nucleus. The inventors also identified AKR1B10 as a significantly upregulated protein in HCC, which was validated by additional IHC. This finding is in keeping with a previous study, where a random-based gene fishing approach identified AKR1B10 as a significantly up-regulated gene in HCC compared to non-neoplastic liver tissue [28].

The inventors were also interested in molecules that were specifically up-regulated in CCs. Three marker proteins (Tubulin-beta 3 chain, Periostin, Collagen alpha-1(XII) chain) were up-regulated in CC compared to normal bile duct. Tubulin-beta 3 is the major constituent of microtubules and plays a critical role in proper axon guidance and maintenance. Periostin induces cell attachment and spreading and plays a role in cell adhesion. Collagen alpha-1(XII) interacts with type I collagen-containing fibrils, which are known to be overexpressed in invasive breast carcinoma [11]. Increased deposition and aberrant cross-linking of collagen is associated with the development of invasive breast cancer, the result of which contributes to stiffening of the extracellular matrix and is a factor that has been shown to drive progression of in situ disease [11]. The overexpression of these three marker proteins in CCs, and their known functional roles in biology and pathology means they will be useful markers of CC.

The three types of CCs are histologically very similar. Only a small number of markers that show significant difference in abundance between peripheral and hilar CCs have been identified [29]. The invention provides at least 5 such marker proteins, which may represent different underlying carcinogenetic processes. PSC-associated CC is supposedly different from conventional CC in underlying molecular events. However, these two types of CCs are histologically almost identical with no reliable molecular discriminators. No oncogenes or tumor suppressor genes specifically involved in PSC-associated carcinogenesis have been identified to the best of the inventors' knowledge. The inventors have identified 5 significantly modulated marker proteins between these two tissue types, all less abundant in PSC-associated CC.

In conclusion, the inventors have shown that the combination of laser microdissection and LC-MS/MS allows comprehensive proteomic profiling of tumor cell subpopulations and is applicable to FFPE archival tissue. The inventors have identified biomarkers, in particular Collagen alpha 1 (XVIII) chain, Plastin-3, AKR1B10, Fibronectin, Beta 3 tubulin, Asporin, 14-3-3 protein eta and Dihydropyrimidinase-related protein 3, to be used in the distinction between non-neoplastic and neoplastic hepatocytes and biliary epithelial cells, to refine grading of tumor differentiation, in the differential diagnosis of primary liver tumors, and to investigate the pathogenesis of sub-types of cholangiocarcinoma.

### 5. REFERENCES

1. Bosman F T, Carneiro F, Hruban R H, Theise N D. WHO Classification of Tumors of the Digestive System, 4th Ed. Lyom: IARC; 2010.
2. El-Serag H B. Hepatocellular carcinoma. N Engl J Med. 2011:365; 1118-27.
3. Blechacz B, Komuta M, Roskams T, Gores G J. Clinical diagnosis and staging of cholangiocarcinoma. Nat Rev Gastroenterol Hepatol. 2011:8; 512-22.
4. Alison M R. Liver stem cells: implications for hepatocarcinogenesis. Stem Cell Rev. 2005; 1:253-60.
5. Marquardt J U, Factor V M, Thorgeirsson S S. Epigenetic regulation of cancer stem cells in liver cancer: current concepts and clinical implications. J Hepatol. 2010:53; 568-77.
6. Zen C, Zen Y, Mitry R R, Corbeil D, Karbanova J, O'Grady J, Karani J, Kane P, Heaton N, Portmann B C, Quaglia A. Mixed phenotype hepatocellular carcinoma after transarterial chemoembolization and liver transplantation. Liver Transpl. 2011:17; 943-54.
7. Aebersold R, Mann M. Mass spectrometry-based proteomics. Nature. 2003; 422:198-207.
8. Ong S E, Mann M. Mass spectrometry-based proteomics turns quantitative. Nat Chem Biol. 2005; 1:252-62.
9. Shi S R, Liu C, Balgley B M, Lee C, Taylor C R. Protein extraction from formalin-fixed, paraffin-embedded tissue sections: quality evaluation by mass spectrometry. J Histochem Cytochem. 2006; 54:739-43.
10. Krizman D B & Burrows J. Use of Formalin-Fixed, Paraffin-Embedded Tissue for Proteomic Biomarker Discovery. Proteomics for Biomarker Discovery: Methods and Protocols, Methods in Molecular Biology. 2013: vol. 1002, p85-92.
11. Bateman N W, Sun M, Bhargava R, Hood B L, Darfler M M, Kovatich A J, Hooke J A, Krizman D B, Conrads T P. Differential proteomic analysis of late-stage and recurrent breast cancer from formalin-fixed paraffin-embedded tissues. J Proteome Res. 2011:10(3):1323-32.
12. Patel V, Hood B L, Molinolo A A, Lee N H, Conrads T P, Braisted J C, Krizman D B, Veenstra T D, Gutkind J S. Proteomic analysis of laser-captured paraffin-embedded tissues: a molecular portrait of head and neck cancer progression. Clin Cancer Res. 2008. 14(4):1002-14.
13. Azimzadeh O, Scherthan H, Yentrapalli R, Barjaktarovic Z, Ueffing M, Conrad M, Neff F, Calzada-Wack J, Aubele M, Buske C, Atkinson M J, Hauck S M, Tapio S. Label-free protein profiling of formalin-fixed paraffin-embedded (FFPE) heart tissue reveals immediate mitochondrial impairment after ionising radiation. J Proteomics. 2012; 75(8):2384-95.
14. Bantscheff M, Schirle M, Sweetman G, Rick J, Kuster B. Quantitative mass spectrometry in proteomics: a critical review. Anal Bioanal Chem. 2007; 389:1017-31.
15. Liu H, Sadygov R G, Yates J R. 3rd. A model for random sampling and estimation of relative protein abundance in shotgun proteomics. Anal Chem. 2004; 76:4193-201.
16. Bruix J, Sherman M, Llovet J M, Beaugrand M, Lencioni R, Burroughs A K, Christensen E, Pagliaro L, Colombo M, Rodes J; EASL Panel of Experts on HCC. Clinical management of hepatocellular carcinoma. Conclusions of the Barcelona-2000 EASL conference. European Association for the Study of the Liver. J Hepatol. 2001; 35:421-30.
17. Kettaneh, N., Berglund, S., and Wold, S., (2005), PCA and PLS with Very Large Data Sets, Computational Statistics & Data Analysis, 48, 69-85.
18. Bar-Joseph, Z., Gifford, D. K., and Jaakkola, T. S. (2001). Fast optimal leaf ordering for hierarchical clustering. Bioinformatics 17, Suppl 1:S22-9. PMID: 11472989.
19. Cui, X., Churchill, G. A. (2003). Statistical tests for differential expression in cDNA microarray experiments. Genome Biology 4, 210.
20. Best, C. J. M., Gillespie, J. W., Yi, Y., Chandramouli, G. V. R., Perlmutter, M. A., Gathright, Y., Erickson, H. S., Georgevich, L., Tangrea, M. A., Duray, P. H., Gonzalez, S., Velasco, A., Linehan, W. M., Matusik, R. J., Price, D. K., Figg, W. D., Emmert-Buck, M. R., and Chuaqui, R. F. (2005). Molecular alterations in primary prostate cancer after androgen ablation therapy. Clinical Cancer Research 11, 6823-6834.
21. Chu P G, Weiss L M. Keratin expression in human tissues and neoplasms. Histopathology 2002; 40; 403-39.
22. Vilana R, Forner A, Bianchi L, Garcia-Criado A, Rimola J, de Lope C R, Reig M, Ayuso C, Brd C, Bruix J. Intrahepatic peripheral cholangiocarcinoma in cirrhosis patients may display a vascular pattern similar to hepatocellular carcinoma on contrast-enhanced ultrasound. Hepatology 2010; 51; 2020-9.
23. Sempoux C, Jibara G, Ward S C, Fan C, Qin L, Roayaie S, Fiel M I, Schwartz M, Thung S N. Intrahepatic cholangiocarcinoma: new insights in pathology. Semin Liver Dis. 2011; 31; 49-60.
24. Liu C, Xiao G Q, Yan L N, Li B, Jiang L, Wen T F, Wang W T, Xu M Q, Yang J Y. Value of a-fetoprotein in association with clinicopathological features of hepatocellular carcinoma. World J Gastroenterol. 2013; 19:1811-9.
25. PorcellAI, De Young B R, Proca D M, Frankel W L. Immunohistochemical analysis of hepatocellular and adenocarcinoma in the liver: MOC31 compares favorably with other putative markers. Mod Pathol. 2000; 13:773-8.
26. Wang Z, Nesland J M, Suo Z, Trope C G, Holm R. The prognostic value of 14-3-3 isoforms in vulvar squamous cell carcinoma cases: 14-3-3β and ε are independent prognostic factors for these tumors. PLoS One. 2011; 6:e24843.
27. Yang X, Cao W, Lin H, Zhang W, Lin W, Cao L, Zhen H, Huo J, Zhang X. Isoform-specific expression of 14-3-3 proteins in human astrocytoma. J Neurol Sci. 2009; 276:54-9.
28. Heringlake S, Hofdmann M, Fiebeler A, Manns M P, Schmiegel W, Tannapfel A. Identification and expression analysis of the aldo-ketoreductasel-B10 gene in primary malignant liver tumors. J Hepatol. 2010; 52:220-7.
29. Guedj N, Zhan Q, Perigny M, Rautou P E, Degos F, Belghiti J, Farges O, Bedossa P, Paradis V. Comparative protein expression profiles of hilar and peripheral hepatic cholangiocarcinomas. J Hepatol. 2009; 51; 93-101.

1-63. (canceled)

64. A method for the diagnosis or prognostic monitoring of a liver tumor in an individual, said method comprising:
(a) determining a presence or level of expression of a Collagen alpha 1 (XVIII) chain and at least one marker protein selected from a plurality of marker proteins represented by any one of Table 1A, or Tables 2 to 11, in a liver cell obtained from the individual;
(b) identifying a cellular phenotype of the liver cell; and
(c) selecting a diagnosis or prognosis based on the cellular phenotype of the liver cell.

65. The method according to claim 64, wherein the cellular phenotype is selected from normal liver epithelium cells (hepatocytes), normal biliary epithelium cells (cholangiocytes), hepatocellular carcinoma cells, peripheral cholangiocellular carcinoma cells or hilar cholangiocellular carcinoma cells.

66. The method according to claim 65, wherein the liver cell is a liver tumor cell.

67. The method according to claim 64, wherein the plurality of marker proteins is selected from a biomarker panel represented by at least one of Table 5 or Table 7, and the cellular phenotype is selected from hepatocellular carcinoma cells and cholangiocellular carcinoma cells.

68. The method according to claim 67, wherein the plurality of marker proteins is selected from part A of Table 5.

69. The method according to claim 68, wherein the liver cell is obtained from a liver tumor biopsy sample.

70. The method according to claim 69, wherein the liver tumor biopsy sample is obtained from a patient having previously been treated with a transarterial chemoembolization.

71. The method according to claim 67, wherein the plurality of marker proteins is selected from a biomarker panel represented by Table 7.

72. The method according to claim 71, wherein the plurality of marker proteins is selected from a biomarker panel represented by part A of Table 7.

73. The method according to claim 64, wherein the step of determining the level of expression of the Collagen alpha 1 (XVIII) chain and the at least one marker protein selected from a plurality of marker proteins comprises:
(a) contacting the liver cell with a plurality of binding members, wherein each binding member selectively binds to said Collagen alpha 1 (XVIII) chain or said at least one marker protein, to form a complex; and
(b) detecting and/or quantifying the complex.

74. The method according to claim 73, wherein the specific binding member is an antibody or antibody fragment which selectively binds to at least one of said Collagen alpha 1 (XVIII) chain and said at least one marker protein.

75. The method according to claim 64, wherein the step of determining the level of expression of the Collagen alpha 1 (XVIII) chain and the at least one marker protein is performed by mass spectrometry.

76. The method according to claim 64, wherein the step of determining the level of expression of the Collagen alpha 1 (XVIII) chain and the at least one marker protein is performed by Selected Reaction Monitoring using one or more transitions for protein-derived peptides; and comprises comparing a peptide level in the liver cell with a peptide level previously determined to represent a cellular phenotype.

77. The method according to claim 76, wherein comparing the peptide level includes determining the amount of protein-derived peptides from the liver cell with known amounts of corresponding synthetic peptides, wherein the synthetic peptides are identical in sequence to the peptides obtained from the liver cell except for a label.

78. The method according to claim 77, wherein the label is a tag of a different mass or a heavy isotope.

79. A method for determining a treatment regimen for an individual having a liver tumor, said method comprising:
(a) determining a presence or level of expression of a Collagen alpha 1 (XVIII) chain and at least one marker protein selected from a plurality of marker proteins as represented by any one of Table 1A, or Tables 2 to 11, in a liver tumor cell obtained from said individual;
(b) identifying a cellular phenotype of the liver tumor cell; and
(c) selecting a treatment regimen based on the cellular phenotype of the liver tumor cell.

80. The method according to claim 64, wherein the marker protein comprises at least one of Plastin-3, AKR1B10, Fibronectin, Beta 3 tubulin, Asporin, 14-3-3 protein eta, Dihydropyrimidinase-related protein 3, or a combination thereof.

81. The method according to claim 80, wherein the marker protein comprises at least one of AKR1B10 or Beta 3 tubulin.

82. A kit for determining a cellular phenotype of a liver cell in vitro, said kit comprising:
(a) a set of reference peptides in an assay compatible format wherein each peptide in the set is uniquely representative of a Collagen alpha 1 (XVIII) chain and at least one marker protein selected from a plurality of marker proteins as represented by any one of Table 1A, or Tables 2 to 11; and, optionally
(b) one or more components selected from the group consisting of washing solutions, diluents and buffers,
wherein the kit is configured to allow a user to determine a presence or level of expression of a plurality of marker proteins or fragments thereof selected from a biomarker panel represented by any one of Table 1A, or Tables 2 to 11, in a cell.

83. The kit according to claim 82, wherein the set of reference peptides comprises a plurality of synthetic peptides, each having a sequence identical to a fragment of one of the plurality of marker proteins as represented by any one of Table 1A, or Tables 2 to 11, said fragment resulting from digestion of a protein by trypsin, ArgC, AspN or Lys-C, wherein one or more of the plurality of synthetic peptides comprises a label and the label is a heavy isotope.
